(19) United States
(12) Patent Application Publication
(10) Pub. No.: US 2026/0308149 A1
(43) Pub. Date: Oct. 8, 2026

(54) NOVEL CARBOXAMIDE DERIVATIVES

(71) Applicant: ANGELINI PHARMA S.P.A., Roma (IT)

(72) Inventors: Sylvain LEBRETON, Durham, NC (US); Aaron GERLACH, Durham, NC (US); Roger Lluis REDONDO PENA, Basel (CH); Roger David NORCROSS, Basel (CH); Martin SMRCINA, Durham, NC (US); Anil NAIR, Durham, NC (US); David MOWREY, Durham, NC (US); Mark CHAPMAN, Durham, NC (US); Ashley BANKS, Durham, NC (US); Matthew D. FULLER, Durham, NC (US); James DAVIDSON, Durham, NC (US); Nicholas Gareth Morse DAVIES, Durham, NC (US); Alena SAFAROVA, Durham, NC (US)

(73) Assignee: ANGELINI PHARMA S.P.A., Roma (IT)

(21) Appl. No.: 19/480,859

(22) PCT Filed: May 8, 2024
(86) PCT No.: PCT/US2024/028252

§ 371 Date: Nov. 3, 2025

Related U.S. Application Data
(60) Provisional application No. 63/465,008, filed on May 9, 2023.

(51) Int. Cl. C07D 405/04; A61K 31/165; A61K 31/17; A61K 31/277; A61K 31/337; A61K 31/341; A61K 31/343; A61K 31/351; A61K 31/352; A61K 31/357; A61K 31/365; A61K 31/381; A61K 31/4406; A61K 31/4427; C07B 59/00; C07C 233/15; C07C 233/19; C07C 233/22; C07C 233/60; C07C 255/44; C07C 275/18; C07C 275/26; C07C 275/34; C07D 213/56; C07D 213/61; C07D 213/64; C07D 305/06; C07D 305/08; C07D 307/33; C07D 307/52; C07D 307/94; C07D 317/28; C07D 333/20; C07D 407/12

(52) U.S. Cl. CPC C07D 405/04; A61K 31/165; A61K 31/17; A61K 31/277; A61K 31/337; A61K 31/341; A61K 31/343; A61K 31/351; A61K 31/352; A61K 31/357; A61K 31/365; A61K 31/381; A61K 31/4406; A61K 31/4427; C07B 59/001; C07C 233/15; C07C 233/19; C07C 233/22; C07C 233/60; C07C 255/44; C07C 275/18; C07C 275/26; C07C 275/34; C07D 213/56; C07D 213/61; C07D 213/64; C07D 305/06; C07D 305/08; C07D 307/33; C07D 307/52; C07D 307/94; C07D 317/28; C07D 333/20; C07D 407/12; C07B 2200/05; C07C 2601/02; C07C 2601/04; C07C 2601/08; C07C 2601/10; C07C 2601/14; C07C 2602/42; C07C 2602/44; C07C 2602/50

(57) ABSTRACT

Bicyclopentanyl compounds having the formula (I), or a solvate or a pharmaceutically acceptable salt thereof:

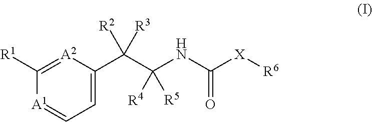

wherein R¹, R², R³, R⁴, R⁵, R⁶, A¹, A², and X are as defined herein, compositions including the compounds, processes of manufacturing the compounds, and methods of using the compounds. The compound may be used for treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

### FIELD OF THE PRESENT INVENTION

The present invention relates to novel carboxamide compounds useful as Kv7.2 enhancers (or positive modulators), their manufacture, pharmaceutical compositions, kits comprising the compounds, and their use as medicaments for the therapeutic and/or prophylactic treatment of a disorder, disease, or disability associated with Kv7.2. These disorders, diseases, or disabilities can be selected from behavioral disorders, mood disorders, neurodevelopmental disorders, intellectual disability, epilepsies, neurodegenerative diseases, pain, migraine, and tinnitus.

### BACKGROUND OF THE PRESENT INVENTION

The potassium channel family 7, or Q, contains five proteins that in humans are encoded by the genes KCNQ1, KCNQ2, KCNQ3, KCNQ4, and KCNQ5. The KCNQ proteins form homo- and hetero-tetrameric channels that respond to membrane voltage changes and open to let potassium ions flow out of cell membranes. Homomeric Kv7.2 channels as well as heteromeric Kv7.2 and Kv7.3 channels have been investigated because of their unique distribution and their potential role as primary regulators of neuronal excitability in many CNS and PNS pathways (Wang et al., 1998). KCNQ2 channels control the neuronal resting membrane potential, the spike frequency adaptation of neuronal firing, and presynaptic release. Impairment in their function leads to network instability even when lost exclusively in inhibitory neurons (Soh et al., 2018).

A significant percentage of childhood epilepsies are associated with KCNQ2 mutations (Lee et al., 2019). Human genetic studies identify de novo mutations in KCNQ2 as the third most robust link to epileptic encephalopathy (EE) (Zhao et al., 2020). Whether primary or secondary EEs, seizure activity worsens clinical outcomes and alters normal neurodevelopment (von Deimling, Helbig and Marsh, 2017).

Pediatric epilepsies affect about 1 in 200 children (Waaler et al., 2000) driving cognitive, behavioral and neurological deficits (Simkin and Kiskinis, 2018). In the case of specific pathogenic KCNQ2 mutations, despite most individuals eventually becoming seizure-free, developmental delays are experienced by the majority of patients (Kato et al., 2013). Targeting Kv7 channels offers a genetically validated target against epilepsy with a differentiated mode of action amongst anti-epileptics (Gunthorpe, Large and Sankar, 2012). Kv7.2 enhancers show the potential to transform neurodevelopmental trajectories by treating the neural network instability responsible for EEs (Kessi et al., 2020).

The connection between epilepsy and autism is robust (Srivastava and Sahin, 2017) and derives from the convergent phenotypes driven by a multitude of small genetic contributors, in combination with environmental factors. KCNQ2 is one of the top 5 ion channels associated with Autism Spectrum Disorder (ASD) and one of the top 30 of all de novo mutations known in ASD (Zhao et al., 2020).

Another defining feature of ASD, Atypical Sensory Processing (ASP) (Thye et al., 2018), is also driven by convergent genetics as seen in co-twin-control studies (Neufeld et al., 2021). The biology responsible for increased sensory sensitivity has been studied in preclinical models. There, multi-sensory neuronal hyper-excitability emerges regardless of the genetic manipulation that originally drives pathological neurodevelopment. Some genes whose manipulation leads to sensory sensitivity include CNTNAP2 (Peñagarikano et al., 2011), SHANK3 (Holder and Quach, 2016) and GABRB3 (Tanaka et al., 2012). Kv7.2 enhancers show the potential to correct neurodevelopmental trajectories in ASD by normalizing network stability, neural information processing, and sensory abnormalities, ultimately responsible for atypical social and repetitive behaviors in ASD. It is also interesting that KCNQ2 knock-out mice show repetitive behaviors and aberrant exploratory and social behaviors (Kim et al., 2019)

Kv7.2 enhancers also showed promise in syndromic neurodevelopmental disorders in part because of the prevalence and impact of epilepsies (Budisteanu et al., 2020). For example, epilepsy is prevalent (>80%) in Angleman syndrome, mostly starting before 3 years of age (Fiumara et al., 2010).

Another neurodevelopmental disorder, Dup15q syndrome (Dup15q), is caused by the partial duplication of Chromosome 15 that confers a considerable risk for autism spectrum disorder, epilepsy, and intellectual disability. Dup15q patient-derived induced pluripotent cells show KCNQ2 anomalies, and Retigabine, a pan-Kv7 channel opener, partially corrects their phenotype (Fink et al., 2018). Epilepsies are central to Dup15q, with Kv7.2 enhancers showing potential to transform this neurodevelopmental disorder.

In Fragile X syndrome, about 15% of individuals experience epilepsy (Berry-Kravis, 2002) together with abnormal sensory processing (McCullagh et al., 2020). KCNQ2 (Kv7.2 gene) is downregulated in the absence of Fragile X Mental Retardation Protein (FMRP) in rodent models (Zhang et al., 2018). Therefore, Kv7.2 enhancers could positively impact Fragile X by acting on both epilepsies and sensory processing.

Infantile epilepsies are associated with intellectual disabilities, and KCNQ2 de novo mutations are significantly associated with intellectual disability (Zhao et al., 2020). Kv7.2 enhancement may address the underlying biology that exacerbates the disability.

For all these neurodevelopmental disorders, early diagnosis and the identification of the correct antiepileptic treatment is at the core of the strategies aiming at normalizing neurodevelopmental trajectories.

Within behavioral disorders. Kv7.2 enhancers showed promise in attention-deficit hyperactivity disorder (ADHD) as well as major depressive disorder (MDD, depression). Some patients with KCNQ2 mutations and mild epilepsy phenotype, show cognitive delay and ADHD (Lee et al., 2019). Kv7.2 enhancers were suggested to treat the neural network instability and the behavioral impulsivity linked to ADHD. In the MDD space, Retigabine (Kv7 opener) showed antidepressant efficacy in patients by acting on the brain's reward centers (Tan et al., 2018). The significant reduction in depressive symptoms observed with retigabine places Kv7.2 enhancers as therapeutic candidates in MDD.

The therapeutic potential of Kv7.2 enhancers in pain sensitivity is supported by the localization of Kv7.2 channels in dorsal root ganglia and their established role in pain perception (Brown and Passmore. 2009). Non-selective Kv7.2 enhancers showed efficacy in reducing the excitability of human peripheral axons (Lang et al., 2008). Retigabine has already shown some efficacy in preclinical pain models (Korsgaard et al., 2005; Xu et al., 2010; Wu et al., 2017). Retigabine also shows efficacy in controlling spreading depression, a wave of cellular depolarization associated with migraines (Aiba and Noebels, 2021).

Within sensory abnormalities, aberrant plasticity of KCNQ2 channels is strongly linked to the induction of tinnitus (Li, Choi and Tzounopoulos, 2013). This link is based on the localization of Kv7.2 channels in the cochlea (Jin et al., 2009) and how cochlear damage depends on neuronal excitability driven by the closure of Kv7.2 channels (Liu, Glowatzki and Fuchs, 2015). Retigabine prevents the development of tinnitus in preclinical models (Li. Choi and Tzounopoulos. 2013). As evidence in support of how KCNQ2 pathologies are connected between indications, it is interesting to find that tinnitus and hyperacusis are more prevalent in ASD than in the general population (Danesh et al., 2015).

In neurodegenerative diseases, dysregulated K+ homeostasis in chronic neuro-inflammatory conditions is central to disease progression. For example, in amyotrophic lateral sclerosis (ALS), a fatal neurodegenerative disease of the motor nervous system (Hardiman et al., 2017), diverse genetics converge onto motorneuron excitotoxicity (Kanai et al., 2006; Pasinelli and Brown. 2006) and specifically axonal hyperexcitability predicts survival (Kanai et al., 2012). Patient-derived motor neurons show membrane hyperexcitability and the tool compound Retigabine (pan-Kv7 enhancer) rescues the phenotype (Wainger et al., 2014). The motorneuron hyperexcitability was found early in presymptomatic in vivo systems (Kuo et al., 2004) where it is a contributor to disease progression. Recently, clinical trials in ALS with Retigabine showed efficacy on functional biomarkers of ALS (Wainger et al., 2021) and preclinically protects against peripheral neuropathy (Nodera et al., 2011).

In Alzheimer's disease (AD), neuronal hyperexcitability and network instability (Frere and Slutsky, 2018) are early features of both IPSC models of sporadic AD (Ghatak et al., 2019), and genetic in vivo models (Palop et al., 2007; Kazim et al., 2017; Styr and Slutsky. 2018). Network instability worsens proteinopathy (Dolev et al., 2013; Frere and Slutsky, 2018) with consequences for patients (Vossel et al., 2013; Lam et al., 2017).

Because motor neuron and cortical neuron degeneration can be meaningfully slowed down by reducing aberrant neuronal activity, Kv7.2 enhancement could be an effective way to stop such aberrant activity, changing the neurodegenerative trajectory of the disease.

Therefore, enhancing the activity of Kv7.2 is a promising strategy for the treatment or prevention of diseases associated with Kv7.2. These include neurodevelopmental disorders like autism and Fragile X, epilepsy, intellectual disability, depression, attention deficit hyperactivity disorder, motor neuron excitability, pain, migraine, and sensory processing disorders.

WO2020/163268 relates to pyridine urea derivatives as KCNQ potentiators.

U.S. Pat. No. 5,384,330 relates to pharmacologically active 1,2,4-triaminobenzene derivatives modulating potassium ion channels Kv7.2-Kv7.5 (KCNQ2-KCNQ5) for the treatment of drug-resistant epilepsy. The compounds showed tolerability issues and other side effects.

To date, no agents acting on Kv7.2 are approved for the treatment of any of the diseases, disorders, or disabilities described herein, and thus there remains a need for modulators of Kv7.2 which provide a therapeutic benefit. Further, it would be beneficial to have modulators of Kv7.2 which are highly selective over other Kv7 channels. There is a need for Kv7.2 modulators which provide for a combination of favorable pharmacological properties, such as for example potency, selectivity, and metabolic stability.

It is, therefore, an object of this invention to provide selective Kv7.2 enhancers with favorable pharmacological properties useful as Kv7.2 enhancers (or positive modulators) for the therapeutic and/or prophylactic treatment of disorders, diseases, or disabilities associated with Kv7.2.

### REFERENCES

Aiba. 1, and Noebels. J. L. (2021) “Kcnq2/Kv7.2 controls the threshold and bihemispheric symmetry of cortical spreading depolarization,” Brain: a journal of neurology. doi: 10.1093/brain/awab141.
Berry-Kravis, E. (2002) “Epilepsy in fragile X syndrome.” Developmental medicine and child neurology. Wiley Online Library, 44(11), pp. 724-728.
Brown, D. A, and Passmore, G. M. (2009) “Neural KCNQ (Kv7) channels,” British journal of pharmacology. 156(8). pp. 1185-1195.
Budisteanu, M. et al. (2020) “Treatment of Epilepsy Associated with Common Chromosomal Developmental Diseases,” Open life sciences. Walter de Gruyter GmbH, 15(1). pp. 21-29.
Danesh, A. A. et al. (2015) “Tinnitus and hyperacusis in autism spectrum disorders with emphasis on high functioning individuals diagnosed with Asperger's Syndrome,” International journal of pediatric otorhinolaryngology, 79(10), pp. 1683-1688.
von Deimling, M., Helbig, I, and Marsh, E. D. (2017) “Epileptic Encephalopathies-Clinical Syndromes and Pathophysiological Concepts,” Current neurology and neuroscience reports, 17(2), p. 10.
Dolev, I. et al. (2013) “Spike bursts increase amyloid-β 40/42 ratio by inducing a presenilin-1 conformational change,” Nature neuroscience, 16(5), pp. 587-595.
Fink, J. J. et al. (2018) “Hvperexcitable phenotypes in iPSC-derived neurons from patients with 15q11-q13 duplication syndrome, a genetic form of autism,” BioRxiv. biorxiv.org. Available at: https://www.biorxiv.org/content/10.1101/286336v1.abstract.
Fiumara, A. et al. (2010) “Epilepsy in patients with Angelman syndrome.” Italian Journal of Pediatrics, p. 31. doi: 10.1186/1824-7288-36-31.
Frere, S, and Slutsky, I. (2018) “Alzheimer's Disease: From Firing Instability to Homeostasis Network Collapse,” Neuron, 97(1), pp. 32-58.
Ghatak, S. et al. (2019) “Mechanisms of hyperexcitability in Alzheimer's disease hiPSC-derived neurons and cerebral organoids vs isogenic controls,” eLife, 8. doi: 10.7554/eLife.50333.
Gunthorpe, M. J., Large, C. H, and Sankar, R. (2012) “The mechanism of action of retigabine (ezogabine), a first-in-class K+ channel opener for the treatment of epilepsy,” Epilepsia, 53(3), pp. 412-424.
Hardiman, O. et al. (2017) “Amyotrophic lateral sclerosis,” Nature reviews. Disease primers, 3, p. 17071.
Holder. J. L., Jr and Quach, M. M. (2016) “The spectrum of epilepsy and electroencephalographic abnormalities due to SHANK3 loss-of-function mutations,” Epilepsia, 57(10), pp. 1651-1659.
Jin. Z. et al. (2009) “Expression and localization of K channels KCNQ2 and KCNQ3 in the mammalian cochlea,” Audiology & neuro-otology, 14(2), pp. 98-105.
Kanai, K. et al. (2006) “Altered axonal excitability properties in amyotrophic lateral sclerosis: impaired potassium channel function related to disease stage,” Brain: a journal of neurology, 129(Pt 4), pp. 953-962.
Kanai, K. et al. (2012) “Motor axonal excitability properties are strong predictors for survival in amyotrophic lateral sclerosis,” Journal of neurology, neurosurgery, and psychiatry, 83(7), pp. 734-738.
Kato, M. et al. (2013) “Clinical spectrum of early onset epileptic encephalopathies caused by KCNQ2 mutation,” Epilepsia, 54(7), pp. 1282-1287.
Kazim, S. F. et al. (2017) “Early-Onset Network Hyperexcitability in Presymptomatic Alzheimer's Disease Transgenic Mice Is Suppressed by Passive Immunization with Anti-Human APP/Ap Antibody and by mGluR5 Blockade,” Frontiers in aging neuroscience, 9, p. 71.
Kessi, M. et al. (2020) “Intellectual Disability and Potassium Channelopathies: A Systematic Review,” Frontiers in genetics, 11, p. 614.
Kim, E. C. et al. (2019) “Heterozygous loss of epilepsy gene KCNQ2 alters social, repetitive, and exploratory behaviors,” Genes, Brain and Behavior. doi: 10.1111/gbb.12599.
Korsgaard, M. P. G. et al. (2005) “Anxiolytic effects of Maxipost (BMS-204352) and retigabine via activation of neuronal Kv7 channels,” The Journal of pharmacology and experimental therapeutics, 314(1), pp. 282-292.
Kuo, J. J. et al. (2004) “Hyperexcitability of cultured spinal motoneurons from presymptomatic ALS mice,” Journal of neurophysiology. physiology.org, 91(1), pp. 571-575.
Lam, A. D. et al. (2017) “Silent hippocampal seizures and spikes identified by foramen ovale electrodes in Alzheimer's disease,” Nature medicine, 23(6), pp. 678-680.
Lang. P. M. et al. (2008) “Retigabine reduces the excitability of unmyelinated peripheral human axons,” Neuropharmacology, 54(8), pp. 1271-1278.
Lee, I.-C. et al. (2019) “KCNQ2 mutations in childhood nonlesional epilepsy: Variable phenotypes and a novel mutation in a case series,” Molecular genetics & genomic medicine, 7(7), p. e00816.
Li, S., Choi, V, and Tzounopoulos, T. (2013) “Pathogenic plasticity of Kv7.2/3 channel activity is essential for the induction of tinnitus,” Proceedings of the National Academy of Sciences of the United States of America. National Academy of Sciences. 110(24), pp. 9980-9985.
Liu, C., Glowatzki, E, and Fuchs, P. A. (2015) “Unmyelinated type II afferent neurons report cochlear damage,” Proceedings of the National Academy of Sciences of the United States of America, 112(47), pp. 14723-14727.
McCullagh, E. A. et al. (2020) “Mechanisms underlying auditory processing deficits in Fragile X syndrome,” FASEB journal: official publication of the Federation of American Societies for Experimental Biology. doi: 10.1096/fj.201902435R.
Neufeld, I. et al. (2021) “A co-twin-control study of altered sensory processing in autism,” Autism: the international journal of research and practice, 25(5), pp. 1422-1432.
Ng, F. L et al. (2011). Expression and function of the K+ channel KCNQ genes in human arteries. British Journal of Pharmacology, 162(1). 42-53.
Nodera, H. et al. (2011) “Neuroprotective effects of Kv7 channel agonist, retigabine, for cisplatin-induced peripheral neuropathy,” Neuroscience letters, 505(3), pp. 223-227.
Palop, J. J. et al. (2007) “Aberrant excitatory neuronal activity and compensatory remodeling of inhibitory hippocampal circuits in mouse models of Alzheimer's disease,” Neuron, 55(5), pp. 697-711.
Pasinelli. P, and Brown, R. H. (2006) “Molecular biology of amyotrophic lateral sclerosis: insights from genetics,” Nature reviews. Neuroscience, 7(9), pp. 710-723.
Peñagarikano, O. et al. (2011) “Absence of CNTNAP2 leads to epilepsy, neuronal migration abnormalities, and core autism-related deficits,” Cell, 147(1), pp. 235-246.
Simkin, D, and Kiskinis, E. (2018) “Modeling Pediatric Epilepsy Through iPSC-Based Technologies,” Epilepsy currents/American Epilepsy Society, 18(4), pp. 240-245.
Soh, H. et al. (2018) “Deletion of KCNQ2/3 potassium channels from PV+ interneurons leads to homeostatic potentiation of excitatory transmission,” eLife, 7. doi: 10.7554/eLife.38617.
Srivastava, S, and Sahin. M. (2017) “Autism spectrum disorder and epileptic encephalopathy: common causes, many questions,” Journal of neurodevelopmental disorders, 9, p. 23.
Styr, B, and Slutsky, I. (2018) “Imbalance between firing homeostasis and synaptic plasticity drives early-phase Alzheimer's disease,” Nature neuroscience, 21(4). pp. 463-473.
Tan, A. et al. (2018) “Effects of the KCNQ channel opener ezogabine on functional connectivity of the ventral striatum and clinical symptoms in patients with major depressive disorder,” Molecular psychiatry. doi: 10.1038/s41380-018-0283-2.
Tanaka, M. et al. (2012) “GABRB3, Epilepsy, and Neurodevelopment,” in Noebels, J. L. et al. (eds.) Jasper's Basic Mechanisms of the Epilepsies. Bethesda (MD): National Center for Biotechnology Information (US).
Thye. M. D. et al. (2018) “The impact of atypical sensory processing on social impairments in autism spectrum disorder,” Developmental cognitive neuroscience. Elsevier, 29, pp. 151-167.
Vossel, K. A. et al. (2013) “Seizures and epileptiform activity in the early stages of Alzheimer disease,” JAMA neurology, 70(9), pp. 1158-1166.
Waaler, P. E. et al. (2000) “Prevalence, classification, and severity of epilepsy in children in western Norway,” Epilepsia, 41(7), pp. 802-810.
Wainger, B. J. et al. (2014) “Intrinsic membrane hyperexcitability of amyotrophic lateral sclerosis patient-derived motor neurons.” Cell reports, 7(1). pp. 1-11.
Wainger, B. J. et al. (2021) “Effect of Ezogabine on Cortical and Spinal Motor Neuron Excitability in Amyotrophic Lateral Sclerosis: A Randomized Clinical Trial,” JAMA neurology, 78(2), pp. 186-196.
Wang, H. S. et al. (1998) “KCNQ2 and KCNQ3 potassium channel subunits: molecular correlates of the M-channel,” Science. American Association for the Advancement of Science, 282(5395), pp. 1890-1893.
Wu, Z. et al. (2017) “Activation of KCNQ Channels Suppresses Spontaneous Activity in Dorsal Root Ganglion Neurons and Reduces Chronic Pain after Spinal Cord Injury,” Journal of neurotrauma, 34(6), pp. 1260-1270.
Xu, W. et al. (2010) “Activation of voltage-gated KCNQ/Kv7 channels by anticonvulsant retigabine attenuates mechanical allodynia of inflammatory temporomandibular joint in rats,” Molecular pain, 6, p. 49.
Zhang, F. et al. (2018) “Fragile X mental retardation protein modulates the stability of its m6A-marked messenger RNA targets,” Human molecular genetics. Oxford Academic, 27(22), pp. 3936-3950.
Zhao, G. et al. (2020) “Gene4Denovo: an integrated database and analytic platform for de novo mutations in humans,” Nucleic acids research. Oxford Academic, 48(D1), pp. D913-D926.

### SUMMARY OF THE PRESENT INVENTION

In some aspects, provided herein is a compound of formula (I), or a solvate, or a pharmaceutically acceptable salt thereof:

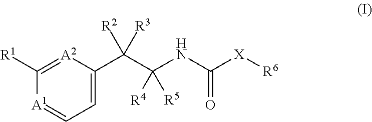

wherein R¹, R², R³, R⁴, R⁵, R⁶, A¹, A² and X are as defined herein.

In a further aspect, the present invention provides a pharmaceutical composition comprising a compounds of formula (I), or a solvate, or a pharmaceutically acceptable salt thereof.

In a further aspect, the present invention provides a compound of formula (I), or a solvate, or a pharmaceutically acceptable salt thereof, or a pharmaceutical composition comprising the same, for use as therapeutically active substance.

In a further aspect, the present invention provides a compound of formula (I), or a solvate, or a pharmaceutically acceptable salt thereof, or a pharmaceutical composition comprising the same, for use in the therapeutic and/or prophylactic treatment of a disorder, disease, or disability associated with Kv7.2.

In a further aspect, the present invention provides the use of a compound of formula (I), or a solvate, or a pharmaceutically acceptable salt thereof, or a pharmaceutical composition comprising the same, in the therapeutic and/or prophylactic treatment of a disorder, disease, or disability associated with Kv7.2.

In a further aspect, the present invention provides the use of a compound of formula (I), or a solvate, or a pharmaceutically acceptable salt thereof, or a pharmaceutical composition comprising the same, for the manufacture of a medicament for the therapeutic and/or prophylactic treatment of a disorder, disease, or disability associated with Kv7.2.

In a further aspect, the present invention provides a method for the therapeutic and/or prophylactic treatment of a disorder, disease, or disability associated with Kv7.2, which method comprises administering a therapeutically effective amount of a compound of formula (I), or a solvate, or a pharmaceutically acceptable salt thereof, or a pharmaceutical composition comprising the same.

In a further aspect, the present invention provides a kit for use in the therapeutic and/or prophylactic treatment of a disorder, disease, or disability associated with Kv7.2, comprising:
a) a compound of formula (I), or a solvate, or a pharmaceutically acceptable salt thereof, or a pharmaceutical composition or a pharmaceutical composition for use comprising the same; and
b) instructions for use.

The compounds of formula (I), or a solvate, or a pharmaceutically acceptable salts thereof, as described herein, provide for a combination of favorable pharmacological properties, such as for example potency, selectivity, and metabolic stability. A reasonable metabolic stability is important to ensure a suitable pharmacological half life, which is best achieved with compounds that have a human liver microsomal clearance <20 μL/min/mg. Selectivity within the Kv7 family is desirable to avoid actions on tissues without therapeutic potential for the indications described in this invention. For example, actions on Kv7.4 and Kv7.5 in skeletal and smooth muscle impact the function of human arteries, where KCNQ2 expression is minimal or undetected in these tissues (Ng et al 2011).

### DETAILED DESCRIPTION OF THE PRESENT INVENTION

### Definitions

Features, integers, characteristics, compounds, chemical moieties or groups described in conjunction with a particular aspect, embodiment or example of the present invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The present invention is not restricted to the details of any embodiments explicitly disclosed herein. Any embodiment described in this application can be combined with any other embodiment. The present invention extends to any novel one, or any novel combination, of the features disclosed in this specification (including any accompanying claims, and abstract), or to any novel one, or any novel combination, of the any embodiment, or any steps of any method or process so disclosed.

All publications, patent applications, patents, and other references mentioned herein are incorporated by reference in their entirety.

The nomenclature used in this application is based on IUPAC systematic nomenclature, unless indicated otherwise.

Any open valency appearing on a carbon, oxygen, sulfur or nitrogen atom in the structures herein indicates the presence of a hydrogen, unless indicated otherwise.

“Administer”, “administered”, or “administering” when used for the therapeutic and/or prophylactic treatment of disorders, diseases, or disabilities as described herein means the giving of a compound of this invention to a patient or subject by any method, e.g. by infusion, inhalation, injection, pate, suppository, or tablet, etc.

As used herein, the terms “including”, “containing”, and “comprising” are used in their open, non-limiting sense.

The articles “a” and “an” as used in this disclosure may refer to one or more than one (e.g., to at least one) of the grammatical object of the article. By way of example, “an element” may mean one element or more than one element.

The term “substituent” denotes an atom or a group of atoms replacing a hydrogen atom on the parent molecule.

As described herein, chemical groups within the present disclosure may be “unsubstituted” or “substituted” with one or more substituents (e.g., 1, 2, 3, 4, or 5), such as those illustrated generally herein, or as exemplified by particular classes, subclasses, and species of the present disclosure. In general, the term “substituted” refers to the replacement of a hydrogen atom in a given structure with a specified substituent. In some embodiments, more than one hydrogen atom is replaced with a specified substituent (e.g, when two hydrogen atoms are replaced with one oxo substituent). Combinations of substituents envisioned by the present disclosure are typically those that result in the formation of stable or chemically feasible compounds. In one embodiment, an optionally substituted group has one substituent. In another embodiment, an optionally substituted group has two substituents. In another embodiment, an optionally substituted group has three substituents. In another embodiment, an optionally substituted group has substituents as described herein. As used herein, the term “unsubstituted” may mean that the specified group bears no substituents beyond the moiety recited (e.g., where valency is satisfied by hydrogen).

The terms “effective amount” or “therapeutically effective amount” refers to an amount of a compound of this invention, in particular of compounds of formula (I), or a solvate or a pharmaceutically acceptable salt thereof, or a pharmaceutical composition of the afore-mentioned, being sufficient to produce a desired therapeutic outcome, such as reducing the severity of duration of, stabilizing the severity of, or eliminating one or more signs, symptoms or causes of a disease, disorder, or disability. For therapeutic use, beneficial or desired results may include, for example, decreasing one or more symptoms resulting from the disease, disorder, or disability (biochemical, histologic and/or behavioral), including its complications and intermediate pathological phenotypes presenting during development of the disease, disorder, or disability, increasing the quality of life of those suffering from the disease, disorder, or disability, decreasing the dose of other medications required to treat the disease, disorder, or disability, enhancing effect of another medication, delaying the progression of the disease, disorder, or disability and/or prolonging survival of patients.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like.

Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins, and the like.

The term “excipient” or “pharmaceutical excipients” as used herein refers to any pharmaceutically acceptable excipient that may be used in the production of a drug or pharmaceutical composition, such as a tablet containing a compound as described herein (or tautomer or pharmaceutically acceptable salt) as an active ingredient. Various substances may be embraced by the term excipient, including without limitation any substance used as a diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Disintegrant refers to excipients that expand and dissolve when wet causing the tablet to break apart in the body and release the active ingredient for absorption. Examples include cross-linked polymers like crospovidone, croscarmellose sodium, etc, and modified starches like sodium starch glycolate. Filler refers to excipients that fill out the size of a tablet by increasing the bulk volume. Fillers make it possible for the final product to have the proper volume for patient handling. Examples of fillers are plant cellulose, lactose, starch, mannitol, etc. Specific examples are lactose monohydrate like Pharmatose 200M, microcrystalline cellulose (MCC) like Avicel PH101, or Avicel PH102 and spray dried lactose like Fast Flo 316™. Binders refers to excipients that hold the ingredients in a tablet together. Binders ensure that tablets and granules can be formed with required mechanical strength. Examples of binders are, polyvinlypyrrolidon (PV), hydroxypropyl methylcellulose (HPMC), hydroxypropylcellulose (HPC), cellulose, sugar alcohols like sorbitol, proteins like gelatin and polymers like PVP, e.g, copovidone (PVP/VA 64), PEG, etc. Lubricants refer to excipients that prevent ingredients from clumping together and from sticking to the tablet punches or capsule filling machine. Lubricants also ensure that tablet formation and ejection can occur with low fraction between active ingredient and wall. Examples of lubricants are minerals like talc or silica and fats like stearin, magnesium stearate, etc. Coatings may include, e.g., cellulose acetate phthalate, ethylcellulose, gellan gum, maltodextrin, enteric coatings, etc.; compression/encapsulation aids include e.g, calcium carbonate, dextrose, fructose dc (dc—“directly compressible”), honey dc, lactose (anhydrate or monohydrate; optionally in combination with aspartame, cellulose, or microcrystalline cellulose), starch dc, sucrose, etc. Creams or lotions include, e.g., maltodextrin, carrageenans, etc. Materials for chewable tablets include, e.g. dextrose, fructose dc, lactose (monohydrate, optionally in combination with aspartame or cellulose), etc. Suspending/gelling agents include, e.g., carrageenan, sodium starch glycolate, xanthan gum, etc. Sweeteners include, e.g., aspartame, dextrose, fructose dc, sorbitol, sucrose dc, etc. Wet granulation agents include, e.g., calcium carbonate, maltodextrin, microcrystalline cellulose, etc. In some cases, the term “excipient” encompasses pharmaceutically acceptable carriers. The skilled person knows suitable pharmaceutical compositions to be used in the treatment of patients and how to produce them.

The term a “patient” or “subject” may encompass both mammals and non-mammals. Examples of mammals may include, but are not limited to, any member of the class Mammalia: humans; nonhuman primates such as chimpanzees, monkeys, baboons, or rhesus monkeys, as well as other apes and monkey species; farm animals such as cattle, horses, sheep, goats, and swine; companion animals such as rabbits, dogs, and cats; laboratory animals including rodents, such as rats, mice and guinea pigs; and the like. Examples of non-mammals include, but are not limited to, birds, fish, and the like. “Patient” or “subject” may include both human and animals. In some preferred embodiments, the “patient” or “subject” is a human.

As used herein, the terms “treat” or “treatment” are meant to indicate a postponement of development of one or more disease(s), disorder(s), or disability(ies); preventing the development of one or more disease(s), disorder(s), or disability(ies); and/or reducing severity of one or more symptoms of a disease, disorder, or disability that will or are expected to develop. Thus, these terms may include ameliorating one or more existing disease, disorder, or disability symptoms; preventing one or more additional symptoms; ameliorating or preventing the underlying causes of one or more symptoms; inhibiting the diseases, disorder, or disability, e.g., arresting the development of the diseases, disorder, or disability; relieving the diseases, disorder, or disability; causing regression of the diseases, disorder, or disability: relieving a symptom caused by the diseases, disorder, or disability; or stopping or alleviating the symptoms of the diseases, disorder, or disability.

Compounds of the present invention and disclosure may exist as solvates. The term “solvate” may refer to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water. In some embodiments, solvates are excluded.

The term “prophylaxis” as used herein includes: preventing or delaying the appearance of clinical symptoms of diseases, disorder, or disability developing in a patient or subject, especially a human, that may be afflicted with or predisposed to the disease, disorder, or disability as described herein, but does not yet experience or display clinical or subclinical symptoms of the disease, disorder, or disability.

As used herein, the term “about,” when referring to a value is meant to encompass variations of, for example, in some embodiments ±20%, in some embodiments ±10%, in some embodiments 5%, in some embodiments ±1%, in some embodiments ±0.5%, and in some embodiments 0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the present invention. The upper and lower limits of these small ranges which may independently be included in the smaller ranges is also encompassed within the present invention, subject to any specifically excluded limit in the stated range. Where the stated range includes one or both of the limits, ranges excluding either or both of those included limits are also included in the present invention.

Numerical ranges, as used herein, may include sequential integers. For example, a range expressed as “from 0 to 5” would include 0, 1, 2, 3, 4, and 5.

A “metabolite” is a product produced through metabolism in the body of a specified compound or salt thereof. Metabolites of a compound may be identified using routine techniques known in the art and their activities determined using tests such as those described herein. Such products may result e.g. from the oxidation, reduction, hydrolysis, amidation, deamidation, esterification, deesterification, enzymatic cleavage, and the like, of the administered compound. Accordingly, the present invention includes metabolites of compounds of the present invention, including compounds produced by a process comprising contacting a compound of this invention with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

The terms “compound of this invention,” and “compounds of the present invention” “compounds of the present invention” and similar expressions include compounds of formula (I), compounds selected from any of formulae (II)-(XV) etc., compounds from Tables 1, compounds provided elsewhere in this application, etc., stereoisomers, geometric isomers, solvates, pharmaceutically acceptable salts, tautomers, metabolites, prodrugs, polymorphs; and mixtures thereof. In some embodiments, solvates are not included.

The symbols “ [symbol] ”, “ [symbol] ” or “ [symbol] ” drawn at the end of a bond or through a bond each refer to the point of attachment of a functional group or other chemical moiety to the rest of the molecule of which it is a part. Thus, for example:

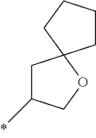

means that the substituent is attached to the rest of the molecule as shown

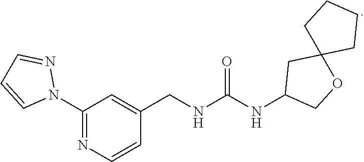 [SMILES: O=C(NCc1ccnc(-n2cccn2)c1)NC1COC2(CCCC2)C1]

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent. “Optionally substituted” means that a compound can be unsubstituted or substituted as defined herein. “The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen. In an addition, e.g. in a compound in which e.g. R² and R³ are independently selected from hydrogen and hydroxyC₁₋₆alkyl, both R² and R³ can be hydrogen, or both can be hydroxyC₁₋₆alkyl, or one of R² and R³ can be hydrogen and the other one hydroxyC₁₋₆alkyl.

In this application the units ul, uMol, C etc. mean μl, μMol, ° C. etc.

The term “EC₅₀” in this application is defined as: the agonistic effect of a compound can be determined by testing the compound in an in vitro assay as described herein, whereby the effect of the compound is measured across a range of compound concentrations. The resulting data is plotted as a concentration response curve, which typically follows a sigmoidal function, whereby the concentration of the compound is plotted on the x axis and the response (agonistic effect) is plotted on the v axis. The term “EC₅₀” is the “half maximal effective concentration” and denotes the concentration of a particular compound required to obtain 50% of the maximum response (Eₘₐₓ) which is observed for that compound in the given in vitro assay.

A compound of this invention may exist in one or more stereoisomeric forms (e.g., it contains one or more asymmetric carbon atoms). The individual stereoisomers (enantiomers and diastereomers) and mixtures of these are included within the scope of the subject matter disclosed herein.

Likewise, it is understood that a compound or salt may exist in tautomeric forms other than that shown in the formula and these are also included within the scope of the subject matter disclosed herein. It is to be understood that the subject matter disclosed herein includes combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

A compound of this invention can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “chiral” refers to molecules which have the property of non-superimposability of the mirror image partner, while the term “achiral” refers to molecules which are superimposable on their mirror image partner. Chiral separation of a racemate to its enantiomeric components may be performed to separate the eutomer and the distomer.

The term “stereoisomers” refers to compounds, which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel. E, and Wilen, S., “Stereochemistry of Organic Compounds”, John Wiley & Sons, Inc., New York. 1994. The compounds of the present invention may contain asymmetric or chiral centers, and therefore exist in different stereoisomeric forms. It is intended that all stereoisomeric forms of the compounds of the present invention, including but not limited to, diastereomers, enantiomers and atropisomers, as well as mixtures thereof such as racemic mixtures, form part of the present invention. Any organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process.

The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

It should be understood that individual enantiomers and diastereomers are included in the tables below by compound name, and their corresponding structures can be readily determined therefrom. In some instances, the enantiomers or diastereomers are identified by their respective properties, for example, retention times on a chiral HPLC or their biological activities (e.g., as described further in the Examples), and the absolute stereo configurations of one or more chiral centers are arbitrarily assigned (e.g., stereochemistry of all chiral centers is arbitrarily assigned, or stereochemistry of one chiral center is known and remaining chiral centers arbitrarily assigned, etc.).

In some embodiments of the present invention only one of the possible enantiomers are used. In other embodiments mixtures of the possible enantiomers having different percentages for each component are used.

In some embodiments, the compounds of this invention are isotopically-labeled by having one or more atoms therein replaced by an atom having a different atomic mass or mass number. Such isotopically-labeled (e.g., radiolabeled) compounds of formula (I), or a solvate or a pharmaceutically acceptable salts thereof, are considered to be within the scope of this disclosure. Examples of isotopes that can be incorporated into the compounds of this invention, include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of this invention for example, those incorporating a radioactive isotope, are useful in drug and/or substrate tissue distribution studies. The radioactive isotopes tritium, i.e. 3H, and carbon-14, i.e., ¹⁴C, are particularly useful for this purpose in view of their ease of incorporation and ready means of detection. For example, a compound of this invention can be enriched with 1, 2, 5, 10, 25, 50, 75, 90, 95, or 99 percent of a given isotope.

In some aspects, it is particularly envisaged that compounds of formula (I), or a solvate or a pharmaceutically acceptable salts thereof, have one or more hydrogen atoms in the structure substituted by ²H (deuterium), ³H (tritium), preferably ²H (deuterium). Any of the hydrogen atoms in the structures may be substituted in this way.

Substitution with positron emitting isotopes, such as ³H, ¹¹C, ¹⁸F, ¹⁵O and ¹³N, can be useful in Positron Emission Topography (PET) studies for examining substrate receptor occupancy. Isotopically-labeled compounds of this invention can generally be prepared by conventional techniques known to those skilled in the art or by processes analogous to those described in the Examples as set out below using an appropriate isotopically-labeled reagent in place of the non-labeled reagent previously employed.

In one embodiment, the present invention provides pharmaceutically acceptable salts of the compounds of this invention especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I), or solvates or pharmaceutically acceptable salts thereof, as described herein (i.e., as “free bases” or “free acids”, respectively).

When a bond in a compound of this invention is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

The term “alkyl” refers to a mono- or multivalent, e.g., a mono- or bivalent, linear or branched saturated hydrocarbon group, in particular a hydrocarbon group of 1 to 6 carbon atoms (“C₁₋₆alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of methyl (CH₃—), ethyl (CH₃CH₂—), n-propyl (CH₃CH₂CH₂—). 2-propyl (isopropyl, (CH₃)₂CH₂—), n-butyl (CH₃CH₂CH₂CH₂—), iso-butyl ((CH₃)₂CH₂CH₂—), sec-butyl (CH₃CH(CH₃)CH₂—), and tert-butyl ((CH₃)₃C—). Preferred, yet non-limiting, examples of alkyl are tert-butyl ((CH₃)₃C—) or methyl (CH₃—). A preferred, yet non-limiting, example of alkyl is methyl (CH₃—). Another preferred, yet non-limiting, example of alkyl is tert-butyl ((CH₃)₃C—).

The term “alkoxy” refers to an alkyl group, as previously defined, attached to the parent molecular moiety via an oxygen atom. The alkoxy group preferably contains 1 to 6 carbon atoms (“C₁₋₆alkoxy”). e.g. 1, 2, 3, 4, 5, or 6 carbon atoms. In other embodiments, the alkoxy group contains 1 to 4 carbon atoms. In still other embodiments, the alkoxy group contains 1 to 3 carbon atoms. Some non-limiting examples of alkoxy groups include CH₃O— (methoxy), CH₃CH₂O— (ethoxy), CH₃CH₂CH₂O— (n-propoxy), and (CH₃)₃CO— (tert-butoxy). A particularly preferred, yet non-limiting, example of alkoxy is methoxy (CH₃O—).

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cyano” refers to a —CN (nitrile) group.

The term “hydroxy” or “hydroxyl” refers to an OH group.

The term “haloalkyl” refers to an alkyl group wherein at least one of the hydrogen atoms of the alkyl group has been replaced by one or more halogen atoms. Preferably, “haloalkyl” refers to a C₁₋₆alkyl group wherein 1, 2 or 3 hydrogen atoms of the alkyl group have been replaced by a halogen atom, i.e. haloalkyl includes monohaloalkyl, dihaloalkyl, trihaloalkyl, perhaloalkyl and the like. Halogen atoms may be fluoro (F), chloro (Cl), or bromo (Br). Particularly preferred, yet non-limiting, examples of “halogen” within haloalkyl are fluoro (F) and chloro (Cl). More preferably, haloalkyl is substituted with fluoro (F). Preferred, yet non-limiting, examples of haloalkyl are (CH₃)₂FC— (1-fluoro-isopropyl), CF₃CH₂— (2,2,2-trifluoroethyl), CH₃CF₂— (1,1-difluoroethyl), CF₃— (trifluoromethyl), CH₂F— (fluoromethyl), or CHF₂— (difluromethyl). Particularly preferred are CH₃CF₂—, and CF₃—.

The term “haloalkoxy”, as used herein, refers to an alkoxy group, wherein at least one of the hydrogen atoms has been replaced by halogen atoms. Preferably, “haloalkoxy” refers to an alkoxy group, preferably a C₁₋₆alkoxy group, wherein 1, 2, or 3 hydrogen atoms of the alkyl group have been replaced by a halogen atom, i.e. haloalkoxy includes monohaloalkoxy, dihaloalkoxy, trihaloalkoxy, perhaloaloxy and the like. Halogen atoms may be fluoro (F), chloro (Cl) or bromo (Br). Particularly preferred, yet non-limiting, examples of “halogen” are fluoro (F) and chloro (Cl). More preferably, haloalkoxy is substituted with fluoro (F). Preferred, yet non-limiting, examples of haloalkoxy are CHF₂O—, CH₂FO—, CF₃CH₂O—, CF₂HCH₂O—, CH₃CF₂CH₂O—, and CH₃CFHCH₂O—. Also preferred, yet non-limiting, examples of haloalkoxy are haloC₁₋₆alkoxy selected from CHF₂O—, CH₂FO—, CF₃O—, FCH₂CFHCH₂O—, CH₂FCH₂O—, and CF₃CH₂O—.

Particularly preferred are CHF₂O—, CF₃CH₂O—, CF₃O—, CH₂FO—, or FCH₂CFHCH₂O—.

The term “hydroxyalkyl” refers to an alkyl group, preferably with one to six C-atoms, wherein at least one of the hydrogen atoms of the alkyl group has been replaced by one or more hydroxy. Preferably, “hydroxyalkyl” refers to an alkyl group, preferably a C₁₋₆alkyl, wherein 1, 2 or 3 hydrogen atoms of the alkyl group have been replaced by hydroxy, i.e. hydroxyalkyl includes monohydroxyalkyl, dihydroxalkyl, trihydroxyalkyl, perhydroxyalkyl and the like. More preferably, “hydroxyalkyl” refers to an alkyl group wherein one hydrogen atom has been replaced by hydroxy. Particularly preferred, yet not limiting examples of hydroxyalkyl are HOCH₂— (hydroxymethyl), and HOCH₂CH₂— (hydroxyethyl).

The term “heterocycloalkyl” denotes a monovalent saturated or partly unsaturated mono- or bicyclic heterocyclic ring system of 4 to 6 ring atoms, comprising 1, 2, or 3 ring heteroatoms selected independently from N, O and S, the remaining ring atoms being carbon. Preferably, “heterocycloalkyl” refers to a saturated monocyclic 4-6 membered heterocycloalkyl comprising one O-atoms, with non-limiting examples oxetanyl, oxolanyl, and oxanyl. The heterocycloalkyl can be substituted as described herein, e.g, with halogen.

The term “cycloalkyl” refers to to a monovalent non-aromatic, saturated or partially unsaturated ring having 3 to 10 carbon atoms (C₃-C₁₀) as a monocyclic ring or 7 to 10 carbon atoms as a bicyclic ring. In some embodiments, cycloakyl is a monocyclic 4-6 membered cycloalkenyl group with one non-aromatic double bond. In some embodiments, cycloalkenyl is selected from cyclobutenyl, cyclopentenyl, and cyclohexenyl. Polycyclic and bicyclic cycloalkyl groups may include fused, or bridged polycyclic or bicyclic moieties, wherein each ring is a saturated or partially unsaturated, non-aromatic hydrocarbon. Cycloalkyl comprises bicyclic cycloalkyl referring to 4-10 membered bridged cycloalkyl moieties consisting of two saturated carbocycles having two carbon atoms in common, i.e., the bridge separating the two rings is either a single bond or a chain of one or two ring atoms. Bicyclic carbocycles having 7 to 10 atoms can be arranged, e.g., as a bicyclo[4,5], [5,5], [5,6] or [6,6] system, and bicyclic carbocycles having 9 or 10 ring atoms can be arranged as a bicyclo[5,6] or [6,6] system, or as bridged systems such as bicyclo[2.2.1]heptane, bicyclo[2.2.2]octane and bicyclo[3.2.2]nonane. Preferably, “cycloalkyl” refers to a saturated monocyclic 3-6 membered cycloalkyl. More preferably, saturated monocyclic 3-6 membered cycloalkyl is selected from cyclopropyl, cyclobutyl, cyclopentyl, and cyclohexyl. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The term “cyanoC₁₋₆alkoxy” refers to an alkoxy group, preferably with one to six C-atoms, wherein at least one of the hydrogen atoms of the alkoxy group has been replaced by one or more cyano. Preferably, “cyanoC₁₋₆alkoxy” refers to an alkyl group, preferably a C₁₋₆alkoxy, wherein 1, 2 or 3 hydrogen atoms of the alkoxy group have been replaced by cyano. More preferably, “cyanoC₁₋₆alkoxy” refers to an C₁₋₆alkoxy group wherein one hydrogen atom has been replaced by cyano. Particularly preferred, yet not limiting examples of cyanoC₁₋₆alkoxy are NC—CH₂—O—, NC—CH₂CH₂—O— and NC—CH₂CH₂CH₂—O—.

The term “C₃₋₅cycloalkylC₁₋₆alkoxy” as used herein refers to an alkoxy group, wherein at least one of the hydrogen atoms was replaced by a C₃₋₅cycloalkyl group. Preferably, “C₃₋₅cycloalkylC₁₋₆alkoxy” refers to an alkoxy group wherein one, two, or three hydrogen atoms of the alkoxy group have been replaced by a C₃₋₅cycloalkyl group. Preferably, “C₃₋₅cycloalkylC₁₋₆alkoxy” refers to an alkoxy group wherein one hydrogen atom of the alkoxy group has been replaced by a C₃₋₅cycloalkyl group. Preferably, the alkoxy group comprises one C-atom. Preferred, yet not-limiting, examples are 2-cyclopropylmethoxy and 2-cyclobutylmethoxy.

The term “heteroaryl” as used herein refers to a 5 or 6 membered monocyclic, aromatic group comprising at least one ring heteroatom. In some embodiments, the heteroatom is independently selected from the group consisting of N-atoms, O-atoms, and S-atoms. The number of ring atoms refer to the sum of carbon and heteroatoms in the one ring. In some embodiment, heteroaryl is a 5 or 6 membered moncyclic, aromatic group with two N-atoms. In some embodiments, heteroaryl is a 5 or 6 membered monocyclic aromatic group comprising one N-atom. In some embodiments, heteroaryl is a 5 or 6 membered monocyclic aromatic group comprising one N-atom and one O-atom. Examples of 5 membered heteroaryl groups include, but are not limited to, pyrrolyl pyrazolyl, imidazolyl, oxazolyl, triazolyl, or furanyl. In some preferred embodiments, 5 membered heteroaryl is pyrazolyl, imidazolyl, or oxazolyl. In some more preferred embodiments. 5 membered heteroaryl is pyrazolyl or imidazolyl. Examples of 6 membered heteroaryl groups include, but are not limited to, pyrimidinyl, pyridinyl, pyrazinyl, or pyridazinyl. In some more preferred embodiments, 6 membered heteroaryl is pyrazinyl, pyridinyl or pyrimidinyl.

The term “hydroxyC₂₋₄alkenyl” refers to an alkenyl group wherein at least one of the hydrogen atoms of the alkenyl group has been replaced by one or more hydroxy groups. Preferably, “hydroxyalkenyl” refers to a C₂₋₄alkenyl group wherein 1, 2 or 3 hydrogen atoms of the alkenyl group have been replaced by a hydroxy group, i.e. hydroxyalkenyl includes monohydroxyalkenyl, dihydroxyalkenyl, trihydroxyalkenyl, perhydroxyalkenyl and the like. A preferred, yet non-limiting, example of hydroxyalkenyl is HO—CH₂—CH═CH—.

The term “7-11 membered spirocylic cyloalkyl” as used herein refers to a bicyclic spirocyclic cycloakyl comprising 7, 8, 9, 10, or 11 C-atoms with two saturated non-aromatic rings. In some embodiments, spirocyclic cycloalkyl comprises a tricyclic spiro-cycloakyl comprising 9-11 C-atoms. In some embodiments, the ring directly attached to the molecule is not a 4 membered ring. In some examples, the 7-10 membered spirocyclic cycloalkyl is selected from spiro[3.3]heptanyl, spiro[2.3]hexanyl, spiro[3.4]octanyl optionally substituted as described herein.

The term “7-11 membered oxo-spiro-heterocycloalkyl” as used herein refers to bicyclic oxa-spiro-heteroccyloalkyls comprising 7, 8, 9, 10, or 11 ring atoms, comprising two saturated monocyclic rings, wherein one rings comprise one O-atom. In some embodiments, the O-atom is in the ring directly attached to the molecule. In some embodiments, the ring directly attached to the molecule is not a 4 membered ring. In some embodiments, the 7-11 membered oxa-spiro-heterocycloalkyl is selected from oxaspiroheptanyl, oxaspirooctanyl, oxaspirononanyl, oxaspirodecanyl, and oxaspiroundecanyl. In some preferred embodiments, the oxa-spiro-heterocycloalkyl comprises one O-atom to form a 9-10 membered oxa-spiro-heterocycloalkyl. Non-limiting examples of 9-10 membered oxa-spiro-heterocycloalkyl are selected from oxaspirononanyl and oxaspirodecanyl. All oxo-spiro-heterocycloalkyls can be substituted as described herein.

The term “mood disorder” as used herein relates to a mental health problem that primarily affects a person's emotional state. It is a disorder in which a person experiences long periods of extreme happiness, extreme sadness or both. Two of the most common mood disorders are depression and bipolar disorder.

The term “depression” as used herein relates to a mood disorder that causes a persistent feeling of sadness and loss of interest. It is also known as major depressive disorder (MDD).

The term “behavioral disorder” relates to disorders that involve a pattern of disruptive behaviors in children that last for at least 6 months and cause problems in school, at home and in social situations. Behavioral disorders involve a pattern of disruptive behaviors in children that last for at least 6 months and cause problems in school, at home and in social situations. The most important behavioral disorder is Attention deficit hyperactivity disorder” (ADHD).

The term “Attention deficit hyperactivity disorder” (ADHD) as used herein relates to a behavioral disorder characterized by inattention, or excessive activity and impulsivity. ADHD occurs more frequently in people with epilepsy than in the general population. Children with ADHD have an increased risk of seizures, with approximately 14% of children with ADHD developing seizures.

The term “developmental disorder” or “neurodevelopmental disorder” as used herein relates to a group of conditions caused by an impairment in physical, learning, language, or behavior areas. These conditions begin during the developmental period, may impact day-to-day functioning, and can last through a person's lifetime. Examples of neurodevelopment disorders include Autism Spectrum Disorder (“ASD”) and syndromic developmental disorders.

The term “Autism Spectrum Disorder (ASD)” as used herein relates to a developmental disorder of variable severity that is characterized by difficulty in social interaction and communication and by restricted or repetitive patterns of thought and behavior. ASD encompasses mostly idiopathic but also syndromic forms and is currently diagnosed according to the diagnostic and statistical manual version 5 (DSM V).

The term “syndromic developmental disorder” as used herein relates to a development disorder with a clinically defined pattern of somatic abnormalities and a neurobehavioral phenotype that may include ASD. The diagnosis is typically confirmed by targeted genetic testing. Examples for syndromic development disorders include Dup15q syndrome (Dup15q). Fragile X syndrome (FXS) and Angelman syndrome.

The term “Dup15q syndrome” or “Duq15q” as used herein relates to the common name for chromosome 15q11.2-q13.1 duplication syndrome. This is a syndromic development disorder, caused by the partial duplication of Chromosome 15, which confers a strong risk for autism spectrum disorder, epilepsy and intellectual disability.

The term “Fragile X syndrome” (FXS) as used herein relates to a genetic disorder characterized by mild-to-moderate intellectual disability. This disorder is typically caused by an expansion of the CGG triplet repeat within the FMRT (fragile X mental retardation 1) gene on the X chromosome.

The term “Angelman syndrome” as used herein relates to a genetic disorder that mainly affects the nervous system due to a lack of function of part of chromosome 15 inherited from a person's mother. Characteristic features of this condition include delayed development, intellectual disability, severe speech impairment, and problems with movement and balance (ataxia). Most affected children also have recurrent seizures (epilepsy).

The term “Intellectual disability” (ID) used herein relates to a generalized neurodevelopmental disorder characterized by significantly impaired intellectual and adaptive functioning. It is defined by an IQ under 70, in addition to deficits in two or more adaptive behaviors that affect everyday, general living. ID is also known as a general learning disability and formerly known as mental retardation (MR).

The term “epilepsy” used herein relates to a neurological disorder marked by sudden recurrent episodes of sensory disturbance, loss of consciousness, or convulsions, associated with abnormal electrical activity in the brain. Examples of epilepsies include broad pediatric epilepsies, West syndrome, Ohtahara syndrome and epileptic encephalopathy.

The term “neurodegenerative diseases” used herein relates to diseases that are related to e progressive loss of structure or function of neurons, including the death of neurons. Examples of neurodegenerative diseases include, but are not limited to, Alzheimer's disease and motor neuron diseases.

The term “motor neuron disease” used herein relates to a group of rare neurodegenerative disorders that selectively affect motor neurons. Examples of motor neuron diseases include, but are not limited to, amyotrophic lateral sclerosis (ALS).

The term “pain” as used herein relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain.

The term “migraine” as used herein relates to a moderate to severe headache disorder, causing throbbing or pulsating pain for hours or days.

The term “Tinnitus” as used herein relates to a symptom characterized by the perception of sound when no corresponding external sound is present.

Any disease, disorder, or disability described herein also includes any state or condition related to such disease, disorder, or disability.

### Compounds of the Present Invention

All compound names of compound structures were generated using OpenEye Lexichem, Version 1.2.0, OpenEye Scientific Software, Santa Fe, NM, USA: www.eyesopen.com.

Features, integers, characteristics, compounds, chemical moieties or groups described in conjunction with a particular aspect, embodiment or example of the present invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The present invention is not restricted to the details of any embodiments described herein. Any embodiment described in this application can be combined with any other embodiment. For example, any embodiment herein relating to the compounds of this invention can be combined with any embodiment of pharmaceutical compositions, kits, medical use, or method of treatment. The present invention extends to any novel one, or any novel combination, of the features disclosed in this specification (including any accompanying claims, abstract and drawings), or to any novel one, or any novel combination, of the steps of any method or process so disclosed.

In some embodiments of this invention, compounds of formula (I) have the structures depicted from any one of formulae (II)-(XV), etc. as described herein.

The present invention provides a compound of formula (I), or a solvate, or a pharmaceutically acceptable salt thereof:

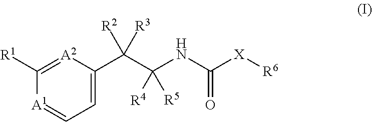

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxyl, and halogen;
R² and R³ are independently selected from hydrogen, hydroxy, C₁₋₆alkyl, C₁₋₆alkoxy, hydroxyC₁₋₆alkyl, C₃₋₅cycloalkylC₁₋₆alkoxy, cyano, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen or halogen:
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

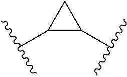 [SMILES: *C1CC1*]

R⁶ is selected from 3-10 membered cycloalkyl, 7-11 membered spiro-cycloalkyl. 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

In this application the group

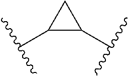 [SMILES: *C1CC1*]

for X means that the bonds are not part of X and e.g. X is cyclopropyl incorporated into the compound of formula (I) as shown in the exemplary compound with the structure:

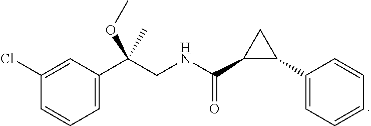 [SMILES: CO[C@@](C)(CNC(=O)[C@H]1C[C@@H]1c1ccccc1)c1cccc(Cl)c1]

In one embodiment the present invention provides a compound of formula (I), or a solvate, or a pharmaceutically acceptable salt thereof, wherein (i) only A¹; (ii) only A²: (iii) A¹ and A² both are N; or (iv) A¹ is CR^{a} and A² is CR^{b}.

In one embodiment the present invention provides a compound of formula (I), or a solvate, or a pharmaceutically acceptable salt thereof, wherein only one of A¹ and A² is N and the other one is CR^{a} or CR^{b}.

In one preferred embodiment the present invention provides a compound of formula (I), or a solvate, or a pharmaceutically acceptable salt thereof, wherein A¹ is CR^{a} and A² is CR^{b}.

In one preferred embodiment the present invention provides a compound of formula (I), or a solvate, or a pharmaceutically acceptable salt thereof, wherein R^{a} and R^{b} are hydrogen.

In one preferred embodiment the present invention provides a compound of formula (I), or a solvate, or a pharmaceutically acceptable salt thereof, wherein R^{a} is F and R^{b} is hydrogen.

In one embodiment the present invention provides a compound of formula (II) with the formula:

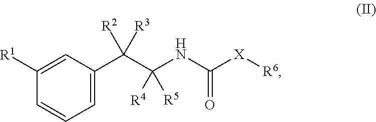

or a solvate, or a pharmaceutically acceptable salt thereof, wherein R¹, R², R³, R⁴, R⁵, X and R⁶ are as described herein.

In one embodiment the present invention provides a compound of formula (I), or a solvate, or a pharmaceutically acceptable salt thereof, wherein R¹ is halogen.

In one embodiment the present invention provides a compound of formula (I), or a solvate, or a pharmaceutically acceptable salt thereof, wherein R¹ is halogen selected from F, Cl and Br.

In one embodiment the present invention provides a compound of formula (I), or a solvate, or a pharmaceutically acceptable salt thereof, wherein R¹ is halogen selected from Cl and Br.

In one preferred embodiment the present invention provides a compound of formula (I), or a solvate, or a pharmaceutically acceptable salt thereof, wherein R¹ is C₁.

In one preferred embodiment the present invention provides a compound of formula (I), or a solvate, or a pharmaceutically acceptable salt thereof, wherein R¹ is cyano or haloC₁₋₆alkoxy.

In one preferred embodiment the invention provides a compound of formula (I), or a solvate, or a pharmaceutically acceptable salt thereof, wherein R¹ is cyano.

In one preferred embodiment the presents invention provides a compound of formula (I), or a solvate, or a pharmaceutically acceptable salt thereof, wherein R¹ is haloC₁₋₆alkoxy.

In one preferred embodiment the present invention provides a compound of formula (I), or a solvate, or a pharmaceutically acceptable salt thereof, wherein R¹ is haloC₁₋₆alkoxy selected from CHF₂O—, CH₂FO—, CF₃CH₂O—, CF₂HCH₂O—, CH₃CF₂CH₂O—, and CH₃CFHCH₂O—.

In one preferred embodiment the present invention provides a compound of formula (I), or a solvate, or a pharmaceutically acceptable salt thereof, wherein R¹ is CHF₂O—.

In one embodiment the present invention provides a compound of formula (III), with the formula:

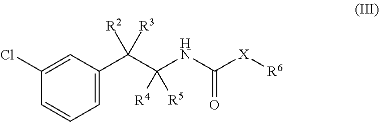

or a solvate, or a pharmaceutically acceptable salt thereof, wherein R², R³, R⁴, R⁵, X and R⁶ are as described herein.

In one preferred embodiment the present invention provides a compound of formula (IV) with the formula:

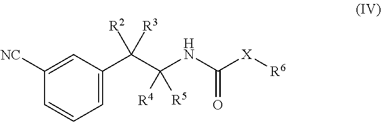

or a solvate, or a pharmaceutically acceptable salt thereof, wherein R², R³, R⁴, R⁵, X and R⁶ are as described herein.

In one preferred embodiment the present invention provides a compound of formula (V) with the formula.

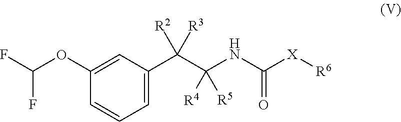

or a solvate, or a pharmaceutically acceptable salt thereof, wherein R², R³, R⁴, R⁵, X and R⁶ are as described herein.

In one preferred embodiment the present invention provides a compound of formula (I), or a solvate, or a pharmaceutically acceptable salt thereof, wherein R² is hydroxy and R³ is C₁₋₆alkyl or R² is C₁₋₆alkoxy and R³ is C₁₋₆alkyl.

In one preferred embodiment the present invention provides a compound of formula (I), or a solvate, or a pharmaceutically acceptable salt thereof, wherein A¹ is CR^{a}, A² is CR^{b}, R^{a} and R^{b} are hydrogen, R¹ is selected from C₁, cyano, and CHF₂O—, R² is C₁₋₆alkoxy and R³ is C₁₋₆alkyl.

In one preferred embodiment the present invention provides a compound of formula (I), or a solvate, or a pharmaceutically acceptable salt thereof, wherein A¹ is CR^{a}, A² is CR^{b}, R^{a} and R^{b} are hydrogen, R¹ is Cl, R² is C₁₋₆alkoxy and R³ is C₁₋₆alkyl.

In one preferred embodiment the present invention provides a compound of formula (I), or a solvate, or a pharmaceutically acceptable salt thereof, wherein A¹ is CR^{a}, A² is CR^{b}, R^{a} and R^{b} are hydrogen, R¹ is cyano, R² is C₁₋₆alkoxy and R³ is C₁₋₆alkyl.

In one preferred embodiment the present invention provides a compound of formula (I), or a solvate, or a pharmaceutically acceptable salt thereof, wherein A¹ is CR^{a}, A² is CR^{b}, R^{a} and R^{b} are hydrogen, R¹ is CHF₂O—, R² is C₁₋₆alkoxy and R³ is C₁₋₆alkyl.

In one embodiment the present invention provides a compound of formula (I), or a solvate, or a pharmaceutically acceptable salt thereof, wherein R² is C₁₋₆alkyl, wherein one or more hydrogen atoms are replaced by D.

In one embodiment the present invention provides a compound of formula (I), or a solvate, or a pharmaceutically acceptable salt thereof, wherein R² is —OCD₃ and R³ is C₁₋₆alkyl.

In one embodiment the present invention provides a compound of formula (I), or a solvate, or a pharmaceutically acceptable salt thereof, wherein R² is hydroxy or —OCD₃ and R³ is methyl.

In one embodiment the present invention provides a compound of formula (I), or a solvate, or a pharmaceutically acceptable salt thereof wherein R² is methoxy and R³ is methyl.

In one embodiment the present invention provides a compound of formula (VI) with the formula:

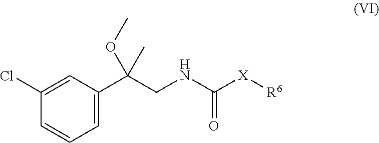

or a solvate, or a pharmaceutically acceptable salt thereof, wherein X and R⁶ are as described herein.

In one embodiment the present invention provides a compound of formula (I), or a solvate, or a pharmaceutically acceptable salt thereof, wherein R² and R³ together with the C-atom they are attached to form a 4-6 membered heterocycloalkyl or a 3-6 membered cycloalkyl, wherein such heterocyclalkyl or cycloalkyl are optionally substituted with 1 to 4 halogen or oxo.

In one embodiment the present invention provides a compound of formula (I), or a solvate, or a pharmaceutically acceptable salt thereof, wherein R² and R³ together with the C-atom they are attached to form a 4-6 membered heterocycloalkyl, wherein such heterocyclalkyl is optionally substituted with 1 to 4 halogen or oxo.

In one embodiment the present invention provides a compound of formula (I), or a solvate or a pharmaceutically acceptable salt thereof, wherein the heterocycloalkyl formed comprises one or two S- or O-atoms.

In one embodiment the present invention provides a compound of formula (I), or a solvate or a pharmaceutically acceptable salt thereof, wherein the heterocycloalkyl formed comprises one or two O-atoms.

In one embodiment the present invention provides a compound of formula (I), or a solvate or a pharmaceutically acceptable salt thereof, wherein the heterocycloalkyl formed comprises two O-atoms.

In one embodiment the present invention provides a compound of formula (I), or a solvate or a pharmaceutically acceptable salt thereof, wherein the heterocycloalkyl comprises one O-atom.

In one embodiment the present invention provides a compound of formula (I), or a solvate or a pharmaceutically acceptable salt thereof, wherein R⁴ and R⁵ are H.

In one embodiment the present invention provides a compound of formula (I), or a solvate or a pharmaceutically acceptable salt thereof, wherein the linker comprising R², R³, R⁴ and R⁵ has the following structure:

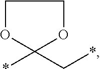

optionally substituted as described herein.

In one embodiment the present invention provides a compound of formula (I), or a solvate or a pharmaceutically acceptable salt thereof, wherein the linker comprising R², R³, R⁴ and R⁵ has the following structure:

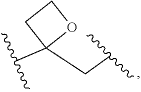 [SMILES: *CC1(*)CCO1]

optionally substituted as described herein.

In one embodiment the present invention provides a compound of formula (I), or a solvate or a pharmaceutically acceptable salt thereof, wherein the linker comprising R², R³, R⁴ and R⁵ has the following structure:

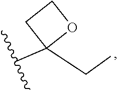 [SMILES: *C1(CC)CCO1]

optionally substituted as described herein.

In one embodiment the present invention provides a compound of formula (I) or a solvate, or a pharmaceutically acceptable salt thereof, wherein the heterocycloalkyl rings described above are substituted with two F.

The bonds linking the ring with the molecule are not part of the ring formed by R² and R³ and the carbon atom they are attached to.

In one embodiment the present invention provides a compound of formula (I), or a solvate, or a pharmaceutically acceptable salt thereof, wherein the heterocycloalkyl rings described above are substituted with one oxo.

In one embodiment the present invention provides a compound of formula (VII) with the formula:

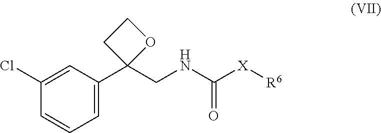

or a solvate, or a pharmaceutically acceptable salt thereof, wherein X and R⁶ are as described herein. The heterocycloalkyl ring can be substituted as described herein.

In one embodiment the present invention provides a compound of formula (VIII) with the formula:

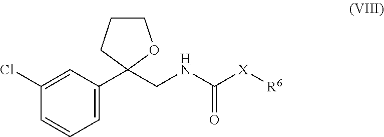

or a solvate, or a pharmaceutically acceptable salt thereof, wherein X and R⁶ are as described herein. The heterocycloalkyl ring can be substituted as described herein.

In one embodiment the present invention provides a compound of formula (I), or a solvate, or a pharmaceutically acceptable salt thereof, wherein R² and R³ together with the C-atom they are attached to form a 3-6 membered cycloalkyl, wherein such cycloalkyl is optionally substituted with 1 to 4 halogen or oxo.

In one embodiment the present invention provides a compound of formula (I), or a solvate, or a pharmaceutically acceptable salt thereof, wherein such cycloalkyl formed is selected from cyclopropyl, cyclobutyl, cyclopentyl, and cyclohexyl, which are optionally substituted as described above.

In one embodiment the present invention provides a compound of formula (I), or a solvate, or a pharmaceutically acceptable salt thereof, wherein such cycloalkyl formed is selected from cyclopropyl or cyclobutyl, which are optionally substituted as described above.

In one embodiment the present invention provides a compound of formula (I), or a solvate, or a pharmaceutically acceptable salt thereof, wherein such cycloalkyl formed is unsubstituted cyclopropyl and the linker has the structure:

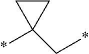 [SMILES: *CC1(*)CC1]

The bonds linking the ring with the molecule are not part of the ring formed by R² and R³ and the carbon atom they are attached to.

In one embodiment the present invention provides a compound of formula (IX) with the formula:

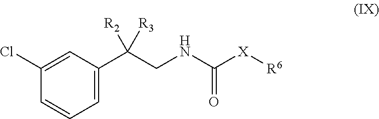

or a solvate, or a pharmaceutically acceptable salt thereof, wherein R², R³, X and R⁶ are as described herein.

In one embodiment the present invention provides a compound of formula (X) with the formula:

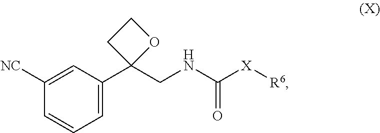

or a solvate, or a pharmaceutically acceptable salt thereof, wherein X and R⁶ are as described herein.

In one embodiment the present invention provides a compound of formula (XI) with the formula:

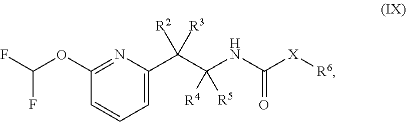

or a solvate, or a pharmaceutically acceptable salt thereof, wherein R², R³, R⁴, R⁵. X, and R⁶ are as described herein.

In one embodiment the present invention provides a compound of formula (XII) with the formula:

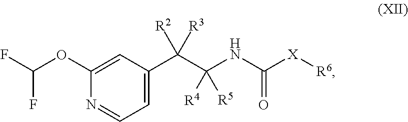

or a solvate, or a pharmaceutically acceptable salt thereof, wherein R², R³, R⁴, R⁵, X, and R⁶ are as described herein.

In one embodiment the present invention provides a compound of formula (XIII) with the formula:

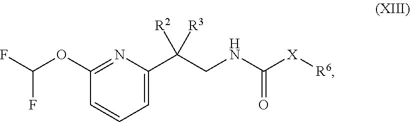

or a solvate, or a pharmaceutically acceptable salt thereof, wherein R², R³, X, and R⁶ are as described herein.

In one embodiment the present invention provides a compound of formula (XIV) with the formula:

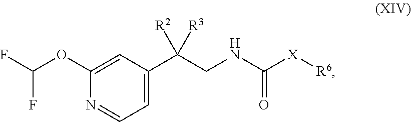

or a solvate, or a pharmaceutically acceptable salt thereof, wherein R², R³, X, and R⁶ are as described herein.

In one embodiment the present invention provides a compound of formula (I), or a solvate or a pharmaceutically acceptable salt thereof, wherein X is selected from: NH, CH₂, CH₂CH₂, CH₂CH₂CH₂, absent, and

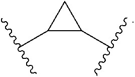 [SMILES: *C1CC1*]

In one embodiment the present invention provides a compound of formula (I), or a solvate, or a pharmaceutically acceptable salt thereof, wherein X is selected from: CH₂, CH₂CH₂, CH₂CH₂CH₂, absent, and

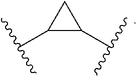 [SMILES: *C1CC1*]

In this application the group

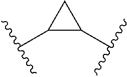 [SMILES: *C1CC1*]

for X means that X is cyclopropyl incorporated into the compound of formula (I) as shown in the exemplary compound with the structure:

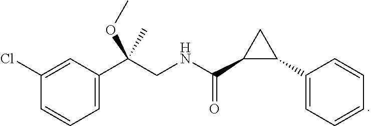 [SMILES: CO[C@@](C)(CNC(=O)[C@H]1C[C@@H]1c1ccccc1)c1cccc(Cl)c1]

In one embodiment the present invention provides a compound of formula (I), or a solvate, or a pharmaceutically acceptable salt thereof, wherein X is selected from: CH₂, CH₂CH₂, CH₂CH₂CH₂, absent, and

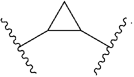 [SMILES: *C1CC1*]

In one embodiment the present invention provides a compound of formula (I), or a solvate, or a pharmaceutically acceptable salt thereof, wherein X is NH.

In one embodiment the present invention provides a compound of formula (I), or a solvate, or a pharmaceutically acceptable salt thereof, wherein X is CH2.

In one embodiment the present invention provides a compound of formula (I), or a solvate, or a pharmaceutically acceptable salt thereof, wherein X is absent.

In one embodiment the present invention provides a a compound of formula (I), or a solvate, or a pharmaceutically acceptable salt thereof, wherein X is

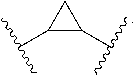 [SMILES: *C1CC1*]

In one embodiment the present invention provides a compound of formula (I), or a solvate, or a pharmaceutically acceptable salt thereof, wherein R⁶ is selected from C₁₋₆alkyl, haloC₁₋₆alkyl, heterocycloalkyl, and heteroaryl, wherein heterocycloalkyl and heteroaryl are optionally substituted with one, two, or three substituents independently selected from halogen, haloC₁₋₆alkyl, C₁₋₆alkyl, C₁₋₆alkoxy, and haloC₁₋₆alkoxy.

In one embodiment the present invention provides a compound of formula (I), or a solvate, or a pharmaceutically acceptable salt thereof, wherein R⁶ is selected from a 3-6 membered saturated monocyclic cycloalkyl, a 7-11 membered spiro-cycloalkyl, or phenyl, wherein saturated monocyclic cycloalkyl, spiro-cycloalkyl, or phenyl are optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate, or a pharmaceutically acceptable salt thereof, wherein R⁶ is a 7-11 membered spiro-cycloalkyl which is optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate, or a pharmaceutically acceptable salt thereof, wherein the spiro-cycloalkyl is unsubstituted.

In one preferred embodiment the present invention provides a compound of formula (I), or a solvate, or a pharmaceutically acceptable salt thereof, wherein the 7-11 membered spiro-cyloalkyl is a bicyclic spiro-cycloakyl comprising 7, 8, 9, 10, or 11 C-atoms with two saturated non-aromatic rings.

In one preferred embodiment the present invention provides a compound of formula (I), or a solvate, or a pharmaceutically acceptable salt thereof, wherein the 7-11 membered spiro-cyloalkyl is a tricyclic spiro-cycloakyl comprising 7, 8, 9, 10, or 11 C-atoms with two saturated non-aromatic rings.

In one preferred embodiment the present invention provides a compound of formula (I), or a solvate, or a pharmaceutically acceptable salt thereof, wherein the 7-11 membered spiro-cyloalkyl is a tricyclic spiro-cycloakyl comprising 9-11 C-atoms with three saturated non-aromatic rings with the structure:

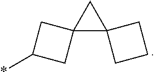 [SMILES: *C1CC2(C1)CC21CCC1]

In one preferred embodiment the present invention provides a compound of formula (I), or a solvate, or a pharmaceutically acceptable salt thereof, wherein the ring directly attached to the molecule is not a 4 membered ring.

In one preferred embodiment the present invention provides a compound of formula (I), or a solvate, or a pharmaceutically acceptable salt thereof, wherein the 7-11 membered spiro-cycloalkyl is selected from spiro[3.3]heptanyl, spiro[2.3]hexanyl, spiro[3.4]octanyl optionally substituted as described herein.

In one preferred embodiment the present invention provides a compound of formula (I), or a solvate, or a pharmaceutically acceptable salt thereof, wherein the R⁶ is a 7-11 membered spiro-cycloalkyl and X is absent.

In one preferred embodiment the present invention provides a compound of formula (I), or a solvate, or a pharmaceutically acceptable salt thereof, wherein the oxo-spiro-heterocycloalkyl is optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate, or a pharmaceutically acceptable salt thereof, wherein the oxo-spiro-heterocycloalkyl is unsubstituted.

In one preferred embodiment the present invention provides a compound of formula (I), or a solvate, or a pharmaceutically acceptable salt thereof, wherein the 7-11 membered oxo-spiro-heterocycloalkyl is a bicyclic oxa-spiro-heteroccyloalkyls comprising 7, 8, 9, 10, or 11 ring atoms, comprising two saturated monocyclic rings, wherein one rings comprise one O-atom.

In one preferred embodiment the present invention provides a compound of formula (I), or a solvate, or a pharmaceutically acceptable salt thereof, wherein the O-atom is in the ring directly attached to the molecule.

In one preferred embodiment the present invention provides a compound of formula (I), or a solvate, or a pharmaceutically acceptable salt thereof, wherein the ring directly attached to the molecule is not a 4 membered ring.

In one preferred embodiment the present invention provides a compound of formula (I), or a solvate, or a pharmaceutically acceptable salt thereof, wherein the 7-11 membered oxa-spiro-heterocycloalkyl is selected from oxaspiroheptanyl, oxaspirooctanyl, oxaspirononanyl, oxaspirodecanyl, and oxaspiroundecanyl.

In one preferred embodiment the present invention provides a compound of formula (I), or a solvate, or a pharmaceutically acceptable salt thereof, wherein the oxa-spiro-heterocycloalkyl comprises one O-atom to form a 9-10 membered oxa-spiro-heterocycloalkyl.

In one preferred embodiment the present invention provides a compound of formula (I), or a solvate, or a pharmaceutically acceptable salt thereof, wherein the 9-10 membered oxa-spiro-heterocycloalkyl is selected from oxaspirononanyl and oxaspirodecanyl.

In one preferred embodiment the present invention provides a compound of formula (I), or a solvate, or a pharmaceutically acceptable salt thereof, wherein the R⁶ is a 7-11 membered oxa-spiro-cycloalkyl and X is absent.

In one embodiment the present invention provides a compound of formula (I), or a solvate, or a pharmaceutically acceptable salt thereof, wherein R⁶ is a 3-10 membered cycloalkyl which cycloalkyl is optionally substituted with one, two, or three substituents independently selected from halogen, haloC₁₋₆alkyl, C₁₋₆alkyl, C₁₋₆alkoxy, and haloC₁₋₆alkoxy.

In one embodiment the present invention provides a compound of formula (I), or a solvate, or a pharmaceutically acceptable salt thereof, wherein R⁶ is a 3-10 membered cycloalkyl selected from a saturated monocyclic 4-6 membered cycloalkyl, a monocyclic 4-6 membered cycloalkenyl, and 4-10 membered bridged cycloalkyl, which are optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate or a pharmaceutically acceptable salt thereof, wherein R⁶ is a saturated monocyclic 4-6 membered cycloalkyl, which cycloalkyl is optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate or a pharmaceutically acceptable salt thereof, wherein R⁶ is a saturated monocyclic 4-6 membered cycloalkyl, which saturated monocyclic cycloalkyl is optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment the present invention provides a compound of formula (I), or a solvate or a pharmaceutically acceptable salt thereof, wherein R⁶ is a saturated monocyclic 4-6 membered cycloalkyl, which saturated monocyclic cycloalkyl is optionally substituted with one or two halogen.

In one preferred embodiment the present invention provides a compound of formula (I), or a solvate or a pharmaceutically acceptable salt thereof, wherein R⁶ is a saturated monocyclic 4-6 membered cycloalkyl, which saturated monocyclic cycloalkyl is unsubstituted.

In one preferred embodiment the present invention provides a compound of formula (I), or a solvate or a pharmaceutically acceptable salt thereof, wherein R⁶ is a saturated monocyclic 4-6 membered cycloalkyl, which saturated monocyclic cycloalkyl is optionally substituted with one halogen.

In one embodiment, the present invention provides a compound of formula (I), or a solvate or a pharmaceutically acceptable salt thereof, wherein R⁶ is a saturated monocyclic 4-6 membered cycloalkyl, which saturated monocyclic cycloalkyl is optionally substituted with one F.

In one embodiment, the present invention provides a compound of formula (I), or a solvate or a pharmaceutically acceptable salt thereof, wherein R⁶ is a 4-10 membered bridged cycloalkyl, which is optionally substituted as described herein.

In one embodiment, the present invention provides a compound of formula (I), or a solvate or a pharmaceutically acceptable salt thereof, wherein R⁶ is an unsubstituted 4-10 membered bridged cycloalkyl, which is cycloalkyl is optionally substituted with one, two, or three substituents independently selected from halogen, haloC₁₋₆alkyl, C₁₋₆alkyl, C₁₋₆alkoxy, and haloC₁₋₆alkoxy.

In one embodiment, the present invention provides a compound of formula (I), or a solvate or a pharmaceutically acceptable salt thereof, wherein R⁶ is unsubstituted bicyclo[2.2.1]heptanyl.

In one embodiment, the present invention provides a compound of formula (I), or a solvate or a pharmaceutically acceptable salt thereof, wherein R⁶ is a monocyclic 4-6 membered cycloalkenyl with one non-aromatic double bond, which is optionally substituted with one, two, or three substituents independently selected from halogen, haloC₁₋₆alkyl, C₁₋₆alkyl, C₁₋₆alkoxy, and haloC₁₋₆alkoxy.

In one embodiment, the present invention provides a compound of formula (I), or a solvate or a pharmaceutically acceptable salt thereof, wherein R⁶ is an unsubstituted monocyclic 4-6 membered cycloalkenyl with one non-aromatic double bond.

In one preferred embodiment, the present invention provides a compound of formula (I), or a solvate or a pharmaceutically acceptable salt thereof, wherein R⁶ is phenyl which is optionally substituted with one, two, or three substituents independently selected from halogen, haloC₁₋₆alkyl, C₁₋₆alkyl, C₁₋₆alkoxy, and haloC₁₋₆alkoxy.

In one preferred embodiment, the present invention provides a compound of formula (I), or a solvate or a pharmaceutically acceptable salt thereof, wherein R⁶ is unsubstituted phenyl.

In one preferred embodiment, the present invention provides a compound of formula (I), or a solvate or a pharmaceutically acceptable salt thereof, wherein R⁶ is phenyl substituted with C₁.

In one preferred embodiment the present invention provides a compound of formula (I), or a solvate, or a pharmaceutically acceptable salt thereof:

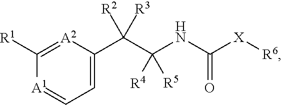

wherein
A¹ is CR^{a};
A² is CR^{b};
R^{a} is hydrogen;
R^{b} is hydrogen;
R¹ is selected from C₁, cyano, and CHF₂O—;
R² and R³ are C₁₋₆alkyl, C₁₋₆alkoxy, or R² and R³ together with the carbon atom they are attached to form a 4-6 membered heterocycloalkyl, wherein such heterocycloalkyl is optionally substituted with one or two F— or oxo;
R⁴ and R⁵ are hydrogen;
X is selected from

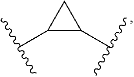 [SMILES: *C1CC1*]

NHCH₂, absent, CH₂, and CH₂CH₂; and
   R⁶ is a 3-6 membered a saturated monocyclic cycloalkyl, a 7-11 membered spiro-cycloalkyl, or phenyl, wherein saturated monocyclic cycloalkyl, spiro-cycloalkyl, or phenyl are optionally substituted with one, two, or three substituents independently selected from halogen, haloC₁₋₆alkyl, C₁₋₆alkyl, C₁₋₆alkoxy, and haloC₁₋₆alkoxy.

In one embodiment, the application provides compounds with the following formulaes:

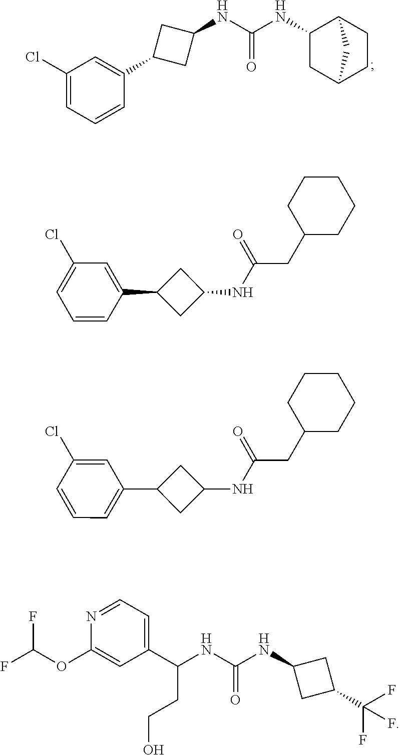 [SMILES: O=C(N[C@H]1C[C@@H]2CC[C@H]1C2)N[C@H]1C[C@H](c2cccc(Cl)c2)C1 ; O=C(CC1CCCCC1)N[C@H]1C[C@H](c2cccc(Cl)c2)C1 ; O=C(CC1CCCCC1)NC1CC(c2cccc(Cl)c2)C1 ; O=C(NC(CCO)c1ccnc(OC(F)F)c1)N[C@H]1C[C@H](C(F)(F)F)C1]

In one preferred embodiment the present invention provides a compound with the structure

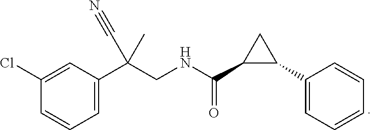 [SMILES: CC(C#N)(CNC(=O)[C@H]1C[C@@H]1c1ccccc1)c1cccc(Cl)c1]

In one embodiment the present invention provides a compound of formula (I), or a solvate or a pharmaceutically acceptable salt thereof as described herein, wherein such compounds of formula (I) show Kv7.2 EC50 values and/or Kv7.5_7.3/Kv7.2 selectivity ratios in the range of: (i) EC50<3 μM, Select>10×, or (ii) EC50<1 μM, Select>30×.

All of the features disclosed in this specification (including any accompanying claims, abstract and drawings) relating to any embodiment so disclosed, may be combined in any combination, except combinations where at least some of such features and/or steps are mutually exclusive. The present invention is not restricted to the details of any embodiments described herein. Any embodiment described in this application can be combined with any other embodiment.

Exemplary formula (I) compounds in Table 1 were made, characterized, and tested for activation of Kv7.2 (EC₅₀ less than 10 micromolar, μM) and Kv7.2 activation max % according to the methods of this invention, and have the following structures and corresponding names (OpenEve Lexichem, Version 1.2.0, OpenEye Scientific Software, Santa Fe, NM, USA; https://www.eyesopen.com/lexichem-tk).

In some embodiments, the compound is selected from Table 1, or a solvate or a pharmaceutically acceptable salt thereof.

TABLE 1

| | | | Kv7.2 |
|---|---|---|---|
| | | | EC₅₀ |
| Example | Name | Structure | (μM) |
| 1 | rac-(1S,2S)-N-[2-(3- chlorophenyl)-2-methoxy- propyl]-2-phenyl- cyclopropanecarboxamide | 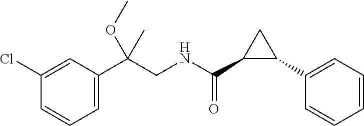 [SMILES: COC(C)(CNC(=O)[C@H]1C[C@@H]1c1ccccc1)c1cccc(Cl)c1] | 0.17 |
| 2 | (1S,2S)-N-[(2R)-2-(3- chlorophenyl)-2-methoxy- propyl]-2-phenyl- cyclopropanecarboxamide | 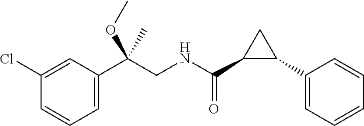 [SMILES: CO[C@@](C)(CNC(=O)[C@H]1C[C@@H]1c1ccccc1)c1cccc(Cl)c1] | 0.06 |
| 3 | 1-[2-(3-chlorophenyl)-2- methoxy-propyl]-3-cyclopentyl- urea | 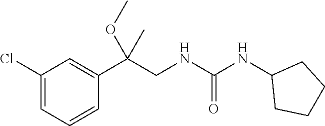 [SMILES: COC(C)(CNC(=O)NC1CCCC1)c1cccc(Cl)c1] | 2.34 |
| 4 | 1-[2-(3-chlorophenyl)-2- methoxy-ethyl]-3-norbornan-2- yl-urea | 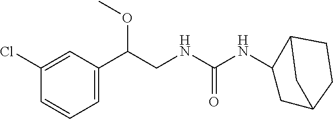 [SMILES: COC(CNC(=O)NC1CC2CCC1C2)c1cccc(Cl)c1] | 0.50 |
| 5 | 1-[2-(3-chlorophenyl)-2- methoxy-propyl]-3-(3- methoxyphenyl)urea | 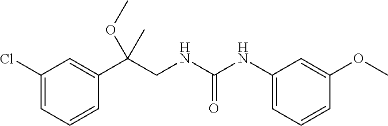 [SMILES: COc1cccc(NC(=O)NCC(C)(OC)c2cccc(Cl)c2)c1] | 1.04 |
| 6 | 1-[2-(3-chlorophenyl)-2- methoxy-propyl]-3-(m- tolyl)urea | 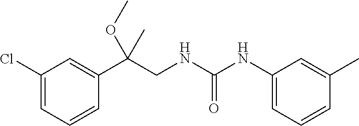 [SMILES: COC(C)(CNC(=O)Nc1cccc(C)c1)c1cccc(Cl)c1] | 2.41 |
| 7 | 1-[[1-(3- chlorophenyl)cyclopropyl]meth- yl]-3-cyclopentyl-urea | 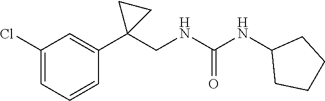 [SMILES: O=C(NCC1(c2cccc(Cl)c2)CC1)NC1CCCC1] | 2.72 |
| 8 | 1-butyl-3-[2-(3-chlorophenyl)- 2-methoxy-propyl]urea | 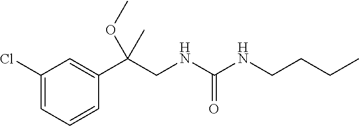 [SMILES: CCCCNC(=O)NCC(C)(OC)c1cccc(Cl)c1] | 3.65 |
| 9 | 1-[2-(3-chlorophenyl)-2- methoxy-propyl]-3-(2- furylmethyl)urea | 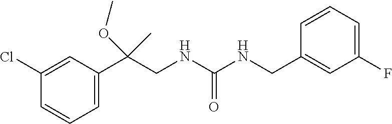 [SMILES: COC(C)(CNC(=O)NCc1cccc(F)c1)c1cccc(Cl)c1] | 2.46 |
| 10 | 1-[2-(3-chlorophenyl)-2- methoxy-propyl]-3-[(3- fluorophenyl)methyl]urea | 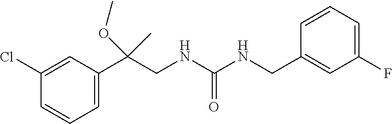 [SMILES: COC(C)(CNC(=O)NCc1cccc(F)c1)c1cccc(Cl)c1] | 0.76 |
| 11 | 1-[2-(3-chlorophenyl)-2- methoxy-propyl]-3-(m- tolylmethyl)urea | 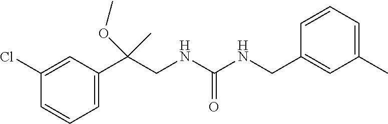 [SMILES: COC(C)(CNC(=O)NCc1cccc(C)c1)c1cccc(Cl)c1] | 1.43 |
| 12 | 1-[[1-(3- chlorophenyl)cyclopropyl]meth- yl]-3-(1-oxaspiro[4.4]nonan-3- yl)urea | 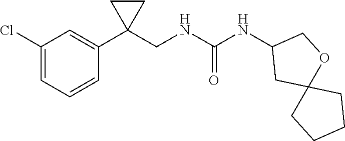 [SMILES: O=C(NCC1(c2cccc(Cl)c2)CC1)NC1COC2(CCCC2)C1] | 2.92 |
| 13 | 1-[2-(3-chlorophenyl)-2- methoxy-propyl]-3-(1- oxaspiro[4.4]nonan-3-yl)urea | 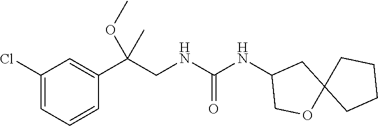 [SMILES: COC(C)(CNC(=O)NC1COC2(CCCC2)C1)c1cccc(Cl)c1] | 3.55 |
| 14 | 1-[2-(3-chlorophenyl)-2- methoxy-propyl]-3-(3- ethoxyphenyl)urea | 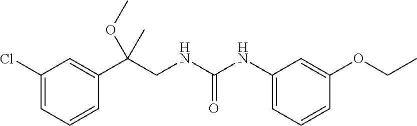 [SMILES: CCOc1cccc(NC(=O)NCC(C)(OC)c2cccc(Cl)c2)c1] | 0.60 |
| 15 | 1-[[1-(3- chlorophenyl)cyclopropyl]meth- yl]-3-(tetrahydropyran-2- ylmethyl)urea | 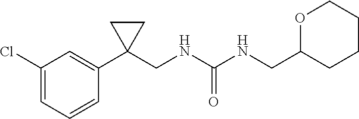 [SMILES: O=C(NCC1CCCCO1)NCC1(c2cccc(Cl)c2)CC1] | 6.92 |
| 16 | 1-[(2R)-2-(3-chlorophenyl)-2- methoxy-propyl]-3-cyclopentyl- urea | 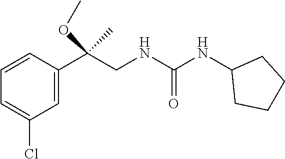 [SMILES: CO[C@@](C)(CNC(=O)NC1CCCC1)c1cccc(Cl)c1] | 2.87 |
| 17 | 1-[(2R)-2-(3-chlorophenyl)-2- methoxy-propyl]-3-(3- methoxyphenyl)urea | 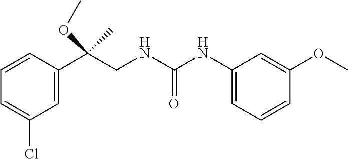 [SMILES: COc1cccc(NC(=O)NC[C@](C)(OC)c2cccc(Cl)c2)c1] | 1.67 |
| 18 | 1-[2-(3-chlorophenyl)-2- (cyclopropoxy)ethyl]-3- cyclopentyl-urea | 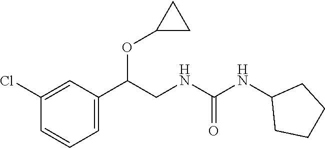 [SMILES: O=C(NCC(OC1CC1)c1cccc(Cl)c1)NC1CCCC1] | 4.58 |
| 19 | N-[2-(3-chlorophenyl)-2- methoxy-propyl]-2-cyclohexyl- acetamide | 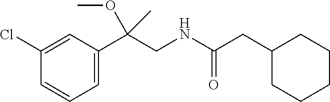 [SMILES: COC(C)(CNC(=O)CC1CCCCC1)c1cccc(Cl)c1] | 0.62 |
| 20 | rac-(1R,2R)-N-[2-(3- chlorophenyl)-2-methoxy- ethyl]-2-phenyl- cyclopropanecarboxamide | 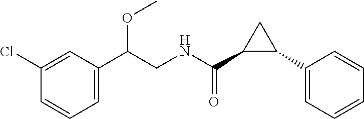 [SMILES: COC(CNC(=O)[C@H]1C[C@@H]1c1ccccc1)c1cccc(Cl)c1] | 0.97 |
| 21 | 1-[2-(3-chlorophenyl)-2- methoxy-propyl]-3- (cyclopentylmethyl)urea | 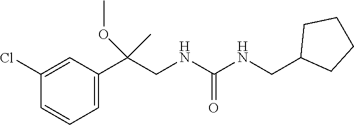 [SMILES: COC(C)(CNC(=O)NCC1CCCC1)c1cccc(Cl)c1] | 0.55 |
| 22 | 1-[2-(3-chlorophenyl)-2- methoxy-ethyl]-3-(m-tolyl)urea | 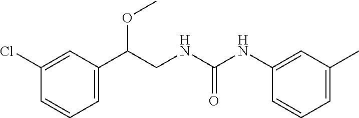 [SMILES: COC(CNC(=O)Nc1cccc(C)c1)c1cccc(Cl)c1] | 1.99 |
| 23 | 1-[2-(3-chlorophenyl)-2- methoxy-propyl]-3- (cyclopropylmethyl)urea | 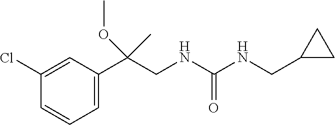 [SMILES: COC(C)(CNC(=O)NCC1CC1)c1cccc(Cl)c1] | 8.20 |
| 24 | 1-[2-(3-bromophenyl)-2- isopropoxy-ethyl]-3- cyclopentyl-urea | 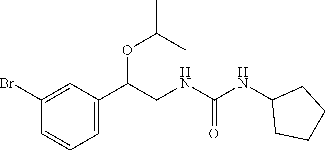 [SMILES: CC(C)OC(CNC(=O)NC1CCCC1)c1cccc(Br)c1] | 2.92 |
| 25 | 1-[2-(3-chlorophenyl)-2- methoxy-propyl]-3-[rac- (1S,2S,4R)-norbornan-2-yl]urea | 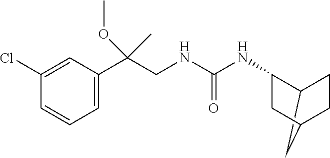 [SMILES: COC(C)(CNC(=O)N[C@H]1CC2CCC1C2)c1cccc(Cl)c1] | 0.17 |
| 26 | 1-[2-(3-chlorophenyl)-2- (trideuteriomethoxy)propyl]-3- cyclopentyl-urea | 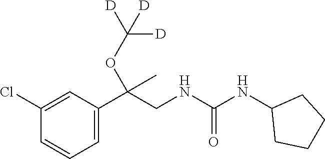 [SMILES: [2H]C([2H])([2H])OC(C)(CNC(=O)NC1CCCC1)c1cccc(Cl)c1] | 4.01 |
| 27 | 1-(3-methoxyphenyl)-3-[[2-(m- tolyl)-1,3-dioxolan-2- yl]methyl]urea | 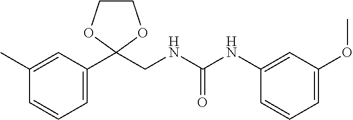 | 3.27 |
| 28 | 1-[2-(3-bromophenyl)-2- ethoxy-ethyl]-3-cyclopentyl- urea | 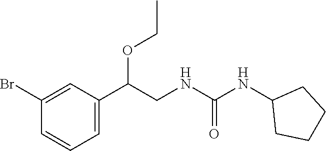 [SMILES: CCOC(CNC(=O)NC1CCCC1)c1cccc(Br)c1] | 2.58 |
| 29 | 1-[2-(3-chlorophenyl)-2- methoxy-propyl]-3-[3- (trifluoromethoxy)phenyl]urea | 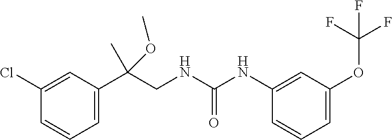 [SMILES: COC(C)(CNC(=O)Nc1cccc(OC(F)(F)F)c1)c1cccc(Cl)c1] | 5.69 |
| 30 | 1-[2-(3-chlorophenyl)-2- methoxy-propyl]-3-cyclopent- 3-en-1-yl-urea | 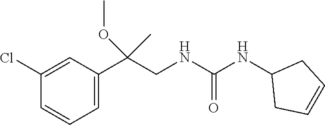 [SMILES: COC(C)(CNC(=O)NC1CC=CC1)c1cccc(Cl)c1] | 3.27 |
| 31 | rac-(1S,2S)-N-[2-cyano-2-[3- (trifluoromethyl)phenyl]propyl]- 2-phenyl- cyclopropanecarboxamide | 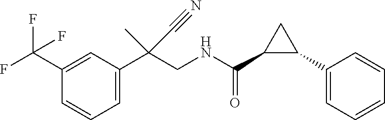 [SMILES: CC(C#N)(CNC(=O)[C@H]1C[C@@H]1c1ccccc1)c1cccc(C(F)(F)F)c1] | 2.49 |
| 32 | 1-[2-(3-chlorophenyl)-2- methoxy-propyl]-3-(3- phenylpropyl)urea | 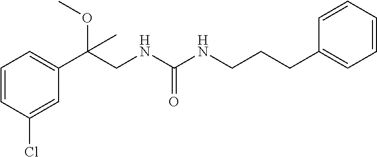 [SMILES: COC(C)(CNC(=O)NCCCc1ccccc1)c1cccc(Cl)c1] | 1.98 |
| 33 | 1-[2-(3-chlorophenyl)-2- methoxy-propyl]-3-(2- thienylmethyl)urea | 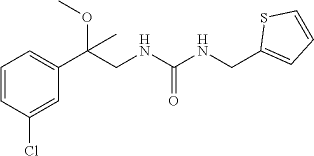 [SMILES: COC(C)(CNC(=O)NCc1cccs1)c1cccc(Cl)c1] | 1.86 |
| 34 | 1-[2-(3-chlorophenyl)-2- methoxy-propyl]-3-[2-(4- methoxyphenyl)ethyl]urea | 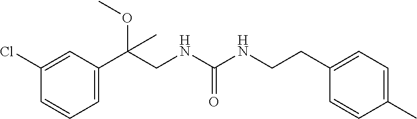 [SMILES: COC(C)(CNC(=O)NCCc1ccc(C)cc1)c1cccc(Cl)c1] | 3.94 |
| 35 | 1-[2-(3-chlorophenyl)-2- methoxy-propyl]-3-[rac- (1S,4R)-norbornan-2-yl]urea | 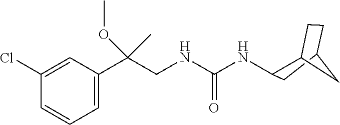 [SMILES: COC(C)(CNC(=O)NC1CC2CCC1C2)c1cccc(Cl)c1] | 1.35 |
| 36 | N-[2-(2-chlorophenyl)-2- methoxy-ethyl]-2-(1- naphthyl)acetamide | 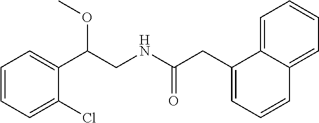 [SMILES: COC(CNC(=O)Cc1cccc2ccccc12)c1ccccc1Cl] | 1.58 |
| 37 | rac-(1S,2S)-N-[2-(3- chlorophenyl)-2-cyano-propyl]- 2-phenyl- cyclopropanecarboxamide | 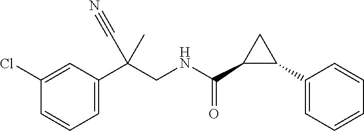 [SMILES: CC(C#N)(CNC(=O)[C@H]1C[C@@H]1c1ccccc1)c1cccc(Cl)c1] | 1.32 |
| 38 | 1-[3-(1r,3r)-(3- chlorophenyl)cyclobutyl]-3- [(1SR,2SR,4RS)-norbornan-2- yl]urea | 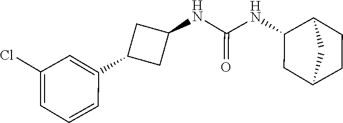 [SMILES: O=C(N[C@H]1C[C@@H]2CC[C@H]1C2)N[C@H]1C[C@H](c2cccc(Cl)c2)C1] | 0.83 |
| 39 | (1S,2S)-N-[[(2R)-2-(3- chlorophenyl)oxetan-2- yl]methyl]-2-phenyl- cyclopropanecarboxamide | 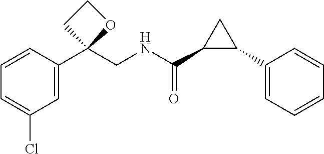 [SMILES: O=C(NC[C@]1(c2cccc(Cl)c2)CCO1)[C@H]1C[C@@H]1c1ccccc1] | 0.73 |
| 40 | (1S,2S)-N-[[(2S)-2-(3- chlorophenyl)oxetan-2- yl]methyl]-2-phenyl- cyclopropanecarboxamide | 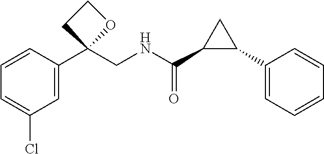 [SMILES: O=C(NC[C@@]1(c2cccc(Cl)c2)CCO1)[C@H]1C[C@@H]1c1ccccc1] | 0.11 |
| 41 | (1S,2S)-N-[[(2R)-2-(3- chlorophenyl)-3,3-difluoro- oxetan-2-yl]methyl]-2-phenyl- cyclopropanecarboxamide | 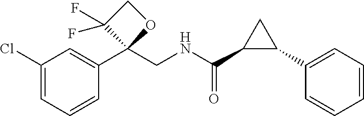 [SMILES: O=C(NC[C@@]1(c2cccc(Cl)c2)OCC1(F)F)[C@H]1C[C@@H]1c1ccccc1] | 0.05 |
| 42 | (1S,2S)-N-[[(2S)-2-(3- chlorophenyl)-3,3-difluoro- oxetan-2-yl]methyl]-2-phenyl- cyclopropanecarboxamide | 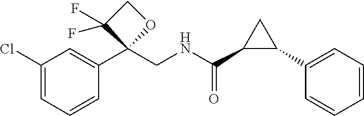 [SMILES: O=C(NC[C@]1(c2cccc(Cl)c2)OCC1(F)F)[C@H]1C[C@@H]1c1ccccc1] | 0.63 |
| 43 | N-[2-(3-chlorophenyl)-2- methoxy-propyl]-2-(2- fluorophenyl)cyclopropanecarbox- amide | 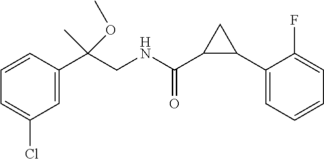 [SMILES: COC(C)(CNC(=O)C1CC1c1ccccc1F)c1cccc(Cl)c1] | 0.11 |
| 44 | 2-(4-chlorophenyl)-N-[2-(3- chlorophenyl)-2-methoxy- propyl]cyclopropanecarboxamide | 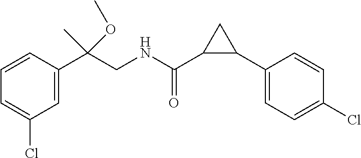 [SMILES: COC(C)(CNC(=O)C1CC1c1ccc(Cl)cc1)c1cccc(Cl)c1] | 0.90 |
| 45 | 1-[2-(3-chlorophenyl)-2-(2,2,2- trifluoroethoxy)ethyl]-3-[rac- (1R,4S)-norbornan-2-yl]urea | 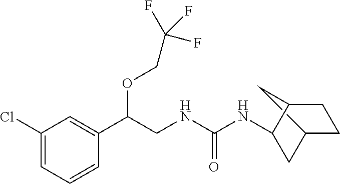 [SMILES: O=C(NCC(OCC(F)(F)F)c1cccc(Cl)c1)NC1CC2CCC1CC2] | 0.79 |
| 46 | 1-[2-(3-chlorophenyl)-2- (cyclobutoxy)ethyl]-3-[rac- (1R,4S)-norbornan-2-yl]urea | 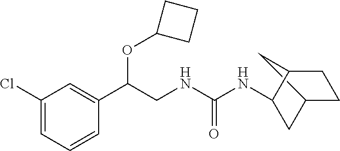 [SMILES: O=C(NCC(OC1CCC1)c1cccc(Cl)c1)NC1CC2CCC1CC2] | 3.35 |
| 47 | (E)-N-[2-(3-chlorophenyl)-2- methoxy-propyl]-3-(2- fluorophenyl)prop-2-enamide | 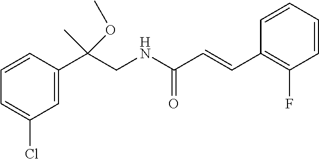 [SMILES: COC(C)(CNC(=O)/C=C/c1ccccc1F)c1cccc(Cl)c1] | 7.51 |
| 48 | (1SR,2SR)-N-[(2R)-2-(3- chlorophenyl)-2-methoxy- propyl]-2-(3- pyridyl)cyclopropanecarboxamide | 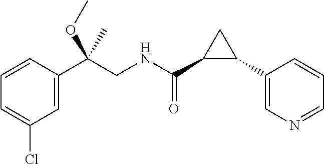 [SMILES: CO[C@@](C)(CNC(=O)[C@H]1C[C@@H]1c1cccnc1)c1cccc(Cl)c1] | 9.61 |
| 49 | (1S,2S)-N-[[(2S)-2-(3- cyanophenyl)oxetan-2- yl]methyl]-2-phenyl- cyclopropanecarboxamide | 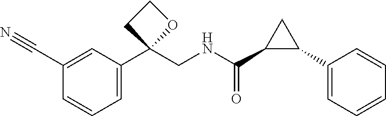 [SMILES: N#Cc1cccc([C@]2(CNC(=O)[C@H]3C[C@@H]3c3ccccc3)CCO2)c1] | 0.04 |
| 50 | 1-[[2-(3-chlorophenyl)oxetan-2- yl]methyl]-3-(3- methoxyphenyl)urea | 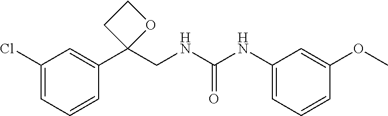 [SMILES: COc1cccc(NC(=O)NCC2(c3cccc(Cl)c3)CCO2)c1] | 1.65 |
| 51 | (1S,2S)-N-[(2RS)-2-(2-chloro-4- pyridyl)-2-methoxy-propyl]-2- phenyl- cyclopropanecarboxamide | 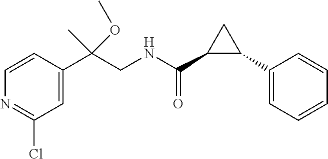 [SMILES: COC(C)(CNC(=O)[C@H]1C[C@@H]1c1ccccc1)c1ccnc(Cl)c1] | 0.94 |
| 52 | (1S,2S)-N-[(2RS)-2-methoxy-2- (2-methoxy-4-pyridyl)propyl]- 2-phenyl- cyclopropanecarboxamide | 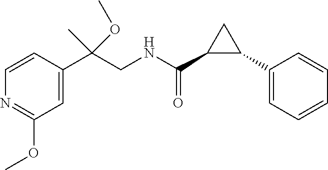 [SMILES: COc1cc(C(C)(CNC(=O)[C@H]2C[C@@H]2c2ccccc2)OC)ccn1] | 9.42 |
| 53 | N-[2-(3-chlorophenyl)-2- methoxy-propyl]-3-cyclopentyl- propanamide | 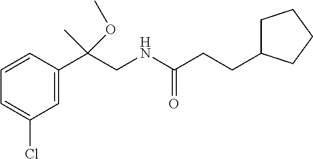 [SMILES: COC(C)(CNC(=O)CCC1CCCC1)c1cccc(Cl)c1] | 0.89 |
| 54 | 1-[[2-(3-chlorophenyl)oxetan-2- yl]methyl]-3-[rac-(1S,2S,4R)- norbornan-2-yl]urea | 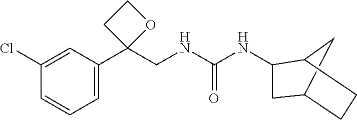 [SMILES: O=C(NCC1(c2cccc(Cl)c2)CCO1)NC1CC2CCC1C2] | 0.19 |
| 55 | (1S,2S)-N-[[(2R)-2-(3- cyanophenyl)oxetan-2- yl]methyl]-2-phenyl- cyclopropanecarboxamide | 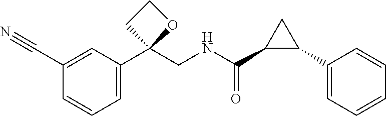 [SMILES: N#Cc1cccc([C@@]2(CNC(=O)[C@H]3C[C@@H]3c3ccccc3)CCO2)c1] | 0.79 |
| 56 | N-[3-(1r,3r)-(3- chlorophenyl)cyclobutyl]-2- cyclohexyl-acetamide | 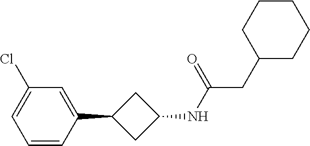 [SMILES: O=C(CC1CCCCC1)N[C@H]1C[C@H](c2cccc(Cl)c2)C1] | 3.19 |
| 57 | N-[[2-(3-chlorophenyl)oxetan- 2-yl]methyl]-2-cyclohexyl- acetamide | 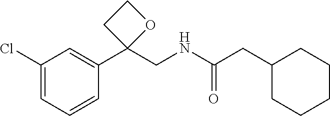 [SMILES: O=C(CC1CCCCC1)NCC1(c2cccc(Cl)c2)CCO1] | 0.43 |
| 58 | 1-(3-chlorophenyl)-3-[2-(3- chlorophenyl)oxetan-2- yl]methyl]urea | 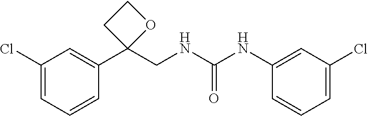 [SMILES: O=C(NCC1(c2cccc(Cl)c2)CCO1)Nc1cccc(Cl)c1] | 0.81 |
| 59 | 1-[[2-(3-chlorophenyl)oxetan-2- yl]methyl]-3- (cyclopentylmethyl)urea | 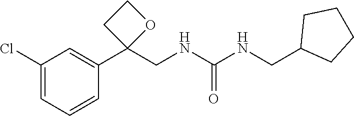 [SMILES: O=C(NCC1CCCC1)NCC1(c2cccc(Cl)c2)CCO1] | 0.32 |
| 60 | 1-[[2-(3-chlorophenyl)oxetan-2- yl]methyl]-3-(2,2- dimethylpropyl)urea | 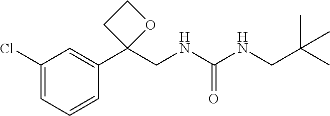 [SMILES: CC(C)(C)CNC(=O)NCC1(c2cccc(Cl)c2)CCO1] | 1.05 |
| 61 | N-112-(3-chlorophenyl)oxetan- 2- yl]methyl]cyclohexanecarboxa- mide | 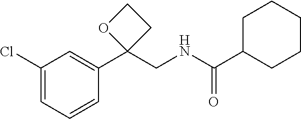 [SMILES: O=C(NCC1(c2cccc(Cl)c2)CCO1)C1CCCCC1] | 7.60 |
| 62 | 1-[(3-chlorophenyl)methyl]-3- [[2-(3-chlorophenyl)oxetan-2- yl]methyl]urea | 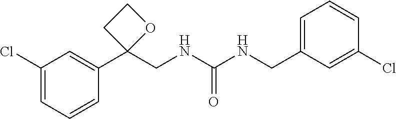 [SMILES: O=C(NCc1cccc(Cl)c1)NCC1(c2cccc(Cl)c2)CCO1] | 0.42 |
| 63 | 2-(1-bicyclo[1.1.1]pentanyl)-N- [2-(3-chlorophenyl)oxetan-2- yl]methyl]acetamide | 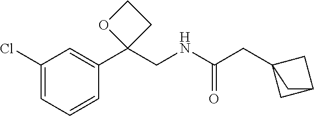 [SMILES: O=C(CC12CC(C1)C2)NCC1(c2cccc(Cl)c2)CCO1] | 5.19 |
| 64 | N-[[2-(3-chlorophenyl)oxetan- 2-yl]methyl]spiro[3.3]heptane- 2-carboxamide | 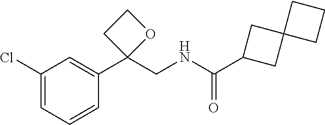 [SMILES: O=C(NCC1(c2cccc(Cl)c2)CCO1)C1CC2(CCC2)C1] | 2.75 |
| 65 | N-[[2-(3-chlorophenyl)oxetan- 2-yl]methyl]-2-(4,4- difluorocyclohexyl)acetamide | 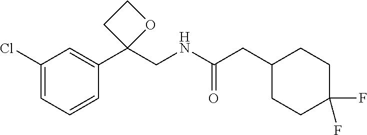 [SMILES: O=C(CC1CCC(F)(F)CC1)NCC1(c2cccc(Cl)c2)CCO1] | 1.92 |
| 66 | (1S,2S)-N-[[2-(3-chlorophenyl)- 5-oxo-tetrahydrofuran-2- yl]methyl]-2-phenyl- cyclopropanecarboxamide | 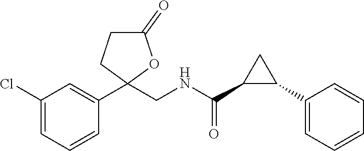 [SMILES: O=C1CCC(CNC(=O)[C@H]2C[C@@H]2c2ccccc2)(c2cccc(Cl)c2)O1] | 0.08 |
| 67 | (1S,2S)-N-[[2-[2- (difluoromethoxy)-4- pyridyl]oxetan-2-yl]methyl]-2- phenyl- cyclopropanecarboxamide | 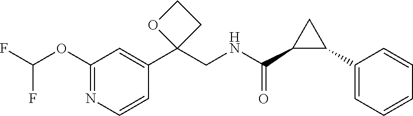 [SMILES: O=C(NCC1(c2ccnc(OC(F)F)c2)CCO1)[C@H]1C[C@@H]1c1ccccc1] | 0.41 |
| 68 | (1S,2S)-N-[[3-(3- chlorophenyl)oxetan-3- yl]methyl]-2-phenyl- cyclopropanecarboxamide | 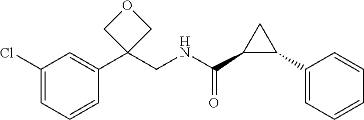 [SMILES: O=C(NCC1(c2cccc(Cl)c2)COC1)[C@H]1C[C@@H]1c1ccccc1] | 0.20 |
| 69 | N-[(2R)-2-(3-chlorophenyl)-2- methoxy-propyl]-4-hydroxy- 5,5-dimethyl-hexanamide | 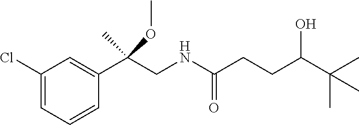 [SMILES: CO[C@@](C)(CNC(=O)CCC(O)C(C)(C)C)c1cccc(Cl)c1] | 6.29 |
| 70 | (1S,2S)-N-[(2S)-2-(3- chlorophenyl)-2- (cyanomethoxy)ethyl]-2- phenyl- cyclopropanecarboxamide | 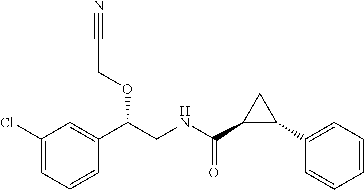 [SMILES: N#CCO[C@H](CNC(=O)[C@H]1C[C@@H]1c1ccccc1)c1cccc(Cl)c1] | 0.65 |
| 71 | 1-(cyclopentylmethyl)-3-[rel- (2S)-2-(3-chlorophenyl)-2- methoxy-propyl]urea | 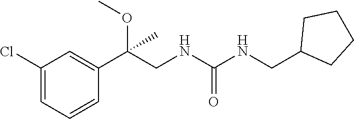 [SMILES: CO[C@@](C)(CNC(=O)NCC1CCCC1)c1cccc(Cl)c1] | 0.53 |
| 72 | 1-[[3-(3-chlorophenyl)oxetan-3- yl]methyl]-3- (cyclopentylmethyl)urea | 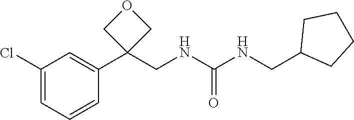 [SMILES: O=C(NCC1CCCC1)NCC1(c2cccc(Cl)c2)COC1] | 0.87 |
| 73 | N-[2-(3-chlorophenyl)-2- (cyanomethoxy)ethyl]-2- cyclopentyl-acetamide | 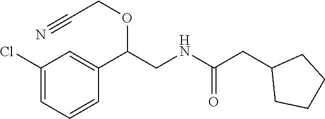 [SMILES: N#CCOC(CNC(=O)CC1CCCC1)c1cccc(Cl)c1] | 2.00 |
| 74 | (1S,2S)-N-[2-(3-chlorophenyl)- 3-hydroxy-propyl]-2-phenyl- cyclopropanecarboxamide | 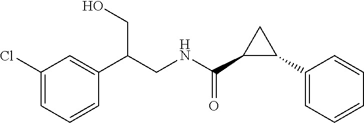 [SMILES: O=C(NCC(CO)c1cccc(Cl)c1)[C@H]1C[C@@H]1c1ccccc1] | 3.10 |
| 75 | 1-[[2-(3-chlorophenyl)-5-oxo- tetrahydrofuran-2-yl]methyl]-3- (cyclopentylmethyl)urea | 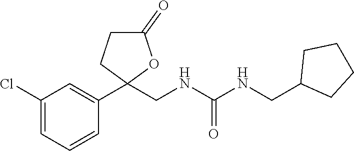 [SMILES: O=C(NCC1CCCC1)NCC1(c2cccc(Cl)c2)CCC(=O)O1] | 0.29 |
| 76 | (1S,2S)-N-[[2-(6-chloro-2- pyridyl)oxetan-2-yl]methyl]-2- phenyl- cyclopropanecarboxamide | 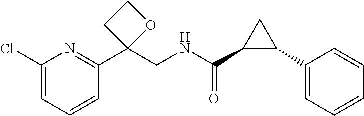 [SMILES: O=C(NCC1(c2cccc(Cl)n2)CCO1)[C@H]1C[C@@H]1c1ccccc1] | 1.74 |
| 77 | (1S,2S)-N-[(E)-2-(3- chlorophenyl)-4-hydroxy-but-2- enyl]-2-phenyl- cyclopropanecarboxamide | 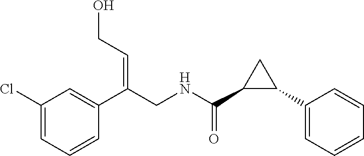 [SMILES: O=C(NC/C(=C/CO)c1cccc(Cl)c1)[C@H]1C[C@@H]1c1ccccc1] | 0.34 |
| 78 | 1-[(E)-2-(3-chlorophenyl)-4- hydroxy-but-2-enyl]-3- (cyclopentylmethyl)urea | 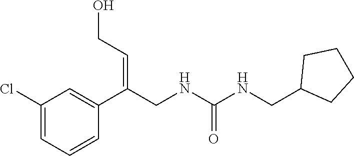 [SMILES: O=C(NC/C(=C/CO)c1cccc(Cl)c1)NCC1CCCC1] | 6.79 |
| 79 | (1RS,2SR)-N-[(2R)-2-(3- chlorophenyl)-2-methoxy- propyl]-2-cyclopropyl- cyclopropanecarboxamide | 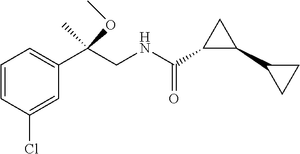 [SMILES: CO[C@@](C)(CNC(=O)[C@@H]1C[C@H]1C1CC1)c1cccc(Cl)c1] | 1.12 |
| 80 | (1RS,2SR)-N-[(2R)-2-(3- chlorophenyl)-2-methoxy- propyl]-2-cyclopentyl- cyclopropanecarboxamide | 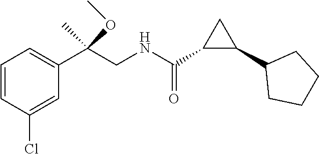 [SMILES: CO[C@@](C)(CNC(=O)[C@@H]1C[C@H]1C1CCCC1)c1cccc(Cl)c1] | 0.22 |
| 81 | (1SR,2SR)-N-[(2R)-2-(3- chlorophenyl)-2-methoxy- propyl]-2-cyclopentyl- cyclopropanecarboxamide | 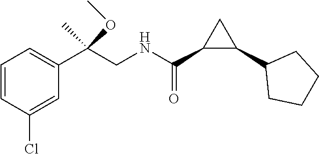 [SMILES: CO[C@@](C)(CNC(=O)[C@H]1C[C@H]1C1CCCC1)c1cccc(Cl)c1] | 0.50 |
| 82 | N-[(2R)-2-(3-chlorophenyl)-2- methoxy- propyl]spiro[2.5]octane-2- carboxamide | 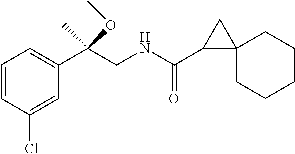 [SMILES: CO[C@@](C)(CNC(=O)C1CC12CCCCC2)c1cccc(Cl)c1] | 0.29 |
| 83 | N-[(2R)-2-(3-chlorophenyl)-2- methoxy-propyl]-6,6-difluoro- spiro[2.5]octane-2-carboxamide | 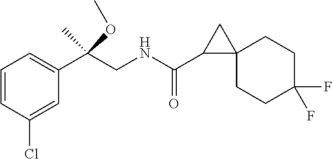 [SMILES: CO[C@@](C)(CNC(=O)C1CC12CCC(F)(F)CC2)c1cccc(Cl)c1] | 0.38 |
| 84 | N-[(2R)-2-(3-chlorophenyl)-2- methoxy-propyl]-6- oxaspiro[2.5]octane-2- carboxamide | 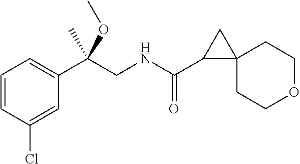 [SMILES: CO[C@@](C)(CNC(=O)C1CC12CCOCC2)c1cccc(Cl)c1] | 0.13 |
| 85 | (1S,2S)-N-[[3-(3- cyanophenyl)oxetan-3- yl]methyl]-2-phenyl- cyclopropanecarboxamide | 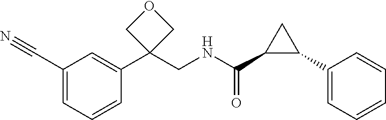 [SMILES: N#Cc1cccc(C2(CNC(=O)[C@H]3C[C@@H]3c3ccccc3)COC2)c1] | 1.14 |
| 86 | (1S,2S)-N-[1-[1-(3- chlorophenyl)cyclopropyl]-2- hydroxy-ethyl]-2-phenyl- cyclopropanecarboxamide | 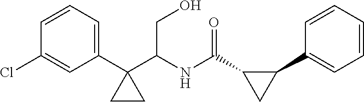 [SMILES: O=C(NC(CO)C1(c2cccc(Cl)c2)CC1)[C@H]1C[C@@H]1c1ccccc1] | 5.62 |
| 87 | (1S,2S)-N-[[2-(3-cyanophenyl)- 3,3-difluoro-oxetan-2- yl]methyl]-2-phenyl- cyclopropanecarboxamide | 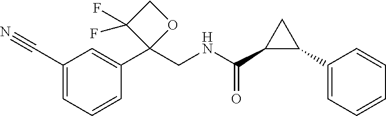 [SMILES: N#Cc1cccc(C2(CNC(=O)[C@H]3C[C@@H]3c3ccccc3)OCC2(F)F)c1] | 0.06 |
| 88 | (1S,2S)-N-[[2-(3-cyanophenyl)- 5-oxo-tetrahydrofuran-2- yl]methyl]-2-phenyl- cyclopropanecarboxamide | 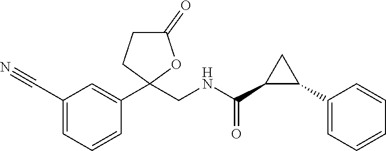 [SMILES: N#Cc1cccc(C2(CNC(=O)[C@H]3C[C@@H]3c3ccccc3)CCC(=O)O2)c1] | 0.20 |
| 89 | N-[[3-(3-cyanophenyl)oxetan-3- yl]methyl]-2-cyclohexyl- acetamide | 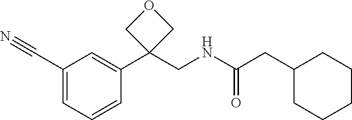 [SMILES: N#Cc1cccc(C2(CNC(=O)CC3CCCCC3)COC2)c1] | 2.79 |
| 90 | N-[[2-(3-cyanophenyl)-5-oxo- tetrahydrofuran-2-yl]methyl]-2- cyclohexyl-acetamide | 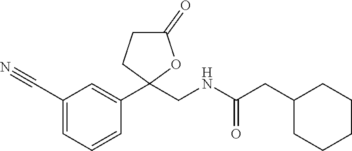 [SMILES: N#Cc1cccc(C2(CNC(=O)CC3CCCCC3)CCC(=O)O2)c1] | 0.96 |
| 91 | N-[[2-(3-cyanophenyl)-3,3- difluoro-oxetan-2-yl]methyl]-2- cyclohexyl-acetamide | 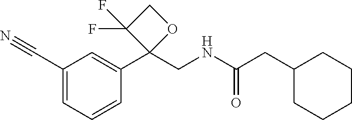 [SMILES: N#Cc1cccc(C2(CNC(=O)CC3CCCCC3)OCC2(F)F)c1] | 1.12 |
| 92 | (1S,2S)-N-[2-(3-chlorophenyl)- 4-hydroxy-butyl]-2-phenyl- cyclopropanecarboxamide | 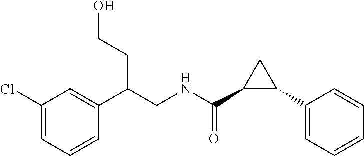 [SMILES: O=C(NCC(CCO)c1cccc(Cl)c1)[C@H]1C[C@@H]1c1ccccc1] | 1.81 |
| 93 | N-[[(2S)-2-(3- cyanophenyl)oxetan-2- yl]methyl]-2-cyclohexyl- acetamide | 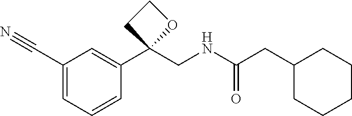 [SMILES: N#Cc1cccc([C@]2(CNC(=O)CC3CCCCC3)CCO2)c1] | 5.43 |
| 94 | N-[[(2R)-2-(3- cyanophenyl)oxetan-2- yl]methyl]-2-cycloheptyl- acetamide | 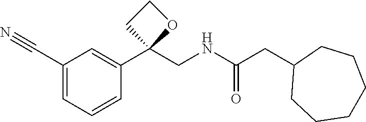 [SMILES: N#Cc1cccc([C@@]2(CNC(=O)CC3CCCCCC3)CCO2)c1] | 5.62 |
| 95 | (1S,2S)-N-[[(2S)-2-(3- cyanophenyl)oxetan-2- yl]methyl]-2- (trifluoromethyl)cyclopropanecar- boxamide | 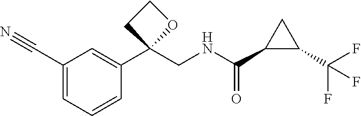 [SMILES: N#Cc1cccc([C@]2(CNC(=O)[C@H]3C[C@@H]3C(F)(F)F)CCO2)c1] | 2.55 |
| 96 | N-[[(2S)-2-(3- cyanophenyl)oxetan-2- yl]methyl]-2,2-difluoro- spiro[2.3]hexane-1- carboxamide | 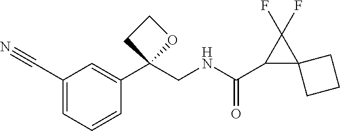 [SMILES: N#Cc1cccc([C@]2(CNC(=O)C3C(F)(F)C34CCC4)CCO2)c1] | 8.54 |
| 97 | N-[[(2R)-2-(3- cyanophenyl)oxetan-2- yl]methyl]-2-norbornan-2-yl- acetamide | 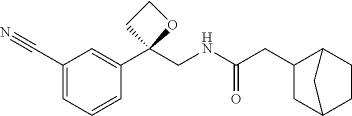 [SMILES: N#Cc1cccc([C@@]2(CNC(=O)CC3CC4CCC3C4)CCO2)c1] | 1.28 |
| 98 | N-[[(2R)-2-(3- cyanophenyl)oxetan-2- yl]methyl]bicyclo[2.2.2]octane- 2-carboxamide | 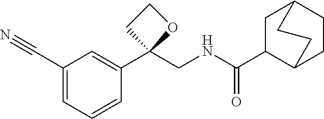 [SMILES: N#Cc1cccc([C@@]2(CNC(=O)C3CC4CCC3CC4)CCO2)c1] | 6.65 |
| 99 | (1S,2S)-N-[[2-[6- (difluoromethoxy)-2- pyridyl]oxetan-2-yl]methyl]-2- phenyl- cyclopropanecarboxamide | 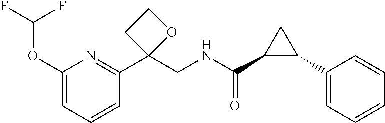 [SMILES: O=C(NCC1(c2cccc(OC(F)F)n2)CCO1)[C@H]1C[C@@H]1c1ccccc1] | 0.23 |
| 100 | N-[[(2S)-2-(3- cyanophenyl)oxetan-2- yl]methyl]spiro[3.3]heptane-3- carboxamide | 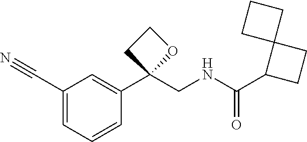 [SMILES: N#Cc1cccc([C@]2(CNC(=O)C3CCC34CCC4)CCO2)c1] | 9.76 |
| 101 | (1SR,2RS)-N-[[(2S)-2-(3- cyanophenyl)oxetan-2- yl]methyl]-2-cyclobutyl- cyclopropanecarboxamide | 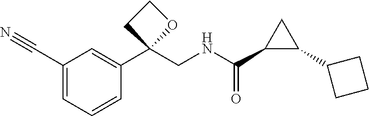 [SMILES: N#Cc1cccc([C@]2(CNC(=O)[C@H]3C[C@@H]3C3CCC3)CCO2)c1] | 0.26 |
| 102 | N-[[(2S)-2-(3- cyanophenyl)oxetan-2- yl]methyl]spiro[3.4]octane-7- carboxamide | 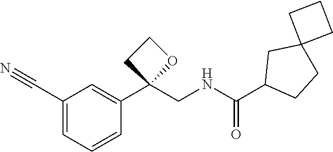 [SMILES: N#Cc1cccc([C@]2(CNC(=O)C3CCC4(CCC4)C3)CCO2)c1] | 3.01 |
| 103 | N-[[(2S)-2-(3- cyanophenyl)oxetan-2- yl]methyl]-3-cyclopentyl-2- fluoro-propanamide | 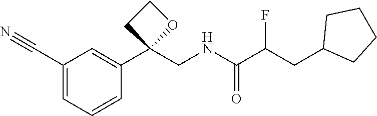 [SMILES: N#Cc1cccc([C@]2(CNC(=O)C(F)CC3CCCC3)CCO2)c1] | 6.14 |
| 104 | N-[[(2S)-2-(3- cyanophenyl)oxetan-2- yl]methyl]spiro[3.4]octane-8- carboxamide | 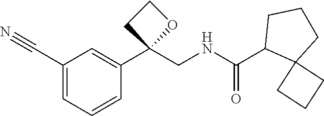 [SMILES: N#Cc1cccc([C@]2(CNC(=O)C3CCCC34CCC4)CCO2)c1] | 0.51 |
| 105 | N-[[(2S)-2-(3- cyanophenyl)oxetan-2- yl]methyl]dispiro[3.0.3⁵.1⁴]nonane- 7-carboxamide | 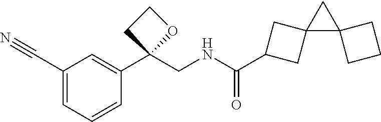 [SMILES: N#Cc1cccc([C@]2(CNC(=O)C3CC4(C3)CC43CCC3)CCO2)c1] | 0.91 |
| 106 | N-[[(2S)-2-(3- cyanophenyl)oxetan-2- yl]methyl]-6- oxaspiro[2.5]octane-2- carboxamide | 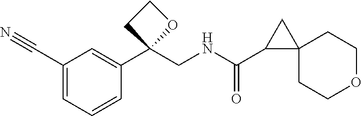 [SMILES: N#Cc1cccc([C@]2(CNC(=O)C3CC34CCOCC4)CCO2)c1] | 4.08 |
| 107 | (1SR,2SR)-N-[[(2S)-2-(3- cyanophenyl)oxetan-2- yl]methyl]-2-(4- fluorophenyl)cyclopropanecarbox- amide | 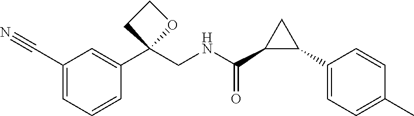 [SMILES: Cc1ccc([C@H]2C[C@@H]2C(=O)NC[C@@]2(c3cccc(C#N)c3)CCO2)cc1] | 0.13 |
| 108 | N-[[(2S)-2-(3- cyanophenyl)oxetan-2- yl]methyl]-2-norbornan-2-yl- acetamide | 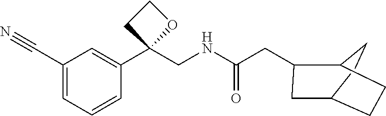 [SMILES: N#Cc1cccc([C@]2(CNC(=O)CC3CC4CCC3CC4)CCO2)c1] | 0.41 |
| 109 | N-[[(2S)-2-(3- cyanophenyl)oxetan-2- yl]methyl]-2-norbornan-1-yl- acetamide | 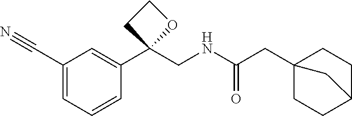 [SMILES: N#Cc1cccc([C@]2(CNC(=O)CC34CCC(CC3)C4)CCO2)c1] | 0.71 |
| 110 | (1RS,2RS)-2-tert-butyl-N- [[(2S)-2-(3- cyanophenyl)oxetan-2- yl]methyl]cyclopropanecarboxa- mide | 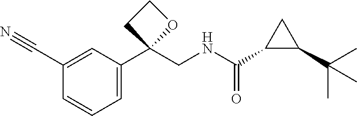 [SMILES: CC(C)(C)[C@@H]1C[C@H]1C(=O)NC[C@@]1(c2cccc(C#N)c2)CCO1] | 0.49 |
| 111 | N-[[(2S)-2-(3- cyanophenyl)oxetan-2- yl]methyl]-2-(2- hydroxynorbornan-2- yl)acetamide | 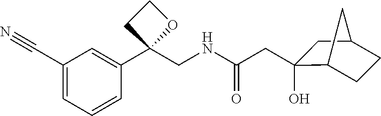 [SMILES: N#Cc1cccc([C@]2(CNC(=O)CC3(O)CC4CCC3C4)CCO2)c1] | 2.01 |
| 112 | (1S,2R)-N-[[(2S)-2-(3- cyanophenyl)oxetan-2- yl]methyl]-2-cyclopentyl- cyclopropanecarboxamide | 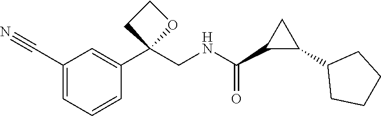 | 0.09 |
| 113 | (1SR,2SR)-N-[[(2S)-2-(3- cyanophenyl)oxetan-2- yl]methyl]-2-(1,1- difluoroethyl)cyclopropanecarbox- amide | 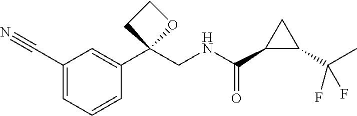 [SMILES: CC(F)(F)[C@H]1C[C@@H]1C(=O)NC[C@@]1(c2cccc(C#N)c2)CCO1] | 2.18 |
| 114 | (1SR,2SR)-2-tert-butoxy-N- [[(2S)-2-(3- cyanophenyl)oxetan-2- yl]methyl]cyclopropanecarboxa- mide | 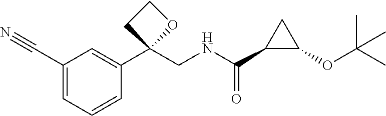 [SMILES: CC(C)(C)O[C@H]1C[C@@H]1C(=O)NC[C@@]1(c2cccc(C#N)c2)CCO1] | 7.52 |
| 115 | N-[2-[6-(difluoromethoxy)-2- pyridyl]oxetan-2-yl]methyl]-6- oxaspiro[2.5]octane-2- carboxamide | 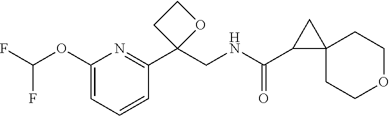 [SMILES: O=C(NCC1(c2cccc(OC(F)F)n2)CCO1)C1CC12CCOCC2] | 2.82 |
| 116 | N-[[(2S)-2-(3- cyanophenyl)oxetan-2- yl]methyl]-2-spiro[3.3]heptan- 2-yl-acetamide | 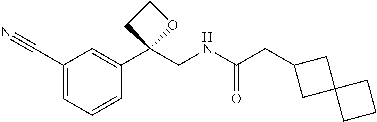 [SMILES: N#Cc1cccc([C@]2(CNC(=O)CC3CC4(CCC4)C3)CCO2)c1] | 0.61 |
| 117 | 2-cyclohexyl-N-[[2-[2- (difluoromethoxy)-4- pyridyl]oxetan-2- yl]methyl]acetamide | 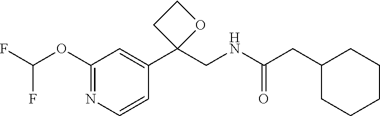 [SMILES: O=C(CC1CCCCC1)NCC1(c2ccnc(OC(F)F)c2)CCO1] | 4.33 |
| 118 | N-[[2-[2-(difluoromethoxy)-4- pyridyl]oxetan-2-y1]methyl]- 6,6-difluoro-spiro[2.5]octane-2- carboxamide | 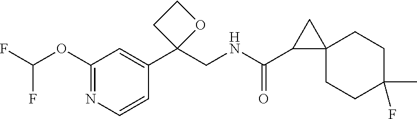 [SMILES: CC1(F)CCC2(CC1)CC2C(=O)NCC1(c2ccnc(OC(F)F)c2)CCO1] | 5.52 |
| 119 | 2-cyclohexyl-N-[2-[6- (difluoromethoxy)-2- pyridyl]oxetan-2- yl]methyl]acetamide | 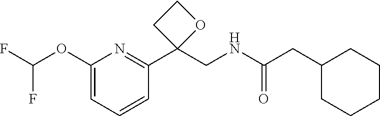 [SMILES: O=C(CC1CCCCC1)NCC1(c2cccc(OC(F)F)n2)CCO1] | 0.66 |
| 120 | N-[[2-[6-(difluoromethoxy)-2- pyridyl]oxetan-2-yl]methyl]- 6,6-difluoro-spiro[2.5]octane-2- carboxamide | 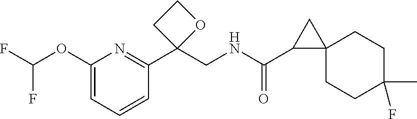 [SMILES: CC1(F)CCC2(CC1)CC2C(=O)NCC1(c2cccc(OC(F)F)n2)CCO1] | 0.26 |
| 121 | (1S,2S)-N-[[2-(2-ethyl-4- pyridyl)oxetan-2-yl]methyl]-2- phenyl- cyclopropanecarboxamide | 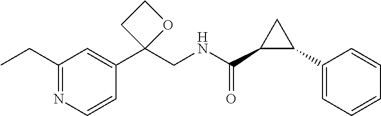 [SMILES: CCc1cc(C2(CNC(=O)[C@H]3C[C@@H]3c3ccccc3)CCO2)ccn1] | 2.51 |
| 122 | N-[[2-[2-(difluoromethoxy)-4- pyridyl]oxetan-2- yl]methyl]spiro[2.5]octane-2- carboxamide | 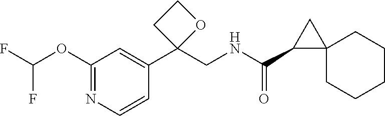 [SMILES: O=C(NCC1(c2ccnc(OC(F)F)c2)CCO1)[C@H]1CC12CCCCC2] | 0.39 |
| 123 | N-[[2-[6-(difluoromethoxy)-2- pyridyl]oxetan-2- yl]methyl]spiro[2.5]octane-2- carboxamide | 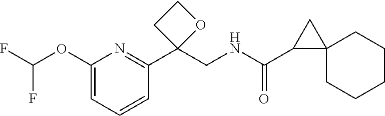 [SMILES: O=C(NCC1(c2cccc(OC(F)F)n2)CCO1)C1CC12CCCCC2] | 0.17 |
| 124 | (1SR,2SR)-N-[[(2S)-2-(3- cyanophenyl)oxetan-2- yl]methyl]-2-(2- pyridyl)cyclopropanecarboxamide | 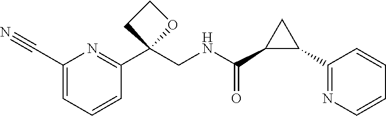 [SMILES: N#Cc1cccc([C@]2(CNC(=O)[C@H]3C[C@@H]3c3ccccn3)CCO2)n1] | 6.01 |
| 125 | (1S,2S)-N-[[2-(3-cyano-4- fluoro-phenyl)oxetan-2- yl]methyl]-2-phenyl- cyclopropanecarboxamide | 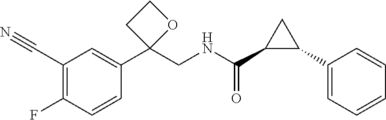 [SMILES: N#Cc1cc(C2(CNC(=O)[C@H]3C[C@@H]3c3ccccc3)CCO2)ccc1F] | 2.19 |
| 126 | (1SR,2SR)-N-[[(2S)-2-(3- cyanophenyl)oxetan-2- yl]methyl]-2-ethyl- cyclopropanecarboxamide | 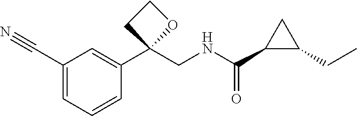 [SMILES: CC[C@H]1C[C@@H]1C(=O)NC[C@@]1(c2cccc(C#N)c2)CCO1] | 4.98 |
| 127 | (1SR,2RS)-N-[[(2S)-2-(3- cyanophenyl)oxetan-2- yl]methyl]-2-(3,3- difluorocyclobutyl)cyclopropane- carboxamide | 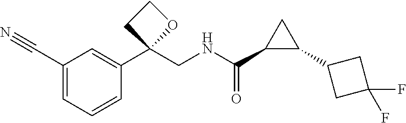 [SMILES: N#Cc1cccc([C@]2(CNC(=O)[C@H]3C[C@@H]3C3CC(F)(F)C3)CCO2)c1] | 1.04 |
| 128 | (1S,2S)-2-(4-bromophenyl)-N- [[(2S)-2-(3- cyanophenyl)oxetan-2- yl]methyl]cyclopropanecarboxa- mide | 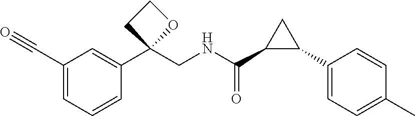 | 0.47 |
| 129 | (1S,2S)-N-[[2-[2- (difluoromethoxy)-4- pyridyl]oxetan-2-yl]methyl]-2- (trifluoromethyl)cyclopropanecar- boxamide | 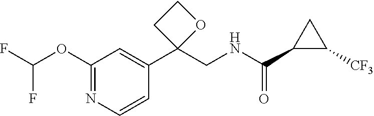 [SMILES: O=C(NCC1(c2ccnc(OC(F)F)c2)CCO1)[C@H]1C[C@@H]1C(F)(F)F] | 4.24 |
| 130 | (1S,2S)-N-[[2-[6- (difluoromethoxy)-2- pyridyl]oxetan-2-yl]methyl]-2- (trifluoromethyl)cyclopropanecar- boxamide | 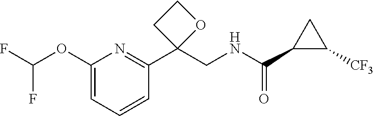 [SMILES: O=C(NCC1(c2cccc(OC(F)F)n2)CCO1)[C@H]1C[C@@H]1C(F)(F)F] | 0.68 |
| 131 | N-[[(2S)-2-(3- cyanophenyl)oxetan-2- yl]methyl]-2-fluoro-2- norbornan-2-yl-acetamide | 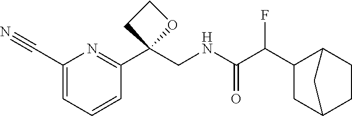 [SMILES: N#Cc1cccc([C@]2(CNC(=O)C(F)C3CC4CCC3C4)CCO2)n1] | 5.95 |
| 132 | N-[[2-[6-(difluoromethoxy)-2- pyridyl]oxetan-2-yl]methyl]-2- [1- (trifluoromethyl)cyclopropyl]ace- tamide | 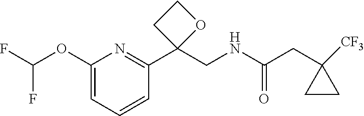 [SMILES: O=C(CC1(C(F)(F)F)CC1)NCC1(c2cccc(OC(F)F)n2)CCO1] | 8.24 |
| 133 | N-[[(2S)-2-(3- cyanophenyl)oxetan-2- yl]methyl]-2-(5-methyl-2- furyl)cyclopropanecarboxamide | 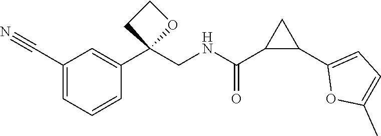 [SMILES: Cc1ccc(C2CC2C(=O)NC[C@@]2(c3cccc(C#N)c3)CCO2)o1] | 0.50 |
| 134 | (1SR,2SR)-N-[[(2S)-2-(3- cyanophenyl)oxetan-2- yl]methyl]-2-prop-1-ynyl- cyclopropanecarboxamide | 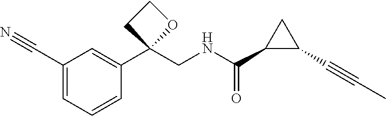 [SMILES: CC#C[C@H]1C[C@@H]1C(=O)NC[C@@]1(c2cccc(C#N)c2)CCO1] | 1.70 |
| 135 | N-[[(2S)-2-(3- cyanophenyl)oxetan-2- yl]methyl]-2-pyrrol-1-yl- cyclopropanecarboxamide | 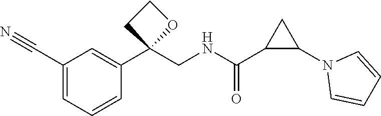 [SMILES: N#Cc1cccc([C@]2(CNC(=O)C3CC3n3cccc3)CCO2)c1] | 1.14 |
| 136 | N-[[(2S)-2-(3- cyanophenyl)oxetan-2- yl]methyl]-2-(4,4- difluorocyclohexyl)cyclopropane- carboxamide | 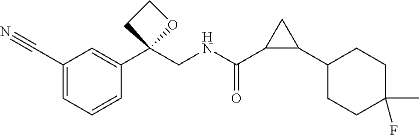 [SMILES: CC1(F)CCC(C2CC2C(=O)NC[C@@]2(c3cccc(C#N)c3)CCO2)CC1] | 0.40 |
| 137 | (1SR,2RS)-N-[[(2S)-2-(3- cyanophenyl)oxetan-2- yl]methyl]-2-prop-1-ynyl- cyclopropanecarboxamide | 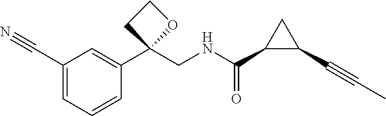 [SMILES: CC#C[C@@H]1C[C@@H]1C(=O)NC[C@@]1(c2cccc(C#N)c2)CCO1] | 1.11 |

The compounds of the present invention have shown to be agents acting on Kv7.2 and are therefore useful for the treatment and/or prophylaxis of any of the diseases, disorders, or disabilities described herein. They are in particular useful for the therapeutic and/or prophylactic treatment of a disorder, disease, or disabilities associated with Kv7.2. More particularly, they are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus. The behavioral disorder is for example Attention Deficit Hyperactivity Disorder (ADHD). The mood disorder is for example depression. The neurodevelopment disorder is for example autism spectrum disorder (ASD) or a syndromic developmental disorder. The syndromic developmental disorder is for example Dup15q syndrome (Dup15q), Fragile X syndrome (FXS), and Angelman syndrome. The epilepsies are for example broad pediatric epilepsy, West syndrome, Ohtahara syndrome and epileptic encephalopathy. Neurodegenerative diseases are for example Alzheimer's disease, or motor neuron diseases.

The compounds of the present invention are therefore useful Kv7.2 modulators that provide for favorable pharmacological properties, such as potency, and selectivity.

Compounds having one or more of the following combinations of features are found to display particularly beneficial Kv7.2 EC₅₀ values and/or Kv7,5_7.3/Kv7.2 selectivity ratios in the range of: (i) EC₅₀<3 μM, and selectivity >10×, or (ii) EC₅₀<1 μM, and selectivity>30×.

In some embodiments of this invention, compounds of formula (I) have the structures depicted from any of formulae (II)-(XV) etc. as described herein.

Measurements have shown that compounds of formula (I), or solvates or pharmaceutical salts thereof, have favorable pharmacological properties as described herein if the Kv7.2 EC₅₀<3 μM and selectivity ratio Kv7.5_7.3/Kv7.2>10×.

In some embodiments of the present invention, in the compounds of this invention one or more hydrogen atoms is (are) replaced by a deuterium. It has been found that deuteration of the compounds of this invention offer the advantage of retaining the pharmacological profile of their hydrogen counterparts while positively impacting their metabolic outcome. Selective replacement of one or more hydrogen with deuterium, in the compounds of the present invention, improves the pharmaceutical profile of compounds of this invention, e.g. by reducing the amount of undesired metabolites when compared to its all hydrogen counterparts and by lowering rates of metabolism, and hence increasing half-life.

Methods for incorporation of deuterium into compounds are well established. Using metabolic studies established in the art, the compound of the present invention can be tested to identify sites for selective placement of a deuterium isotope, which isotope will not be metabolized or be metabolized at a lower rate.

### Pharmaceutical Compositions and Administration

In one embodiment, the present invention provides a pharmaceutical composition comprising a compound of this invention as described herein.

In one embodiment, the present invention provides a pharmaceutical composition comprising a compound of this invention as described herein and one or more pharmaceutically acceptable excipients.

In one embodiment, the present invention provides a pharmaceutical composition additionally comprising one or more pharmaceutical excipients selected form diluent, filler, extender, binder, disintegrant, glidant, humectant, coating, emulsifier or dispersing agent, compression/encapsulation aid, cream or lotion, lubricant, solution for parenteral administration, material for chewable tablets, sweetener or flavoring, suspending/gelling agent, and wet granulation agent.

In a particular embodiment, the present invention provides pharmaceutical compositions as described above which are in particular useful for the therapeutic and/or prophylactic treatment of a disorder, disease, or disabilities associated with Kv7.2.

More particularly, the pharmaceutical compositions as described herein are useful for the therapeutic and/or prophylactic treatment of a disorder, disease, or disabilities associated with Kv7.2, wherein the diseases, disorders, or disabilities are selected from behavioral disorders, mood disorders, neurodevelopmental disorders, intellectual disability, epilepsies, neurodegenerative diseases, pain, migraine, and tinnitus.

The compounds of this invention can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of this invention can be processed with pharmaceutically inert, inorganic or organic adjuvants for the production of tablets, coated tablets, dragees and hard gelatin capsules. Lactose, corn starch, or derivatives thereof, talc, stearic acid or its salts etc, can be used, for example, as such adjuvants for tablets, dragées, or hard gelatin capsules.

Suitable adjuvants for soft gelatin capsules are, for example, vegetable oils, waxes, fats, semi-solid substances, or liquid polyols, etc.

Suitable adjuvants for the production of solutions and syrups are, for example, water, polyols, saccharose, invert sugar, or glucose, etc.

Suitable adjuvants for injection solutions are, for example, water, alcohols, polyols, glycerol, or vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Provided herein are pharmaceutical compositions comprising a compound of this invention. In some embodiments the pharmaceutical compositions comprise one or more pharmaceutically acceptable excipients. Conventional procedures for the selection and preparation of suitable pharmaceutical compositions are described in, for example, “Pharmaceuticals—The Science of Dosage Form Designs,” M. E. Aulton, Churchill Livingstone, 1988, which is hereby incorporated by reference in its entirety.

Further provided is a process for the preparation of a pharmaceutical composition, comprising combining one or more compounds of this invention.

Further provided is a process for the preparation of a pharmaceutical composition, comprising combining one or more compounds of this invention with one or more pharmaceutically acceptable excipients. Pharmaceutical compositions may be prepared, for example, according to conventional dissolution, mixing, granulating, or coating methods, or combinations thereof. Such pharmaceutically acceptable excipients may include, for example, sugars (e.g., lactose, glucose, sucrose); starches (e.g., corn starch, potato starch); cellulose and its derivatives (e.g., sodium carboxymethyl cellulose, ethyl cellulose, cellulose acetate); powdered tragacanth; malt; gelatin; talc; cocoa butter and suppository waxes; oils (e.g., peanut oil, cottonseed oil, safflower oil, sesame oil, olive oil, corn oil, soybean oil); glycols (e.g., propylene glycol); polyethylene glycols (PEG); esters (e.g., ethyl oleate, ethyl laurate); agar; buffering agents (e.g., magnesium hydroxide, aluminum hydroxide); alginic acid; pyrogen-free water; isotonic saline; Ringer's solution; ethyl alcohol; phosphate buffer solutions; non-toxic compatible lubricants (e.g., sodium lauryl sulfate, magnesium stearate); coloring agents; releasing agents; coating agents; sweetening; and flavoring and perfuming agents. Preservatives and antioxidants can also be present in the pharmaceutical composition, according to the judgment of the formulator.

Depending on the intended mode of administration, the disclosed pharmaceutical compositions can be in solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

These modes may include systemic or local administration such as oral, nasal, parenteral (as by intravenous (both bolus and infusion), intramuscular, or subcutaneous injection), transdermal, vaginal, buccal, rectal, or topical (as by powders, ointments, or drops) administration modes. These modes may also include intracistemally, intraperitoneally, as an oral or nasal spray, or as a liquid aerosol or dry powder pharmaceutical composition for inhalation. In some embodiments, the pharmaceutical composition provided herein comprises one or more disclosed compounds, tautomers thereof, and/or pharmaceutically acceptable salts thereof, and is for oral administration. In other embodiments, the pharmaceutical composition is for intravenous administration.

Solid dosage forms for oral administration may include capsules (e.g., soft and hard-filled gelatin capsules), tablets, pills, powders, and granules. Solid dosage forms may be prepared, in some embodiments, with one or more coatings and/or shells such as release controlling coatings, for example enteric coatings. Solid dosage forms may be formulated to release the one or more disclosed compounds (or solvate, tautomer, or pharmaceutically acceptable salt thereof) only, or mostly, or preferentially in a certain part of the gastrointestinal tract, optionally in a delayed manner. Solid dosage forms may also include, for example, micro-encapsulated forms.

Liquid dosage forms for oral administration may include, for example, pharmaceutically acceptable emulsions, microemulsions, solutions, suspensions, syrups, and elixirs. Such liquid compositions may include, for example, a pharmaceutically acceptable excipient such as water or other solvents, solubilizing agents, emulsifiers, oils, polyethylene glycols and fatty acid esters, adjuvants, sweetening agents, flavoring agents, or perfuming agents, or any combinations thereof. Injectable pharmaceutical compositions include, for example, sterile injectable aqueous compositions (e.g., solutions, suspensions, or emulsions), or oleaginous suspensions.

Injectable pharmaceutical compositions may comprise, in some embodiments, one or more solvents and/or diluents, such as water, Ringer's solution, U.S.P, and isotonic sodium chloride solution, sterile fixed oils, fatty acid, or any combinations thereof. In some embodiments, an injectable pharmaceutical composition may be prepared as a lyophilized powder, for example a lyophilized powder that is to be mixed with a liquid diluent prior to injection.

In some embodiments, it may be desirable to prolong the effect of one or more compounds as disclosed herein, or pharmaceutically acceptable salt thereof, from administration through subcutaneous or intramuscular injection. Such delay may be accomplished, for example, through the use of a liquid suspension of crystalline or amorphous material with poor water solubility; or dissolving or suspending the compound, or solvate, tautomer, or pharmaceutically acceptable salt thereof, in an oil vehicle; or through an injectable depot form comprising microencapsule matrixes comprising one or more biodegradable polymers.

Pharmaceutical compositions for rectal or vaginal administration may include suppositories that can be prepared, for example using a suitable non-irritating excipient such as cocoa butter, polyethylene glycol, or a suppository wax: or using a fatty emulsion or suspension.

Dosage forms for topical or transdermal administration may include, for example, ointments, pastes, creams, lotions, gels, powders, solutions, sprays, inhalants, or patches. Ophthalmic pharmaceutical compositions and ear drops may also be prepared.

The pharmaceutical compositions provided herein may be packaged in unit-dose or multidose containers, for example sealed ampoules or vials, and may be stored in a freeze-dried (lyophilized) condition requiring only the addition of the sterile liquid excipient (e.g., diluent, carrier, for example water) for injection immediately prior to use. Extemporaneous injection solutions and suspensions may be prepared from sterile powders, granules, or tablets of the kind described herein. Unit dosage formulations include those containing a daily dose or unit daily sub-dose, or an appropriate fraction thereof, of the active ingredient.

The subject matter further provides veterinary compositions comprising at least one active ingredient as herein defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient.

These veterinary compositions may be administered parenterally, orally or by any other desired route.

### Medical Use

The compounds of this invention or pharmaceutical compositions comprising the same, as described herein, may be useful as pharmaceuticals for the therapeutic and/or prophylactic treatment of a disorder, disease or disability associated with Kv7.2 in a subject in need thereof.

In one embodiment, the present invention provides a method for the therapeutic and/or prophylactic treatment of a disorder, disease, or disability associated with Kv7.2 in a subject in need thereof, comprising administering an effective amount of compounds of this invention or pharmaceutical compositions thereof, as described herein.

In one embodiment, the present invention provides a method for the therapeutic and/or prophylactic treatment of a disorder, disease, or disability associated with Kv7.2 in a subject in need thereof, comprising administering an effective amount of the compounds of this invention, or pharmaceutical compositions comprising the same, as described herein, wherein the pharmaceutical composition further comprises one or more pharmaceutically acceptable excipients.

In one embodiment, the present invention provides a method for the therapeutic and/or prophylactic treatment of a disorder, disease, or disability associated with Kv7.2 in a subject in need thereof, comprising a compound of formula (I), or a solvate, or a pharmaceutically acceptable salt thereof:

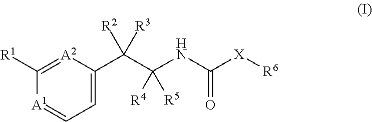

wherein
A¹ is N or CR^{a},
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

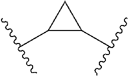 [SMILES: *C1CC1*]

R⁶ is selected from 3-10 membered cycloalkyl, 7-11 membered spiro-cycloalkyl. 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy

In one embodiment, the present invention provides a method for the therapeutic and/or prophylactic treatment of a disorder, disease, or disability associated with Kv7.2 in a subject in need thereof, comprising a compound of this invention, in particular a compound selected from formulae (I), and (II)-(XV), or a solvate or a pharmaceutically acceptable salt thereof, any exemplified compound, any embodiment, or combinations of embodiments, as described herein.

In one embodiment, the present invention provides a method for the therapeutic and/or prophylactic treatment of a disorder, disease, or disability associated with Kv7.2 in a subject in need thereof, comprising a compound selected from any list of compounds as described herein, or solvates, or a pharmaceutically acceptable salt thereof.

In one embodiment, the present invention provides a method for the therapeutic and/or prophylactic treatment of a disorder, disease, or disability associated with Kv7.2 in a subject in need thereof, comprising a compound selected from Table 1, or provided elsewhere in this application, or a solvate, or a pharmaceutically acceptable salt thereof.

In one embodiment, the present invention provides a method for the therapeutic and/or prophylactic treatment of a disorder, disease, or disability associated with Kv7.2 in a subject in need thereof, comprising a compound selected from formulae (I), and (II)-(XV), or a solvate, or a pharmaceutically acceptable salt thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the above method for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the above method for the therapeutic and/or prophylactic treatment of a disorder, disease, or disability associated with Kv7.2 in a subject in need thereof, wherein the mood disorder is depression.

In one embodiment, the present invention provides the above method for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the above method for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the above method for the therapeutic and/or prophylactic treatment of a disorder, disease, or disability associated with Kv7.2 in a subject in need thereof wherein the epilepsies are selected from broad pediatric epilepsy, West syndrome, Ohtahara syndrome, and epileptic encephalopathy.

In one embodiment, the present invention provides the above method for the therapeutic and/or prophylactic treatment of a disorder, disease, or disability associated with Kv7.2 in a subject in need thereof wherein the neurodegenerative diseases are selected from Alzheimer's disease and motor neuron diseases.

In one embodiment, the present invention provides a method for the therapeutic and/or prophylactic treatment of a disorder, disease, or disability associated with Kv7.2 in a subject in need thereof, comprising administering an effective amount of pharmaceutical compositions as described herein.

Further provided herein is a compound, a solvate, a pharmaceutically acceptable salt or a pharmaceutical composition thereof, as described herein, for use as therapeutically active substance.

In one embodiment, the present invention provides the use of a compound of formula (I), or a solvate, or a pharmaceutically acceptable salt thereof:

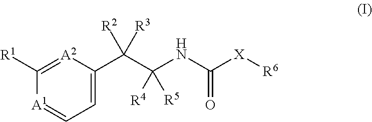

wherein
A¹ is N or CR^{a},
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen; R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

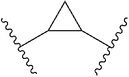 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; as therapeutically active substance.

In one embodiment, the present invention provides the use of a compound of this invention, in particular a compound selected from formulae (I), and (II)-(XV), or a solvate, or a pharmaceutically acceptable salt thereof, any exemplified compound, any embodiment, or combinations of embodiments, as described herein, as therapeutically active substance.

In one embodiment, the present invention provides the use of a compound selected from Table 1 or provided elsewhere in this application, or a solvate, or a pharmaceutically acceptable salt thereof, as therapeutically active substance.

In one embodiment, the present invention provides the use of a compound selected from any list of compounds described herein, or a solvate or a pharmaceutically acceptable salt thereof, as therapeutically active substance.

Further provided herein is a compound, a solvate, a pharmaceutically acceptable salt, or a pharmaceutical composition thereof, for use in the therapeutic and/or prophylactic treatment of a disorder, disease, or disability associated with Kv7.2 in a subject in need thereof.

In one embodiment, the present invention provides a compound of formula (I), or a solvate, or a pharmaceutically acceptable salt thereof.

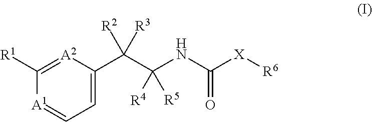

wherein
A¹ is N or CR^{a},
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

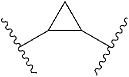 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; for use in the therapeutic and/or prophylactic treatment of a disorder, disease, or disability associated with Kv7.2 in a subject in need thereof.

In one embodiment, the present invention provides a compound of this invention, in particular a compound selected from formulae (I), and (II)-(XV), or a solvate or a pharmaceutically acceptable salt thereof, any exemplified compound, any embodiment, or combinations of embodiments, as described herein, for use in the therapeutic and/or prophylactic treatment of a disorder, disease, or disability associated with Kv7.2 in a subject in need thereof.

In one embodiment, the present invention provides a compound selected from Table 1 or provided elsewhere in this application, or a solvatem or a pharmaceutically acceptable salt thereof, for use in the therapeutic and/or prophylactic treatment of a disorder, disease, or disability associated with Kv7.2 in a subject in need thereof.

In one embodiment, the present invention provides a compound selected from any list of compounds of this invention, for use in the therapeutic and/or prophylactic treatment of a disorder, disease, or disability associated with Kv7.2 in a subject in need thereof.

In one embodiment, the present invention provides a compound of this invention for use in the therapeutic and/or prophylactic treatment of a disorder, disease, or disability associated with Kv7.2 in a subject in need thereof, wherein such disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a compound of this invention for use in the therapeutic and/or prophylactic treatment of a disorder, disease, or disability associated with Kv7.2 in a subject in need thereof, wherein the mood disorder is depression.

In one embodiment, the present invention provides a compound of this invention, for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a compound of this invention for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

Further provided herein is the use of a compound of this invention in the therapeutic and/or prophylactic treatment of a disorder, disease, or disability associated with Kv7.2 in a subject in need thereof.

In one embodiment, the present invention provides the use of a compound of formula (I), or a solvate or a pharmaceutically acceptable salt thereof:

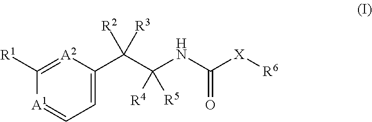

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen:
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

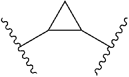 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; for the therapeutic and/or prophylactic treatment of a disorder, disease, or disability associated with Kv7.2 in a subject in need thereof.

In one embodiment, the present invention provides the use of a compound of this invention, in particular a compound selected from formulae (I), and (II)-(XV), or solvate, or a pharmaceutically acceptable salt thereof, any exemplified compound, any embodiment, or combinations of embodiments, as described herein, as described herein, for the therapeutic and/or prophylactic treatment of a disorder, disease, or disability associated with Kv7.2 in a subject in need thereof.

In one embodiment, the present invention provides the use of a compound selected from Table 1, or provided elsewhere in this application, or solvate, or a pharmaceutically acceptable salt thereof, for the therapeutic and/or prophylactic treatment of a disorder, disease, or disability associated with Kv7.2 in a subject in need thereof.

In one embodiment, the present invention provides the use a compound selected from any list of compounds described herein, or a solvate or a pharmaceutically acceptable salt thereof, for the therapeutic and/or prophylactic treatment of a disorder, disease, or disability associated with Kv7.2 in a subject in need thereof.

In one embodiment, the present invention provides for the use of a compound of this invention in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides for the use of a compound of this invention in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides for the use of a compound of this invention in the therapeutic and/or prophylactic treatment of a disorder, disease, or disability associated with Kv7.2 in a subject in need thereof, wherein the mood disorder is depression.

In one embodiment, the present invention provides for the use of a compound of this invention in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides for the use of a compound of this invention in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides for the use of a compound of this invention in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides for the use of a compound of this invention in the therapeutic and/or prophylactic treatment of a disorder, disease, or disability associated with Kv7.2 in a subject in need thereof, the neurodegenerative diseases are selected from Alzheimer's disease and motor neuron diseases.

Further provided herein is the use of a a compound of this invention or a pharmaceutical composition comprising the same, for the manufacture of a medicament for the therapeutic and/or prophylactic treatment of a disorder, disease, or disability associated with Kv7.2 in a subject in need thereof.

In one embodiment, the present invention provides the use of a compound of formula (I), or a solvate, or a pharmaceutically acceptable salt thereof:

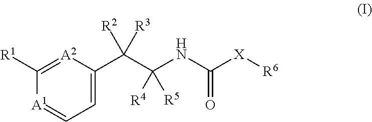

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

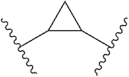 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; 45 for the manufacture of a medicament for use in the therapeutic and/or prophylactic treatment of a disorder, disease, or disability associated with Kv7.2 in a subject in need thereof.

In one embodiment, the present invention provides the use of a compound of this invention, in particular a compound selected from formulae (I), and (II)-(XV), or a solvate, or a pharmaceutically acceptable salt thereof, any exemplified compound, any embodiment, or combinations of embodiments, as described herein, for the manufacture of a medicament for use in the therapeutic and/or prophylactic treatment of a disorder, disease, or disability associated with Kv7.2 in a subject in need thereof.

In one embodiment, the present invention provides the use a compound selected from Table 1, or provided elsewhere in this application, or a solvate, or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound selected from any list of compounds described herein, or a solvate or a pharmaceutically acceptable salt thereof, for the manufacture of a medicament for use in the therapeutic and/or prophylactic treatment of a disorder, disease, or disability associated with Kv7.2 in a subject in need thereof.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the disorder, disease, or disability associated with Kv7.2 is selected from behavioral disorders, mood disorders, neurodevelopmental disorders, intellectual disability, epilepsies, neurodegenerative diseases, pain, migraine, and tinnitus.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the mood disorder is depression.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides the use of a compound of this invention for the manufacture of a medicament for use in the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides the use of a compound of this invention for the manufacture of a medicament for use in the therapeutic and/or prophylactic treatment of a disorder, disease, or disability associated with Kv7.2 in a subject in need thereof, the neurodegenerative diseases are selected from Alzheimer's disease and motor neuron diseases.

Further provided a pharmaceutical composition comprising a compound as described herein, or solvate or a pharmaceutically acceptable salt thereof.

Further provided a pharmaceutical composition comprising a compound as described herein, or solvate or a pharmaceutically acceptable salt thereof, wherein the pharmaceutical composition further comprises one or more pharmaceutically acceptable excipients.

In one particularly preferred embodiment, the present invention provides a pharmaceutical composition comprising a compound of formula (I), or a solvate, or a pharmaceutically acceptable salt thereof:

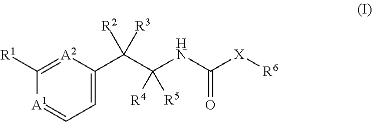

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

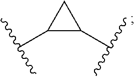 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

In one particularly preferred embodiment, the present invention provides a pharmaceutical composition comprising a compound of formula (I), or a solvate, or a pharmaceutically acceptable salt thereof:

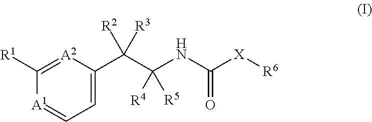

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

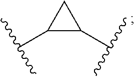 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; and wherein the pharmaceutical composition further comprises one or more pharmaceutically acceptable excipients.

In one particularly preferred embodiment, the present invention provides a pharmaceutical composition comprising a compound of this invention, in particular selected from formulae (I), and (II)-(XV), or a solvate, or a pharmaceutically acceptable salt thereof, as described herein, for use in the therapeutic and/or prophylactic treatment of a disorder, disease, or disability associated with Kv7.2 in a subject in need thereof.

In one particularly preferred embodiment, the present invention provides a pharmaceutical composition comprising a compound selected from formulae (I), and (II)-(XV), or a solvate, or a pharmaceutically acceptable salt thereof, as described herein, for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one particularly preferred embodiment, the present invention provides a pharmaceutical composition comprising a compound of this invention for use in the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the epilepsies are selected from broad pediatric epilepsy, West syndrome. Ohtahara syndrome, and epileptic encephalopathy.

In one embodiment, the present invention provides a pharmaceutical composition comprising a compound of this invention for use in the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from formulae (I), and (II)-(XV), or a solvate, or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, or as provided elsewhere in this application, or a solvate, or a pharmaceutically acceptable salt thereof.

In one embodiment, the present invention provides a pharmaceutical composition as described above comprising a compound selected from any list of compounds described herein, or a solvate, or a pharmaceutically acceptable salt thereof.

Further provided a pharmaceutical composition comprising a compound of this invention for use in a method of the therapeutic and/or prophylactic treatment of disorder, disease or disability associated with Kv7.2 in a subject in need thereof.

Further provided a pharmaceutical composition comprising a compound of this invention, and one or more pharmaceutically acceptable excipients, for use in a method of the therapeutic and/or prophylactic treatment of disorder, disease or disability associated with Kv7.2 in a subject in need thereof.

In one embodiment, the present invention provides a pharmaceutical composition for use in a method of treatment comprising a compound of formula (I), or a solvate, or a pharmaceutically acceptable salt thereof:

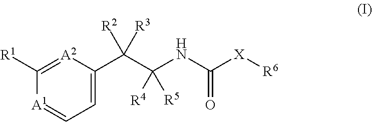

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

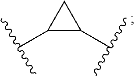 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

In one embodiment, the present invention provides a pharmaceutical composition for use in a method of treatment comprising a compound of formula (I), or a solvate, or a pharmaceutically acceptable salt thereof:

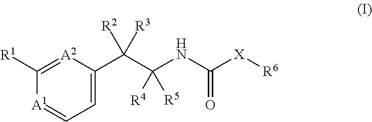

wherein
A¹ is N or CR^{a}
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxy-C₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

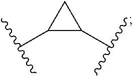 [SMILES: *C1CC1*]

R⁶ is selected from 3-10 membered cycloalkyl, 7-11 membered spiro-cycloalkyl. 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; and wherein the pharmaceutical composition further comprises one or more pharmaceutically acceptable excipients.

In one embodiment, the present invention provides a pharmaceutical composition for use in a method of treatment comprising a compound of formula (II), or a solvate, or a pharmaceutically acceptable salt thereof:

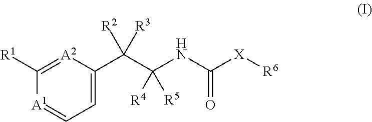

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

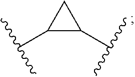 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

In one embodiment, the present invention provides a pharmaceutical composition for use in a method of treatment comprising a compound of formula (II), or a solvate, or a pharmaceutically acceptable salt thereof:

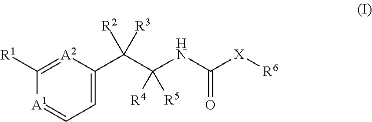

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

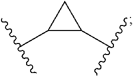 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; and wherein the pharmaceutical composition further comprises one or more pharmaceutically acceptable excipients.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition for use in a method of treatment comprises a compound of this invention for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the mood disorder is depression.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition for use in a method of treatment comprising a compound of this invention for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I), and (II)-(XV), or a solvate or a pharmaceutically acceptable salt thereof, any exemplified compound, any embodiment, or combinations of embodiments, as described herein.

In one embodiment, the present invention provides a pharmaceutical composition as described above comprising a compound selected from Table 1, or as described elsewhere in this application, or a solvate or a pharmaceutically acceptable salt thereof.

In one embodiment, the present invention provides a pharmaceutical composition as described above comprising a compound selected from any list of compounds described herein, or a solvate or a pharmaceutically acceptable salt thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease, or disability associated with Kv7.2 in a subject in need thereof.

Further provided a pharmaceutical composition comprising a compound, or a solvate or a pharmaceutically acceptable salt thereof, as described herein, for use in the manufacture of a medicament for the therapeutic and/or prophylactic treatment of a disorder, disease or disability associated with Kv7.2 in a subject in need thereof, wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof:

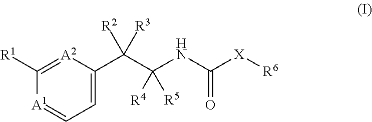

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

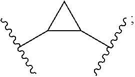 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate, or a pharmaceutically acceptable salt thereof:

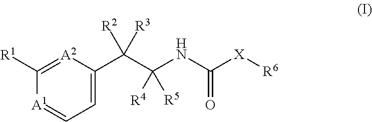

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

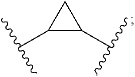 [SMILES: *C1CC1*]

R⁶ is selected from 3-10 membered cycloalkyl, 7-11 membered spiro-cycloalkyl, 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy; and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of any one of formulae (II)-(XV), or a solvate or a pharmaceutically acceptable salt thereof.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of any one of formula (II)-(XV), or a solvate, or a pharmaceutically acceptable salt thereof; and wherein the pharmaceutical composition further comprises one or more pharmaceutically acceptable excipients.

In one embodiment, the present invention provides a pharmaceutical composition for use in the manufacture of a medicament for the therapeutic and/or prophylactic treatment of a disorder, disease, or disability associated with Kv7.2 in a subject in need thereof, comprising a compound of formula (I), or a solvate or a pharmaceutically acceptable salt thereof, as described herein, for use in the therapeutic and/or prophylactic treatment of a disorder, disease, or disability associated with Kv7.2 in a subject in need thereof.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

In one embodiment, the present invention provides a pharmaceutical composition a compound of this invention for use in manufacture of a medicament for the therapeutic and/or prophylactic treatment of a disorder, disease, or disability associated with Kv7.2 in a subject in need thereof, wherein the behavioral disorder is Attention Deficit Hyperactivity Disorder (ADHD).

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the mood disorder is depression.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodevelopment disorder is selected from autism spectrum disorder (ASD) and syndromic developmental disorders.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the syndromic developmental disorder is selected from Dup15q syndrome (Dup15q), Fragile X syndrome (FXS) and Angelman syndrome.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the epilepsies are selected from broad pediatric epilepsy, West syndrome, Ohtahara syndrome, and epileptic encephalopathy.

In one embodiment, the present invention provides a pharmaceutical composition comprising a compound of this invention for use in manufacture of a medicament for the therapeutic and/or prophylactic treatment of a disorder, disease, or disability associated with Kv7.2 in a subject in need thereof, wherein the neurodegenerative diseases are selected from Alzheimer's disease and motor neuron diseases.

In one embodiment, the present invention provides a pharmaceutical composition as described above comprising a compound selected from any of formulae (I), and (II)-(XV), or a solvate or a pharmaceutically acceptable salt thereof, any exemplified compound, any embodiment, or combinations of embodiments, as described herein.

In one embodiment, the present invention provides a pharmaceutical composition as described above comprising a compound is selected from Table 1, or provided elsewhere in this application, or a solvate, or a pharmaceutically acceptable salt thereof.

In one embodiment, the pharmaceutical composition as described above comprises a compound selected from any list of compounds described herein, or a solvate or a pharmaceutically acceptable salt thereof.

### Combination Therapy

Compounds of the present invention may be combined with one or more other compounds of the present invention or one or more other therapeutic agent as any combination thereof, in the treatment of the diseases, disorders, or disabilities provided herein. For example, a compound of the present invention may be administered simultaneously, sequentially or separately in combination with other therapeutic agents known to be useful for the treatment of a disease or disorder selected from those recited herein.

In some embodiments, compounds of the present invention may be combined with another therapeutically active agent having a synergistic effect in the treatment of any diseases, disorders, or disabilities described herein.

As used herein “combination” refers to any mixture or permutation of one or more compounds of the present invention and one or more other compounds of the present invention or one or more additional therapeutic agent. Unless the context makes clear otherwise, “combination” may include simultaneous or sequentially delivery of a compound of the present invention with one or more therapeutic agents. Unless the context makes clear otherwise, “combination” may include dosage forms of a compound of the present invention with another therapeutic agent. Unless the context makes clear otherwise, “combination” may include routes of administration of a compound of the present invention with another therapeutic agent. Unless the context makes clear otherwise, “combination” may include formulations of a compound of the present invention with another therapeutic agent. Dosage forms, routes of administration and pharmaceutical compositions include, but are not limited to, those described herein.

### Articles of Manufacture

In one embodiment, the present invention provides an article of manufacture, or “kit”, containing materials useful for the treatment of the disorder, disease, or disability described herein is provided.

In one embodiment of the present invention, the kit comprises a container comprising a compound of this invention, as described in any embodiment of this invention.

In one embodiment, the present invention provides a kit comprising a container comprising a compound of this invention, or a pharmaceutical composition thereof, as described herein.

In one embodiment, the present invention provides a kit comprising a compound of this invention, any exemplified compound, any embodiment, or combinations of embodiments, or pharmaceutical composition thereof, as described herein.

In one embodiment, the present invention provides a kit, wherein the compound is selected from formulae (I), and (II)-(XV), or a solvate, a pharmaceutically acceptable salt, or a pharmaceutical composition thereof, as described herein.

In one embodiment, the present invention provides a kit, wherein the compound is selected from Table 1, or provided elsewhere in this application, a solvate, a pharmaceutically acceptable salt, or a pharmaceutical composition thereof, as described herein.

In one embodiment, the present invention provides a kit for use in the treatment of a disorder, disease, or disability associated with Kv7.2, comprising:
a) a first pharmaceutical composition comprising a compound of this invention; and
b) instructions for use.

In one embodiment, the present invention provides a kit for use in the therapeutic and/or prophylactic treatment of a disorder, disease, or disability associated with Kv7.2 comprising:
a) a compound of this invention, or a pharmaceutical composition, or a pharmaceutical composition for use thereof; as described herein; and
b) instructions for use.

In one embodiment, the present invention provides a kit for use in the therapeutic and/or prophylactic treatment of a disorder, disease, or disability associated with Kv7.2 selected from behavioral disorders, mood disorders, neurodevelopmental disorders, intellectual disability, epilepsies, neurodegenerative diseases, pain, migraine, and tinnitus, comprising:
a) a first pharmaceutical composition comprising a compound of formula (I), or a solvate, or a pharmaceutically acceptable salt, a pharmaceutical composition, or a pharmaceutical composition for use; as described herein:

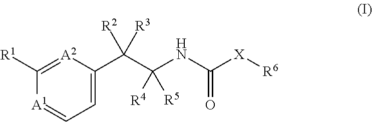

wherein
A¹ is N or CR^{a};
A² is N or CR^{b};
R^{a} is hydrogen or halogen;
R¹ is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₆cycloalkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, and CHFCH₂, and

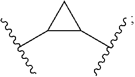 [SMILES: *C1CC1*]

R⁶ is selected from 3-10 membered cycloalkyl, 7-11 membered spiro-cycloalkyl. 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy;
and
b) instructions for use.

In one embodiment, the present invention provides a kit for use in the therapeutic and/or prophylactic treatment of a disorder, disease, or disability associated with Kv7.2. These disorders, diseases or disabilities can be selected from behavioral disorders, mood disorders, neurodevelopmental disorders, intellectual disability, epilepsies, neurodegenerative diseases, pain, migraine, and tinnitus, comprising a compound, a pharmaceutical composition, or a pharmaceutical composition for use thereof, as described herein.

In one embodiment, the present invention provides a kit for use as described herein, wherein the behavioral disorder Attention Deficit Hyperactivity Disorder (ADHD).

In one embodiment, the present invention provides a kit for use as described herein, wherein the mood disorder is depression.

In one embodiment, the present invention provides a kit for use as described herein, wherein the neurodevelopment disorder is selected from autism spectrum disorder (ASD) and syndromic developmental disorders.

In one embodiment, the present invention provides a kit for use as described herein, wherein the syndromic developmental disorder is selected from Dup15q syndrome (Dup15q), Fragile X syndrome (FXS), and Angelman syndrome.

In one embodiment, the present invention provides a kit for use as described herein, wherein the epilepsies are selected from broad pediatric epilepsy, West syndrome, Ohtahara syndrome, and epileptic encephalopathy.

In one embodiment, the present invention provides a kit for use as described herein, wherein the neurodegenerative diseases are selected from Alzheimer's disease, and motor neuron diseases.

In one embodiment, the present invention provides a kit for use as described herein, wherein the kit further comprises a label or package insert, on or associated with the container.

The term “package insert” is used to refer to instructions customarily included in commercial packages of therapeutic products, that contain information about the indications, usage, dosage, administration, contraindications and/or warnings concerning the use of such therapeutic products.

Suitable containers include, e.g., bottles, vials, syringes, blister pack, etc. The container may be formed from a variety of materials such as glass or plastic.

The container may hold a compound of this invention or a formulation thereof which is effective for treating the condition and may have a sterile access port (e.g., the container may be an intravenous solution bag or a vial having a stopper pierceable by a hypodermic injection needle). At least one active agent in the composition is a compound of this invention. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of this invention can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit further comprises directions for the administration of the compounds of this invention and, if present, the second pharmaceutical formulation. For example, if the kit comprises a first composition comprising a compound of this invention, and a second pharmaceutical formulation, the kit may further comprise directions for the simultaneous, sequential or separate administration of the first and second pharmaceutical compositions to a patient in need thereof.

In one embodiment, the present invention provides a kit for use as described herein, wherein the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, e.g. in the form of numbers, letters, or other markings or with a calendar insert, designating the days in the treatment schedule in which the dosages can be administered.

In one embodiment, the present invention provides a kit for use as described herein, wherein the kit comprises (a) a first container with a compound of this invention contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one embodiment, the present invention provides a kit for use as described herein, wherein the kit comprises a composition of this invention and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Embodiment I-1. A compound of formula (I), or a solvate, or a pharmaceutically acceptable salt thereof:

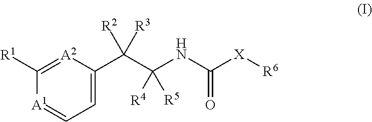

wherein
A¹ is N or CR^{a};
A² is N or CR^{b};
R^{a} is hydrogen or halogen;
R^{b} is hydrogen or halogen;
R¹ is selected from hydrogen, cyano, haloC₁₋₆alkyl, C₁₋₆alkoxy, haloC₁₋₆alkoxy, and halogen;
R² and R³ are independently selected from hydrogen, hydroxy, C₁₋₆alkyl, C₁₋₆alkoxy, hydroxyC₁₋₆alkyl, C₃₋₅cycloalkylC₁₋₆alkoxy, cyano, —OCD₃, cyanoC₁₋₆alkoxy, haloC₁₋₆alkoxy, hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is absent, and R² is ═CHCH₂OH;
R⁴ is hydrogen, hydroxyC₁₋₆alkyl, or halogen;
R⁵ is hydrogen, or halogen;
   X is selected from NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), absent, CHFCH₂, and

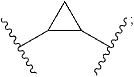 [SMILES: *C1CC1*]

R⁶ is selected from 3-10 membered cycloalkyl, 7-11 membered spiro-cycloalkyl. 7-11 membered oxo-spiro-heterocycloalkyl, aryl, heteroaryl, C₁₋₆alkyl, —C≡CCH₃, 4-10 membered heterocycloalkyl, and haloC₁₋₆alkyl, wherein cycloalkyl, spiro-cycloalkyl, oxa-spiro-cycloalkyl, aryl, heteroaryl, or heterocycloalkyl are optionally substituted with one, two, or three substituents independently selected from halogen, haloC₁₋₆alkyl, C₁₋₆alkyl, C₁₋₆alkoxy, and haloC₁₋₆alkoxy.

Embodiment I-2. A compound according to embodiment I-1, wherein (i) only A¹; (ii) only A²; (iii) A¹ and A² both are N; or (iv) A¹ is CR^{a} and A² is CR^{b}.

Embodiment I-3. A compound according to embodiment I-2, wherein only A¹ is N or only A² is N.

Embodiment I-4. A compound according to embodiment I-1, wherein A¹ is CR^{a} and A² is CR^{b}.

Embodiment I-5. A compound according to embodiment I-4, wherein R^{a} and R^{b} are hydrogen.

Embodiment I-6. A compound according to any of embodiments I-1 to I-5, wherein R¹ is halogen.

Embodiment I-7. A compound according to embodiment I-6, wherein R¹ is C₁.

Embodiment I-8. A compound according to any one of embodiment I-1 to I-5, wherein R¹ is cyano or CHF₂O—.

Embodiment I-9. A compound according any one of embodiment I-1 to I-8, wherein R² is hydroxy and R³ is C₁₋₆alkyl or R² is C₁₋₆alkoxy and R³ is C₁₋₆alkyl.

Embodiment I-10. A compound according to embodiment I-9, wherein R² is hydroxy and R³ is methyl.

Embodiment I-11. A compound according to embodiment I-9, wherein R² is methoxy and R³ is methyl.

Embodiment I-12. A compound according to any one of embodiments I-1 to I-8, wherein R² and R³ together with the C-atom they are attached to form a 4-6 membered heterocycloalkyl optionally substituted with one or two halogen.

Embodiment I-13. A compound according to embodiment I-12, wherein R² and R³ together with the C-atom they are attached to form a 4-6 membered heterocycloalkyl and wherein the heterocycloalkyl is

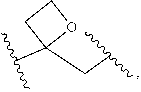 [SMILES: *CC1(*)CCO1]

which is optionally substituted with two F.

Embodiment I-14. A compound according to any one of embodiments I-1 to I-13, wherein R⁴ and R⁵ are H.

Embodiment I-15. A compound according to any one of embodiments I-1 to I-14, wherein X is selected from
X is selected from CH₂, CH₂CH₂, CH₂CH₂CH₂, absent, and

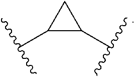 [SMILES: *C1CC1*]

Embodiment I-16. A compound according to embodiment I-15, wherein X is

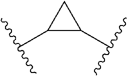 [SMILES: *C1CC1*]

Embodiment I-17. A compound according to any one of embodiments I-1 to I-16, wherein R⁶ is selected from 3-10 membered cycloalkyl, aryl, 7-11 membered spiro-cycloalkyl, and 7-11 membered oxa-spiro-cycloalkyl.

Embodiment I-18. A compound according to embodiment I-17, wherein R⁶ is aryl is phenyl, 3-10 membered cycloalkyl is 4-6 membered saturated monocyclic cycloalkyl, or 7-10 membered bridged cycloalkyl, wherein such phenyl, cycloalkyl, or bridged cycloalkyl are optionally substituted as with one or two halogen or oxo.

Embodiment I-19. A compound according to any one of embodiments I-1 to I-18, selected from the group consisting of:

| Example | Name | Structure |
|---|---|---|
| 1 | rac-(1S,2S)-N-[2-(3-chlorophenyl)- 2-methoxy-propyl]-2-phenyl- cyclopropanecarboxamide | 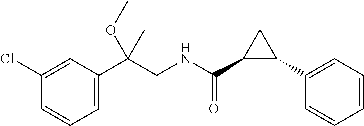 [SMILES: COC(C)(CNC(=O)[C@H]1C[C@@H]1c1ccccc1)c1cccc(Cl)c1] |
| 2 | (1S,2S)-N-[(2R)-2-(3-chlorophenyl)- 2-methoxy-propyl]-2-phenyl- cyclopropanecarboxamide | 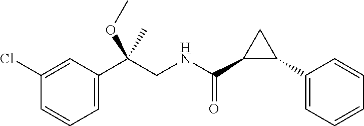 [SMILES: CO[C@@](C)(CNC(=O)[C@H]1C[C@@H]1c1ccccc1)c1cccc(Cl)c1] |
| 3 | 1-[2-(3-chlorophenyl)-2-methoxy- propyl]-3-cyclopentyl-urea | 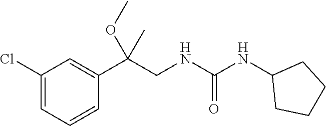 [SMILES: COC(C)(CNC(=O)NC1CCCC1)c1cccc(Cl)c1] |
| 4 | 1-[2-(3-chlorophenyl)-2-methoxy- ethyl]-3-norbornan-2-yl-urea | 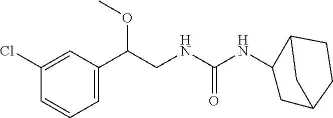 [SMILES: COC(CNC(=O)NC1CC2CCC1C2)c1cccc(Cl)c1] |
| 5 | 1-[2-(3-chlorophenyl)-2-methoxy- propyl]-3-(3-methoxyphenyl)urea | 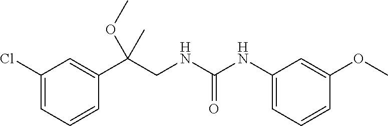 [SMILES: COc1cccc(NC(=O)NCC(C)(OC)c2cccc(Cl)c2)c1] |
| 6 | 1-[2-(3-chlorophenyl)-2-methoxy- propyl]-3-(m-tolyl)urea | 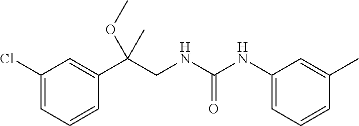 [SMILES: COC(C)(CNC(=O)Nc1cccc(C)c1)c1cccc(Cl)c1] |
| 7 | 1-[[1-(3- chlorophenyl)cyclopropyl]methyl]- 3-cyclopentyl-urea | 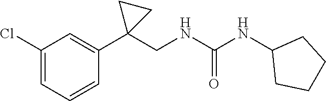 [SMILES: O=C(NCC1(c2cccc(Cl)c2)CC1)NC1CCCC1] |
| 8 | 1-butyl-3-[2-(3-chlorophenyl)-2- methoxy-propyl]urea | 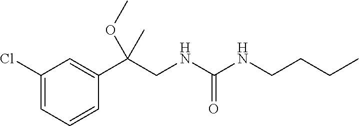 [SMILES: CCCCNC(=O)NCC(C)(OC)c1cccc(Cl)c1] |
| 9 | 1-[2-(3-chlorophenyl)-2-methoxy- propyl]-3-(2-furylmethyl)urea | 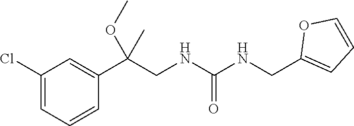 [SMILES: COC(C)(CNC(=O)NCc1ccco1)c1cccc(Cl)c1] |
| 10 | 1-[2-(3-chlorophenyl)-2-methoxy- propyl]-3-[(3- fluorophenyl)methyl]urea | 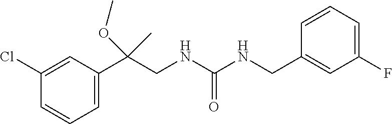 [SMILES: COC(C)(CNC(=O)NCc1cccc(F)c1)c1cccc(Cl)c1] |
| 11 | 1-[2-(3-chlorophenyl)-2-methoxy- propyl]-3-(m-tolylmethyl)urea | 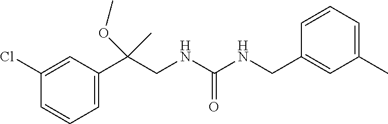 [SMILES: COC(C)(CNC(=O)NCc1cccc(C)c1)c1cccc(Cl)c1] |
| 12 | 1-[[1-(3- chlorophenyl)cyclopropyl]methyl]- 3-(1-oxaspiro[4.4]nonan-3-yl)urea | 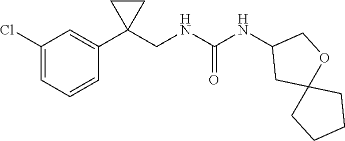 [SMILES: O=C(NCC1(c2cccc(Cl)c2)CC1)NC1COC2(CCCC2)C1] |
| 13 | 1-[2-(3-chlorophenyl)-2-methoxy- propyl]-3-(1-oxaspiro[4.4]nonan-3- yl)urea | 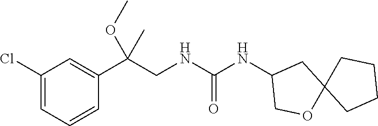 [SMILES: COC(C)(CNC(=O)NC1COC2(CCCC2)C1)c1cccc(Cl)c1] |
| 14 | 1-[2-(3-chlorophenyl)-2-methoxy- propyl]-3-(3-ethoxyphenyl)urea | 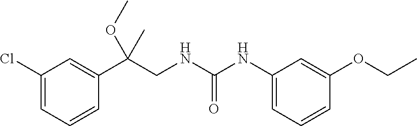 [SMILES: CCOc1cccc(NC(=O)NCC(C)(OC)c2cccc(Cl)c2)c1] |
| 15 | 1-[[1-(3- chlorophenyl)cyclopropyl]methyl]- 3-(tetrahydropyran-2-ylmethyl)urea | 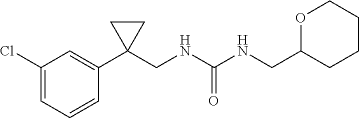 [SMILES: O=C(NCC1CCCCO1)NCC1(c2cccc(Cl)c2)CC1] |
| 16 | 1-[(2R)-2-(3-chlorophenyl)-2- methoxy-propyl]-3-cyclopentyl-urea | 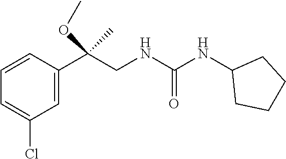 [SMILES: CO[C@@](C)(CNC(=O)NC1CCCC1)c1cccc(Cl)c1] |
| 17 | 1-[(2R)-2-(3-chlorophenyl)-2- methoxy-propyl]-3-(3- methoxyphenyl)urea | 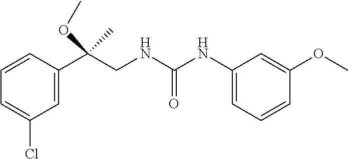 [SMILES: COc1cccc(NC(=O)NC[C@](C)(OC)c2cccc(Cl)c2)c1] |
| 18 | 1-[2-(3-chlorophenyl)-2- (cyclopropoxy)ethyl]-3-cyclopentyl- urea | 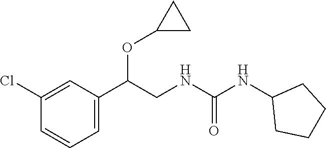 [SMILES: O=C(NCC(OC1CC1)c1cccc(Cl)c1)NC1CCCC1] |
| 19 | N-[2-(3-chlorophenyl)-2-methoxy- propyl]-2-cyclohexyl-acetamide | 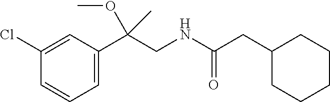 [SMILES: COC(C)(CNC(=O)CC1CCCCC1)c1cccc(Cl)c1] |
| 20 | rac-(1R,2R)-N-[2-(3-chlorophenyl)- 2-methoxy-ethyl]-2-phenyl- cyclopropanecarboxamide | 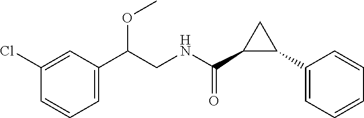 [SMILES: COC(CNC(=O)[C@H]1C[C@@H]1c1ccccc1)c1cccc(Cl)c1] |
| 21 | 1-[2-(3-chlorophenyl)-2-methoxy- propyl]-3-(cyclopentylmethyl)urea | 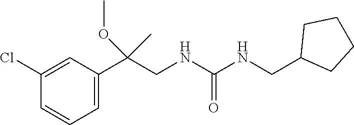 [SMILES: COC(C)(CNC(=O)NCC1CCCC1)c1cccc(Cl)c1] |
| 22 | 1-[2-(3-chlorophenyl)-2-methoxy- ethyl]-3-(m-tolyl)urea | 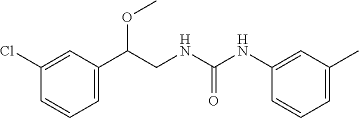 [SMILES: COC(CNC(=O)Nc1cccc(C)c1)c1cccc(Cl)c1] |
| 23 | 1-[2-(3-chlorophenyl)-2-methoxy- propyl]-3-(cyclopropylmethyl)urea | 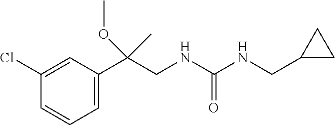 [SMILES: COC(C)(CNC(=O)NCC1CC1)c1cccc(Cl)c1] |
| 24 | 1-[2-(3-bromophenyl)-2-isopropoxy- ethyl]-3-cyclopentyl-urea | 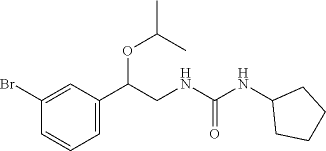 [SMILES: CC(C)OC(CNC(=O)NC1CCCC1)c1cccc(Br)c1] |
| 25 | 1-[2-(3-chlorophenyl)-2-methoxy- propyl]-3-[rac-(1S,2S,4R)- norbornan-2-yl]urea | 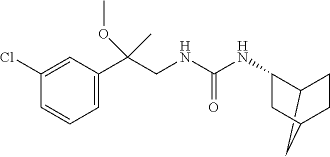 [SMILES: COC(C)(CNC(=O)N[C@H]1CC2CCC1C2)c1cccc(Cl)c1] |
| 26 | 1-[2-(3-chlorophenyl)-2- (trideuteriomethoxy)propyl]-3- cyclopentyl-urea | 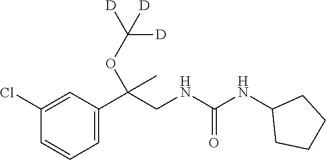 [SMILES: [2H]C([2H])([2H])OC(C)(CNC(=O)NC1CCCC1)c1cccc(Cl)c1] |
| 27 | 1-(3-methoxyphenyl)-3-[[2-(m- tolyl)-1,3-dioxolan-2-yl]methyl]urea | 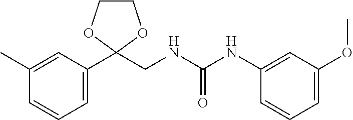 |
| 28 | 1-[2-(3-bromophenyl)-2-ethoxy- ethyl]-3-cyclopentyl-urea | 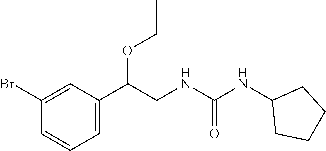 [SMILES: CCOC(CNC(=O)NC1CCCC1)c1cccc(Br)c1] |
| 29 | 1-[2-(3-chlorophenyl)-2-methoxy- propyl]-3-[3- (trifluoromethoxy)phenyl]urea | 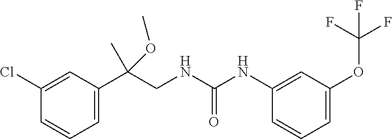 [SMILES: COC(C)(CNC(=O)Nc1cccc(OC(F)(F)F)c1)c1cccc(Cl)c1] |
| 30 | 1-[2-(3-chlorophenyl)-2-methoxy- propyl]-3-cyclopent-3-en-1-yl-urea | 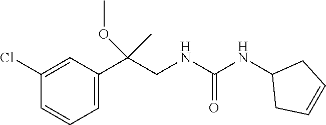 [SMILES: COC(C)(CNC(=O)NC1CC=CC1)c1cccc(Cl)c1] |
| 31 | rac-(1S,2S)-N-[2-cyano-2-[3- (trifluoromethyl)phenyl]propyl]-2- phenyl-cyclopropanecarboxamide | 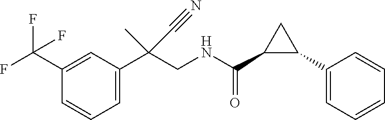 [SMILES: CC(C#N)(CNC(=O)[C@H]1C[C@@H]1c1ccccc1)c1cccc(C(F)(F)F)c1] |
| 32 | 1-[2-(3-chlorophenyl)-2-methoxy- propyl]-3-(3-phenylpropyl)urea | 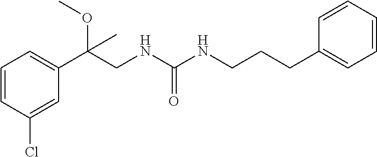 [SMILES: COC(C)(CNC(=O)NCCCc1ccccc1)c1cccc(Cl)c1] |
| 33 | 1-[2-(3-chlorophenyl)-2-methoxy- propyl]-3-(2-thienylmethyl)urea | 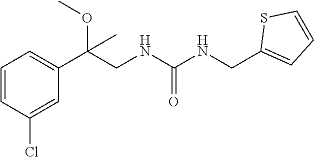 [SMILES: COC(C)(CNC(=O)NCc1cccs1)c1cccc(Cl)c1] |
| 34 | 1-[2-(3-chlorophenyl)-2-methoxy- propyl]-3-[2-(4- methoxyphenyl)ethyl]urea | 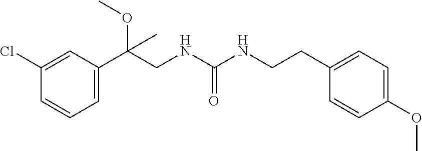 [SMILES: COc1ccc(CCNC(=O)NCC(C)(OC)c2cccc(Cl)c2)cc1] |
| 35 | 1-[2-(3-chlorophenyl)-2-methoxy- propyl]-3-[rac-(1S,4R)-norbornan-2- yl]urea | 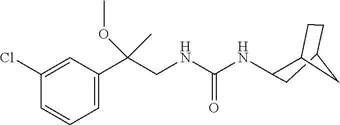 [SMILES: COC(C)(CNC(=O)NC1CC2CCC1C2)c1cccc(Cl)c1] |
| 36 | N-[2-(2-chlorophenyl)-2-methoxy- ethyl]-2-(1-naphthyl)acetamide | 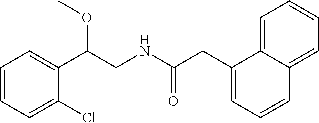 [SMILES: COC(CNC(=O)Cc1cccc2ccccc12)c1ccccc1Cl] |
| 37 | rac-(1S,2S)-N-[2-(3-chlorophenyl)- 2-cyano-propyl]-2-phenyl- cyclopropanecarboxamide | 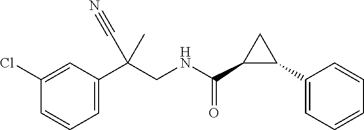 [SMILES: CC(C#N)(CNC(=O)[C@H]1C[C@@H]1c1ccccc1)c1cccc(Cl)c1] |
| 38 | 1-[3-(1r,3r)-(3- chlorophenyl)cyclobutyl]-3- [(1SR,2SR,4RS)-norbornan-2-yl] urea | 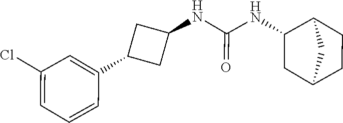 [SMILES: O=C(N[C@H]1C[C@@H]2CC[C@H]1C2)N[C@H]1C[C@H](c2cccc(Cl)c2)C1] |
| 39 | (1S,2S)-N-[[(2R)-2-(3- chlorophenyl)oxetan-2-yl]methyl]-2- phenyl-cyclopropanecarboxamide | 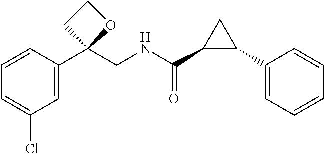 [SMILES: O=C(NC[C@]1(c2cccc(Cl)c2)CCO1)[C@H]1C[C@@H]1c1ccccc1] |
| 40 | (1S,2S)-N-[[(2S)-2-(3- chlorophenyl)oxetan-2-yl]methyl]-2- phenyl-cyclopropanecarboxamide | 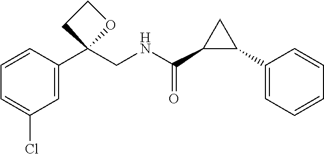 [SMILES: O=C(NC[C@@]1(c2cccc(Cl)c2)CCO1)[C@H]1C[C@@H]1c1ccccc1] |
| 41 | (1S,2S)-N-[[(2R)-2-(3- chlorophenyl)-3,3-difluoro-oxetan-2- yl]methyl]-2-phenyl- cyclopropanecarboxamide | 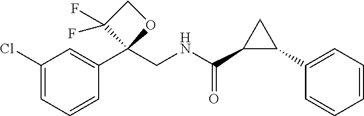 [SMILES: O=C(NC[C@@]1(c2cccc(Cl)c2)OCC1(F)F)[C@H]1C[C@@H]1c1ccccc1] |
| 42 | (1S,2S)-N-[[(2S)-2-(3-chlorophenyl)- 3,3-difluoro-oxetan-2-yl]methyl]-2- phenyl-cyclopropanecarboxamide | 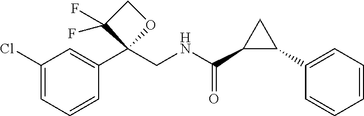 [SMILES: O=C(NC[C@]1(c2cccc(Cl)c2)OCC1(F)F)[C@H]1C[C@@H]1c1ccccc1] |
| 43 | N-[2-(3-chlorophenyl)-2-methoxy- propyl]-2-(2- fluorophenyl)cyclopropanecarboxamide | 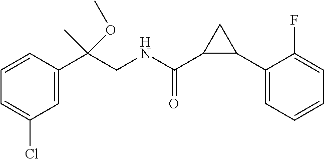 [SMILES: COC(C)(CNC(=O)C1CC1c1ccccc1F)c1cccc(Cl)c1] |
| 44 | 2-(4-chlorophenyl)-N-[2-(3- chlorophenyl)-2-methoxy- propyl]cyclopropanecarboxamide | 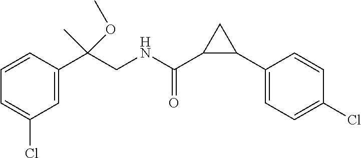 [SMILES: COC(C)(CNC(=O)C1CC1c1ccc(Cl)cc1)c1cccc(Cl)c1] |
| 45 | 1-[2-(3-chlorophenyl)-2-(2,2,2- trifluoroethoxy)ethyl]-3-[rac- (1R,4S)-norbornan-2-yl]urea | 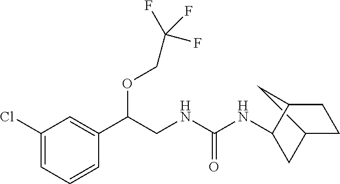 [SMILES: O=C(NCC(OCC(F)(F)F)c1cccc(Cl)c1)NC1CC2CCC1CC2] |
| 46 | 1-[2-(3-chlorophenyl)-2- (cyclobutoxy)ethyl]-3-[rac-(1R,4S)- norbornan-2-yl]urea | 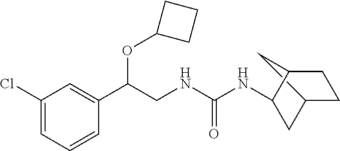 [SMILES: O=C(NCC(OC1CCC1)c1cccc(Cl)c1)NC1CC2CCC1CC2] |
| 47 | (E)-N-[2-(3-chlorophenyl)-2- methoxy-propyl]-3-(2- fluorophenyl)prop-2-enamide | 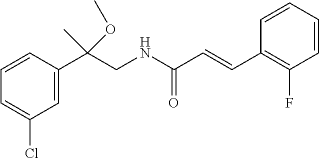 [SMILES: COC(C)(CNC(=O)/C=C/c1ccccc1F)c1cccc(Cl)c1] |
| 48 | (1S,2SR)-N-[(2R)-2-(3- chlorophenyl)-2-methoxy-propyl]-2- (3-pyridyl)cyclopropanecarboxamide | 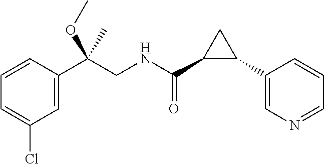 [SMILES: CO[C@@](C)(CNC(=O)[C@H]1C[C@@H]1c1cccnc1)c1cccc(Cl)c1] |
| 49 | (1S,2S)-N-[[(2S)-2-(3- cyanophenyl)oxetan-2-yl]methyl]-2- phenyl-cyclopropanecarboxamide | 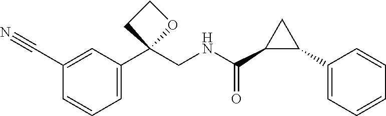 [SMILES: N#Cc1cccc([C@]2(CNC(=O)[C@H]3C[C@@H]3c3ccccc3)CCO2)c1] |
| 50 | 1-[[2-(3-chlorophenyl)oxetan-2- yl]methyl]-3-(3-methoxyphenyl)urea | 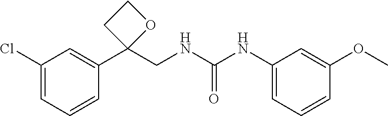 [SMILES: COc1cccc(NC(=O)NCC2(c3cccc(Cl)c3)CCO2)c1] |
| 51 | (1S,2S)-N-[(2RS)-2-(2-chloro-4- pyridyl)-2-methoxy-propyl]-2- phenyl-cyclopropanecarboxamide | 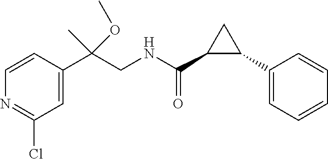 [SMILES: COC(C)(CNC(=O)[C@H]1C[C@@H]1c1ccccc1)c1ccnc(Cl)c1] |
| 52 | (1S,2S)-N-[(2RS)-2-methoxy-2-(2- methoxy-4-pyridyl)propyl]-2- phenyl-cyclopropanecarboxamide | 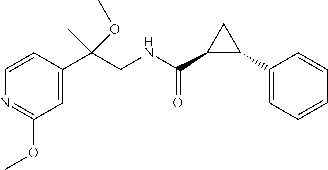 [SMILES: COc1cc(C(C)(CNC(=O)[C@H]2C[C@@H]2c2ccccc2)OC)ccn1] |
| 53 | N-[2-(3-chlorophenyl)-2-methoxy- propyl]-3-cyclopentyl-propanamide | 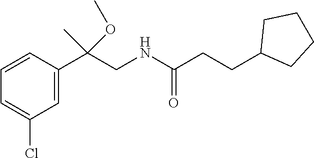 [SMILES: COC(C)(CNC(=O)CCC1CCCC1)c1cccc(Cl)c1] |
| 54 | 1-[[2-(3-chlorophenyl)oxetan-2- yl]methyl]-3-[rac-(1S,2S,4R)- norbornan-2-yl]urea | 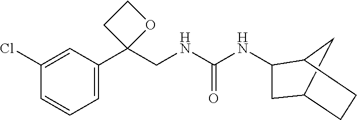 [SMILES: O=C(NCC1(c2cccc(Cl)c2)CCO1)NC1CC2CCC1C2] |
| 55 | (1S,2S)-N-[[(2R)-2-(3- cyanophenyl)oxetan-2-yl]methyl]-2- phenyl-cyclopropanecarboxamide | 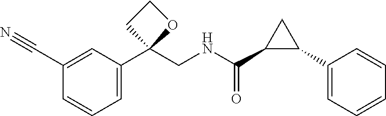 [SMILES: N#Cc1cccc([C@@]2(CNC(=O)[C@H]3C[C@@H]3c3ccccc3)CCO2)c1] |
| 56 | N-[3-(1r,3r)-(3- chlorophenyl)cyclobutyl]-2- cyclohexyl-acetamide | 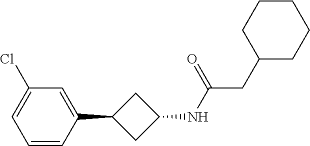 [SMILES: O=C(CC1CCCCC1)N[C@H]1C[C@H](c2cccc(Cl)c2)C1] |
| 57 | N-[[2-(3-chlorophenyl)oxetan-2- yl]methyl]-2-cyclohexyl-acetamide | 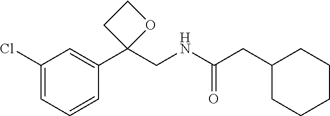 [SMILES: O=C(CC1CCCCC1)NCC1(c2cccc(Cl)c2)CCO1] |
| 58 | 1-(3-chlorophenyl)-3-[[2-(3- chlorophenyl)oxetan-2- yl]methyl]urea | 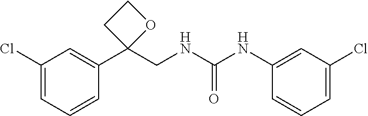 [SMILES: O=C(NCC1(c2cccc(Cl)c2)CCO1)Nc1cccc(Cl)c1] |
| 59 | 1-[[2-(3-chlorophenyl)oxetan-2- yl]methyl]-3- (cyclopentylmethyl)urea | 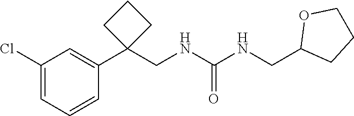 [SMILES: O=C(NCC1CCCO1)NCC1(c2cccc(Cl)c2)CCC1] |
| 60 | 1-[[2-(3-chlorophenyl)oxetan-2- yl]methyl]-3-(2,2- dimethylpropyl)urea | 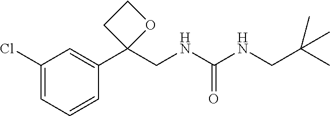 [SMILES: CC(C)(C)CNC(=O)NCC1(c2cccc(Cl)c2)CCO1] |
| 61 | N-[[2-(3-chlorophenyl)oxetan-2- yl]methyl]cyclohexanecarboxamide | 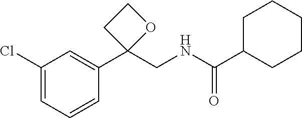 [SMILES: O=C(NCC1(c2cccc(Cl)c2)CCO1)C1CCCCC1] |
| 62 | 1-[(3-chlorophenyl)methyl]-3-[[2-(3- chlorophenyl)oxetan-2- yl]methyl]urea | 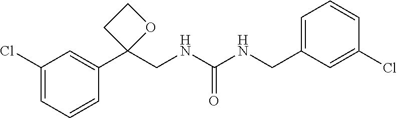 [SMILES: O=C(NCc1cccc(Cl)c1)NCC1(c2cccc(Cl)c2)CCO1] |
| 63 | 2-(1-bicyclo[1.1.1]pentanyl)-N-[[2- (3-chlorophenyl)oxetan-2- yl]methyl]acetamide | 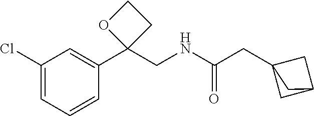 [SMILES: O=C(CC12CC(C1)C2)NCC1(c2cccc(Cl)c2)CCO1] |
| 64 | N-[[2-(3-chlorophenyl)oxetan-2- yl]methyl]spiro[3.3]heptane-2- carboxamide | 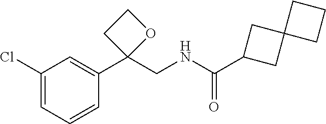 [SMILES: O=C(NCC1(c2cccc(Cl)c2)CCO1)C1CC2(CCC2)C1] |
| 65 | N-[[2-(3-chlorophenyl)oxetan-2- yl]methyl]-2-(4,4- difluorocyclohexyl)acetamide | 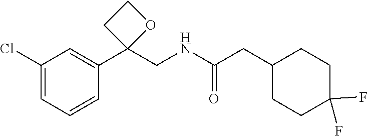 [SMILES: O=C(CC1CCC(F)(F)CC1)NCC1(c2cccc(Cl)c2)CCO1] |
| 66 | (1S,2S)-N-[[2-(3-chlorophenyl)-5- oxo-tetrahydrofuran-2-yl]methyl]-2- phenyl-cyclopropanecarboxamide | 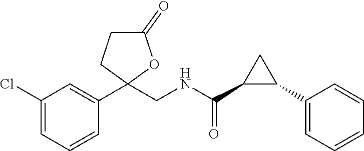 [SMILES: O=C1CCC(CNC(=O)[C@H]2C[C@@H]2c2ccccc2)(c2cccc(Cl)c2)O1] |
| 67 | (1S,2S)-N-[[2-[2-(difluoromethoxy)- 4-pyridyl]oxetan-2-yl]methyl]-2- phenyl-cyclopropanecarboxamide | 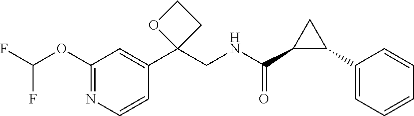 [SMILES: O=C(NCC1(c2ccnc(OC(F)F)c2)CCO1)[C@H]1C[C@@H]1c1ccccc1] |
| 68 | (1S,2S)-N-[[3-(3- chlorophenyl)oxetan-3-yl]methyl]-2- phenyl-cyclopropanecarboxamide | 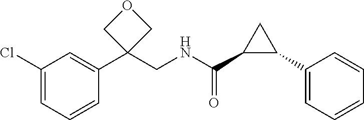 [SMILES: O=C(NCC1(c2cccc(Cl)c2)COC1)[C@H]1C[C@@H]1c1ccccc1] |
| 69 | N-[(2R)-2-(3-chlorophenyl)-2- methoxy-propyl]-4-hydroxy-5,5- dimethyl-hexanamide | 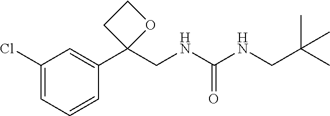 [SMILES: CC(C)(C)CNC(=O)NCC1(c2cccc(Cl)c2)CCO1] |
| 70 | (1S,2S)-N-[(2S)-2-(3-chlorophenyl)- 2-(cyanomethoxy)ethyl]-2-phenyl- cyclopropanecarboxamide | 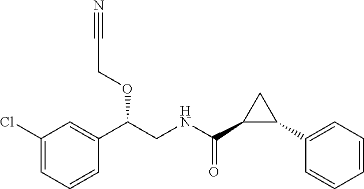 [SMILES: N#CCO[C@H](CNC(=O)[C@H]1C[C@@H]1c1ccccc1)c1cccc(Cl)c1] |
| 71 | 1-(cyclopentylmethyl)-3-[rel-(2S)-2- (3-chlorophenyl)-2-methoxy- propyl]urea | 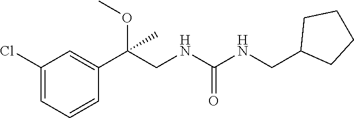 [SMILES: CO[C@@](C)(CNC(=O)NCC1CCCC1)c1cccc(Cl)c1] |
| 72 | 1-[[3-(3-chlorophenyl)oxetan-3- yl]methyl]-3- (cyclopentylmethyl)urea | 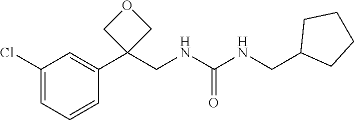 [SMILES: O=C(NCC1CCCC1)NCC1(c2cccc(Cl)c2)COC1] |
| 73 | N-[2-(3-chlorophenyl)-2- (cyanomethoxy)ethyl]-2- cyclopentyl-acetamide | 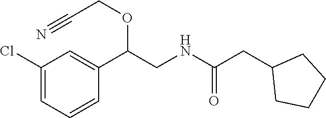 [SMILES: N#CCOC(CNC(=O)CC1CCCC1)c1cccc(Cl)c1] |
| 74 | (1S,2S)-N-[2-(3-chlorophenyl)-3- hydroxy-propyl]-2-phenyl- cyclopropanecarboxamide | 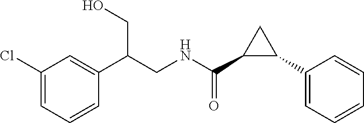 [SMILES: O=C(NCC(CO)c1cccc(Cl)c1)[C@H]1C[C@@H]1c1ccccc1] |
| 75 | 1-[[2-(3-chlorophenyl)-5-oxo- tetrahydrofuran-2-yl]methyl]-3- (cyclopentylmethyl)urea | 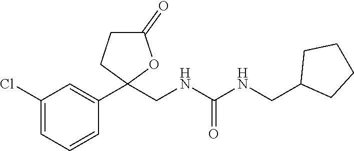 [SMILES: O=C(NCC1CCCC1)NCC1(c2cccc(Cl)c2)CCC(=O)O1] |
| 76 | (1S,2S)-N-[[2-(6-chloro-2- pyridyl)oxetan-2-yl]methyl]-2- phenyl-cyclopropanecarboxamide | 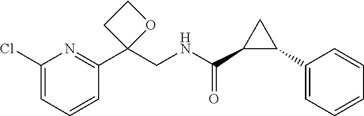 [SMILES: O=C(NCC1(c2cccc(Cl)n2)CCO1)[C@H]1C[C@@H]1c1ccccc1] |
| 77 | (1S,2S)-N-[(E)-2-(3-chlorophenyl)- 4-hydroxy-but-2-enyl]-2-phenyl- cyclopropanecarboxamide | 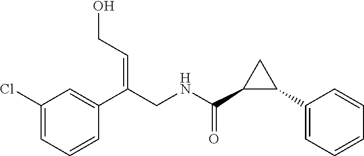 [SMILES: O=C(NC/C(=C/CO)c1cccc(Cl)c1)[C@H]1C[C@@H]1c1ccccc1] |
| 78 | 1-[(E)-2-(3-chlorophenyl)-4- hydroxy-but-2-enyl]-3- (cyclopentylmethyl)urea | 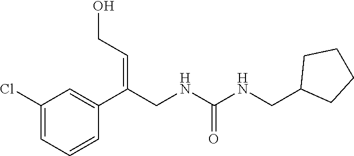 [SMILES: O=C(NC/C(=C/CO)c1cccc(Cl)c1)NCC1CCCC1] |
| 79 | (1RS,2SR)-N-[(2R)-2-(3- chlorophenyl)-2-methoxy-propyl]-2- cyclopropyl- cyclopropanecarboxamide | 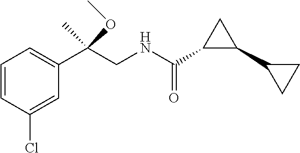 [SMILES: CO[C@@](C)(CNC(=O)[C@@H]1C[C@H]1C1CC1)c1cccc(Cl)c1] |
| 80 | (1RS,2SR)-N-[(2R)-2-(3- chlorophenyl)-2-methoxy-propyl]-2- cyclopentyl- cyclopropanecarboxamide | 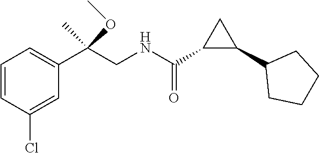 [SMILES: CO[C@@](C)(CNC(=O)[C@@H]1C[C@H]1C1CCCC1)c1cccc(Cl)c1] |
| 81 | (1SR,2SR)-N-[(2R)-2-(3- chlorophenyl)-2-methoxy-propyl]-2- cyclopentyl- cyclopropanecarboxamide | 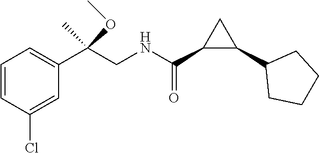 [SMILES: CO[C@@](C)(CNC(=O)[C@H]1C[C@H]1C1CCCC1)c1cccc(Cl)c1] |
| 82 | N-[(2R)-2-(3-chlorophenyl)-2- methoxy-propyl]spiro[2.5]octane-2- carboxamide | 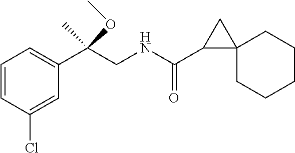 [SMILES: CO[C@@](C)(CNC(=O)C1CC12CCCCC2)c1cccc(Cl)c1] |
| 83 | N-[(2R)-2-(3-chlorophenyl)-2- methoxy-propyl]-6,6-difluoro- spiro[2.5]octane-2-carboxamide | 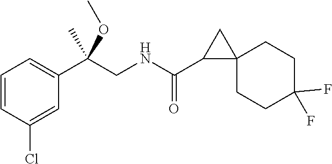 [SMILES: CO[C@@](C)(CNC(=O)C1CC12CCC(F)(F)CC2)c1cccc(Cl)c1] |
| 84 | N-[(2R)-2-(3-chlorophenyl)-2- methoxy-propyl]-6- oxaspiro[2.5]octane-2-carboxamide | 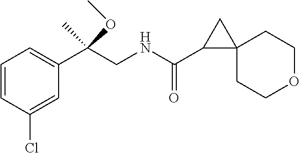 [SMILES: CO[C@@](C)(CNC(=O)C1CC12CCOCC2)c1cccc(Cl)c1] |
| 85 | (1S,2S)-N-[[3-(3- cyanophenyl)oxetan-3-yl]methyl]-2- phenyl-cyclopropanecarboxamide | 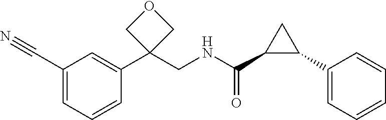 [SMILES: N#Cc1cccc(C2(CNC(=O)[C@H]3C[C@@H]3c3ccccc3)COC2)c1] |
| 86 | (1S,2S)-N-[1-[1-(3- chlorophenyl)cyclopropyl]-2- hydroxy-ethyl]-2-phenyl- cyclopropanecarboxamide | 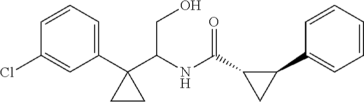 [SMILES: O=C(NC(CO)C1(c2cccc(Cl)c2)CC1)[C@H]1C[C@@H]1c1ccccc1] |
| 87 | (1S,2S)-N-[[2-(3-cyanophenyl)-3,3- difluoro-oxetan-2-yl]methyl]-2- phenyl-cyclopropanecarboxamide | 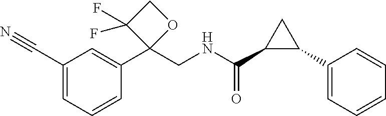 [SMILES: N#Cc1cccc(C2(CNC(=O)[C@H]3C[C@@H]3c3ccccc3)OCC2(F)F)c1] |
| 88 | (1S,2S)-N-[[2-(3-cyanophenyl)-5- oxo-tetrahydrofuran-2-yl]methyl]-2- phenyl-cyclopropanecarboxamide | 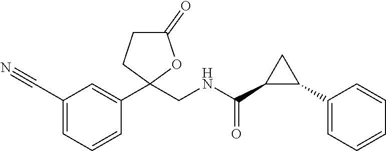 [SMILES: N#Cc1cccc(C2(CNC(=O)[C@H]3C[C@@H]3c3ccccc3)CCC(=O)O2)c1] |
| 89 | N-[[3-(3-cyanophenyl)oxetan-3- yl]methyl]-2-cyclohexyl-acetamide | 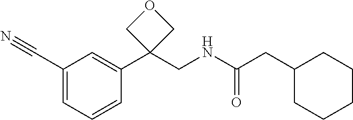 [SMILES: N#Cc1cccc(C2(CNC(=O)CC3CCCCC3)COC2)c1] |
| 90 | N-[[2-(3-cyanophenyl)-5-oxo- tetrahydrofuran-2-yl]methyl]-2- cyclohexyl-acetamide | 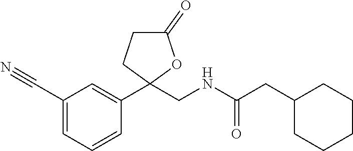 [SMILES: N#Cc1cccc(C2(CNC(=O)CC3CCCCC3)CCC(=O)O2)c1] |
| 91 | N-[[2-(3-cyanophenyl)-3,3-difluoro- oxetan-2-yl]methyl]-2-cyclohexyl- acetamide | 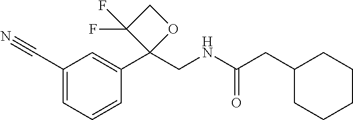 [SMILES: N#Cc1cccc(C2(CNC(=O)CC3CCCCC3)OCC2(F)F)c1] |
| 92 | (1S,2S)-N-[2-(3-chlorophenyl)-4- hydroxy-butyl]-2-phenyl- cyclopropanecarboxamide | 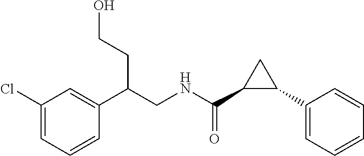 [SMILES: O=C(NCC(CCO)c1cccc(Cl)c1)[C@H]1C[C@@H]1c1ccccc1] |
| 93 | N-[[(2S)-2-(3-cyanophenyl)oxetan-2- yl]methyl]-2-cyclohexyl-acetamide | 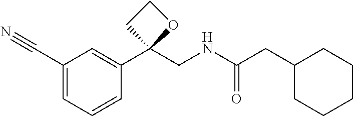 [SMILES: N#Cc1cccc([C@@]2(CNC(=O)CC3CCCCC3)CCO2)c1] |
| 94 | N-[[(2R)-2-(3-cyanophenyl)oxetan- 2-yl]methyl]-2-cycloheptyl- acetamide | 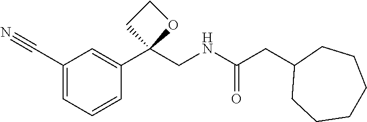 [SMILES: N#Cc1cccc([C@@]2(CNC(=O)CC3CCCCCC3)CCO2)c1] |
| 95 | (1S,2S)-N-[[(2S)-2-(3- cyanophenyl)oxetan-2-yl]methyl]-2- (trifluoromethyl)cyclopropane- carboxamide | 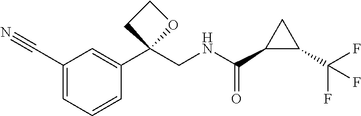 [SMILES: N#Cc1cccc([C@]2(CNC(=O)[C@H]3C[C@@H]3C(F)(F)F)CCO2)c1] |
| 96 | N-[[(2S)-2-(3-cyanophenyl)oxetan-2- yl]methyl]-2,2-difluoro- spiro[2.3]hexane-1-carboxamide | 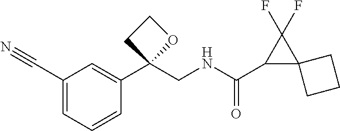 [SMILES: N#Cc1cccc([C@]2(CNC(=O)C3C(F)(F)C34CCC4)CCO2)c1] |
| 97 | N-[[(2R)-2-(3-cyanophenyl)oxetan- 2-yl]methyl]-2-norbornan-2-yl- acetamide | 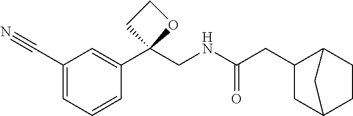 [SMILES: N#Cc1cccc([C@@]2(CNC(=O)CC3CC4CCC3C4)CCO2)c1] |
| 98 | N-[[(2R)-2-(3-cyanophenyl)oxetan- 2-yl]methyl]bicyclo[2.2.2]octane-2- carboxamide | 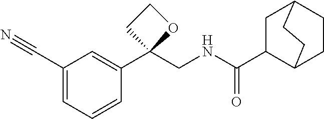 [SMILES: N#Cc1cccc([C@@]2(CNC(=O)C3CC4CCC3CC4)CCO2)c1] |
| 99 | (1S,2S)-N-[[2-[6-(difluoromethoxy)- 2-pyridyl]oxetan-2-yl]methyl]-2- phenyl-cyclopropanecarboxamide | 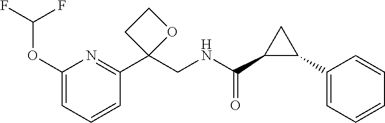 [SMILES: O=C(NCC1(c2cccc(OC(F)F)n2)CCO1)[C@H]1C[C@@H]1c1ccccc1] |
| 100 | N-[[(2S)-2-(3-cyanophenyl)oxetan-2- yl]methyl]spiro[3.3]heptane-3- carboxamide | 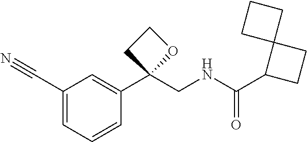 [SMILES: N#Cc1cccc([C@]2(CNC(=O)C3CCC34CCC4)CCO2)c1] |
| 101 | (1SR,2RS)-N-[[(2S)-2-(3- cyanophenyl)oxetan-2-yl]methyl]-2- cyclobutyl- cyclopropanecarboxamide | 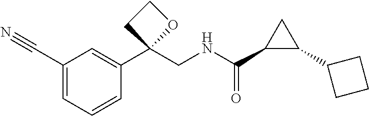 [SMILES: N#Cc1cccc([C@]2(CNC(=O)[C@H]3C[C@@H]3C3CCC3)CCO2)c1] |
| 102 | N-[[(2S)-2-(3-cyanophenyl)oxetan-2- yl]methyl]spiro[3.4]octane-7- carboxamide | 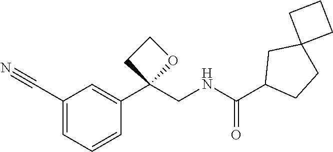 [SMILES: N#Cc1cccc([C@]2(CNC(=O)C3CCC4(CCC4)C3)CCO2)c1] |
| 103 | N-[[(2S)-2-(3-cyanophenyl)oxetan-2- yl]methyl]-3-cyclopentyl-2-fluoro- propanamide | 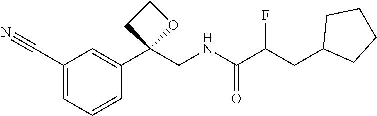 [SMILES: N#Cc1cccc([C@]2(CNC(=O)C(F)CC3CCCC3)CCO2)c1] |
| 104 | N-[[(2S)-2-(3-cyanophenyl)oxetan-2- yl]methyl]spiro[3.4]octane-8- carboxamide | 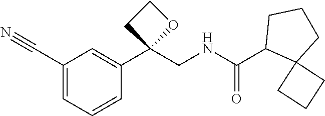 [SMILES: N#Cc1cccc([C@]2(CNC(=O)C3CCCC34CCC4)CCO2)c1] |
| 105 | N-[[(2S)-2-(3-cyanophenyl)oxetan-2- yl]methyl]dispiro[3.0.3⁵.1⁴]nonane- 7-carboxamide | 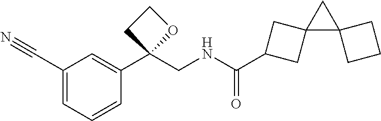 [SMILES: N#Cc1cccc([C@]2(CNC(=O)C3CC4(C3)CC43CCC3)CCO2)c1] |
| 106 | N-[[(2S)-2-(3-cyanophenyl)oxetan-2- yl]methyl]-6-oxaspiro[2.5]octane-2- carboxamide | 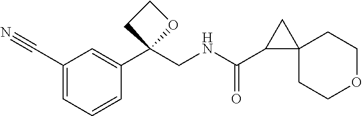 [SMILES: N#Cc1cccc([C@]2(CNC(=O)C3CC34CCOCC4)CCO2)c1] |
| 107 | (1SR,2SR)-N-[[(2S)-2-(3- cyanophenyl)oxetan-2-yl]methyl]-2- (4- fluorophenyl)cyclopropanecarboxamide | 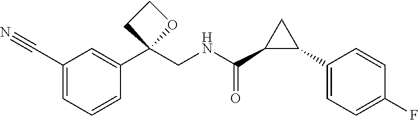 [SMILES: N#Cc1cccc([C@]2(CNC(=O)[C@H]3C[C@@H]3c3ccc(F)cc3)CCO2)c1] |
| 108 | N-[[(2S)-2-(3-cyanophenyl)oxetan-2- yl]methyl]-2-norbornan-2-yl- acetamide | 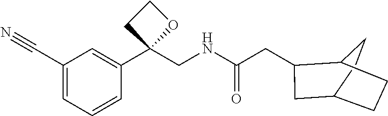 [SMILES: N#Cc1cccc([C@]2(CNC(=O)CC3CC4CCC3CC4)CCO2)c1] |
| 109 | N-[[(2S)-2-(3-cyanophenyl)oxetan-2- yl]methyl]-2-norbornan-1-yl- acetamide | 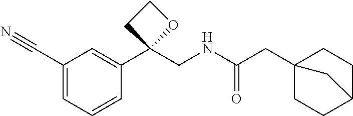 [SMILES: N#Cc1cccc([C@]2(CNC(=O)CC34CCC(CC3)C4)CCO2)c1] |
| 110 | (1RS,2RS)-2-tert-butyl-N-[[(2S)-2- (3-cyanophenyl)oxetan-2- yl]methyl]cyclopropanecarboxamide | 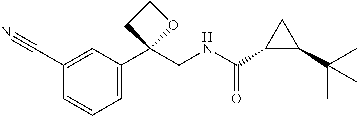 [SMILES: CC(C)(C)[C@@H]1C[C@H]1C(=O)NC[C@@]1(c2cccc(C#N)c2)CCO1] |
| 111 | N-[[(2S)-2-(3-cyanophenyl)oxetan-2- yl]methyl]-2-(2-hydroxynorbornan- 2-yl)acetamide | 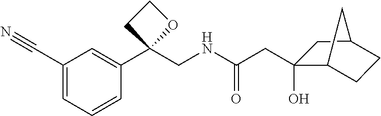 [SMILES: N#Cc1cccc([C@]2(CNC(=O)CC3(O)CC4CCC3C4)CCO2)c1] |
| 112 | (1S,2R)-N-[[(2S)-2-(3- cyanophenyl)oxetan-2-yl]methyl]-2- cyclopentyl- cyclopropanecarboxamide | 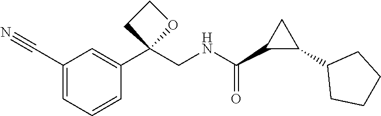 |
| 113 | (1SR,2SR)-N-[[(2S)-2-(3- cyanophenyl)oxetan-2-yl]methyl]-2- (1,1- difluoroethyl)cyclopropane- carboxamide | 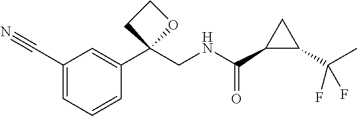 [SMILES: CC(F)(F)[C@H]1C[C@@H]1C(=O)NC[C@@]1(c2cccc(C#N)c2)CCO1] |
| 114 | (1SR,2SR)-2-tert-butoxy-N-[[(2S)-2- (3-cyanophenyl)oxetan-2- yl]methyl]cyclopropanecarboxamide | 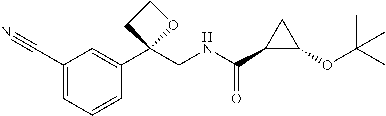 [SMILES: CC(C)(C)O[C@H]1C[C@@H]1C(=O)NC[C@@]1(c2cccc(C#N)c2)CCO1] |
| 115 | N-[[2-[6-(difluoromethoxy)-2- pyridyl]oxetan-2-yl]methyl]-6- oxaspiro[2.5]octane-2-carboxamide | 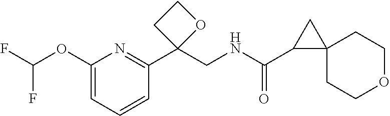 [SMILES: O=C(NCC1(c2cccc(OC(F)F)n2)CCO1)C1CC12CCOCC2] |
| 116 | N-[[(2S)-2-(3-cyanophenyl)oxetan-2- yl]methyl]-2-spiro[3.3]heptan-2-yl- acetamide | 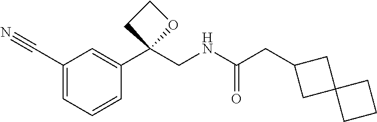 [SMILES: N#Cc1cccc([C@]2(CNC(=O)CC3CC4(CCC4)C3)CCO2)c1] |
| 117 | 2-cyclohexyl-N-[[2-[2- (difluoromethoxy)-4-pyridyl]oxetan- 2-yl]methyl]acetamide | 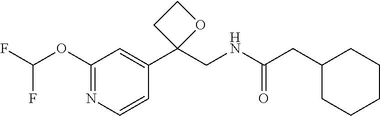 [SMILES: O=C(CC1CCCCC1)NCC1(c2ccnc(OC(F)F)c2)CCO1] |
| 118 | N-[[2-[2-(difluoromethoxy)-4- pyridyl]oxetan-2-yl]methyl]-6,6- difluoro-spiro[2.5]octane-2- carboxamide | 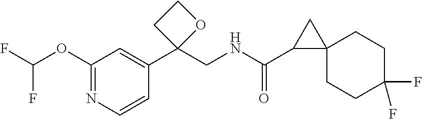 [SMILES: O=C(NCC1(c2ccnc(OC(F)F)c2)CCO1)C1CC12CCC(F)(F)CC2] |
| 119 | 2-cyclohexyl-N-[[2-[6- (difluoromethoxy)-2-pyridyl]oxetan- 2-yl]methyl]acetamide | 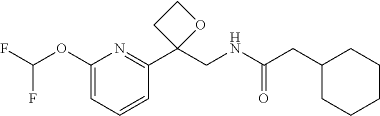 [SMILES: O=C(CC1CCCCC1)NCC1(c2cccc(OC(F)F)n2)CCO1] |
| 120 | N-[[2-[6-(difluoromethoxy)-2- pyridyl]oxetan-2-yl]methyl]-6,6- difluoro-spiro[2.5]octane-2- carboxamide | 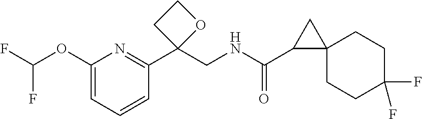 [SMILES: O=C(NCC1(c2cccc(OC(F)F)n2)CCO1)C1CC12CCC(F)(F)CC2] |
| 121 | (1S,2S)-N-[[2-(2-ethyl-4- pyridyl)oxetan-2-yl]methyl]-2- phenyl-cyclopropanecarboxamide | 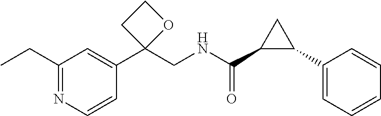 [SMILES: CCc1cc(C2(CNC(=O)[C@H]3C[C@@H]3c3ccccc3)CCO2)ccn1] |
| 122 | N-[[2-[2-(difluoromethoxy)-4- pyridyl]oxetan-2- yl]methyl]spiro[2.5]octane-2- carboxamide | 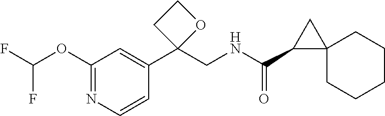 [SMILES: O=C(NCC1(c2ccnc(OC(F)F)c2)CCO1)[C@H]1CC12CCCCC2] |
| 123 | N-[[2-[6-(difluoromethoxy)-2- pyridyl]oxetan-2- yl]methyl]spiro[2.5]octane-2- carboxamide | 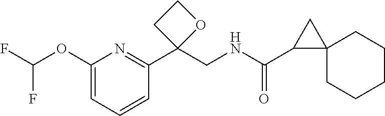 [SMILES: O=C(NCC1(c2cccc(OC(F)F)n2)CCO1)C1CC12CCCCC2] |
| 124 | (1SR,2SR)-N-[[(2S)-2-(3- cyanophenyl)oxetan-2-yl]methyl]-2- (2-pyridyl)cyclopropanecarboxamide | 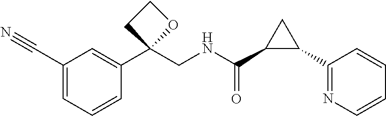 [SMILES: N#Cc1cccc([C@]2(CNC(=O)[C@H]3C[C@@H]3c3ccccn3)CCO2)c1] |
| 125 | (1S,2S)-N-[[2-(3-cyano-4-fluoro- phenyl)oxetan-2-yl]methyl]-2- phenyl-cyclopropanecarboxamide | 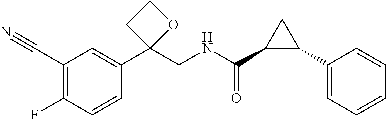 [SMILES: N#Cc1cc(C2(CNC(=O)[C@H]3C[C@@H]3c3ccccc3)CCO2)ccc1F] |
| 126 | (1SR,2SR)-N-[[(2S)-2-(3- cyanophenyl)oxetan-2-yl]methyl]-2- ethyl-cyclopropanecarboxamide | 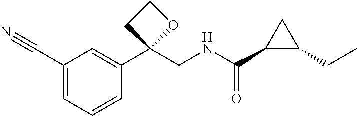 [SMILES: CC[C@H]1C[C@@H]1C(=O)NC[C@@]1(c2cccc(C#N)c2)CCO1] |
| 127 | (1SR,2RS)-N-[[(2S)-2-(3- cyanophenyl)oxetan-2-yl]methyl]-2- (3,3- difluorocyclobutyl) cyclopropanecarboxamide | 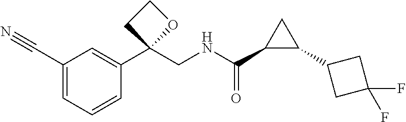 [SMILES: N#Cc1cccc([C@]2(CNC(=O)[C@H]3C[C@@H]3C3CC(F)(F)C3)CCO2)c1] |
| 128 | (1S,2S)-2-(4-bromophenyl)-N-[[(2S)- 2-(3-cyanophenyl)oxetan-2- yl]methyl]cyclopropanecarboxamide | 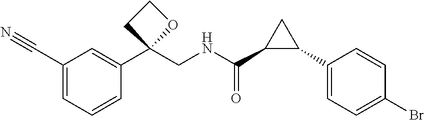 [SMILES: N#Cc1cccc([C@]2(CNC(=O)[C@H]3C[C@@H]3c3ccc(Br)cc3)CCO2)c1] |
| 129 | (1S,2S)-N-[[2-[2-(difluoromethoxy)- 4-pyridyl]oxetan-2-yl]methyl]-2- (trifluoromethyl)cyclopropane- carboxamide | 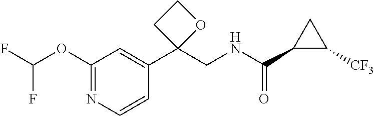 [SMILES: O=C(NCC1(c2ccnc(OC(F)F)c2)CCO1)[C@H]1C[C@@H]1C(F)(F)F] |
| 130 | (1S,2S)-N-[[2-[6-(difluoromethoxy)- 2-pyridyl]oxetan-2-yl]methyl]-2- (trifluoromethyl) cyclopropanecarboxamide | 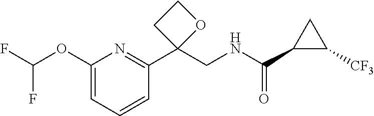 [SMILES: O=C(NCC1(c2cccc(OC(F)F)n2)CCO1)[C@H]1C[C@@H]1C(F)(F)F] |
| 131 | N-[[(2S)-2-(3-cyanophenyl)oxetan-2- yl]methyl]-2-fluoro-2-norbornan-2- yl-acetamide | 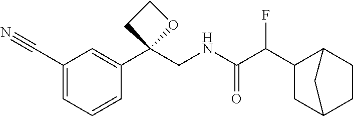 [SMILES: N#Cc1cccc([C@]2(CNC(=O)C(F)C3CC4CCC3C4)CCO2)c1] |
| 132 | N-[[2-[6-(difluoromethoxy)-2- pyridyl]oxetan-2-yl]methyl]-2-[1- (trifluoromethyl)cyclopropyl]acetamide | 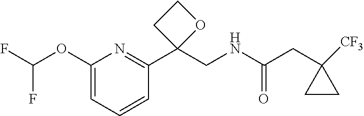 [SMILES: O=C(CC1(C(F)(F)F)CC1)NCC1(c2cccc(OC(F)F)n2)CCO1] |
| 133 | N-[[(2S)-2-(3-cyanophenyl)oxetan-2- yl]methyl]-2-(5-methyl-2- furyl)cyclopropanecarboxamide | 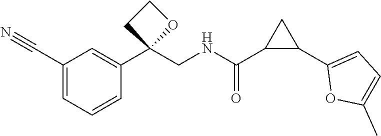 [SMILES: Cc1ccc(C2CC2C(=O)NC[C@@]2(c3cccc(C#N)c3)CCO2)o1] |
| 134 | (1SR,2SR)-N-[[(2S)-2-(3- cyanophenyl)oxetan-2-yl]methyl]-2- prop-1-ynyl- cyclopropanecarboxamide | 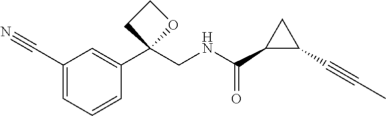 [SMILES: CC#C[C@H]1C[C@@H]1C(=O)NC[C@@]1(c2cccc(C#N)c2)CCO1] |
| 135 | N-[[(2S)-2-(3-cyanophenyl)oxetan-2- yl]methyl]-2-pyrrol-1-yl- cyclopropanecarboxamide | 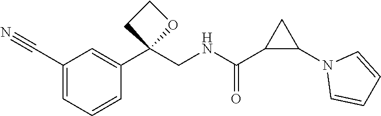 [SMILES: N#Cc1cccc([C@]2(CNC(=O)C3CC3n3cccc3)CCO2)c1] |
| 136 | N-[[(2S)-2-(3-cyanophenyl)oxetan-2- yl]methyl]-2-(4,4- difluorocyclohexyl) cyclopropanecarboxamide | 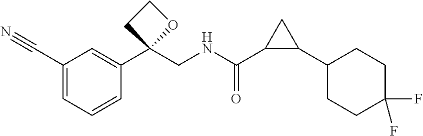 [SMILES: N#Cc1cccc([C@]2(CNC(=O)C3CC3C3CCC(F)(F)CC3)CCO2)c1] |
| 137 | (1SR,2RS)-N-[[(2S)-2-(3- cyanophenyl)oxetan-2-yl]methyl]-2- prop-1-ynyl- cyclopropanecarboxamide | 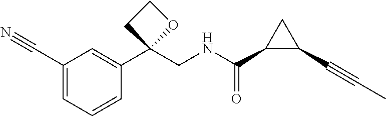 [SMILES: CC#C[C@@H]1C[C@@H]1C(=O)NC[C@@]1(c2cccc(C#N)c2)CCO1] |

Embodiment I-20. A compound of formula (I), or a solvate or a pharmaceutically acceptable salt thereof, according to any one of embodiments I-1 to I-19, wherein such compounds of formula (I) show Kv7.2 EC50 values and/or Kv7.5_7.3Kv7.2 selectivity ratios in the range of: (i) EC50<3 μM, Select>10×, or (ii) EC50<1 μM, Select>30×.

Embodiment I-21. A pharmaceutical composition comprising a compound of formula (I), or a solvate or a pharmaceutically acceptable salt thereof according to any one of embodiments I-1 to I-20.

Embodiment I-22. A compound of formula (I), or a solvate or a pharmaceutically acceptable salt thereof according to any one of embodiments I-1 to I-20; or a pharmaceutical composition according to embodiment I-21; for use in the therapeutic and/or prophylactic treatment of a disorder, disease, or disability associated with Kv7.2 in a subject in need thereof, wherein the disorder, disease, or disability is selected from behavioral disorders, mood disorders, neurodevelopmental disorders, intellectual disability, epilepsies, neurodegenerative diseases, pain, migraine, and tinnitus.

Embodiment I-23. The compound or pharmaceutical composition for use according to embodiment I-22, wherein the disorder, disease, or disability is a behavioral disorder which is Attention Deficit Hyperactivity Disorder (ADHD).

Embodiment I-24. The compound or pharmaceutical composition for use according to embodiment 22, wherein the disorder, disease, or disability is a mood disorder which is depression.

Embodiment I-25. The compound or pharmaceutical composition for use according to embodiment I-22, wherein the disorder, disease, or disability is a neurodevelopment disorder selected from autism spectrum disorder (ASD) and syndromic developmental disorders.

Embodiment I-26. The compound or pharmaceutical composition for use according to embodiment I-22, wherein the disorder, disease, or disability is a syndromic developmental disorder selected from Dup15q syndrome (Dup15q), Fragile X syndrome (FXS) and Angelman syndrome.

Embodiment I-27. The compound or pharmaceutical composition for use according to embodiment 22, wherein the disorder, disease, or disability is an epilepsy selected from broad pediatric epilepsy, West syndrome, Ohtahara syndrome, and epileptic encephalopathy.

Embodiment I-28. The compound or pharmaceutical composition for use according to embodiment I-22, wherein disorder, disease, or disability is a neurodegenerative disease selected from Alzheimer's disease, and motor neuron diseases.

Embodiment I-29. The compound or pharmaceutical composition for use according to any one of embodiments I-21 to I-28, for systemic or local administration such as oral, nasal, parenteral (as by intravenous (both bolus and infusion), intramuscular, or subcutaneous injection), transdermal, vaginal, buccal, rectal, or topical administration modes, intracistemally, intraperitoneally, as an oral or nasal spray, or as a liquid aerosol or dry powder for inhalation.

Embodiment I-30. A compound according to any one of embodiments I-1 to I-20, or a pharmaceutical composition according to embodiment I-21, for use in therapy.

Embodiment I-31. A compound according to any one of embodiments I-1 to I-20, or a pharmaceutical composition according to embodiment 21, or a compound or pharmaceutical composition for use according to any one of embodiment I-22 to I-29, for the manufacture of a medicament for use in the therapeutic and/or prophylactic treatment of a disorder, disease, or disability associated with Kv7.2.

Embodiment I-32. A method for the therapeutic and/or prophylactic treatment of a disorder, disease, or disability associated with Kv7.2, which method comprises administering a therapeutically effective amount of a compound according to any one of embodiments I-1 to I-20, or a pharmaceutical composition according to embodiment I-21, or a pharmaceutical composition for use according to any one of embodiment I-22 to I-29.

Embodiment I-33. A kit for use in the therapeutic and/or prophylactic treatment of a disorder, disease, or disability associated with Kv7.2 comprising:
a) a compound according to any one of embodiments I-1 to I-20, or a pharmaceutical composition according to embodiment I-21, or a pharmaceutical composition for use according to any one of embodiments I-22 to I-29; and
b) instructions for use.

Embodiment I-34. The present invention as described herein.

### Methods of Manufacturing

In a further aspect, the present invention provides a compound of formula (I), or a solvate or a pharmaceutically acceptable salt, when manufactured according to a process described herein.

The preparation of compounds of this invention, in particular compounds selected from formulae (I), and (II)-(XV) may be carried out in sequential or convergent synthetic routes.

Syntheses of the present invention are shown in the following general schemes. The skills required for carrying out the reaction and purification of the resulting products are known to those persons skilled in the art. The substituents and indices used in the following description of the processes have the significance given herein, unless indicated to the contrary.

In any of the embodiments relating to schemes or examples described in this application relating to a compound of this invention, any embodiment can be combined with any other embodiment unless contradictory.

If one of the starting materials, intermediates or compounds of this invention, in particular compounds selected from formulae (I), and (II)-(XV) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of this invention, in particular compounds selected from formulae (I), and (II)-(XV) can be obtained as mixtures of diastereomers or enantiomers, which can be separated by methods well known in the art e.g., chiral HPLC, chiral SFC, or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of this invention will typically lead to the respective diastereomerically/enantiomerically enriched compounds of this invention.

A person skilled in the art will acknowledge that in the synthesis of compounds of this invention, in particular compounds of formulae (I), and (II)-(XV) insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed Engl. 1996, 35, 2056).

A person skilled in the art will acknowledge that the sequence of reactions may be varied depending on reactivity and nature of the intermediates.

In more detail, the compounds of this invention, i.e, compounds selected from any one of formulae (I), and (II)-(XV), or their solvates or pharmaceutically acceptable salts, can be manufactured by the methods given below, by the methods given in the examples or by analogous methods.

Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent.

The described reactions can take place over a wide range of temperatures, and the precise reaction temperature is not critical to the present invention. It is convenient to carry out the described reactions in a temperature range between −78° C. to reflux.

The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis is not described in literature, they can be prepared in analogy to existing procedures for close analogues or as outlined in the experimental section.

### Abbreviations

AcN is acetonitrile
THF is tetrahydrofuran
NEt₃ is triethylamine
DMSO is dimethylsufloxide
EtOAc is ethyl acetate
CDI is 1,1′-carbonyldiimidazole
DCM is dichloromethane
DIPEA is N,N-diisopropylethylamine
DMA is Dimethylacetamide
DMF is N,N-dimethylformamide
HCl is hydrogen chloride
HPLC is high pressure liquid chromatography
LCMS is liquid chromatography mass spectrometry
mCPBA is 3-chloroperoxybenzoic acid
NaHCO₃ is sodium hydrogen carbonate
NaOH is sodium hydroxide
NMI is N-methylimidazole
NMP is N-Methylpyrrolidone
MeOH is methanol
MgSO₄ is magnesium sulfate
Pd(dppf)Cl₂ is 1,1′-bis(di-tert-butylphosphino)ferrocene Palladium dichloride
PYBROP is bromotripyrrolidinophosphonium hexafluorophosphate
o/n is overnight
RT is room temperature
TCFH is Chloro-N,N,N′,N′-tetramethylformamidinium hexafluorophosphate
TLC is thin-layer chromatography
CHO is chinese hamster ovary
CMV is cytomegalovirus
FBS is fetal bovine serum
NEAA is non-essential amino acids
NaCl is sodium chloride
KCl is potassium chloride
CaCl₂ is calcium chloride
MgCl₂ is magnesium chloride
HEPES is 4-(2-hydroxyethyl)-1-piperazine ethanesulfonic acid
NMDG is N-methyl-D-glucamine diatrizoate
EGTA is ethylene glycol-bis(β-aminoethyl ether)-N,N,N′,N′-tetraacetic acid
EDTA is ethylenediaminetetraacetic acid
DPBS is Dulbecco's phosphate-buffered saline
mV is millivolt
TEA is tetraethylammonium
NADPH is nicotinamide adenine dinucleotide phosphate
CLᵢₙₜ is intrinsic clearance.

The present invention provides compounds of formula (I), or a solvate or a pharmaceutically acceptable salts thereof:

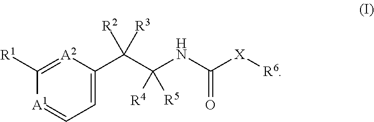

Syntheses of the compounds of the present invention are shown in the following Schemes and in the description of 137 specific examples. The skills required for carrying out the reaction and purification of the resulting products are known to those skilled in the art.

In more detail, the compounds this invention, e.g, compounds selected from formula (I)-(VII), or solvates or pharmaceutically acceptable salts thereof can be manufactured by the methods given in the examples or by analogous methods. Starting materials are either commercially available or can be prepared by methods analogous to the methods given below or by methods known in the art.

### General Procedures

For more information on the general procedures, please refer to the embodiments relating to the process of making compounds of formula (I), or solvates or pharmaceutically acceptable salts thereof as described herein.

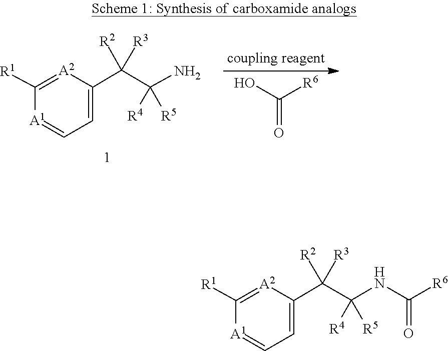

Amine 1 (or the corresponding salt, e.g. HCl) can be reacted with a carboxylic acid with a coupling reagent like HATU or HBTU in the presence of a base (e.g. DIPEA, TEA) or with TCFH and NMI (scheme 1).

Preferred conditions are using TFCH with NMI in acetonitrile at room temperature.

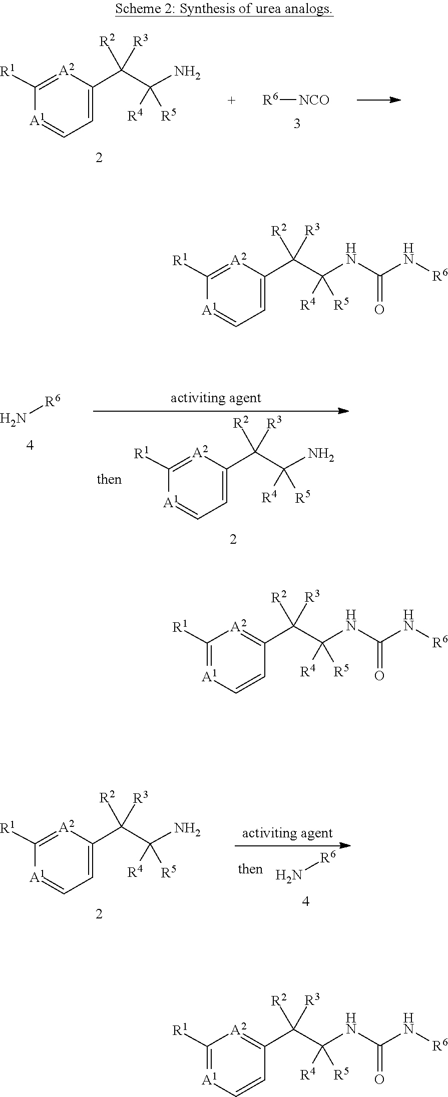

Urea analogs can be synthesized by reacting an amine 2 with an isocyanate 3 in a solvent such as DMF, DCM or ACN. Alternatively, an amine 4 (or 2) can be reacted with an activating reagent, such as CDI, 4-nitrophenyl chloroformate, triphosgene or phenyl chloroformate in a solvent like DMF, DCM or AcN, and in the presence of a suitable base like DIPEA or NEt3, to generate the activated intermediate which can be reacted in situ with a second amine 2 (or 4) (or the corresponding HCl salt) to yield an urea compound. In the case when 4-nitrophenyl chloroformate is used as the activating agent, the carbamate intermediate can be isolated and then be reacted with an amine (or the corresponding HCl salt) in a solvent like DCM or THF to yield the urea product (scheme 2).

Preferred conditions are using CDI with DIPEA as a base and with DCM as a solvent at 0° C. for 45 min and then adding the second amine and stirring at 35° C. for 2-6 hours.

### Isolation and Purification of the Compounds

Isolation and purification of the compounds and intermediates described herein can be carried out, if desired, by any suitable separation or purification procedure such as, for example, filtration, extraction, crystallization, column chromatography, thick-layer chromatography, preparative low or high-pressure liquid chromatography or a combination of these procedures.

However, other equivalent separation or isolation procedures could, of course, also be used. Mixture of chiral compounds of formula (I) can be separated using preparative chiral HPLC purifications.

### EXAMPLES

### Example 1: rac-(1S,2S)—N-[2-(3-chlorophenyl)-2-methoxy-propyl]-2-phenyl-cyclopropanecarboxamide

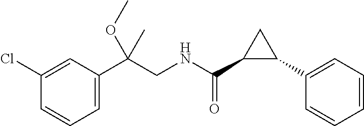 [SMILES: COC(C)(CNC(=O)[C@H]1C[C@@H]1c1ccccc1)c1cccc(Cl)c1]

To a solution of 2-(3-chlorophenyl)-2-methoxypropan-1-amine (20 mg, 0.1 mmol) and rac-(1S,2S)-2-phenylcyclopropane-1-carboxylic acid (19.5 ng, 0.12 mmol) in DMF (0.3 mL) and DIPEA (53 uL, 0.30 mmol) was added HATU (49.5 mg, 0.13 mmol). The reaction was stirred at RT for 2 hours and purified directly by reverse phase HPLC (YMC C18 column 25×250, water and acetonitrile, 0.1% TFA buffer, 5 to 100% gradient) to give the title product as a white solid (23 mg. 66.8% yield).

MS (m/z): 344.3 [M+H]+.

### Example 2: (1S,2S)—N-[(2R)-2-(3-chlorophenyl)-2-methoxy-propyl]-2-phenyl-cyclopropanecarboxamide

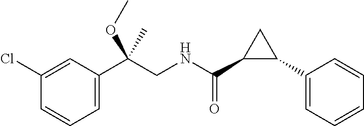 [SMILES: CO[C@@](C)(CNC(=O)[C@H]1C[C@@H]1c1ccccc1)c1cccc(Cl)c1]

The title compound was obtained in analogy to Example 1 as a white powder (70% yield) using (R)-2-(3-chlorophenyl)-2-methoxypropan-1-amine, (1S,2S)-2-phenylcyclopropane-1-carboxylic acid and HATU.

MS (m/z): 342.2 [M+H]+.

### Example 3: 1-[2-(3-chlorophenyl)-2-methoxy-propyl]-3-cyclopentyl-urea

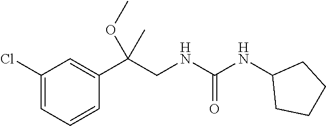 [SMILES: COC(C)(CNC(=O)NC1CCCC1)c1cccc(Cl)c1]

The compound can be prepared by methods similar to those described herein.

MS (m/z): 311.5 [M+H]+.

### Example 4: 1-[2-(3-chlorophenyl)-2-methoxy-ethyl]-3-norboman-2-yl-urea

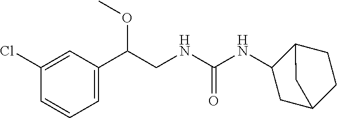 [SMILES: COC(CNC(=O)NC1CC2CCC1C2)c1cccc(Cl)c1]

To a solution of norboman-2-amine (22.2 mg, 0.2 mmol) in DCM (2.0 mL) in an ice batch was added DIPEA (200 ul) and N,N′-carbonyldiimidazole (32.4 mg, 0.2 mmol). The reaction was stirred at 0 C for 1 hour, then (2-(3-chlorophenyl)-2-methoxyethan-1-amine hydrochloride (20.5 mg. 0.12 mmol) was added. The reaction was stirred at 35 C for 15 hrs. The solvent was removed in vacuo. DMF (2 ml) was added and purification by reverse-phase HPLC (YMC C18 column 25×250, water and acetonitrile, 0.1% TFA buffer, 5 to 100% gradient) afforded the title compound as a white solid (39 mg, 64% yield).

MS (m/z): 323.3 [M+H]+.

### Example 5: 1-[2-(3-chlorophenyl)-2-methoxy-propyl]-3-(3-methoxyphenyl)urea

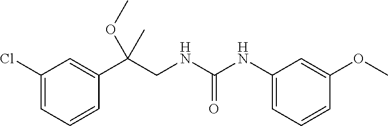 [SMILES: COc1cccc(NC(=O)NCC(C)(OC)c2cccc(Cl)c2)c1]

The compound can be prepared by methods similar to those described herein.

MS (m/z): 340.1 [M+H]+.

### Example 6: 1-[2-(3-chlorophenyl)-2-methoxy-propyl]-3-(m-tolyl)urea

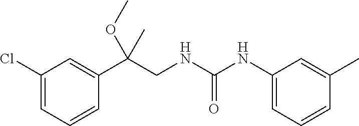 [SMILES: COC(C)(CNC(=O)Nc1cccc(C)c1)c1cccc(Cl)c1]

A solution of 2-(3-chlorophenyl)-2-methoxypropan-1-amine (20 mg, 0.1 mmol) and m-tolyl isocyanate (26.7 mg, 0.2 mmol) in DMF (2 mL) was heated at 50 C for 1 hour. The reaction was purified directly by reverse-phase HPLC (YMC C18 column 25×250, water and acetonitrile, 0.1% TFA buffer, 5 to 100% gradient) to give the title product (11 mg, 33% yield) as a white solid.

MS (m/z): 332.9 [M+H]+.

### Example 7: 1-[[1-(3-chlorophenyl)cyclopropyl]methyl]-3-cyclopentyl-urea

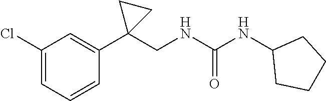 [SMILES: O=C(NCC1(c2cccc(Cl)c2)CC1)NC1CCCC1]

The title compound was obtained in analogy to Example 6 as a white powder (53% yield) using (1-(3-chlorophenyl)cyclopropyl)methanamine and isocyanatocyclopentane.

MS (m/z): 293.5 [M+H]+.

### Example 8: 1-butyl-3-[2-(3-chlorophenyl)-2-methoxy-propyl]urea

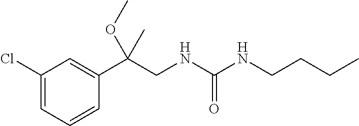 [SMILES: CCCCNC(=O)NCC(C)(OC)c1cccc(Cl)c1]

The title compound was obtained in analogy to Example 6 as a white powder (67% yield) using 2-(3-chlorophenyl)-2-methoxypropan-1-amine and 1-isocyanatobutane.

MS (m/z): 299.5 [M+H]+.

### Example 9: 1-[2-(3-chlorophenyl)-2-methoxy-propyl]-3-(2-furylmethyl)urea

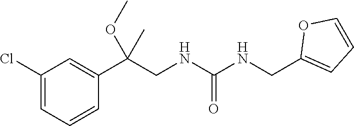 [SMILES: COC(C)(CNC(=O)NCc1ccco1)c1cccc(Cl)c1]

The title compound was obtained in analogy to Example 6 as a white powder (45% yield) using 2-(3-chlorophenyl)-2-methoxypropan-1-amine and 2-(isocyanatomethyl)furan.

MS (m/z): 323.5 [M+H]+.

### Example 10: 1-[2-(3-chlorophenyl)-2-methoxy-propyl]-3-[(3-fluorophenyl)methyl]urea

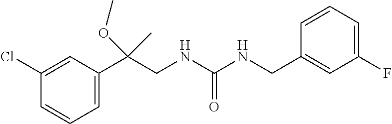 [SMILES: COC(C)(CNC(=O)NCc1cccc(F)c1)c1cccc(Cl)c1]

The title compound was obtained in analogy to Example 6 as a white powder (49% yield) using 2-(3-chlorophenyl)-2-methoxypropan-1-amine and 1-fluoro-3-(isocyanatomethyl)benzene.

MS (m/z): 351.6 [M+H]+.

### Example 11: 1-[2-(3-chlorophenyl)-2-methoxy-propyl]-3-(m-tolylmethyl)urea

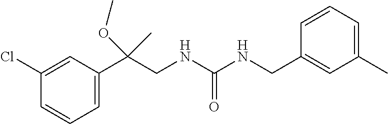 [SMILES: COC(C)(CNC(=O)NCc1cccc(C)c1)c1cccc(Cl)c1]

The title compound was obtained in analogy to Example 6 as a white powder (53% yield) using 2-(3-chlorophenyl)-2-methoxypropan-1-amine and 1-(isocyanatomethyl)-3-methylbenzene.

MS (m/z): 347.5 [M+H]+.

### Example 12: 1-[[1-(3-chlorophenyl)cyclopropyl]methyl]-3-(1-oxaspiro[4.4]nonan-3-yl)urea

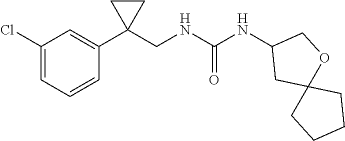 [SMILES: O=C(NCC1(c2cccc(Cl)c2)CC1)NC1COC2(CCCC2)C1]

To (1-(3-chlorophenyl)cyclopropyl)methanamine (36.3 mg, 0.2 mmol) in DCM (0.5 ml) in an ice bath was added phenyl chloroformate (31.3 mg, 0.2 mmol) followed by DIPEA (70 uL, 0.4 mmol). The reaction was stirred at −0° C. for 45 min. 1-oxaspiro[4.4]nonan-3-amine (28.2 mg, 0.2 mmol) was then added and the reaction was stirred at 40° C. for 3 hours. The solvent was removed in vacuo. DMF (2 ml) was added and purification by reverse-phase HPLC (YMC C18 column 25×250, water and acetonitrile, 0.1% TFA buffer, 5 to 100% gradient) afforded the title compound as a white solid (41% yield).

MS (m/z): 349.3 [M+H]+.

### Example 13: 1-[2-(3-chlorophenyl)-2-methoxy-propyl]-3-(1-oxaspiro[4.4]nonan-3-yl)urea

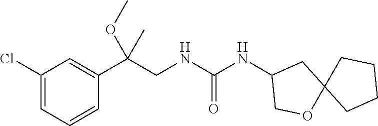 [SMILES: COC(C)(CNC(=O)NC1COC2(CCCC2)C1)c1cccc(Cl)c1]

The title compound was obtained in analogy to Example 12 as a white powder (32% yield) using 2-(3-chlorophenyl)-2-methoxypropan-1-amine, 1-oxaspiro[4.4]nonan-3-amine and phenyl chloroformate.

MS (m/z): 367.3 [M+H]+.

### Example 14: 1-[2-(3-chlorophenyl)-2-methoxy-propyl]-3-(3-ethoxyphenyl)urea

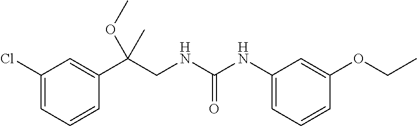 [SMILES: CCOc1cccc(NC(=O)NCC(C)(OC)c2cccc(Cl)c2)c1]

The title compound was obtained in analogy to Example 12 as a white powder (32% yield) using 2-(3-chlorophenyl)-2-methoxypropan-1-amine, 3-ethoxyaniline and phenyl chloroformate.

MS (m/z): 363.5 [M+H]+.

### Example 15: 1-[[1-(3-chlorophenyl)cyclopropyl]methyl]-3-(tetrahydropyran-2-ylmethyl)urea

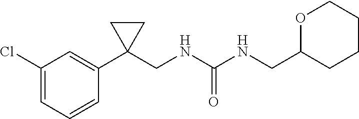 [SMILES: O=C(NCC1CCCCO1)NCC1(c2cccc(Cl)c2)CC1]

The title compound was obtained in analogy to Example 12 as a white powder (19% yield) using (1-(3-chlorophenyl)cyclopropyl)methanamine, (tetrahydro-2H-pyran-2-yl)methanamine and phenyl chloroformate.

MS (m/z): 323.5 [M+H]+.

### Example 16: 1-[(2R)-2-(3-chlorophenyl)-2-methoxy-propyl]-3-cyclopentyl-urea

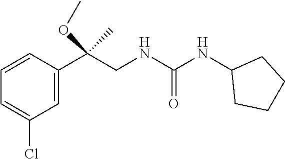 [SMILES: CO[C@@](C)(CNC(=O)NC1CCCC1)c1cccc(Cl)c1]

The title compound was obtained in analogy to Example 6 as a white powder (76% yield) using (R)-2-(3-chlorophenyl)-2-methoxypropan-1-amine and isocyanatocyclopentane.

MS (m/z): 311.3 [M+H]+.

### Example 17: 1-[(2R)-2-(3-chlorophenyl)-2-methoxy-propyl]-3-(3-methoxyphenyl)urea

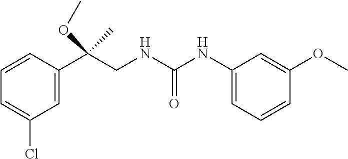 [SMILES: COc1cccc(NC(=O)NC[C@](C)(OC)c2cccc(Cl)c2)c1]

The title compound was obtained in analogy to Example 6 as a white powder (66% yield) using (R)-2-(3-chlorophenyl)-2-methoxypropan-1-amine and 1-isocyanato-3-methoxybenzene.

MS (m/z): 349.3 [M+H]+.

### Example 18: 1-[2-(3-chlorophenyl)-2-(cyclopropoxy)ethyl]-3-cyclopentyl-urea

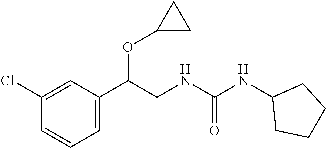 [SMILES: O=C(NCC(OC1CC1)c1cccc(Cl)c1)NC1CCCC1]

### a) 1-(2-(3-chlorophenyl)-2-hydroxyethyl)-3-cyclopentylurea

The title compound was obtained in analogy to Example 6 as a white powder (86% yield) using 2-amino-1-(3-chlorophenyl)ethan-1-ol and isocyanatocyclopentane.

### b) 1-[2-(3-chlorophenyl)-2-(cyclopropoxy)ethyl]-3-cyclopentyl-urea

1-(2-(3-chlorophenyl)-2-hydroxyethyl)-3-cyclopentylurea (32 mg, 0.11 mmol) was dissolved in DMSO (2 mL) and NaH (60%, 20 mg) was added. The reaction mixture was sonicated 2 minute at RT. Then iodocyclopropane (30.4 mg, 0.18 mmol) was added under nitrogen and the reaction was stirred at 55 C for 2 hour. Saturated aq. NH4Cl (10 ml) was added. Extraction with AcOEt (2×20 ml) was carried out. The organic phases were combined, dried over MgSO4 and concentrated. Purification by reverse-phase HPLC (YMC-Pack-ODS-AM column, 250 mm×20 mm I.D., S-5 um, 12 nm, water and acetonitrile, 0.1% TFA buffer, 0 to 60% gradient) afforded the title compound as a white solid (7% yield).

MS (m/z): 322.9 [M+H]+.

### Example 19: N-[2-(3-chlorophenyl)-2-methoxy-propyl]-2-cyclohexyl-acetamide

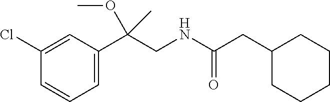 [SMILES: COC(C)(CNC(=O)CC1CCCCC1)c1cccc(Cl)c1]

The title compound was obtained in analogy to Example 1 as a white powder (92% yield) using 2-(3-chlorophenyl)-2-methoxypropan-1-amine, 2-cyclohexylacetic acid and HATU.

MS (m/z): 324.1 [M+H]+.

### Example 20: rac-(1R,2R)—N-[2-(3-chlorophenyl)-2-methoxy-ethyl]-2-phenyl-cyclopropanecarboxamide

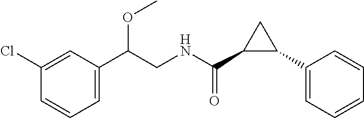 [SMILES: COC(CNC(=O)[C@H]1C[C@@H]1c1ccccc1)c1cccc(Cl)c1]

The title compound was obtained in analogy to Example 1 as a white powder (76% yield) using 2-(3-chlorophenyl)-2-methoxyethan-1-amine hydrochloride, rac-(1R,2R)-2-phenylcyclopropanecarboxylic acid and HATU.

MS (m/z): 330.2 [M+H]+.

### Example 21: 1-[2-(3-chlorophenyl)-2-methoxy-propyl]-3-(cyclopentylmethyl)urea

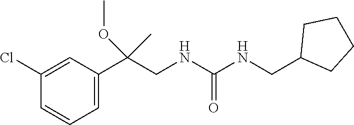 [SMILES: COC(C)(CNC(=O)NCC1CCCC1)c1cccc(Cl)c1]

The title compound was obtained in analogy to Example 12 as a white powder (42% yield) using 2-(3-chlorophenyl)-2-methoxypropan-1-amine, 1-cyclopentylmethanamine and phenyl chloroformate.

MS (m/z): 325.3 [M+H]+.

### Example 22: 1-[2-(3-chlorophenyl)-2-methoxy-ethyl]-3-(m-tolyl)urea

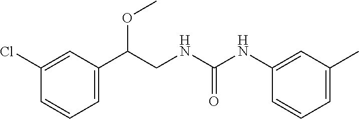 [SMILES: COC(CNC(=O)Nc1cccc(C)c1)c1cccc(Cl)c1]

The title compound was obtained in analogy to Example 6 as a white powder (83% yield) using 2-(3-chlorophenyl)-2-methoxyethan-1-amine hydrochloride and m-tolyl isocyanate.

MS (m/z): 319.3 [M+H]+.

### Example 23: 1-[2-(3-chlorophenyl)-2-methoxy-propyl]-3-(cyclopropylmethyl)urea

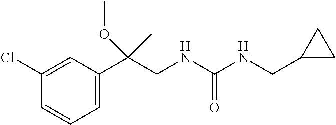 [SMILES: COC(C)(CNC(=O)NCC1CC1)c1cccc(Cl)c1]

The title compound was obtained in analogy to Example 12 as a white powder (32% yield) using 2-(3-chlorophenyl)-2-methoxypropan-1-amine, cyclopropanemethylamine and phenyl chloroformate.

MS (m/z): 297.3 [M+H]+.

### Example 24: 1-[2-(3-bromophenyl)-2-isopropoxy-ethyl]-3-cyclopentyl-urea

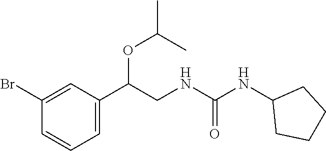 [SMILES: CC(C)OC(CNC(=O)NC1CCCC1)c1cccc(Br)c1]

The title compound was obtained in analogy to Example 18 (6.2% yield) using 1-(2-(3-bromophenyl)-2-hydroxyethyl)-3-cyclopentylurea, 2-iodopropane and sodium hydride.

MS (m/z): 371.0 [M+H]+.

### Example 25: 1-[2-(3-chlorophenyl)-2-methoxy-propyl]-3-[rac-(1S,2S,4R)-norboman-2-yl]urea

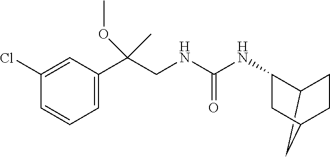 [SMILES: COC(C)(CNC(=O)N[C@H]1CC2CCC1C2)c1cccc(Cl)c1]

The title compound was obtained in analogy to Example 4 (41% yield) using 2-(3-chlorophenyl)-2-methoxypropan-1-amine, rac-(1R,2R,4S)-bicyclo[2.2.1]heptan-2-amine and N,N′-carbonyldiimidazole.

MS (m/z): 337.3 [M+H]+.

### Example 26: 1-[2-(3-chlorophenyl)-2-(trideuteriomethoxy)propyl]-3-cyclopentyl-urea

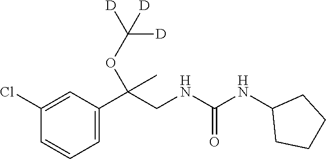 [SMILES: [2H]C([2H])([2H])OC(C)(CNC(=O)NC1CCCC1)c1cccc(Cl)c1]

The title compound was obtained in analogy to Example 18 (4.6% yield) using 1-(2-(3-chlorophenyl)-2-hydroxypropyl)-3-cyclopentylurea, iodomethane-d3 and sodium hydride.

MS (m/z): 314.3 [M+H]+.

### Example 27: 1-(3-methoxyphenyl)-3-[[2-(m-tolyl)-1,3-dioxolan-2-yl]methyl]urea

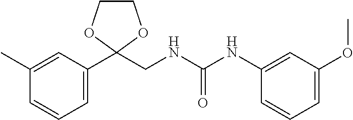

The title compound was obtained in analogy to Example 6 as a white powder (77% yield) using [2-(3-methylphenyl)-1,3-dioxolan-2-yl]methanamine and 3-methoxyphenyl isocyanate.

MS (m/z): 343.5 [M+H]+.

### Example 28: 1-[2-(3-bromophenyl)-2-ethoxy-ethyl]-3-cyclopentyl-urea

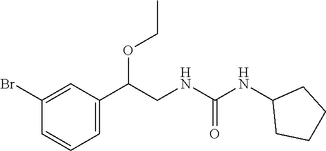 [SMILES: CCOC(CNC(=O)NC1CCCC1)c1cccc(Br)c1]

The title compound was obtained in analogy to Example 18 (6.2% yield) using 1-(2-(3-bromophenyl)-2-hydroxyethyl)-3-cyclopentylurea, iodoethane and sodium hydride.

MS (m/z): 356.9 [M+H]+.

### Example 29: 1-[2-(3-chlorophenyl)-2-methoxy-propyl]-3-[3-(trifluoromethoxy)phenyl]urea

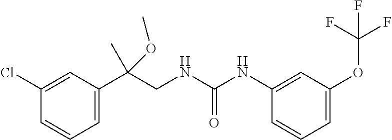 [SMILES: COC(C)(CNC(=O)Nc1cccc(OC(F)(F)F)c1)c1cccc(Cl)c1]

The title compound was obtained in analogy to Example 12 as a white powder (31% yield) using 2-(3-chlorophenyl)-2-methoxypropan-1-amine, 3-(trifluoromethoxy)aniline and phenyl chloroformate.

MS (m/z): 400.3 [M+H]+.

### Example 30: 1-[2-(3-chlorophenyl)-2-methoxy-propyl]-3-cyclopent-3-en-1-yl-urea

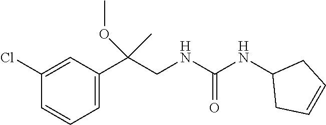 [SMILES: COC(C)(CNC(=O)NC1CC=CC1)c1cccc(Cl)c1]

The title compound was obtained in analogy to Example 4 (59% yield) using 2-(3-chlorophenyl)-2-methoxypropan-1-amine, 1-amino-3-cyclopentene and N,N′-carbonyldiimidazole.

MS (m/z): 309.3 [M+H]+.

### Example 31: rac-(1S,2S)—N-[2-cyano-2-[3-(trifluoromethyl)phenyl]propyl]-2-phenyl-cyclopropanecarboxamide

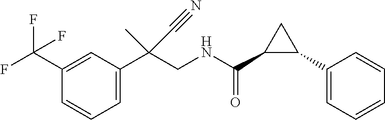 [SMILES: CC(C#N)(CNC(=O)[C@H]1C[C@@H]1c1ccccc1)c1cccc(C(F)(F)F)c1]

### a) 2-(3-(trifluoromethyl)phenyl)propanenitrile

2-[3-(trifluoromethyl)phenyl]acetonitrile (1.851 g, 10 mmol), benzyltriethylammonium chloride (2.28 g, 10 mmol) and a solution of sodium hydroxide (2 g, 50 mmol) in water (10 mL) were combined and shaken vigorously for 30 min. Methyl iodide (0.75 mL, 12 mmol) was added and shaking was continued for 12 h at 45 C. The reaction mixture was diluted with H2O (300 ml), EtOAc (400 ml), and the organic layer was separated. The aqueous layer was extracted with EtOAc(1×) and the organic layers were combined, dried with MgSO4, filtered and evaporated. Purification by flash chromatography on Silica (eluting with hexanes/EtOAc, gradient 0-30% of EtOAc) afforded the product (320 mg. 16% yield).

### b) 3-bromo-2-methyl-2-(3-(trifluoromethyl)phenyl)propanenitrile

2-[3-(trifluoromethyl)phenyl]propanenitrile (320 mg. 1.61 mmol) was dissolved in anhydrous THF (2 mL), cooled to OC and potassium tert-butoxide (234.37 mg, 2.09 mmol) was added. The reaction mixture was stirred at 0 C for 1 h and dibromomethane (0.45 mL, 6.43 mmol) was added. It was stirred at RT for 3 h. Water (50 mL) and EtOAC (100 mL) were added, and the layers were separated. The organic layer was washed with water, brine, dried with MgSO4, then filtered and evaporated. The crude product was obtained (403 mg, 86% yield) and used directly for the next step.

### c) 3-(1,3-dioxoisoindolin-2-yl)-2-methyl-2-(3-(trifluoromethyl)phenyl)propanenitrile

3-bromo-2-methyl-2-[3-(trifluoromethyl)phenyl]propanenitrile (403 mg, 1.38 mmol) was dissolved in NMP (5 mL) and phthalimide potassium (332.2 mg. 1.79 mmol) was added. The reaction mixture was stirred overnight at 120 C. It was then diluted with EtOAc, extracted with water, brine, dried with MgSO4, filtered and evaporated. Purification by flash chromatography on Silica (eluting with hexanes/EtOAc, gradient 0-5% of EtOAc) afforded the product (282 mg, 57% yield).

### d) 3-amino-2-methyl-2-(3-(trifluoromethyl)phenyl)propanenitrile

3-(1,3-dioxoisoindolin-2-yl)-2-methyl-2-[3-(trifluoromethyl)phenyl]propanenitrile (282 mg, 0.79 mmol) was suspended in EtOH (8 mL) and hydrazine monohydrate (394 mg, 7.87 mmol) was added. The reaction mixture was stirred at 60 C for 1 h. A precipitate formed. The reaction mixture was concentrated in vacuo and the residue was dissolved in water, extracted with EtOAc. The organic layer was dried with MgSO4, filtered and concentrated to afford the product as a yellowish oil (141.5 mg, 78.8% yield) which was used for the next step without further purification.

### d) rac-(1S,2S)—N-[2-cyano-2-[3-(trifluoromethyl)phenyl]propyl]-2-phenyl-cyclopropanecarboxamide

The title compound was obtained in analogy to Example 1 as a white powder (37% yield) using 3-amino-2-methyl-2-(3-(trifluoromethyl)phenyl)propanenitrile, rac-(1R,2R)-2-phenylcyclopropanecarboxylic acid and HATU.

MS (m/z): 373.4 [M+H]+.

### Example 32: 1-[2-(3-chlorophenyl)-2-methoxy-propyl]-3-(3-phenylpropyl)urea

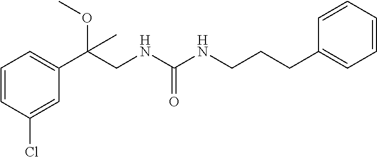 [SMILES: COC(C)(CNC(=O)NCCCc1ccccc1)c1cccc(Cl)c1]

The compound can be prepared by methods similar to those described herein.

MS (m/z): 360.9 [M+H]+.

### Example 33: 1-[2-(3-chlorophenyl)-2-methoxy-propyl]-3-(2-thienylmethyl)urea

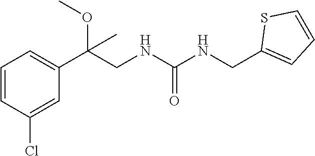 [SMILES: COC(C)(CNC(=O)NCc1cccs1)c1cccc(Cl)c1]

The compound can be prepared by methods similar to those described herein.

MS (m/z): 338.9 [M+H]+.

### Example 34: 1-[2-(3-chlorophenyl)-2-methoxy-propyl]-3-[2-(4-methoxyphenyl)ethyl]urea

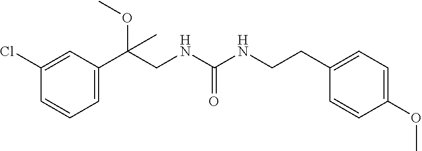 [SMILES: COc1ccc(CCNC(=O)NCC(C)(OC)c2cccc(Cl)c2)cc1]

The compound can be prepared by methods similar to those described herein.

MS (m/z): 376.9 [M+H]+.

### Example 35: 1-[2-(3-chlorophenyl)-2-methoxy-propyl]-3-[rac-(1S,4R)-norbornan-2-yl]urea

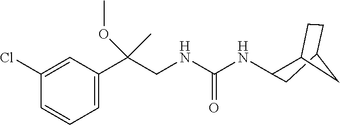 [SMILES: COC(C)(CNC(=O)NC1CC2CCC1C2)c1cccc(Cl)c1]

The title compound was obtained in analogy to Example 4 (65% yield) using 2-(3-chlorophenyl)-2-methoxypropan-1-amine, rac-(1S,2R,4R)-bicyclo[2.2.1]heptan-2-amine and N,N′-carbonyldiimidazole.

MS (m/z): 337.3 [M+H]+.

### Example 36: N-[2-(2-chlorophenyl)-2-methoxy-ethyl]-2-(1-naphthyl)acetamide

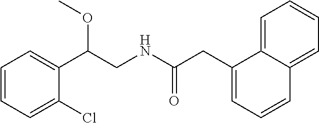 [SMILES: COC(CNC(=O)Cc1cccc2ccccc12)c1ccccc1Cl]

The compound can be prepared by methods similar to those described herein.

MS (m/z): 354.5 [M+H]+.

### Example 37: rac-(1S,2S)—N-[2-(3-chlorophenyl)-2-cyano-propyl]-2-phenyl-cyclopropanecarboxamide

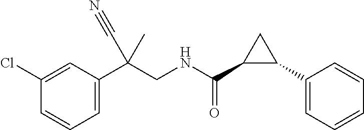 [SMILES: CC(C#N)(CNC(=O)[C@H]1C[C@@H]1c1ccccc1)c1cccc(Cl)c1]

The title compound was obtained in analogy to Example 31 using 2-(3-chlorophenyl)acetonitrile, rac-(1R,2R)-2-phenylcyclopropanecarboxylic acid, and HATU.

MS (m/z): 339.2 [M+H]+.

### Example 38: 1-[3-(1r,3r)-(3-chlorophenyl)cyclobutyl]-3-[(1SR,2SR,4RS)-norboman-2-yl]urea

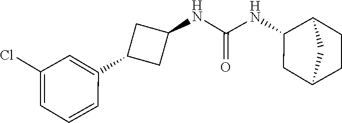 [SMILES: O=C(N[C@H]1C[C@@H]2CC[C@H]1C2)N[C@H]1C[C@H](c2cccc(Cl)c2)C1]

### a) 4-nitrophenyl N-[(1r,3r)-3-(3-chlorophenyl)cyclobutyl]carbamate

4-Nitrophenyl chloroformate (485.1 mg, 2.41 mmol) was stirred in DCM (10 mL) at 0 C under N2. (1r,3r)-3-(3-chlorophenyl)cyclobutan-1-amine hydrochloride (500 mg, 2.29 mmol) and DIPEA (0.88 mL, 5.04 mmol) were combined in DCM (5 mL) to form a suspension. This was added dropwise to the chloroformate solution. The resulting mixture was stirred at 0 C for 1 h under N2, then allowed to warm to RT for 1 h. The reaction was diluted with DCM (30 mL) and washed with sat aq. NaHCO3 (30 mL). The organic layer was washed with brine (30 mL), dried over MgSO4 and concentrated in vacuo to afford a yellow solid (900 mg). Purification by flash chromatography on silica (eluting with Heptane-60% EtOAc/heptane gradient) afforded the desired product as a white solid (370 mg, 46.6%).

### b) 1-[3-(1r,3r)-(3-chlorophenyl)cyclobutyl]-3-[(1SR,2SR,4RS)-norboman-2-yl]urea

To an ice cooled solution of 4-nitrophenyl N-[(1r,3r)-3-(3-chlorophenyl)cyclobutyl]carbamate (100 mg. 0.29 mmol) in DMF (2 mL) was slowly added a mixture of exo-norbornylamine hydrochloride (46.8 mg. 0.32 mmol) and DIPEA (0.11 mL, 0.63 mmol) in DMF (2 mL). After 5 min it was allowed to warm to RT. The reaction was stirred at RT for 2 h. The mixture was then partitioned between EtOAc (2×20 ml) and water (20 ml). The combined organic layers washed with brine (3×20 ml), dried (MgSO4) and evaporated in vacuo to afford a crude oil (150 mg). Purification by flash chromatography on silica (eluting with heptane-50% EtOAc gradient) afforded the desired product as a white solid (75 mg, 81.6%).

MS (m/z): 319.2 [M+H]+.

### Example 39: (1S,2S)—N-[[(2R)-2-(3-chlorophenyl)oxetan-2-yl]methyl]-2-phenyl-cyclopropanecarboxamide

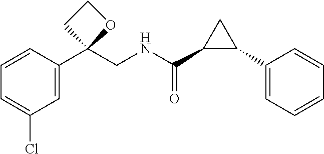 [SMILES: O=C(NC[C@]1(c2cccc(Cl)c2)CCO1)[C@H]1C[C@@H]1c1ccccc1]

The title compound was obtained in analogy to Example 1 as a white solid using [(2R)-2-(3-chlorophenyl)oxetan-2-yl]methanamine, (1S,2S)-2-phenylcyclopropanecarboxylic acid, and HATU.

MS (m/z): 341.9 [M+H]+.

### Example 40: (1S,2S)—N-[[(2S)-2-(3-chlorophenyl)oxetan-2-yl]methyl]-2-phenyl-cyclopropanecarboxamide

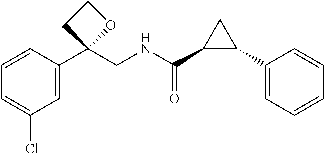 [SMILES: O=C(NC[C@@]1(c2cccc(Cl)c2)CCO1)[C@H]1C[C@@H]1c1ccccc1]

The title compound was obtained in analogy to Example 1 as a white solid using [(2S)-2-(3-chlorophenyl)oxetan-2-yl]methanamine, (1S,2S)-2-phenylcyclopropanecarboxylic acid, and HATU. This is the diastereoisomer of Example 39 isolated by purification on reverse phase HPLC.

MS (m/z): 341.9 [M+H]+.

### Example 41: (1S,2S)—N-[[(2R)-2-(3-chlorophenyl)-3,3-difluoro-oxetan-2-yl]methyl]-2-phenyl-cyclopropanecarboxamide

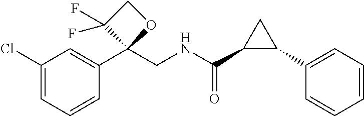 [SMILES: O=C(NC[C@@]1(c2cccc(Cl)c2)OCC1(F)F)[C@H]1C[C@@H]1c1ccccc1]

### a) ((1-(3-chlorophenyl)ethenyl)oxy)trimethylsilane

To a solution of diisopropylamine (5.45 mL, 0.72 g/mL, 38.81 mmol) in THF (125 mL) at −78 C under N2, n-Butyl Lithium (14.23 mL (2.5M), 35.58 mmol) was added followed by a solution of 3′-chloroacetophenone (4.17 mL, 1.2 g/mL, 32.34 mmol) in THF (25 mL) dropwise. After 20 min trimethylchlorosilane (6.16 mL, 0.86 g/mL, 48.51 mmol) dissolved in THF (25 mL) was added slowly. After 10 min the cooling bath was removed and the reaction was stirred at RT for 15 min before it was recooled and aq. NaHCO3 sol. (100 ml) was added. The mixture was allowed to warm to RT. Water (100 ml) and Et2O (100 ml) were added and partitioned. The organic extract was washed with brine (50 ml), dried (MgSO4) and concentrated in vacuo to afford a crude liquid. Purification by flash chromatography on silica eluting with heptane-2% EtOAc/heptane (gradient) afforded the desired product as a colourless liquid (4.5 g, 61.4%).

### b) 3-bromo-1-(3-chlorophenyl)-2, 2-difluoropropan1-one

To a solution of ((1-(3-chlorophenyl)ethenyl)oxy) trimethylsilane (2.01 g, 8.86 mmol) in acetonitrile (9 mL) under N2, [Bromo(difluoro)methyl]trimethylsilane (2.07 mL, 1.31 g/mL, 13.3 mmol) was added followed by tetrabutylammonium bromide (571.5 mg, 1.77 mmol). The reaction mixture was heated at 80 C for 1.5 h, then allowed to cool to RT. N-Bromosuccinimide (2.05 g, 11.52 mmol, 1.3 eq) was then added and stirred for 10 min. Water (80 ml) was added and extracted with heptane (3×50 ml). The combined organics were dried (MgSO4) and evaporated in vacuo to afford a yellow oil (2.5 g). Purification by flash chromatography on silica eluting with heptane-3% EtOAc/heptane (gradient) afforded the desired product as a pale yellow liquid (1.15 g, 45.8%).

### c) 2-(3-chlorophenyl)-3,3-difluorooxetane2-carbonitrile

To a solution of 3-bromo-1-(3-chlorophenyl)-2,2-difluoropropan1-one (750 mg, 2.65 mmol) in DMF (15 mL) under N2, was added potassium cyanide (258.4 mg, 3.97 mmol) was added. The reaction went purple and was left to stir o/n. The reaction mixture was partitioned between EtOAc (2×40 ml) and brine/water (15 ml). The combined organics were washed with brine (3×30 ml), dried (MgSO4) and evaporated in vacuo to afford a crude oil (~750 mg). Purification by flash chromatography on silica eluting with heptane-8% EtOAc (gradient) afforded the desired oxetane product as a colourless liquid (440 mg, 72.4%).

### d) [2-(3-chlorophenyl)-3, 3-difluorooxetan2-yl]methanamine

To a solution of 2-(3-chlorophenyl)-3,3-difluorooxetane2-carbonitrile (300 mg, 1.31 mmol) in THF (6 mL) and water (3 mL) cooled in a salt/ice bath, Cobalt(II) chloride hexahydrate (155.4 mg, 0.65 mmol) was added. The mixture gradually turned purple as the CoCl2 dissolved. Sodium borohydride (494.3 mg, 13.07 mmol) was then added in five portions over 20 min. Black precipitate formed immediately, and gas evolved. The reaction was left open to the atmosphere as lots of gas evolved, then allowed to slowly warm to RT in the cooling bath. It was left to stir o/n. An aq. NH3 solution (3 ml) and water (5 ml) were added to the reaction mixture and stirred vigorously for 10 min. EtOAc (~20 ml) was added and the mixture was passed through a plug of celite. Washing was carried out with further EtOAc and water. The layers were separated, the aqueous layer was washed with further EtOAc (20 ml). The combined organics were dried (MgSO4) and evaporated in vacuo to afford a crude yellow oil (255 mg). Purification by flash chromatography on silica eluting with DCM-5% MeOH/DCM (gradient) afforded the desired product as a yellow oil (53 mg, 17.4%).

### e) (1S,2S)—N-((2-(3-chlorophenyl)-3,3-difluorooxetan2-yl)methyl)-2-phenylcyclopropane1-carboxamide

The title compound was obtained in analogy to Example 1 (65% yield) using [2-(3-chlorophenyl)-3,3-difluorooxetan2-yl]methanamine, (1S,2S)-2-phenylcyclopropane1-carboxylic acid and HATU.

MS (m/z): 378 [M+H]+.

### f)(1S,2S)—N-[[(2R)-2-(3-chlorophenyl)-3,3-difluoro-oxetan-2-yl]methyl]-2-phenyl-cyclopropanecarboxamide

The title compound was obtained via chiral separation of (1S,2S)—N-((2-(3-chlorophenyl)-3,3-difluorooxetan2-yl)methyl)-2-phenylcyclopropane1-carboxamide (100% de, 46.7% yield) as a white solid (Cellulose-4 column, in water:MeCN 48:52, first isomer to elute).

MS (m/z): 378.1 [M+H]+.

### Example 42: (1S,2S)—N-[[(2S)-2-(3-chlorophenyl)-3,3-difluoro-oxetan-2-yl]methyl]-2-phenyl-cyclopropanecarboxamide

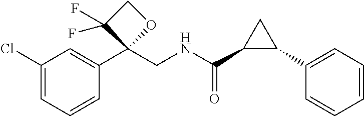 [SMILES: O=C(NC[C@]1(c2cccc(Cl)c2)OCC1(F)F)[C@H]1C[C@@H]1c1ccccc1]

The title compound is the other diastereomer of Example 41 obtained via chiral separation of (1S,2S)—N-((2-(3-chlorophenyl)-3,3-difluorooxetan2-yl)methyl)-2-phenylcyclopropane1-carboxamide (99.5% de, 50.0% yield) as a white solid (Cellulose-4 column, in water:MeCN 48:52, second isomer to elute).

MS (m/z): 378.1 [M+H]+.

### Example 43: N-[2-(3-chlorophenyl)-2-methoxy-propyl]-2-(2-fluorophenyl) cyclopropanecarboxamide

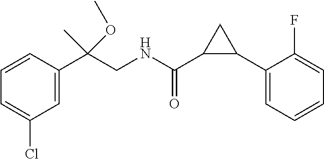 [SMILES: COC(C)(CNC(=O)C1CC1c1ccccc1F)c1cccc(Cl)c1]

The title compound was obtained in analogy to Example 1 (25% yield) as a white solid using 2-(3-chlorophenyl)-2-methoxypropan-1-amine, 2-(2-fluorophenyl)cyclopropane carboxylic acid and HATU.

MS (m/z): 362.5 [M+H]+.

### Example 44: 2-(4-chlorophenyl)-N-[2-(3-chlorophenyl)-2-methoxy-propyl]cyclopropanecarboxamide

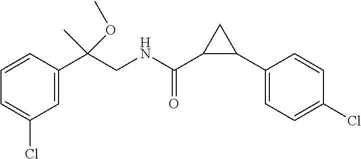 [SMILES: COC(C)(CNC(=O)C1CC1c1ccc(Cl)cc1)c1cccc(Cl)c1]

The title compound was obtained in analogy to Example 1 (12% yield) as a white solid using 2-(3-chlorophenyl)-2-methoxypropan-1-amine, 2-(4-chlorophenyl)cyclopropanecarboxylic acid and HATU.

MS (m/z): 378.4 [M+H]+.

### Example 45: 1-[2-(3-chlorophenyl)-2-(2,2,2-trifluoroethoxy)ethyl]-3-[rac-(1R,4S)-norboman-2-yl]urea

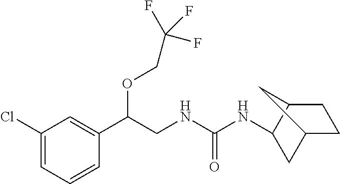 [SMILES: O=C(NCC(OCC(F)(F)F)c1cccc(Cl)c1)NC1CC2CCC1CC2]

The title compound was obtained in analogy to Example 38 (11% yield) as a white solid using 2-(3-chlorophenyl)-2-(2,2,2-trifluoroethoxy)ethan-1-amine, exo-2-aminonorbornane and 4-nitrophenyl chloroformate.

MS (m/z): 391.3 [M+H]+.

### Example 46: 1-[2-(3-chlorophenyl)-2-(cyclobutoxy)ethyl]-3-[rac-(1R,4S)-norboman-2-yl]urea

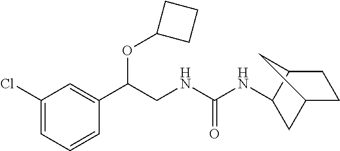 [SMILES: O=C(NCC(OC1CCC1)c1cccc(Cl)c1)NC1CC2CCC1CC2]

The title compound was obtained in analogy to Example 38 (36% yield) as a white solid using 1-(2-amino-1-cyclobutoxyethyl)-3-chlorobenzene, exo-2-aminonorbomane and 4-nitrophenyl chloroformate.

MS (m/z): 363.4 [M+H]+.

### Example 47: (E)-N-[2-(3-chlorophenyl)-2-methoxy-propyl]-3-(2-fluorophenyl)prop-2-enamide

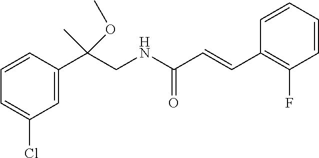 [SMILES: COC(C)(CNC(=O)/C=C/c1ccccc1F)c1cccc(Cl)c1]

The title compound was obtained in analogy to Example 1 (5% yield) as a white solid using 2-(3-chlorophenyl)-2-methoxypropan-1-amine, 2-fluorocinnamic acid and HATU.

MS (m/z): 348.4 [M+H]+.

### Example 48: (1SR,2SR)—N-[(2R)-2-(3-chlorophenyl)-2-methoxy-propyl]-2-(3-pyridyl)cyclopropanecarboxamide

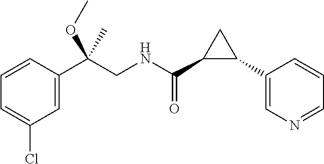 [SMILES: CO[C@@](C)(CNC(=O)[C@H]1C[C@@H]1c1cccnc1)c1cccc(Cl)c1]

The title compound was obtained in analogy to Example 1 (21% yield) as a white solid using (S)-2-(3-chlorophenyl)-2-methoxypropan-1-amine, rac-(1R,2R)-2-(pyridin-3-yl)cyclopropane-1-carboxylic acid and HATU.

MS (m/z): 344.9 [M+H]+.

### Example 49: (1S,2S)—N-[[(2S)-2-(3-cyanophenyl)oxetan-2-yl]methyl]-2-phenyl-cyclopropanecarboxamide

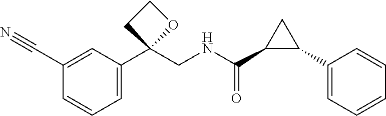 [SMILES: N#Cc1cccc([C@]2(CNC(=O)[C@H]3C[C@@H]3c3ccccc3)CCO2)c1]

The title compound was obtained in analogy to Example 1 (42% yield) as a white solid using 3-[(2S)-2-(aminomethyl)oxetan-2-yl]benzonitrile, (1S,2S)-2-phenylcyclopropane-1-carboxylic acid and HATU, which was purified by flash chromatography on silica eluting with 10-30% AcOEt in hexane (gradient).

MS (m/z): 332.9 [M+H]+.

### Example 50: 1-[[2-(3-chlorophenyl)oxetan-2-yl]methyl]-3-(3-methoxyphenyl)urea

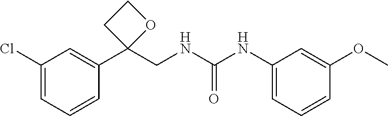 [SMILES: COc1cccc(NC(=O)NCC2(c3cccc(Cl)c3)CCO2)c1]

The title compound was obtained in analogy to Example 4 (42% yield) using [2-(3-chlorophenyl)oxetan-2-yl]methanamine, m-anisidine and N,N′-carbonyldiimidazole.

MS (m/z): 346.9 [M+H]+.

### Example 51: (1S,2S)—N-[2-(2-chloro-4-pyridyl)-2-methoxy-propyl]-2-phenyl-cyclopropanecarboxamide

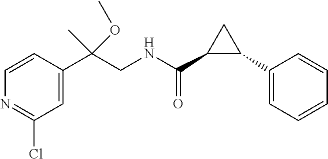 [SMILES: COC(C)(CNC(=O)[C@H]1C[C@@H]1c1ccccc1)c1ccnc(Cl)c1]

The title compound was obtained in analogy to Example 1 (76% yield) as a white solid using 2-(2-chloropyridin-4-yl)-2-methoxypropan-1-amine, (1S,2S)-2-phenylcyclopropane-1-carboxylic acid and HATU.

MS (m/z): 344.9 [M+H]+.

### Example 52: (1S,2S)—N-[2-methoxy-2-(2-methoxy-4-pyridyl)propyl]-2-phenyl-cyclopropanecarboxamide

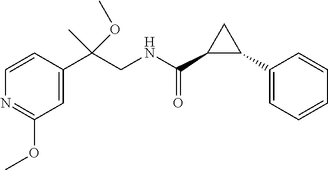 [SMILES: COc1cc(C(C)(CNC(=O)[C@H]2C[C@@H]2c2ccccc2)OC)ccn1]

The title compound was obtained in analogy to Example 1 (82% yield) as a white solid using 2-methoxy-2-(2-methoxypyridin-4-yl)propan-1-amine, (1S,2S)-2-phenylcyclopropane-1-carboxylic acid and HATU.

MS (m/z): 340.9 [M+H]+.

### Example 53: N-[2-(3-chlorophenyl)-2-methoxy-propyl]-3-cyclopentyl-propanamide

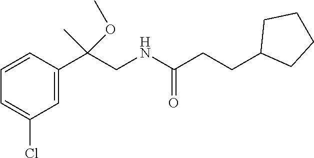 [SMILES: COC(C)(CNC(=O)CCC1CCCC1)c1cccc(Cl)c1]

The title compound was obtained in analogy to Example 1 (10% yield) as a white solid using 2-(3-chlorophenyl)-2-methoxypropan-1-amine, 3-cyclopentylpropionic acid and HATU.

MS (m/z): 324.5 [M+H]+.

### Example 54: 1-[[2-(3-chlorophenyl)oxetan-2-yl]methyl]-3-[rac-(1S,2S,4R)-norboman-2-yl]urea

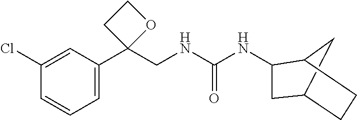 [SMILES: O=C(NCC1(c2cccc(Cl)c2)CCO1)NC1CC2CCC1C2]

The title compound was obtained in analogy to Example 4 (39% yield) using [2-(3-chlorophenyl)oxetan-2-yl]methanamine, exo-2-aminonorbornane and N,N′-carbonyldiimidazole.

MS (m/z): 335.2 [M+H]+.

### Example 55: (1S,2S)—N-[[(2R)-2-(3-cyanophenyl)oxetan-2-yl]methyl]-2-phenyl-cyclopropanecarboxamide

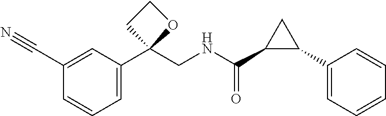 [SMILES: N#Cc1cccc([C@@]2(CNC(=O)[C@H]3C[C@@H]3c3ccccc3)CCO2)c1]

The title compound is the second diastereoisomer isolated from purification of Example 49 (32% yield) as a white solid after silica gel chromatography purification eluting with 10-30% AcOEt in hexane (gradient).

MS (m/z): 332.9 [M+H]+.

### Example 56: N-[3-(1r,3r)-(3-chlorophenyl)cyclobutyl]-2-cyclohexyl-acetamide

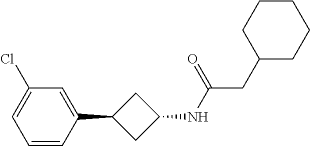 [SMILES: O=C(CC1CCCCC1)N[C@H]1C[C@H](c2cccc(Cl)c2)C1]

The title compound was obtained in analogy to Example 1 (84% yield) as a white solid using (1r,3r)-3-(3-chlorophenyl)cyclobutan1-amine hydrochloride, 3-cyclopentylpropionic acid and HATU.

MS (m/z): 306.2 [M+H]+.

### Example 57: N-[[2-(3-chlorophenyl)oxetan-2-yl]methyl]-2-cyclohexyl-acetamide

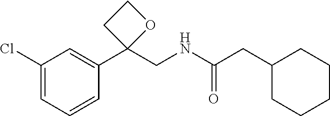 [SMILES: O=C(CC1CCCCC1)NCC1(c2cccc(Cl)c2)CCO1]

The title compound was obtained in analogy to Example 1 (85% yield) as a white solid using [2-(3-chlorophenyl)oxetan2-yl]methanamine, cyclohexylacetic acid and HATU.

MS (m/z): 322.2 [M+H]+.

### Example 58: 1-(3-chlorophenyl)-3-[[2-(3-chlorophenyl)oxetan-2-yl]methyl]urea

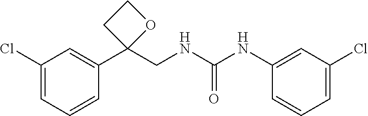 [SMILES: O=C(NCC1(c2cccc(Cl)c2)CCO1)Nc1cccc(Cl)c1]

The title compound was obtained in analogy to Example 4 (15% yield) using [2-(3-chlorophenyl)oxetan-2-yl]methanamine. 3-chloroaniline and N,N′-carbonyldiimidazole.

MS (m/z): 351.1 [M+H]+.

### Example 59: 1-[[2-(3-chlorophenyl)oxetan-2-yl]methyl]-3-(cyclopentylmethyl)urea

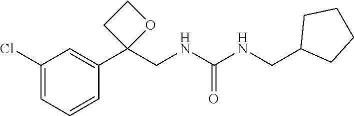 [SMILES: O=C(NCC1CCCC1)NCC1(c2cccc(Cl)c2)CCO1]

The title compound was obtained in analogy to Example 6 (77% yield) using [2-(3-chlorophenyl)oxetan-2-yl]methanamine and (isocyanatomethyl)cyclopentane.

MS (m/z): 323.2 [M+H]+.

### Example 60: 1-[[2-(3-chlorophenyl)oxetan-2-yl]methyl]-3-(2,2-dimethylpropyl)urea

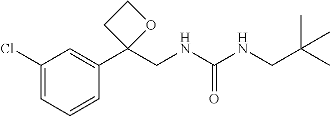 [SMILES: CC(C)(C)CNC(=O)NCC1(c2cccc(Cl)c2)CCO1]

The title compound was obtained in analogy to Example 6 (84% yield) using [2-(3-chlorophenyl)oxetan-2-yl]methanamine and 1-isocyanato-2,2-dimethylpropane.

MS (m/z): 311.2 [M+H]+.

### Example 61: N-[[2-(3-chlorophenyl)oxetan-2-yl]methyl]cyclohexanecarboxamide

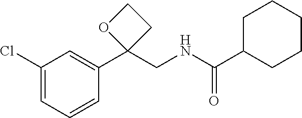 [SMILES: O=C(NCC1(c2cccc(Cl)c2)CCO1)C1CCCCC1]

The title compound was obtained in analogy to Example 1 (39% yield) as a white solid using [2-(3-chlorophenyl)oxetan2-yl]methanamine, cyclohexanoic acid and HATU.

MS (m/z): 308.1 [M+H]+.

### Example 62: 1-[(3-chlorophenyl)methyl]-3-[[2-(3-chlorophenyl)oxetan-2-yl]methyl]urea

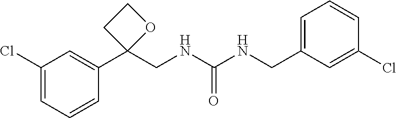 [SMILES: O=C(NCc1cccc(Cl)c1)NCC1(c2cccc(Cl)c2)CCO1]

The title compound was obtained in analogy to Example 6 (38% yield) as a colorless gum using [2-(3-chlorophenyl)oxetan-2-yl]methanamine and 1-chloro-3-(isocyanatomethyl)benzene.

MS (m/z): 365.1 [M+H]+.

### Example 63: 2-(1-bicyclo[1.1.1]pentanyl)-N-[[2-(3-chlorophenyl)oxetan-2-yl]methyl]acetamide

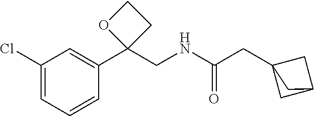 [SMILES: O=C(CC12CC(C1)C2)NCC1(c2cccc(Cl)c2)CCO1]

The title compound was obtained in analogy to Example 1 (75% yield) as a white solid using [2-(3-chlorophenyl)oxetan2-yl]methanamine, bicyclo[1.1.1]pentan1-ylacetic acid and HATU.

MS (m/z): 306.1 [M+H]+.

### Example 64: N-[[2-(3-chlorophenyl)oxetan-2-yl]methyl]spiro[3.3]heptane-2-carboxamide

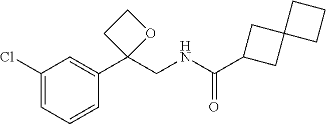 [SMILES: O=C(NCC1(c2cccc(Cl)c2)CCO1)C1CC2(CCC2)C1]

The title compound was obtained in analogy to Example 1 (77% yield) as a white solid using [2-(3-chlorophenyl)oxetan2-yl]methanamine, spiro[3.3]heptane2-carboxylic acid and HATU.

MS (m/z): 320.1 [M+H]+.

### Example 65: N-[[2-(3-chlorophenyl)oxetan-2-yl]methyl]-2-(4,4-difluorocyclohexyl)acetamide

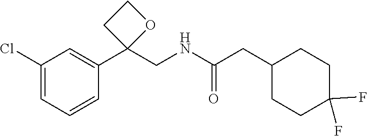 [SMILES: O=C(CC1CCC(F)(F)CC1)NCC1(c2cccc(Cl)c2)CCO1]

The title compound was obtained in analogy to Example 1 (81% yield) as a white solid using [2-(3-chlorophenyl)oxetan2-yl]methanamine. (4,4-difluorocyclohexyl)acetic acid and HATU.

MS (m/z): 358.1 [M+H]+.

### Example 66: (1S,2S)—N-[[2-(3-chlorophenyl)-5-oxo-tetrahydrofuran-2-yl]methyl]-2-phenyl-cyclopropanecarboxamide

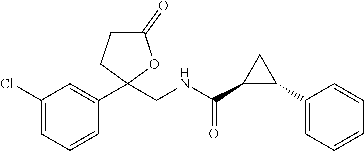 [SMILES: O=C1CCC(CNC(=O)[C@H]2C[C@@H]2c2ccccc2)(c2cccc(Cl)c2)O1]

### a) ethyl 4-bromopent-4-enoate

To a stirred solution of 2,3-dibromo-1-propene (5 g, 25.016 mmol) and CuI (5.72 g, 30.02 mmol) in THF (50 mL) were added LDA (4.02 g, 37.52 mmol) dropwise at −78 C under N2 atmosphere for 1 hour. Later, ethyl acetate (2.64 g, 30.019 mmol) was added, stirred for 1 hour. Upon completion of reaction, the resulting solution was extracted with ethyl acetate. The organic layers were combined, washed with brine, dried over anhydrous sodium sulfate, filtered, and concentrated under vacuum. The residue was purified by silica gel chromatography (ethyl acetate:petroleum ether=1:4) to afford ethyl 4-bromopent-4-enoate (3 g, 57.9% yield) as a yellow oil.

### b) 4-(3-chlorophenyl)pent-4-enoate

To a stirred solution of ethyl 4-bromopent-4-enoate (3 g, 14.49 mmol), Pd(PPh3)₄ (3.35 g, 2.90 mmol) and Cs2CO3 (14.16 g, 43.46 mmol) in Dioxane (20 mL). EtOH (20 mL) and H2O (20 mL) were added 3-chlorophenylboronic acid (3.40 g, 21.73 mmol) dropwise at 80 C under N2 atmosphere for 2 hours. Upon completion of reaction, the resulting solution was extracted with ethyl acetate (3×30 ml). The organic layers were combined, washed with brine, dried over anhydrous sodium sulfate, filtered, and concentrated under vacuum. The residue was purified by silica gel chromatography (ethyl acetate:petroleum ether=1:4) to afford ethyl 4-(3-chlorophenyl)pent-4-enoate (2.2 g, 63.6% yield) as a yellow oil.

### c) 4-(3-chlorophenyl) pent-4-enoic acid

To a stirred solution of ethyl 4-(3-chlorophenyl)pent-4-enoate (2.2 g, 9.22 mmol) in MeOH (20 mL) and H2O (5 mL) were added LiOH (1.10 g, 46.08 mmol) dropwise at 0 C under N2 atmosphere for 2 hours. Upon completion of reaction, the resulting solution was extracted with ethyl acetate (3×30 ml). The organic layers were combined, washed with brine, dried over anhydrous sodium sulfate, filtered, and concentrated under vacuum. The residue was purified by silica gel chromatography (ethyl acetate:petroleum ether=1:4) to afford 4-(3-chlorophenyl) pent-4-enoic acid (1.5 g, 77.3% yield) as a light yellowish oil.

### d) 5-(3-chlorophenyl)-5-(iodomethyl)oxolan-2-one

To a stirred solution of 4-(3-chlorophenyl)pent-4-enoic acid (1.5 g, 7.12 mmol) and DMAP (0.17 g. 1.42 mmol) in DCM (20 mL, 314.601 mmol) were added NIS (3.20 g, 14.24 mmol) dropwise at 20 C under N2 atmosphere for 2 hours. Upon completion of reaction, the resulting solution was extracted with ethyl acetate (3×30 ml). The organic layers were combined, washed with brine, dried over anhydrous sodium sulfate, filtered, and concentrated under vacuum. The residue was purified by silica gel chromatography (ethyl acetate:petroleum ether=1:4) to afford 5-(3-chlorophenyl)-5-(iodomethyl)oxolan-2-one (1.5 g. 62.6% yield) as a light yellowish oil.

### e) 5-(azidomethyl)-5-(3-chlorophenyl)oxolan-2-one

To a stirred solution of 5-(3-chlorophenyl)-5-(iodomethyl)oxolan-2-one (1.5 g, 4.457 mmol) in DMF (20 mL) were added NaN3 (1.45 g, 22.29 mmol) dropwise at 80 C under N2 atmosphere overnight. Upon completion of reaction, the resulting solution was extracted with ethyl acetate (3×30 ml). The organic layers were combined, washed with brine, dried over anhydrous sodium sulfate, filtered and concentrated under vacuum to give the crude product 5-(azidomethyl)-5-(3-chlorophenyl)oxolan-2-one (700 mg, 62.4% yield) as a yellow oil.

### f) 5-(aminomethyl)-5-(3-chlorophenyl) oxolan-2-one

To a stirred solution of 5-(azidomethyl)-5-(3-chlorophenyl)oxolan-2-one (700 mg, 2.78 mmol), Pd/C (1.48 g, 13.91 mmol) and ZnCl2 (568.7 mg, 4.17 mmol) in THF (5 mL) were added HCl (0.1 mL, 3.29 mmol) dropwise at 20 C under H2 atmosphere overnight. Upon completion of reaction, the resulting mixture was filtered and washed with THF (3×30 ml). The filtrate was concentrated under vacuum to give the crude product 5-(aminomethyl)-5-(3-chlorophenyl) oxolan-2-one (420 mg, 66.9% yield) as a light yellowish oil.

### g) (1S,2S)—N-[[2-(3-chlorophenyl)-5-oxo-tetrahydrofuran-2-yl]methyl]-2-phenyl-cyclopropanecarboxamide

To a stirred mixture of 5-(aminomethyl)-5-(3-chlorophenyl)oxolan-2-one (400 mg, 1.77 mmol), (1S,2S)-2-phenylcyclopropane-1-carboxylic acid (287.5 mg, 1.77 mmol) and TCFH (596.8 mg, 2.13 mmol) in ACN (5 mL) was added NMI (218.3 mg, 2.66 mmol) dropwise at room temperature under N2 atmosphere. The resulting mixture was stirred for additional 3 hours. Then it was purified by reverse phase chromatography eluting with MeCN in water (10% to 50% gradient in 10 min) to afford (1S,2S)—N-{[2-(3-chlorophenyl)-5-oxooxolan-2-yl]methyl}-2-phenylcyclopropane-1-carboxamide (8 mg, 1.1% yield) as a white solid.

MS (m/z): 370.1 [M+H]+.

### Example 67: (1S,2S)—N-[[2-[2-(difluoromethoxy)-4-pyridyl]oxetan-2-yl]methyl]-2-phenyl-cyclopropanecarboxamide

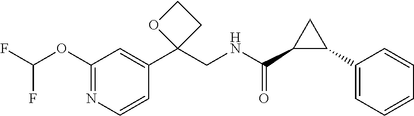 [SMILES: O=C(NCC1(c2ccnc(OC(F)F)c2)CCO1)[C@H]1C[C@@H]1c1ccccc1]

### a) 4-bromo-2-(difluoromethoxy)pyridine

To a stirred solution of 4-bromopyridin-2-ol (20 g, 114.95 mmol) in ACN (200 mL) was added chlorodifluoroacetate (29.8 g. 229.9 mmol) in portions at 80 C under N2 atmosphere, the reaction was stirred at 80 C overnight. Water was added and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the product 4-bromo-2-(difluoromethoxy) pyridine (15 g, 58.3% yield) as light yellow oil.

### b) 1-tert-butyl 3-methyl 2-[2-(difluoromethoxy)pyridin-4-yl]propanedioate

To a stirred solution of 4-bromo-2-(difluoromethoxy)pyridine (15 g, 66.96 mmol) in DMSO (300 mL) was added CuI (1.28 g, 6.7 mmol), K3PO4 (42.64 g, 200.9 mmol), Benzoxazole (1.60 g, 13.39 mmol) and tert-butyl methyl malonate (17.50 g, 100.44 mmol) at 80 C under N2 atmosphere overnight. Upon completion of reaction, water was added, and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the product 1-tert-butyl 3-methyl 2-[2-(difluoromethoxy)pyridin-4-yl]propanedioate (10 g, 47.1%) as light yellowish oil.

### c) methyl 2-[2-(difluoromethoxy)pyridin-4-yl]acetate

To a stirred solution of 1-tert-butyl 3-methyl 2-[2-(difluoromethoxy)pyridin-4-yl]propanedioate (15 g. 47.28 mmol) in DCM (20 mL) was added TFA (20 mL) and the reaction was stirred at 20 C under N2 atmosphere for 2 hours. The reaction mixture was concentrated under vacuum and the residue was purified by silica gel chromatography (Petroleum ether:EtOAc=3:1) to give the product methyl 2-[2-(difluoromethoxy)pyridin-4-yl]acetate (10 g, 97.4%) as yellowish oil.

### d) methyl 2-diazo-2-[2-(difluoromethoxy)pyridin-4-yl]acetate

To a stirred solution of methyl 2-[2-(difluoromethoxy)pyridin-4-yl]acetate (10 g. 46.05 mmol) in ACN (50 mL) was added tosyl azide (10.90 g, 55.26 mmol) and DBU (10.51 g, 69.07 mmol) at −30 C under N2. The reaction was stirred for 2 hours at −30 C. Water was then added, and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the methyl 2-diazo-2-[2-(difluoromethoxy)pyridin-4-yl]acetate (11 g, 98.2%) as yellowish oil.

### e) methyl 2-(2-bromoethoxy)-2-[2-(difluoromethoxy)pyridin-4-yl]acetate

To a stirred solution of 2-bromoethanol (4.52 g, 36.19 mmol) and Rhodium(II) acetate dimer (0.40 g, 0.91 mmol) in Toluene (50 mL) was added methyl 2-diazo-2-[2-(difluoromethoxy)pyridin-4-yl]acetate (11 g, 45.24 mmol) at 80 C under N2 atmosphere. The reaction was stirred at 80 C for 2 hours, then cool down to RT. Water was added, and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the product methyl 2-(2-bromoethoxy)-2-[2-(difluoromethoxy)pyridin-4-yl]acetate (7 g. 45.5%) as light yellowish oil.

### f) methyl 2-[2-(difluoromethoxy)pyridin-4-yl]oxetane-2-carboxylate

To a stirred solution of methyl 2-(2-bromoethoxy)-2-[2-(difluoromethoxy)pyridin-4-yl]acetate (7 g, 20.58 mmol) in THF (800 mL) was added LiHMDS (5.17 g, 30.87 mmol) at 0 C under N2 atmosphere. The reaction was stirred at 0 C for 1 hour. Saturated ammonium chloride solution was added, and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the methyl 2-[2-(difluoromethoxy)pyridin-4-yl]oxetane-2-carboxylate (3 g, 56.2%) as light yellowish oil.

### g) {2-[2-(difluoromethoxy)pyridin-4-yl]oxetan-2-yl}methanol

To a stirred solution of methyl 2-[2-(difluoromethoxy)pyridin-4-yl]oxetane-2-carboxylate (3 g, 11.57 mmol) in THF (30 mL) was added NaBH4 (2.19 g, 57.87 mmol) in portions at 0 C under N2 atmosphere. The reaction was stirred for 2 hours. Saturated ammonium chloride solution was added, and the resulting solution was extracted with EtOAc (3×). The organic layers were combined, washed with brine, dried over anhydrous sodium sulfate, filtered, and concentrated under vacuum. The residue was purified by silica gel chromatography (Petroleum ether:EtOAc=3:1) to give the product {2-[2-(difluoromethoxy)pyridin-4-yl]oxetan-2-yl}methanol (2 g, 74.7%) as light yellowish oil.

### h) {2-[2-(difluoromethoxy)pyridin-4-yl]oxetan-2-yl}methyl 4-methylbenzenesulfonate

To a stirred solution of {2-[2-(difluoromethoxy)pyridin-4-yl]oxetan-2-yl}methanol (2 g, 8.65 mmol), Et3N (2.63 g, 25.95 mmol) and DMAP (0.21 g, 1.73 mmol) in DCM (30 mL) was added TsCl (2.47 g, 12.98 mmol) at 0 C under N2 atmosphere. The reaction was stirred for 2 hours. Water was added, and the resulting solution was extracted with DCM (3×). The organic layers were combined, washed with brine, dried over anhydrous sodium sulfate, filtered, and concentrated under vacuum. The residue was purified by silica gel chromatography (Petroleum ether:EtOAc=3:1) to give the {2-[2-(difluoromethoxy)pyridin-4-yl]oxetan-2-yl}methyl 4-methylbenzenesulfonate (3 g, 90%) as a light yellowish oil.

### i) 4-[2-(azidomethyl)oxetan-2-yl]-2-(difluoromethoxy)pyridine

To a stirred solution of {2-[2-(difluoromethoxy)pyridin-4-yl]oxetan-2-yl}methyl 4-methyl benzene sulfonate (3 g, 7.79 mmol) in DMF (50 mL) was added NaN3 (2.53 g. 38.93 mmol) at 80 C under N2 atmosphere. The reaction was stirred overnight. Water was added, and the resulting solution was extracted with EtOAc (3×). The organic layers were combined, washed with brine, dried over anhydrous sodium sulfate, filtered and concentrated under vacuum to give the product 4-[2-(azidomethyl)oxetan-2-yl]-2-(difluoromethoxy)pyridine (1.5 g, 75.2%) as a light yellowish oil.

### j) 1-{2-[2-(difluoromethoxy)pyridin-4-yl]oxetan-2-yl}methanamine

To a stirred solution of 4-[2-(azidomethyl)oxetan-2-yl]-2-(difluoromethoxy)pyridine (1.5 g, 5.86 mmol) in THF (20 mL) and H2O (5 mL) was added PPh3 (7.68 g, 29.28 mmol) at 20 C under N2 atmosphere. The reaction was stirred for 2 hours. Water was added, and the resulting solution was extracted with EtOAc (3×). The organic layers were combined, washed with brine, dried over anhydrous sodium sulfate, filtered, and concentrated under vacuum. The residue was purified by reverse phase chromatography eluting with MeCN in water (10% to 50% gradient in 10 min) to give the 1-{2-[2-(difluoromethoxy)pyridin-4-yl]oxetan-2-yl}methanamine (500 mg, 37.1%) as a light yellowish oil.

### k) (1S,2S)—N-[[2-[2-(difluoromethoxy)-4-pyridyl]oxetan-2-yl]methyl]-2-phenyl-cyclopropanecarboxamide

To a stirred solution of 1-{2-[2-(difluoromethoxy)pyridin-4-yl]oxetan-2-yl}methanamine (100 mg, 0.43 mmol), (1S,2S)-2-phenylcyclopropane-1-carboxylic acid (84.54 mg, 0.52 mmol) and TCFH (243.75 mg. 0.87 mmol) in ACN (2 mL) was added NMI (71.33 mg, 0.868 mmol) at 20 C under N2 atmosphere. The reaction was stirred at RT for 2 hours. The reaction mixture was concentrated under vacuum. The residue was purified by reverse phase chromatography eluting with MeCN in water (10% to 50% gradient in 10 min) to give the product (1S,2S)—N-[[2-[2-(difluoromethoxy)-4-pyridyl]oxetan-2-yl]methyl]-2-phenyl-cyclopropanecarboxamide (7.8 mg, 4.5% yield) as a reddish brown solid.

MS (m/z): 375.1 [M+H]+.

### Example 68: (1S,2S)—N-[[3-(3-chlorophenyl)oxetan-3-yl]methyl]-2-phenyl-cyclopropanecarboxamide

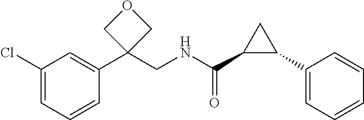 [SMILES: O=C(NCC1(c2cccc(Cl)c2)COC1)[C@H]1C[C@@H]1c1ccccc1]

### a) 3-(nitromethyl)oxetan-3-ol

To a stirred solution of 3-oxetanone (2 g, 27.75 mmol) and nitromethane (2.54 g, 41.63 mmol) in DCM (30 mL) was added Et3N (8.43 g, 83.26 mmol) dropwise at 0 C under N2 atmosphere. The reaction was stirred for 1.5 hours. The resulting solution was extracted with DCM (3×30 ml). The organic layers were combined, washed with brine, dried over anhydrous sodium sulfate, filtered and concentrated under vacuum to afford the crude product 3-(nitromethyl)oxetan-3-ol (2.9 g, 78.5%) as a yellowish oil.

### b) 3-(nitromethylidene) oxetane

To a stirred solution of 3-(nitromethyl) oxetan-3-ol (2.9 g, 21.79 mmol) and Et3N (6.61 g, 65.36 mmol) in DCM (30 mL) was added MsCl (3.74 g, 32.68 mmol) dropwise at 0 C under N2 atmosphere. The reaction was stirred for 1.5 hours. The resulting solution was extracted with DCM (3×30 mL). The organic layers were combined, washed with brine, dried over anhydrous sodium sulfate, filtered, and concentrated under vacuum. The residue was purified by silica gel chromatography (EtOAc:Petroleum ether=1:4) to afford the product 3-(nitromethylidene) oxetane (1.4 g. 55.8%) as a light yellowish solid.

### c) 3-(3-chlorophenyl)-3-(nitromethyl)oxetane

To a stirred solution of 1-bromo-3-chlorobenzene (11.65 g, 60.83 mmol) in THF (50 mL) was added BuLi (24.33 mL, 60.83 mmol, 2.5 M) dropwise at −78 C under N2 atmosphere. Half an hour later, a solution of 3-(nitromethylidene)oxetane (1.4 g, 12.17 mmol) in THF (3 mL) was added. Upon completion of reaction, the resulting solution was poured into a beaker containing ice. The mixture was then extracted with EtOAc (3×80 mL). The organic layers were combined, washed with brine, dried over anhydrous sodium sulfate, filtered, and concentrated under vacuum. The residue was purified by silica gel chromatography (EtOAc:Petroleum ether=1:4) to afford the product 3-(3-chlorophenyl)-3-(nitromethyl)oxetane (400 mg, 14.4%) as a light yellowish oil.

### d) 1-[3-(3-chlorophenyl)oxetan-3-yl]methanamine

To a stirred solution of 3-(3-chlorophenyl)-3-(nitromethyl)oxetane (400 mg, 1.76 mmol) and Pd/C (935 mg, 8.79 mmol) in i-PrOH (20 mL) was added ZnCl2 (479.05 mg. 3.51 mmol) at 25 C under H2 atmosphere. The reaction was stirred overnight. The suspension was then filtered. The filtrate was concentrated under vacuum to give the residue product 1-[3-(3-chlorophenyl)oxetan-3-yl]methanamine (120 mg, 34.6%) as alight yellowish oil.

### e) (1S,2S)—N-[[3-(3-chlorophenyl)oxetan-3-yl]methyl]-2-phenyl-cyclopropanecarboxamide

To a stirred mixture of 1-[3-(3-chlorophenyl) oxetan-3-yl]methanamine (100 mg, 0.51 mmol), (1S,2S)-2-phenylcyclopropane-1-carboxylic acid (82.05 mg, 0.51 mmol) and TCFH (170.34 mg, 0.61 mmol) in ACN (2 mL) was added NMI (62.31 mg, 0.76 mmol) at RT under N2 atmosphere.

The resulting mixture was stirred for 3 hours. The mixture was purified by reverse phase flash chromatography eluting with MeCN in water (10% to 50% gradient in 10 min) to afford (1S,2S)—N-{[3-(3-chlorophenyl) oxetan-3-yl]methyl}-2-phenylcyclopropane-1-carboxamide (16.8 mg, 9.3%) as a reddish brown solid.

MS (m/z): 342.1 [M+H]+.

### Example 69: N-[(2R)-2-(3-chlorophenyl)-2-methoxy-propyl]-4-hydroxy-5,5-dimethyl-hexanamide

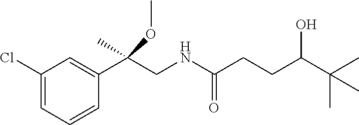 [SMILES: CO[C@@](C)(CNC(=O)CCC(O)C(C)(C)C)c1cccc(Cl)c1]

The title compound was obtained in analogy to Example 1 (510% yield) as a white solid using (R)-2-(3-chlorophenyl)-2-methoxypropan-1-amine, lithio 4-hydroxy-5, 5-dimethylhexanoate and HATU.

MS (m/z): 342.2 [M+H]+.

### Example 70: (1S,2S)—N-[(2S)-2-(3-chlorophenyl)-2-(cyanomethoxy)ethyl]-2-phenyl-cyclopropanecarboxamide

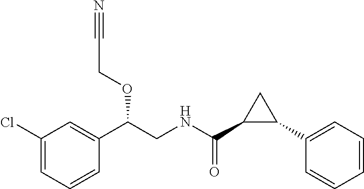 [SMILES: N#CCO[C@H](CNC(=O)[C@H]1C[C@@H]1c1ccccc1)c1cccc(Cl)c1]

### a) (1S,2S)—N-[(2S)-2-(3-chlorophenyl)-2-hydroxyethyl]-2-phenylcyclopropane1-carboxamide

The compound was obtained in analogy to Example 1 (74% yield) as a white solid using (1S)-2-amino-1-(3-chlorophenyl)ethanol hydrochloride, (1S,2S)-2-phenylcyclopropane1-carboxylic acid and HATU.

### b) (1S,2S)—N-[(2S)-2-(3-chlorophenyl)-2-(cyanomethoxy)ethyl]-2-phenyl-cyclopropanecarboxamide

To a flask containing (1S,2S)—N-[(2S)-2-(3-chlorophenyl)-2-hydroxyethyl]-2-phenylcyclopropane1-carboxamide (83 mg, 0.26 mmol), 4A mol. sieves were added, the flask flushed with N2 and DCM (4.5 mL) followed by bromoacetonitrile (73.23 μL, 1.72 g/mL, 1.05 mmol) were added. The reaction was stirred at RT for 1 h. Tetrabutylammonium iodide (0.05 mL, 0.26 mmol, 1 eq) and Silver(I) oxide (243.63 mg, 1.05 mmol) were then added. The flask was wrapped in foil and stirred under N2 overnight. Diluted with DCM and passed through a plug of celite. The filtrate was concentrated and the residue was purified by silica gel chromatography eluting with heptane-40% EtOAc/Heptane (gradient) to afford still impure material (66 mg). This was dissolved in DMSO (2 ml) and purified by reverse phase HPLC to afford the title product as a colourless gum (47 mg, 50.4% yield)

MS (m/z): 355.1 [M+H]+.

### Example 71: 1-(cyclopentylmethyl)-3-[rel-(2S)-2-(3-chlorophenyl)-2-methoxy-propyl]urea

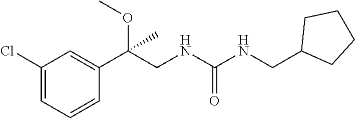 [SMILES: CO[C@@](C)(CNC(=O)NCC1CCCC1)c1cccc(Cl)c1]

The title compound was obtained via chiral separation of Example 21 1-(2-(3-chlorophenyl)-2-methoxypropyl)-3-(cyclopentylmethyl)urea (30% yield) as a white solid (Cellulose-4 column, in water:MeCN 48:52).

MS (m/z): 325.2 [M+H]+.

### Example 72: 1-[[3-(3-chlorophenyl)oxetan-3-yl]methyl]-3-(cyclopentylmethyl)urea

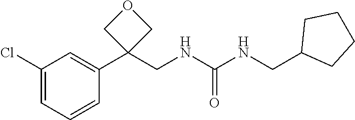 [SMILES: O=C(NCC1CCCC1)NCC1(c2cccc(Cl)c2)COC1]

To a stirred mixture of 1-cyclopentylmethanamine (180.6 mg, 1.82 mmol), Triphosgene (180.16 mg, 0.61 mmol) and DIPEA (980.8 mg, 7.59 mmol) in DCM (5 mL) was added 1-[3-(3-chlorophenyl)oxetan-3-yl]methanamine (300 mg, 1.52 mmol) dropwise at room temperature under N2 atmosphere. The resulting mixture was stirred for 3 hours, then concentrated in vaccuo. The crude was purified by reverse phase chromatography eluting with MeCN in water (10% to 50% gradient in 10 min) to afford the title product (12.5 mg, 2.5%) as a white solid.

MS (m/z): 323.2 [M+H]+.

### Example 73: N-[2-(3-chlorophenyl)-2-(cyanomethoxy)ethyl]-2-cyclopentyl-acetamide

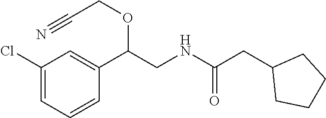 [SMILES: N#CCOC(CNC(=O)CC1CCCC1)c1cccc(Cl)c1]

### a) 2-(3-chlorophenyl)-2-hydroxyacetate

To a stirred solution of (3-chlorophenyl)(hydroxy)acetic acid (10 g, 53.59 mmol) in DCM (25 mL) and MeOH (25 mL) was added TMSCHN2 (6.12 g, 53.59 mmol) at 0 C under N2 atmosphere. The reaction was stirred for 1 hour. Upon completion of reaction, the mixture was concentrated under vacuum. The residue was purified by silica gel chromatography, eluting with Petroleum ether:EtOAc=5:1 to afford methyl 2-(3-chlorophenyl)-2-hydroxyacetate (10 g, 93%) as white solid.

### b) 1-(3-chlorophenyl)ethane-1,2-diol

To a stirred solution of methyl 2-(3-chlorophenyl)-2-hydroxyacetate (10 g, 49.85 mmol) in THF (50 mL) was added NaBH4 (9.43 g, 249.23 mmol) at 0 C under N2 atmosphere. The reaction was stirred for 1 hour. Saturated ammonium chloride solution was added, and the resulting solution was extracted with EtOAc (3×). The organic layers were combined, washed with brine, dried over anhydrous sodium sulfate, filtered, and concentrated under vacuum. The residue was purified by silica gel chromatography, eluted with Petroleum ether:EtOAc=5:1 to afford 1-(3-chlorophenyl)ethane-1,2-diol (8 g, 93%) as light yellowish oil.

### c) 2-[(tert-butyldimethylsilyl)oxy]-1-(3-chlorophenyl)ethanol

To a stirred solution of 1-(3-chlorophenyl)ethane-1,2-diol (8 g, 46.35 mmol) and DMAP (1.13 g, 9.27 mmol) in DCM (40 mL) was added DIPEA (17.97 g, 139.041 mmol) and TBDMSCl (10.48 g, 69.52 mmol) at 0 C. The reaction was stirred for 1 hour. The mixture was concentrated in vaccuo. The residue was purified by silica gel column chromatography, eluted with Petroleum ether:EtOAc=5:1 to afford 2-[(tert-butyldimethylsilyl)oxy]-1-(3-chlorophenyl)ethanol (10 g, 75.2%) as light yellow oil.

### d) 2-{2-[(tert-butyldimethylsilyl)oxy]-1-(3-chlorophenyl)ethoxy}acetamide

To a stirred solution of 2-[(tert-butyldimethylsilyl)oxy]-1-(3-chlorophenyl)ethanol (10 g. 34.86 mmol) in THF (50 mL) was added t-BuOK (7.82 g, 69.72 mmol) at 0 C under N2 atmosphere. The reaction was stirred for 30 minutes, then 2-bromoacetamide (7.21 g, 52.29 mmol) was added, the resulting mixture was stirred for another 2 hours. Upon completion of reaction, the mixture was concentrated under vacuum. The residue was purified by silica gel chromatography, eluted with DCM:MeOH=10:1 to afford 2-{2-[(tert-butyldimethylsilyl)oxy]-1-(3-chlorophenyl)ethoxy}acetamide (4 g, 33.4%) as a light yellowish oil.

### e) 2-{2-[(tert-butyldimethylsilyl)oxy]-1-(3-chlorophenyl)ethoxy}acetonitrile

To a stirred solution of 2-{2-[(tert-butyldimethylsilyl)oxy]-1-(3-chlorophenyl)ethoxy}acetamide (4 g, 11.63 mmol) and Pyridine (1.38 g, 17.45 mmol) in THF (30 mL) was added TFAA (4.89 g, 23.26 mmol) at 20 C. After 1 hour, the reaction mixture was concentrated under vacuum. The residue was purified by silica gel chromatography, eluted with DCM:MeOH=10:1 to afford 2-{2-[(tert-butyldimethylsilyl)oxy]-1-(3-chlorophenyl)ethoxy}acetonitrile (3 g, 79.1%) as a light yellowish oil.

### f) 2-[1-(3-chlorophenyl)-2-hydroxyethoxy]acetonitrile

To a stirred solution of 2-{2-[(tert-butyldimethylsilyl)oxy]-1-(3-chlorophenyl)ethoxy}acetonitrile (3 g, 9.21 mmol) in THF (20 mL) was added TBAF (4.81 g, 18.41 mmol) at 20 C. The reaction was stirred for 1 hour. The reaction mixture was concentrated under vacuum. The residue was purified by silica gel column chromatography, eluted with DCM:MeOH=10:1 to afford 2-[1-(3-chlorophenyl)-2-hydroxyethoxy]acetonitrile (1.8 g, 92.4%) as a light yellowish oil.

### g) 2-[1-(3-chlorophenyl)-2-[(4-methylbenzenesulfonyl)oxy]ethoxy]acetonitrile

To a stirred solution of 2-[1-(3-chlorophenyl)-2-hydroxyethoxy]acetonitrile (1.8 g, 8.51 mmol), DMAP (0.21 g, 1.7 mmol) in DCM (10 mL) was added DIPEA (3.30 g, 25.52 mmol) and TsCl (2.43 g, 12.76 mmol) at OC, the reaction was stirred for 1 hour. The reaction mixture was concentrated under vacuum. The residue was purified by silica gel column chromatography, eluted with DCM:MeOH=10:1 to afford 2-[1-(3-chlorophenyl)-2-[(4-methylbenzenesulfonyl)oxy]ethoxy]acetonitrile (2 g, 64.3%) as alight yellowish oil.

### h) 2-[2-azido-1-(3-chlorophenyl)ethoxy]acetonitrile

To a stirred solution of 2-[1-(3-chlorophenyl)-2-[(4-methylbenzenesulfonyl)oxy]ethoxy]acetonitrile (2 g, 5.47 mmol) in DMF (20 mL) was added NaN3 (1.78 g, 27.34 mmol) in portions at 80 C under N2 atmosphere. The reaction was stirred overnight. Water was added, and the resulting solution was extracted with EtOAc (3×). The organic layers were combined, washed with brine, dried over anhydrous sodium sulfate, filtered and concentrated under vacuum to give the product 2-[2-azido-1-(3-chlorophenyl)ethoxy]acetonitrile (1 g, 77.3%) as light yellow oil.

### i) 2-[2-amino-1-(3-chlorophenyl)ethoxy]acetonitrile

To a stirred solution of 2-[2-azido-1-(3-chlorophenyl)ethoxy]acetonitrile (1 g. 4.23 mmol) in THF (8 mL) and H2O (2 mL) was added PPh3 (5.54 g, 21.13 mmol) in portions at 20 C under N2 atmosphere. The reaction was stirred for 2 hours. The reaction mixture was concentrated under vacuum. The residue was purified by reverse phase chromatography eluting with MeCN in water (10% to 50% gradient) to give 2-[2-amino-1-(3-chlorophenyl)ethoxy]acetonitrile (200 mg, 26.2%) as a light yellowish oil.

### j) N-[2-(3-chlorophenyl)-2-(cyanomethoxy)ethyl]-2-cyclopentyl-acetamide

The compound was obtained in analogy to Example 1 (12% yield) as a white solid using 2-[2-amino-1-(3-chlorophenyl)ethoxy]acetonitrile, cyclopentaneacetic acid and HATU.

MS (m/z): 321.1 [M+H]+.

### Example 74: (1S,2S)—N-[2-(3-chlorophenyl)-3-hydroxy-propyl]-2-phenyl-cyclopropanecarboxamide

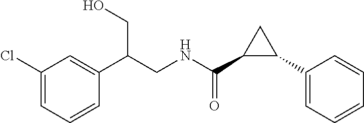 [SMILES: O=C(NCC(CO)c1cccc(Cl)c1)[C@H]1C[C@@H]1c1ccccc1]

### a) 3-amino-2-(3-chlorophenyl)propan-1-ol

To a solution of 2-(3-chlorophenyl)acetonitrile (2.34 g, 15.44 mmol) and ethyl formate (1.72 g, 23.22 mmol) in THF (150 mL) was added NaH (0.48 g, 20.0 mmol) in an ice bath. The resulting mixture was stirred at 0 C for 30 minutes, then the mixture was carefully acidified with dilute HCl and extracted with ethyl acetate. Extracts were washed with water, saturated NaCl solution, dried (MgSO4) and concentrated to afford 3-amino-2-(3-chlorophenyl)propan-1-ol (2.0 g, 69.8%) as a white solid.

### b) 3-amino-2-(3-chlorophenyl)propan-1-ol

To a solution of 2-(3-chlorophenyl)-3-hydroxypropanenitrile (5 g, 27.53 mmol) and Cobalt chloride hexahydrate (9.83 g, 41.32 mmol) in THF (300 mL) was added NaBH4 (8.33 g, 220.18 mmol) in an ice bath. The resulting mixture was stirred at 0 C for 30 minutes. Dilute NH4Cl was carefully added and extracted with ethyl acetate. Extracts were washed with water, saturated NaCl solution, dried (MgSO4) and concentrated under vacuum to give 3-amino-2-(3-chlorophenyl)propan-1-ol (2.5 g. 48.9%) as a white solid.

### c) (1S,2S)—N-[2-(3-chlorophenyl)-3-hydroxy-propyl]-2-phenyl-cyclopropanecarboxamide

To a stirred mixture of 3-amino-2-(3-chlorophenyl)propan-1-ol (80 mg, 0.43 mmol), (1S,2S)-2-phenylcyclopropane-1-carboxylic acid (69.89 mg, 0.43 mmol) and TCFH (241.81 mg, 0.86 mmol) in ACN (20 mL) was added NMI (70.76 mg, 0.86 mmol) dropwise at 0 C under N2 atmosphere. The residue was purified by reverse phase HPLC eluting with MeCN in water (10% to 50% gradient in 20 min) to afford (1S,2S)—N-[2-(3-chlorophenyl)-3-hydroxypropyl]-2-phenylcyclopropane-1-carboxamide (61.8 mg, 43.5%) as a white solid.

MS (m/z): 330.1 [M+H]+.

### Example 75: 1-[[2-(3-chlorophenyl)-5-oxo-tetrahydrofuran-2-yl]methyl]-3-(cyclopentylmethyl)urea

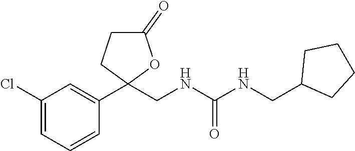 [SMILES: O=C(NCC1CCCC1)NCC1(c2cccc(Cl)c2)CCC(=O)O1]

The compound was obtained in analogy to Example 72 (0.6% yield) as a white solid using 5-(aminomethyl)-5-(3-chlorophenyl) oxolan-2-one (Example 66), 1-cyclopentylmethanamine and triphosgene.

MS (m/z): 351.1 [M+H]+.

### Example 76: (1S,2S)—N-[[2-(6-chloro-2-pyridyl)oxetan-2-yl]methyl]-2-phenyl-cyclopropanecarboxamide

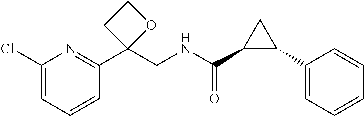 [SMILES: O=C(NCC1(c2cccc(Cl)n2)CCO1)[C@H]1C[C@@H]1c1ccccc1]

The compound was obtained in analogy to Example 67 as a yellowish solid using 2-bromo-6-chloropyridine. (1S,2S)-2-phenylcyclopropane-1-carboxylic acid, and TCFH/NMI.

MS (m/z): 343.1 [M+H]+.

### Example 77: (1S,2S)—N-[(E)-2-(3-chlorophenyl)-4-hydroxy-but-2-enyl]-2-phenyl-cyclopropanecarboxamide

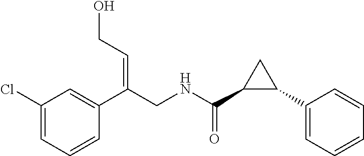 [SMILES: O=C(NC/C(=C/CO)c1cccc(Cl)c1)[C@H]1C[C@@H]1c1ccccc1]

### a) ethyl 4-|(tert-butoxycarbonyl) amino|-3-oxobutanoate

A solution of [(tert-butoxycarbonyl)amino]acetic acid (10 g, 57.08 mmol) and CDI (13.88 g, 85.62 mmol) in THF (200 mL) was stirred for 3 h at RT under N2 atmosphere. To the solution was added MgCl2 (10.87 g, 114.17 mmol) and potassium 3-ethoxy-3-oxopropanoate (19.43 g, 114.17 mmol) at RT. The reaction was stirred for 18 h at 50 under N2 atmosphere. The precipitated solids were collected by filtration and washed with THF (200 ml). The organic layer was washed with NH4Cl (300 ml), NaHCO3 (100 ml), brine (100 ml), dried (MgSO4) and concentrated. The residue was purified by silica gel chromatography, eluted with Petroleum ether:EtOAc (3:7) to afford ethyl 4-[(tert-butoxycarbonyl) amino]-3-oxobutanoate (8.1 g, 57.9%) as a green oil.

### b) ethyl (2Z)-4-[(tert-butoxycarbonyl)amino]-3-[(4-methylbenzenesulfonyl)oxy]but-2-enoate

A solution of ethyl 4-[(tert-butoxycarbonyl)amino]-3-oxobutanoate (8.1 g, 33.02 mmol), LDA (7.08 g, 66.05 mmol) in THF (100 mL) was stirred for 3 h at −50 under N2 atmosphere. 4-methylbenzenesulfonyl 4-methylbenzenesulfonate (14.01 g, 42.93 mmol) was added and the reaction was stirred at −50 for 1 h. Temperature was brought back to RT and it was stirred for 12 h. Water was added (100 ml) and was extracted with EtOAc (100 ml). The organic layer was washed with NaHCO3 (100 ml); 1N NaOH (2×100 ml), brine (2×100 ml), dried (MgSO4) and concentrated. The residue was purified by silica gel chromatography, eluted with Petroleum ether/EtOAc=(3:7) to afford ethyl (2Z)-4-[(tert-butoxycarbonyl)amino]-3-[(4-methylbenzenesulfonyl)oxy]but-2-enoate (7.7 g, 58.4%) as a yellowish solid.

### c) ethyl (2E)-4-[(tert-butoxycarbonyl)amino]-3-(3-chlorophenyl)but-2-enoate

A solution of ethyl (2Z)-4-[(tert-butoxycarbonyl) amino]-3-[(4-methylbenzenesulfonyl)oxy]but-2-enoate (7.7 g, 19.28 mmol), 3-chlorophenylboronic acid (4.52 g, 28.91 mmol), Na2CO3 (6.13 g, 57.83 mmol), PdCl2(PPh3)₂ (1.353 g, 1.93 mmol) in THF (100 mL) and H2O (20 mL) was stirred overnight at 40 under N2 atmosphere. The aqueous layer was extracted with EtOAc (100 ml), and the combined organic layer was washed with aq. NaHCO3 (50 ml). H2O (50 ml), dried (MgSO4) and concentrated. The residue was purified by silica gel chromatography, eluted with Petroleum ether/EtOAc=(2:3) to afford ethyl (2E)-4-[(tert-butoxycarbonyl)amino]-3-(3-chlorophenyl)but-2-enoate (3.6 g, 55.0%) as a red solid.

### d) tert-butyl N-[(2E)-2-(3-chlorophenyl)-4-hydroxybut-2-en-1-yl]carbamate

A solution of ethyl (2E)-4-[(tert-butoxycarbonyl)amino]-3-(3-chlorophenyl)but-2-enoate (3.6 g, 10.59 mmol) and LiAlH4 (1.21 g, 31.78 mmol) in THF (20 mL) was stirred for 10 min at 0 under N2 atmosphere. The reaction was quenched with sodium sulfate decahydrate at 0. The residue product was purified by reverse phase chromatography (eluted with H2O:ACN=3:7) to afford tert-butyl N-[(2E)-2-(3-chlorophenyl)-4-hydroxybut-2-en-1-yl]carbamate (1.54 g, 48.8%) as a white solid.

### e) (2E)-4-amino-3-(3-chlorophenyl)but-2-en-1-ol

A solution of tert-butyl N-[(2E)-2-(3-chlorophenyl)-4-hydroxybut-2-en-1-yl]carbamate (1.5 g, 5.04 mmol) and TFA (15 mL) in DCM (40 mL) was stirred for 1 h at room temperature. The mixture was concentrated and the residue product was purified by reverse phase chromatography (eluted with H2O:ACN=2:3) to afford (2E)-4-amino-3-(3-chlorophenyl)but-2-en-1-ol (0.76 g, 76.3%) as a white oil.

### f) (1S,2S)—N-[(E)-2-(3-chlorophenyl)-4-hydroxy-but-2-enyl]-2-phenyl-cyclopropanecarboxamide

A solution of (2E)-4-amino-3-(3-chlorophenyl)but-2-en-1-ol (100 mg, 0.51 mmol), (1S,2S)-2-phenylcyclopropane-1-carboxylic acid (123.08 mg, 0.76 mmol), TCFH (283.90 mg, 1.01 mmol) and NMI (124.61 mg, 1.52 mmol) in ACN (5 mL) was stirred for 1 h at room temperature under N2 atmosphere. The reaction mixture was concentrated and the residue product was purified by reverse phase chromatography (eluted with water:ACN=3:7) to afford (1S,2S)—N-[(2E)-2-(3-chlorophenyl)-4-hydroxybut-2-en-1-yl]-2-phenylcyclopropane-1-carboxamide (6 mg, 3.5%) as a white solid.

MS (m/z): 342.1 [M+H]+.

### Example 78: 1-[(E)-2-(3-chlorophenyl)-4-hydroxy-but-2-enyl]-3-(cyclopentylmethyl)urea

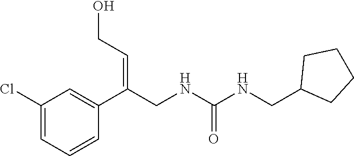 [SMILES: O=C(NC/C(=C/CO)c1cccc(Cl)c1)NCC1CCCC1]

The compound was obtained in analogy to Example 72 (47% yield) as a white solid using (2E)-4-amino-3-(3-chlorophenyl)but-2-en-1-ol (Example 77), 1-cyclopentylmethanamine and triphosgene.

MS (m/z): 323.1 [M+H]+.

### Example 79: (1RS,2SR)—N-[(2R)-2-(3-chlorophenyl)-2-methoxy-propyl]-2-cyclopropyl-cyclopropanecarboxamide

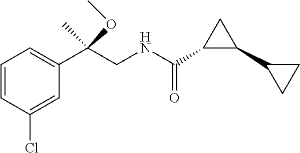 [SMILES: CO[C@@](C)(CNC(=O)[C@@H]1C[C@H]1C1CC1)c1cccc(Cl)c1]

The title compound was obtained in analogy to Example 1 (86% yield) as a colorless gum using (R)-2-(3-chlorophenyl)-2-methoxypropan-1-amine, rac-(1R,2S)-2-cyclopropylcyclopropane-1-carboxylic acid and HATU.

MS (m/z): 308.1 [M+H]+.

### Example 80: (1RS,2SR)—N-[(2R)-2-(3-chlorophenyl)-2-methoxy-propyl]-2-cyclopentyl-cyclopropanecarboxamide

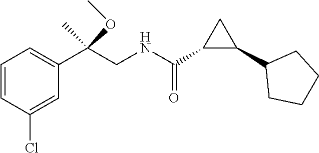 [SMILES: CO[C@@](C)(CNC(=O)[C@@H]1C[C@H]1C1CCCC1)c1cccc(Cl)c1]

The title compound was obtained in analogy to Example 1 (42% yield) as a colorless gum using (R)-2-(3-chlorophenyl)-2-methoxypropan-1-amine, 2-cyclopentylcyclopropane1-carboxylic acid and HATU.

MS (m/z): 336.2 [M+H]+.

### Example 81: (1SR,2SR)—N-[(2R)-2-(3-chlorophenyl)-2-methoxy-propyl]-2-cyclopentyl-cyclopropanecarboxamide

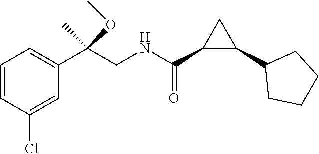 [SMILES: CO[C@@](C)(CNC(=O)[C@H]1C[C@H]1C1CCCC1)c1cccc(Cl)c1]

The title compound is the cis diastereomeric mixture obtained from purification of Example 80 (33% yield).

MS (m/z): 336.2 [M+H]+.

### Example 82: N-[(2R)-2-(3-chlorophenyl)-2-methoxy-propyl]spiro[2.5]octane-2-carboxamide

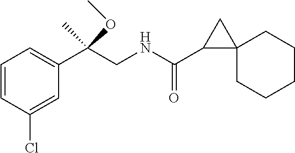 [SMILES: CO[C@@](C)(CNC(=O)C1CC12CCCCC2)c1cccc(Cl)c1]

The title compound was obtained in analogy to Example 1 (92% yield) as a colorless gum using (R)-2-(3-chlorophenyl)-2-methoxypropan-1-amine, spiro[2.5]octane-1-carboxylic acid and HATU.

MS (m/z): 336.2 [M+H]+.

### Example 83: N-[(2R)-2-(3-chlorophenyl)-2-methoxy-propyl]-6,6-difluoro-spiro[2.5]octane-2-carboxamide

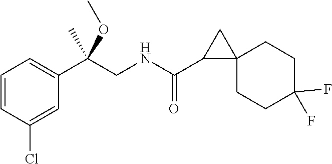 [SMILES: CO[C@@](C)(CNC(=O)C1CC12CCC(F)(F)CC2)c1cccc(Cl)c1]

The title compound was obtained in analogy to Example 1 (63% yield) as a colorless gum using (R)-2-(3-chlorophenyl)-2-methoxypropan-1-amine, 6,6-difluorospiro[2. 5]octane-1-carboxylic acid and HATU.

MS (m/z): 372.2 [M+H]+.

### Example 84: N-[(2R)-2-(3-chlorophenyl)-2-methoxy-propyl]-6-oxaspiro[2.5]octane-2-carboxamide

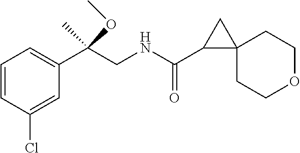 [SMILES: CO[C@@](C)(CNC(=O)C1CC12CCOCC2)c1cccc(Cl)c1]

The title compound was obtained in analogy to Example 1 (52% yield) as a colorless gum using (R)-2-(3-chlorophenyl)-2-methoxypropan-1-amine, 6-oxaspiro[2. 5]octane-1-carboxylic acid and HATU.

MS (m/z): 338.1 [M+H]+.

### Example 85: (1S,2S)—N-[[3-(3-cyanophenyl)oxetan-3-yl]methyl]-2-phenyl-cyclopropanecarboxamide

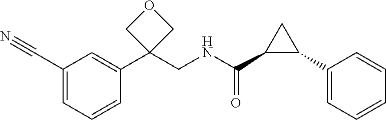 [SMILES: N#Cc1cccc(C2(CNC(=O)[C@H]3C[C@@H]3c3ccccc3)COC2)c1]

The title compound was obtained in analogy to Example 68 using 3-bromobenzonitrile, (1S,2S)-2-phenylcyclopropane-1-carboxylic acid and TCFH/NMI.

MS (m/z): 333.2 [M+H]+.

### Example 86: (1S,2S)—N-[1-[1-(3-chlorophenyl)cyclopropyl]-2-hydroxy-ethyl]-2-phenyl-cyclopropanecarboxamide

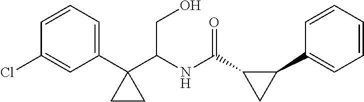 [SMILES: O=C(NC(CO)C1(c2cccc(Cl)c2)CC1)[C@H]1C[C@@H]1c1ccccc1]

To a solution of 2-amino-2-[1-(3-chlorophenyl)cyclopropyl]ethanol (100 mg, 0.472 mmol, 1.00 equiv) in ACN (2 mL) were added (1S,2S)-2-phenylcyclopropane-1-carboxylic acid (76.6 mg, 0.472 mmol), TCFH (397.6 mg. 1.416 mmol, 3 equiv) followed by dropwise addition of NMI (38.8 mg, 0.472 mmol). The mixture was stirred at 25 C for 2 h until reaction completion. The reaction mixture was evaporated under vacuo and the residue was purified by reverse flash chromatography using MeCN in water (20% to 50% gradient in 30 min), to give the desired product (31.8 mg, 18.9%) as a white solid.

MS (m/z): 356.1 [M+H]+.

### Example 87: (1S,2S)—N-[[2-(3-cyanophenyl)-3,3-difluoro-oxetan-2-yl]methyl]-2-phenyl-cyclopropanecarboxamide

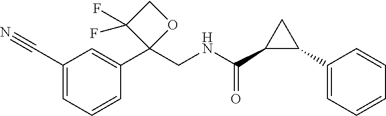 [SMILES: N#Cc1cccc(C2(CNC(=O)[C@H]3C[C@@H]3c3ccccc3)OCC2(F)F)c1]

### a) ((1-(3-bromophenyl)ethenyl)oxy)trimethylsilane

A mixture of m-bromoacetophenone (15 g, 75.36 mmol) and NaI (13.56 g, 90.43 mmol) in ACN (20 mL) was stirred for 5 min at room temperature under N2 atmosphere. To the stirred mixture was added Et3N (9.15 g, 90.43 mmol) and TMSCl (9.82 g, 90.43 mmol) portionwise under N2 atmosphere. The resulting mixture was stirred overnight, then concentrated under vacuum. The residue was purified by silica gel chromatography, eluted with Petroleum ether/EtOAc=2:3 to afford ((1-(3-bromophenyl)ethenyl)oxy)trimethylsilane (7.8 g, 38.2%) as white oil.

### b) 3-bromo-1-(3-bromophenyl)-2,2-difluoropropan-1-one

A solution of ((1-(3-bromophenyl)ethenyl)oxy)trimethylsilane (7.8 g, 28.76 mmol), (bromodifluoromethyl)trimethylsilane (8.76 g, 43.14 mmol), NBS (6.65 g, 37.39 mmol) and TBAB (1.85 g, 5.75 mmol) in ACN (10 mL) was stirred for 2 h at 80 under N2 atmosphere. Solvent was removed under vacuum and the residue was purified by silica gel chromatography, eluted with Petroleum ether/EtOAc=3:7 to afford 3-bromo-1-(3-bromophenyl)-2,2-difluoropropan-1-one (6.9 g, 73.2%) as colorless oil.

### c) 2-(3-bromophenyl)-3,3-difluorooxetane-2-carbonitrile

A solution of 3-bromo-1-(3-bromophenyl)-2,2-difluoropropan-1-one (6.9 g, 21.04 mmol) and KCN (2.06 g, 31.56 mmol) in DMF (5 mL) was stirred overnight at ambient temperature under N2 atmosphere. The resulting mixture was extracted with EtOAc (3×20 ml). The combined organic layers were washed with brine (3×20 ml), dried over anhydrous Na2SO4 and concentrated. The residue was purified by silica gel chromatography, eluted with Petroleum ether/EtOAc=2:3 to afford 2-(3-bromophenyl)-3,3-difluorooxetane-2-carbonitrile (2.2 g, 38.2%) as a yellowish oil.

### d) 1-[2-(3-bromophenyl)-33-difluorooxetan-2-yl]methanamine

A mixture of 2-(3-bromophenyl)-3,3-difluorooxetane-2-carbonitrile (2.2 g, 8.03 mmol), CoCl2.6H2O (2.86 g, 12.04 mmol) and NaBH4 (2.43 g, 64.22 mmol) in MeOH (15 mL) was stirred for 40 min at RT under N2 atmosphere. The solvent was removed under vacuum and the residue was purified by silica gel chromatography, eluted with DCM/MeOH=10:1 to afford 1-[2-(3-bromophenyl)-3,3-difluorooxetan-2-yl]methanamine (376 mg, 16.8%) as a colorless oil.

### e) (1S,2S)—N-[[2-(3-cyanophenyl)-3,3-difluoro-oxetan-2-yl]methyl]-2-phenyl-cyclopropanecarboxamide

The title compound was obtained in analogy to Example 86 (33% yield) as a colorless oil using 1-[2-(3-bromophenyl)-3,3-difluorooxetan-2-yl]methanamine, (1S,2S)-2-phenylcyclopropane-1-carboxylic acid and TCFH/NMI.

MS (m/z): 369.1 [M+H]+.

### Example 88: (1S,2S)—N-[[2-(3-cyanophenyl)-5-oxo-tetrahydrofuran-2-yl]methyl]-2-phenyl-cyclopropanecarboxamide

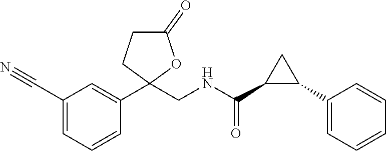 [SMILES: N#Cc1cccc(C2(CNC(=O)[C@H]3C[C@@H]3c3ccccc3)CCC(=O)O2)c1]

The title compound was obtained in analogy to Example 66 as a white solid using 3-cyanophenylboronic acid, (1S,2S)-2-phenylcyclopropane-1-carboxylic acid and TCFH/NMI.

MS (m/z): 361.2 [M+H]+.

### Example 89: N-[[3-(3-cyanophenyl)oxetan-3-yl]methyl]-2-cyclohexyl-acetamide

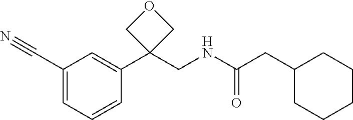 [SMILES: N#Cc1cccc(C2(CNC(=O)CC3CCCCC3)COC2)c1]

The title compound was obtained in analogy to Example 85 (6% yield) using 3-[3-(aminomethyl)oxetan-3-yl]benzonitrile, cyclohexaneacetic acid and TCFH/NMI.

MS (m/z): 313.2 [M+H]+.

### Example 90: N-[[2-(3-cyanophenyl)-5-oxo-tetrahydrofuran-2-yl]methyl]-2-cyclohexyl-acetamide

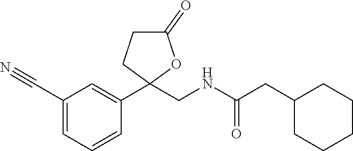 [SMILES: N#Cc1cccc(C2(CNC(=O)CC3CCCCC3)CCC(=O)O2)c1]

The title compound was obtained in analogy to Example 86 (3.2% yield) using 3-[2-(aminomethyl)-5-oxooxolan-2-yl]benzonitrile, cyclohexaneacetic acid and TCFH/NMI.

MS (m/z): 341.2 [M+H]+.

### Example 91: N-[[2-(3-cyanophenyl)-3,3-difluoro-oxetan-2-yl]methyl]-2-cyclohexyl-acetamide

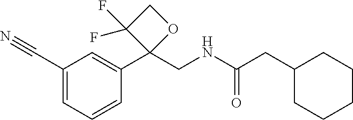 [SMILES: N#Cc1cccc(C2(CNC(=O)CC3CCCCC3)OCC2(F)F)c1]

The title compound was obtained in analogy to Example 87 (26% yield) using 1-[2-(3-bromophenyl)-3,3-difluorooxetan-2-yl]methanamine, cyclohexaneacetic acid and TCFH/NMI.

MS (m/z): 349.2 [M+H]+.

### Example 92: (1S,2S)—N-[2-(3-chlorophenyl)-4-hydroxy-butyl]-2-phenyl-cyclopropanecarboxamide

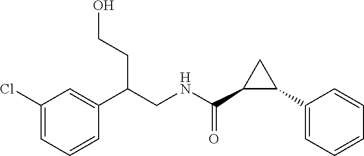 [SMILES: O=C(NCC(CCO)c1cccc(Cl)c1)[C@H]1C[C@@H]1c1ccccc1]

### a) methyl 3-(3-chlorophenyl)-3-cyanopropanoate

To a stirred mixture of 2-(3-chlorophenyl)acetonitrile (10 g, 65.97 mmol) in DMF (100 ml) was added NaH (2.37 g, 98.95 mmol) in portions at 0 C. To the above mixture was added methyl 2-bromoacetate (15.14 g, 98.95 mmol) in portions over 30 min at 0 C. The resulting mixture was stirred for additional 1 h at 0 C. The resulting mixture was extracted with EtOAc (3×200 mL). The combined organic layers were washed with water (3×200 mL), dried over anhydrous Na2SO4 and concentrated under vacuum. The residue was purified by silica gel chromatography, eluted with Petroleum ether/EtOAc (5:1) to afford methyl 3-(3-chlorophenyl)-3-cyanopropanoate (2 g, 13.6%) as a yellowish oil.

### b) 4-amino-3-(3-chlorophenyl)butan-1-ol

To a solution of methyl 3-(3-chlorophenyl)-3-cyanopropanoate (2.9 g, 12.97 mmol) in THF (50 mL) was added LAH (1.23 g, 32.42 mmol) at 0 C in an ice bath. The resulting mixture was stirred at 0 C for 30 minutes. The reaction was quenched with Na2SO4·10H2O at 0 C. The resulting mixture was filtered, the filter cake was washed with THF (3×20 mL). The filtrate was concentrated under reduced pressure. The residue was purified by silica gel chromatography (MeOH:DCM=1:10) to give 4-amino-3-(3-chlorophenyl)butan-1-ol (550 mg. 21.2%) as colorless oil.

### c) (1S,2S)—N-[2-(3-chlorophenyl)-4-hydroxy-butyl]-2-phenyl-cyclopropanecarboxamide

To a stirred mixture of 4-amino-3-(3-chlorophenyl)butan-1-ol (200 mg, 1.0 mmol) and (1S,2S)-2-phenylcyclopropane-1-carboxylic acid (178.69 mg, 1.1 mmol) in DMF was added TCFH (562.06 mg. 2.0 mmol) in portions at room temperature. To the above mixture was added NMI (205.59 mg, 2.51 mmol) dropwise at 0 C under nitrogen atmosphere. The resulting mixture was stirred for additional 60 min at room temperature. The residue was purified by reverse phase HPLC (eluted with 50% to 75% MeOH:Water in 8 min) to give (1S,2S)—N-[2-(3-chlorophenyl)-4-hydroxy-butyl]-2-phenyl-cyclopropanecarboxamide (38.5 mg, 11.2%) as a white solid.

MS (m/z): 344.1 [M+H]+.

### Example 93: N-[[(2S)-2-(3-cyanophenyl)oxetan-2-yl]methyl]-2-cyclohexyl-acetamide

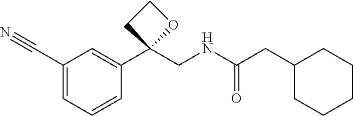 [SMILES: N#Cc1cccc([C@]2(CNC(=O)CC3CCCCC3)CCO2)c1]

The title compound was obtained in analogy to Example 86 (48% yield) as a white solid using 3-[(2S)-2-(aminomethyl)oxetan-2-yl]benzonitrile, cyclohexaneacetic acid and TCFH/NMI.

MS (m/z): 313.2 [M+H]+.

### Example 94: N-[[(2R)-2-(3-cyanophenyl)oxetan-2-yl]methyl]-2-cycloheptyl-acetamide

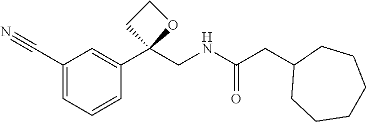 [SMILES: N#Cc1cccc([C@@]2(CNC(=O)CC3CCCCCC3)CCO2)c1]

The title compound was obtained in analogy to Example 86 (36% yield) as a white solid using 3-[(2R)-2-(aminomethyl)oxetan-2-yl]benzonitrile, cycloheptylacetic acid and TCFH/NMI.

MS (m/z): 337.2 [M+H]+.

### Example 95: (1S,2S)—N-[[(2S)-2-(3-cyanophenyl)oxetan-2-yl]methyl]-2-(trifluoromethyl)cyclopropanecarboxamide

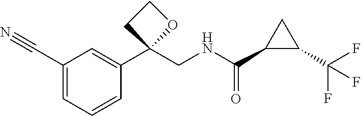 [SMILES: N#Cc1cccc([C@]2(CNC(=O)[C@H]3C[C@@H]3C(F)(F)F)CCO2)c1]

The title compound was obtained in analogy to Example 86 (36% yield) as a white solid using 3-[(2S)-2-(aminomethyl)oxetan-2-yl]benzonitrile, (1S,2S)-2-(trifluoromethyl)cyclopropane-1-carboxylic acid and TCFH/NMI.

MS (m/z): 325.1 [M+H]+.

### Example 96: N-[[(2S)-2-(3-cyanophenyl)oxetan-2-yl]methyl]-2,2-difluoro-spiro[2.3]hexane-1-carboxamide

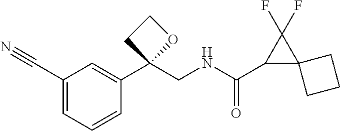 [SMILES: N#Cc1cccc([C@]2(CNC(=O)C3C(F)(F)C34CCC4)CCO2)c1]

The title compound was obtained in analogy to Example 86 (51% yield) as a white oil using 3-[(2S)-2-(aminomethyl)oxetan-2-yl]benzonitrile, 2,2-difluorospiro[2.3]hexane-1-carboxylic acid and TCFH/NMI.

MS (m/z): 333.1 [M+H]+.

### Example 97: N-[[(2R)-2-(3-cyanophenyl)oxetan-2-yl]methyl]-2-norboman-2-yl-acetamide

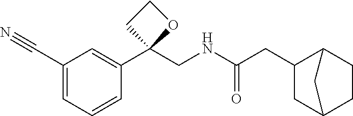 [SMILES: N#Cc1cccc([C@@]2(CNC(=O)CC3CC4CCC3C4)CCO2)c1]

The title compound was obtained in analogy to Example 86 (42% yield) as a white oil using 3-[(2R)-2-(aminomethyl)oxetan-2-yl]benzonitrile, bicyclo[2.2.1]heptan-2-ylacetic acid and TCFH/NMI.

MS (m/z): 325.2 [M+H]+.

### Example 98: N-[[(2R)-2-(3-cyanophenyl)oxetan-2-yl]methyl]bicyclo[2.2.2]octane-2-carboxamide

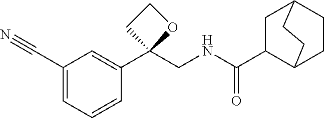 [SMILES: N#Cc1cccc([C@@]2(CNC(=O)C3CC4CCC3CC4)CCO2)c1]

The title compound was obtained in analogy to Example 86 (20% yield) as a white oil using 3-[(2R)-2-(aminomethyl)oxetan-2-yl]benzonitrile, bicyclo[2.2.2]octane-2-carboxylic acid and TCFH/NMI.

MS (m/z): 325.2 [M+H]+.

### Example 99: (1S,2S)—N-[[2-[6-(difluoromethoxy)-2-pyridyl]oxetan-2-yl]methyl]-2-phenyl-cyclopropanecarboxamide

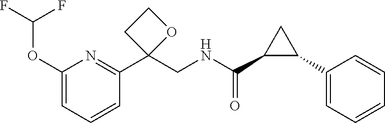 [SMILES: O=C(NCC1(c2cccc(OC(F)F)n2)CCO1)[C@H]1C[C@@H]1c1ccccc1]

1-(2-(6-(difluoromethoxy)pyridin-2-yl)oxetan-2-yl)methanamine was obtained in analogy to Example 67 starting from 6-bromopyridin-2-ol. The titled compound was obtained in analogy to Example 86 (18% yield) as a light yellowish oil using 1-(2-[6-(difluoromethoxy)pyridin-2-yl)oxetan-2-yl)methanamine, (1S,2S)-2-phenylcyclopropane-1-carboxylic acid and TCFH/NMI.

MS (m/z): 375.2 [M+H]+.

### Example 100: N-[[(2S)-2-(3-cyanophenyl)oxetan-2-yl]methyl]spiro[3.3]heptane-3-carboxamide

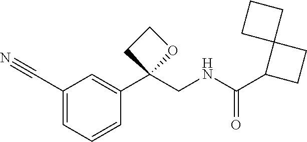 [SMILES: N#Cc1cccc([C@]2(CNC(=O)C3CCC34CCC4)CCO2)c1]

The title compound was obtained in analogy to Example 86 (9.4% yield) as a white solid using 3-[(2S)-2-(aminomethyl)oxetan-2-yl]benzonitrile, spiro[3.3]heptane-1-carboxylic acid and TCFH/NMI.

MS (m/z): 311.2 [M+H]+.

### Example 101: (1SR,2RS)—N-[[(2S)-2-(3-cyanophenyl)oxetan-2-yl]methyl]-2-cyclobutyl-cyclopropanecarboxamide

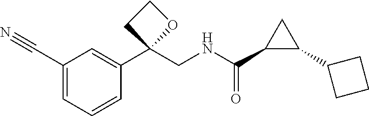 [SMILES: N#Cc1cccc([C@]2(CNC(=O)[C@H]3C[C@@H]3C3CCC3)CCO2)c1]

The title compound was obtained in analogy to Example 86 (23% yield) as a white solid using 3-[(2S)-2-(aminomethyl)oxetan-2-yl]benzonitrile, rac-(1R,2S)-2-cyclobutylcyclopropane-1-carboxylic acid and TCFH/NMI.

MS (m/z): 311.2 [M+H]+.

### Example 102: N-[[(2S)-2-(3-cyanophenyl)oxetan-2-yl]methyl]spiro[3.4]octane-7-carboxamide

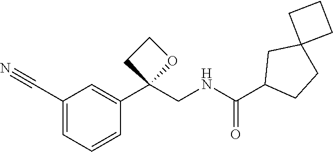 [SMILES: N#Cc1cccc([C@]2(CNC(=O)C3CCC4(CCC4)C3)CCO2)c1]

The title compound was obtained in analogy to Example 86 (44% yield) as a white solid using 3-[(2S)-2-(aminomethyl)oxetan-2-yl]benzonitrile, spiro[3.4]octane-6-carboxylic acid and TCFH/NMI.

MS (m/z): 325.2 [M+H]+.

### Example 103: N-[[(2S)-2-(3-cyanophenyl)oxetan-2-yl]methyl]-3-cyclopentyl-2-fluoro-propanamide

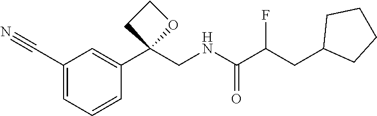 [SMILES: N#Cc1cccc([C@]2(CNC(=O)C(F)CC3CCCC3)CCO2)c1]

The title compound was obtained in analogy to Example 86 (46% yield) as a white solid using 3-[(2S)-2-(aminomethyl)oxetan-2-yl]benzonitrile, 3-cyclopentyl-2-fluoropropanoic acid and TCFH/NMI.

MS (m/z): 331.2 [M+H]+.

### Example 104: N-[[(2S)-2-(3-cyanophenyl)oxetan-2-yl]methyl]spiro[3.4]octane-8-carboxamide

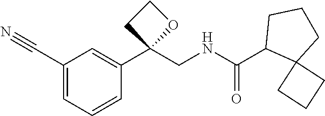 [SMILES: N#Cc1cccc([C@]2(CNC(=O)C3CCCC34CCC4)CCO2)c1]

The title compound was obtained in analogy to Example 86 (23% yield) as a white solid using 3-[(2S)-2-(aminomethyl)oxetan-2-yl]benzonitrile, spiro[3.4]octane-5-carboxylic acid and TCFH/NMI.

MS (m/z): 325.2 [M+H]+.

### Example 105: N-[[(2S)-2-(3-cyanophenyl)oxetan-2-yl]methyl]dispiro[3.0.3⁵.1⁴]nonane-7-carboxamide

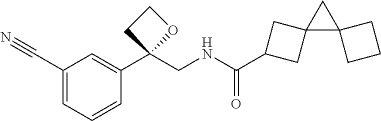 [SMILES: N#Cc1cccc([C@]2(CNC(=O)C3CC4(C3)CC43CCC3)CCO2)c1]

The title compound was obtained in analogy to Example 86 (38% yield) as a light yellowish oil using 3-[(2S)-2-(aminomethyl)oxetan-2-yl]benzonitrile, dispiro[3.0.3⁵.1⁴]nonane-2-carboxylic acid and TCFH/NMI.

MS (m/z): 337.2 [M+H]+.

### Example 106: N-[[(2S)-2-(3-cyanophenyl)oxetan-2-yl]methyl]-6-oxaspiro[2.5]octane-2-carboxamide

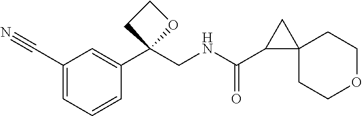 [SMILES: N#Cc1cccc([C@]2(CNC(=O)C3CC34CCOCC4)CCO2)c1]

The title compound was obtained in analogy to Example 86 (34% yield) as a white solid using 3-[(2S)-2-(aminomethyl)oxetan-2-yl]benzonitrile. 6-oxaspiro[2.5]octane-1-carboxylic acid and TCFH/NMI.

MS (m/z): 327.2 [M+H]+.

### Example 107: (1SR,2SR)—N-[[(2S)-2-(3-cyanophenyl)oxetan-2-yl]methyl]-2-(4-fluorophenyl)cyclopropanecarboxamide

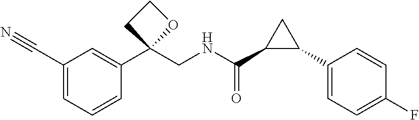 [SMILES: N#Cc1cccc([C@]2(CNC(=O)[C@H]3C[C@@H]3c3ccc(F)cc3)CCO2)c1]

The title compound was obtained in analogy to Example 86 (35% yield) as a white solid using 3-[(2S)-2-(aminomethyl)oxetan-2-yl]benzonitrile, (1S,2S)-2-(4-fluorophenyl)cyclopropane-1-carboxylic acid and TCFH/NMI.

MS (m/z): 351.2 [M+H]+.

### Example 108: N-[[(2S)-2-(3-cyanophenyl)oxetan-2-yl]methyl]-2-norbornan-2-yl-acetamide

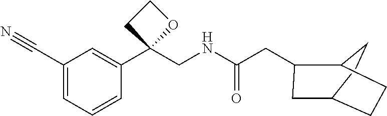 [SMILES: N#Cc1cccc([C@]2(CNC(=O)CC3CC4CCC3CC4)CCO2)c1]

The title compound was obtained in analogy to Example 86 (39% yield) as a white solid using 3-[(2S)-2-(aminomethyl)oxetan-2-yl]benzonitrile, 2-(bicyclo[2.2.1]heptan-2-yl)acetic acid and TCFH/NMI.

MS (m/z): 325.2 [M+H]+.

### Example 109: N-[[(2S)-2-(3-cyanophenyl)oxetan-2-yl]methyl]-2-norbornan-1-yl-acetamide

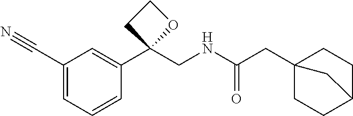 [SMILES: N#Cc1cccc([C@]2(CNC(=O)CC34CCC(CC3)C4)CCO2)c1]

The title compound was obtained in analogy to Example 86 (5.8% yield) as a white oil using 3-[(2S)-2-(aminomethyl)oxetan-2-yl]benzonitrile, 2-(bicyclo[2.2.1]heptan-1-yl)acetic acid and TCFH/NMI.

MS (m/z): 325.2 [M+H]+.

### Example 110: (1RS,2RS)-2-tert-butyl-N-[[(2S)-2-(3-cyanophenyl)oxetan-2-yl]methyl]cyclopropanecarboxamide

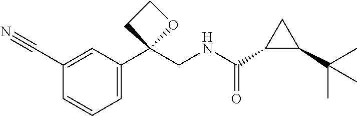 [SMILES: CC(C)(C)[C@@H]1C[C@H]1C(=O)NC[C@@]1(c2cccc(C#N)c2)CCO1]

The title compound was obtained in analogy to Example 86 (38% yield) as a white solid using 3-[(2S)-2-(aminomethyl)oxetan-2-yl]benzonitrile, rac-(1S,2S)-2-tert-butylcyclopropane-1-carboxylic acid and TCFH/NMI.

MS (m/z): 313.2 [M+H]+.

### Example 111: N-[[(2S)-2-(3-cyanophenyl)oxetan-2-yl]methyl]-2-(2-hydroxynorbornan-2-yl)acetamide

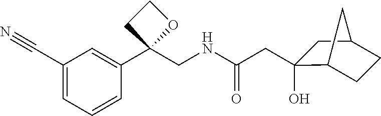 [SMILES: N#Cc1cccc([C@]2(CNC(=O)CC3(O)CC4CCC3C4)CCO2)c1]

The title compound was obtained in analogy to Example 86 (21% yield) as a white solid using 3-[(2S)-2-(aminomethyl)oxetan-2-yl]benzonitrile, 2-(2-hydroxybicyclo[2.2.1]heptan-2-yl)acetic acid and TCFH/NMI.

MS (m/z): 341.2 [M+H]+.

### Example 112: (1S,2R)—N-[[(2S)-2-(3-cyanophenyl)oxetan-2-yl]methyl]-2-cyclopentyl-cyclopropanecarboxamide

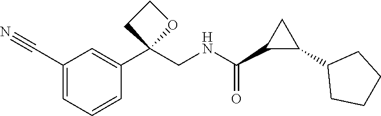

The title compound was obtained in analogy to Example 86 (20% yield) as a white solid using 3-[(2S)-2-(aminomethyl)oxetan-2-yl]benzonitrile. (1S,2R)-2-cyclopentylcyclopropane-1-carboxylic acid and TCFH/NMI.

MS (m/z): 325.2 [M+H]+.

### Example 113: (1SR,2SR)—N-[[(2S)-2-(3-cyanophenyl)oxetan-2-yl]methyl]-2-(1,1-difluoroethyl)cyclopropanecarboxamide

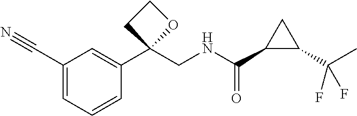 [SMILES: CC(F)(F)[C@H]1C[C@@H]1C(=O)NC[C@@]1(c2cccc(C#N)c2)CCO1]

The title compound was obtained in analogy to Example 86 (20% yield) as a white solid using 3-[(2S)-2-(aminomethyl)oxetan-2-yl]benzonitrile, 2-(1,1-difluoroethyl)cyclopropane-1-carboxylic acid and TCFH/NMI.

MS (m/z): 321.1 [M+H]+.

### Example 114: (1SR,2SR)-2-tert-butoxy-N-[[(2S)-2-(3-cyanophenyl)oxetan-2-yl]methyl]cyclopropanecarboxamide

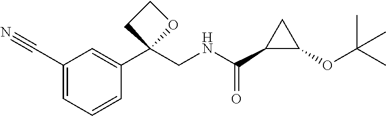 [SMILES: CC(C)(C)O[C@H]1C[C@@H]1C(=O)NC[C@@]1(c2cccc(C#N)c2)CCO1]

The title compound was obtained in analogy to Example 86 (20% yield) as a white solid using 3-[(2S)-2-(aminomethyl)oxetan-2-yl]benzonitrile, rac-(1S,2S)-2-(tert-butoxy)cyclopropane-1-carboxylic acid and TCFH/NMI.

MS (m/z): 329.2 [M+H]+.

### Example 115: N-[[2-[6-(difluoromethoxy)-2-pyridyl]oxetan-2-yl]methyl]-6-oxaspiro[2.5]octane-2-carboxamide

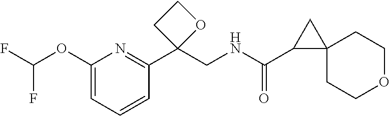 [SMILES: O=C(NCC1(c2cccc(OC(F)F)n2)CCO1)C1CC12CCOCC2]

The title compound was obtained in analogy to Example 86 (40% yield) as a light yellowish oil using 1-{2-[6-(difluoromethoxy)pyridin-2-yl]oxetan-2-yl}methanamine. 6-oxaspiro[2.5]octane-1-carboxylic acid and TCFH/NMI.

MS (m/z): 369.2 [M+H]+.

### Example 116: N-[[(2S)-2-(3-cyanophenyl)oxetan-2-yl]methyl]-2-spiro[3.3]heptan-2-yl-acetamide

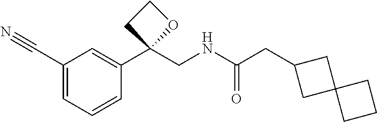 [SMILES: N#Cc1cccc([C@]2(CNC(=O)CC3CC4(CCC4)C3)CCO2)c1]

The title compound was obtained in analogy to Example 86 (13% yield) as a white oil using 3-[(2S)-2-(aminomethyl)oxetan-2-yl]benzonitrile, 2-(spiro[3.3]heptan-2-yl)acetic acid and TCFH/NMI.

MS (m/z): 325.2 [M+H]+.

### Example 117: 2-cyclohexyl-N-[[2-[2-(difluoromethoxy)-4-pyridyl]oxetan-2-yl]methyl]acetamide

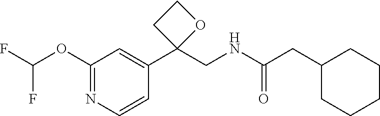 [SMILES: O=C(CC1CCCCC1)NCC1(c2ccnc(OC(F)F)c2)CCO1]

The title compound was obtained in analogy to Example 86 (28% yield) as a white solid using 1-{2-[2-(difluoromethoxy)pyridin-4-yl]oxetan-2-yl}methanamine, cyclohexaneacetic acid and TCFH/NMI.

MS (m/z): 355.2 [M+H]+.

### Example 118: N-[[2-[2-(difluoromethoxy)-4-pyridyl]oxetan-2-yl]methyl]-6,6-difluoro-spiro[2.5]octane-2-carboxamide

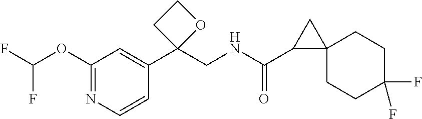 [SMILES: O=C(NCC1(c2ccnc(OC(F)F)c2)CCO1)C1CC12CCC(F)(F)CC2]

The title compound was obtained in analogy to Example 86 (27% yield) as a white solid using 1-{2-[2-(difluoromethoxy)pyridin-4-yl]oxetan-2-yl}methanamine, 6,6-difluorospiro[2.5]octane-1-carboxylic acid and TCFH/NMI.

MS (m/z): 403.2 [M+H]+.

### Example 119: 2-cyclohexyl-N-[[2-[6-(difluoromethoxy)-2-pyridyl]oxetan-2-yl]methyl]acetamide

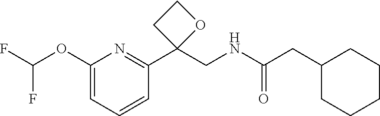 [SMILES: O=C(CC1CCCCC1)NCC1(c2cccc(OC(F)F)n2)CCO1]

The title compound was obtained in analogy to Example 86 (42% yield) as a white solid using 1-{2-[6-(difluoromethoxy)pyridin-2-yl]oxetan-2-yl}methanamine, 2-cyclohexylacetic acid and TCFH/NMI.

MS (m/z): 355.2 [M+H]+.

### Example 120: N-[[2-[6-(difluoromethoxy)-2-pyridyl]oxetan-2-yl]methyl]-6,6-difluoro-spiro[2.5]octane-2-carboxamide

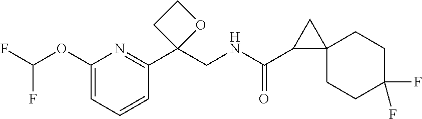 [SMILES: O=C(NCC1(c2cccc(OC(F)F)n2)CCO1)C1CC12CCC(F)(F)CC2]

The title compound was obtained in analogy to Example 86 (61% yield) as a white solid using 1-{2-[6-(difluoromethoxy)pyridin-2-yl]oxetan-2-yl}methanamine, 6,6-difluorospiro[2.5]octane-1-carboxylic acid and TCFH/NMI.

MS (m/z): 403.2 [M+H]+.

### Example 121: (1S,2S)—N-[[2-(2-ethyl-4-pyridyl)oxetan-2-yl]methyl]-2-phenyl-cyclopropanecarboxamide

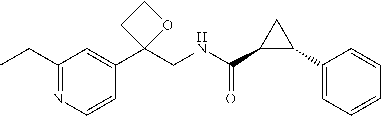 [SMILES: CCc1cc(C2(CNC(=O)[C@H]3C[C@@H]3c3ccccc3)CCO2)ccn1]

The title compound was obtained in analogy to Example 86 (22% yield) as a white solid using 1-[2-(2-ethylpyridin-4-yl)oxetan-2-yl]methanamine. (1S,2S)-2-phenylcyclopropane-1-carboxylic acid and TCFH/NMI.

MS (m/z): 337.2 [M+H]+.

### Example 122: N-[[2-[2-(difluoromethoxy)-4-pyridyl]oxetan-2-yl]methyl]spiro[2.5]octane-2-carboxamide

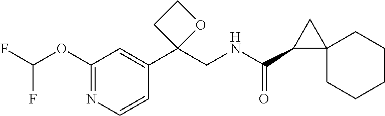 [SMILES: O=C(NCC1(c2ccnc(OC(F)F)c2)CCO1)[C@H]1CC12CCCCC2]

The title compound was obtained in analogy to Example 86 (7% yield) as a white solid using 1-{2-[2-(difluoromethoxy)pyridin-4-yl]oxetan-2-yl}methanamine, spiro[2.5]octane-1-carboxylic acid and TCFH/NMI.

MS (m/z): 367.2 [M+H]+.

### Example 123: N-[[2-[6-(difluoromethoxy)-2-pyridyl]oxetan-2-yl]methyl]spiro[2.5]octane-2-carboxamide

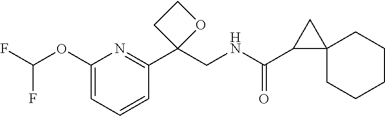 [SMILES: O=C(NCC1(c2cccc(OC(F)F)n2)CCO1)C1CC12CCCCC2]

The title compound was obtained in analogy to Example 86 (53% yield) as a light yellow oil using 1-{2-[6-(difluoromethoxy)pyridin-2-yl]oxetan-2-yl}methanamine, spiro[2.5]octane-1-carboxylic acid and TCFH/NMI.

MS (m/z): 367.2 [M+H]+.

### Example 124: (1SR,2SR)—N-[[(2S)-2-(3-cyanophenyl)oxetan-2-yl]methyl]-2-(2-pyridyl)cyclopropanecarboxamide

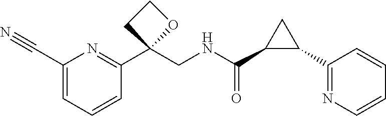 [SMILES: N#Cc1cccc([C@]2(CNC(=O)[C@H]3C[C@@H]3c3ccccn3)CCO2)n1]

The title compound was obtained in analogy to Example 86 (27% yield) as a white oil using 3-[(2S)-2-(aminomethyl)oxetan-2-yl]benzonitrile, rac-(1S,2S)-2-(pyridin-2-yl)cyclopropane-1-carboxylic acid and TCFH/NMI.

MS (m/z): 334.2 [M+H]+.

### Example 125: (1S,2S)—N-[[2-(3-cyano-4-fluoro-phenyl)oxetan-2-yl]methyl]-2-phenyl-cyclopropanecarboxamide

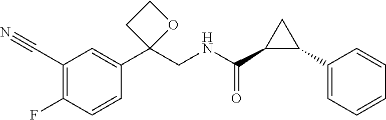 [SMILES: N#Cc1cc(C2(CNC(=O)[C@H]3C[C@@H]3c3ccccc3)CCO2)ccc1F]

The title compound was obtained in analogy to Example 86 (7% yield) as a white solid using 5-[2-(aminomethyl)oxetan-2-yl]-2-fluorobenzonitrile. (1S,2S)-2-phenylcyclopropane-1-carboxylic and TCFH/NMI.

MS (m/z): 351.1 [M+H]+.

### Example 126: (1SR,2SR)—N-[[(2S)-2-(3-cyanophenyl)oxetan-2-yl]methyl]-2-ethyl-cyclopropanecarboxamide

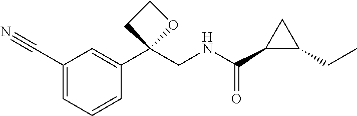 [SMILES: CC[C@H]1C[C@@H]1C(=O)NC[C@@]1(c2cccc(C#N)c2)CCO1]

The title compound was obtained in analogy to Example 86 (19% yield) as a white oil using 3-[(2S)-2-(aminomethyl)oxetan-2-yl]benzonitrile, rac-trans-2-ethylcyclopropane-1-carboxylic acid and TCFH/NMI.

MS (m/z): 285.2 [M+H]+.

### Example 127: (1SR,2RS)—N-[[(2S)-2-(3-cyanophenyl)oxetan-2-yl]methyl]-2-(3,3-difluorocyclobutyl)cyclopropanecarboxamide

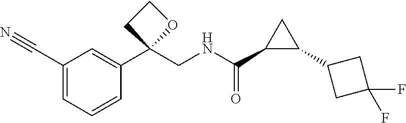 [SMILES: N#Cc1cccc([C@]2(CNC(=O)[C@H]3C[C@@H]3C3CC(F)(F)C3)CCO2)c1]

The title compound was obtained in analogy to Example 86 (17% yield) as a white solid using 3-[(2S)-2-(aminomethyl)oxetan-2-yl]benzonitrile, 2-(3,3-difluorocyclobutyl)cyclopropane-1-carboxylic acid and TCFH/NMI.

MS (m/z): 347.2 [M+H]+.

### Example 128: (1S,2S)-2-(4-bromophenyl)-N-[[(2S)-2-(3-cyanophenyl)oxetan-2-yl]methyl]cyclopropanecarboxamide

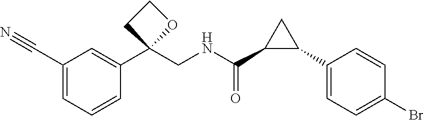 [SMILES: N#Cc1cccc([C@]2(CNC(=O)[C@H]3C[C@@H]3c3ccc(Br)cc3)CCO2)c1]

The title compound was obtained in analogy to Example 86 (24% yield) as a white solid using 3-[(2S)-2-(aminomethyl)oxetan-2-yl]benzonitrile, (1S,2S)-2-(4-bromophenyl)cyclopropane-1-carboxylic acid and TCFH/NMI.

MS (m/z): 411.2 [M+H]+.

### Example 129: (1S,2S)—N-[[2-[2-(difluoromethoxy)-4-pyridyl]oxetan-2-yl]methyl]-2-(trifluoromethyl)cyclopropanecarboxamide

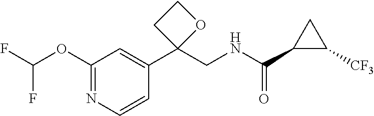 [SMILES: O=C(NCC1(c2ccnc(OC(F)F)c2)CCO1)[C@H]1C[C@@H]1C(F)(F)F]

The title compound was obtained in analogy to Example 86 (54% yield) as a white solid using 1-{2-[2-(difluoromethoxy)pyridin-4-yl]oxetan-2-yl}methanamine, (1S,2S)-2-(trifluoromethyl)cyclopropane-1-carboxylic acid and TCFH/NMI.

MS (m/z): 367.2 [M+H]+.

### Example 130: (1S,2S)—N-[[2-[6-(difluoromethoxy)-2-pyridyl]oxetan-2-yl]methyl]-2-(trifluoromethyl)cyclopropanecarboxamide

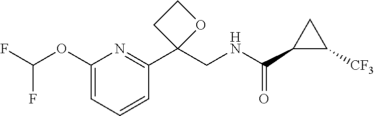 [SMILES: O=C(NCC1(c2cccc(OC(F)F)n2)CCO1)[C@H]1C[C@@H]1C(F)(F)F]

The title compound was obtained in analogy to Example 86 (64% yield) as a light yellowish oil using 1-{2-[6-(difluoromethoxy)pyridin-2-yl]oxetan-2-yl}methanamine, (1S,2S)-2-(trifluoromethyl)cyclopropane-1-carboxylic acid and TCFH/NMI.

MS (m/z): 367.2 [M+H]+.

### Example 131: N-[[(2S)-2-(3-cyanophenyl)oxetan-2-yl]methyl]-2-fluoro-2-norbornan-2-yl-acetamide

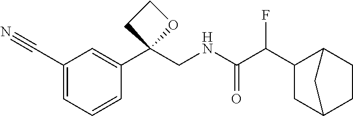 [SMILES: N#Cc1cccc([C@]2(CNC(=O)C(F)C3CC4CCC3C4)CCO2)c1]

### a) methyl 2-[(2E)-bicyclo[2.2.1]heptan-2-ylidene]acetate

To a stirred solution of norcamphor (300 mg. 2.72 mmol) and methyl 2-(dimethoxyphosphoryl)acetate (991.93 mg, 5.45 mmol) in THF (10 mL) was added NaH (217.85 mg, 5.446 mmol, 60%) in portions at 0 C under nitrogen atmosphere. The resulting mixture was stirred for 2 h at room temperature. The reaction was quenched by addition of diluted HCl (10 mL) at 0 C. The aqueous layer was extracted with EtOAc (3×30 mL). The organic layers were combined, washed with brine, dried over anhydrous sodium sulfate, filtered, and concentrated under vacuum. The residue was purified by silica gel chromatography, eluted with Petroleum ether/EtOAc (5:1) to afford methyl 2-[(2E)-bicyclo[2.2.1]heptan-2-ylidene]acetate (170 mg, 37.6%) as a colorless oil.

### b) methyl 2-{bicyclo[2.2.1]heptan-2-yl}acetate

To a solution of methyl 2-[(2E)-bicyclo[2.2.1]heptan-2-ylidene]acetate (5.6 g, 33.69 mmol) in THF (50 mL) at 0 C was added Pd/C (7.17 g, 67.38 mmol) and the mixture was stirred for 3 h at room temperature under hydrogen atmosphere. The resulting mixture was filtered, and the filter cake was washed with THF (3×30 mL). The filtrate was concentrated under reduced pressure to give the crude methyl 2-{bicyclo[2.2.1]heptan-2-yl}acetate (4.0 g, 70.6%) as a colorless oil.

### c) methyl 2-{bicycle [2.2.1]heptan-2-yl}-2-fluoroacetate

To a solution of methyl 2-{bicyclo[2.2.1]heptan-2-yl}acetate (500 mg, 2.97 mmol) in 50 mL THF at −78 C was added dropwise LDA (1.8 mL, 2 M in THF) and the mixture was stirred for 1 hour at −78 C under nitrogen atmosphere, followed by the addition of NFSI (1.12 g, 3.56 mmol) at −78 C. The resulting mixture was stirred for 1 h at −78 C under nitrogen atmosphere and allowed to warm up to room temperature for another 1 hour. The reaction was quenched with sat. NH4Cl (aq.) at OC. The aqueous layer was extracted with EtOAc (3×10 mL). The organic layers were combined, washed with brine, dried over anhydrous sodium sulfate, filtered, and concentrated under vacuum. The residue was purified by silica gel chromatography, eluted with Petroleum ether/EtOAc (2.5:1) to afford methyl 2-{bicycle [2.2.1]heptan-2-yl}-2-fluoroacetate (180 mg, 32.5%) as a yellow oil.

### d) bicyclo[2.2.1]heptan-2-yl(fluoro)acetic acid

To a stirred solution of methyl 2-{bicyclo[2.2.1]heptan-2-yl}-2-fluoroacetate (180 mg, 0.97 mmol) in MeOH (5 mL) was added a solution of LiOH (115.75 mg, 4.84 mmol) in 1 mL of H2O dropwise at 0 C under nitrogen atmosphere. The resulting mixture was stirred for 2 hours at room temperature. The reaction mixture was concentrated under vacuum and diluted HCl solution was added to pH=5. The resulting solution was extracted with ethyl acetate. The organic layers were combined, washed with brine, dried over anhydrous sodium sulfate, filtered, and concentrated under vacuum. This resulted in the crude bicyclo[2.2.1]heptan-2-yl(fluoro)acetic acid (40 mg, 24.0%) as yellow oil.

### e) N-[[(2S)-2-(3-cyanophenyl)oxetan-2-yl]methyl]-2-fluoro-2-norbornan-2-yl-acetamide

The title compound was obtained in analogy to Example 86 (39% yield) as a white solid using 3-[(2S)-2-(aminomethyl)oxetan-2-yl]benzonitrile, bicyclo[2.2.1]heptan-2-yl(fluoro)acetic acid and TCFH/NMI.

MS (m/z): 343.2 [M+H]+.

### Example 132: N-[[2-[6-(difluoromethoxy)-2-pyridyl]oxetan-2-yl]methyl]-2-[1-(trifluoromethyl)cyclopropyl]acetamide

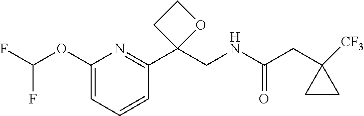 [SMILES: O=C(CC1(C(F)(F)F)CC1)NCC1(c2cccc(OC(F)F)n2)CCO1]

The title compound was obtained in analogy to Example 86 (64% yield) as a white solid using 1-{2-[6-(difluoromethoxy)pyridin-2-yl]oxetan-2-yl}methanamine, 2-(1-(trifluoromethyl)cyclopropyl)acetic acid and TCFH/NMI.

MS (m/z): 381.2 [M+H]+.

### Example 133: N-[[(2S)-2-(3-cyanophenyl)oxetan-2-yl]methyl]-2-(5-methyl-2-furyl)cyclopropanecarboxamide

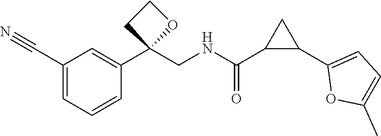 [SMILES: Cc1ccc(C2CC2C(=O)NC[C@@]2(c3cccc(C#N)c3)CCO2)o1]

The title compound was obtained in analogy to Example 86 (7.7% yield) as a white solid using 3-[(2S)-2-(aminomethyl)oxetan-2-yl]benzonitrile, 2-(5-methylfuran-2-yl)cyclopropane-1-carboxylic acid and TCFH/NMI.

MS (m/z): 337.1 [M+H]+.

### Example 134: (1SR,2SR)—N-[[(2S)-2-(3-cyanophenyl)oxetan-2-yl]methyl]-2-prop-1-ynyl-cyclopropanecarboxamide

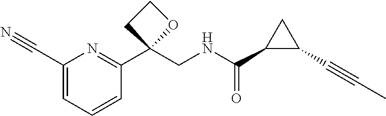 [SMILES: CC#C[C@H]1C[C@@H]1C(=O)NC[C@@]1(c2cccc(C#N)n2)CCO1]

### a) methyl (2E)-hex-2-en-4-ynoate

To a stirred mixture of 2-butyn-1-ol (3.5 g, 49.94 mmol) and methyl 2-(triphenyl-lambda5-phosphanylidene)acetate (20.04 g, 59.92 mmol) in DCM (250 mL) was added MnO2 (43.41 g, 499.33 mmol) in portions at room temperature under N2 atmosphere. The resulting solution was stirred overnight at room temperature. Upon completion of reaction, water was added and the resulting solution was extracted with CH2Cl2 (3×). The organic layers were combined, washed with brine dried over anhydrous sodium sulfate, filtered, and concentrated under vacuum. The residue was purified by silica gel chromatography (Petroleum ether:EtOAc=10:1) to give the product methyl (2E)-hex-2-en-4-ynoate (3.5 g, 56.5%) as a light yellowish oil.

### b) methyl 2-(prop-1-yn-1-yl)cyclopropane-1-carboxylate

To a solution of trimethylsulfoxonium iodide (6.38 g, 28.99 mmol) in DMSO (50 mL) was added t-BuOK (3.254 g, 28.99 mmol) and the mixture was stirred for 1 h at room temperature under nitrogen atmosphere. A solution of methyl (2E)-hex-2-en-4-ynoate (2.4 g. 19.33 mmol) in DMSO (50 mL) at 80 C was treated with the above mixture and the resulting mixture was stirred for 3 hours at 80 C. Upon completion of reaction, diluted HCl was added and the resulting solution was extracted with CH2Cl2 (3×). The organic layers were combined, washed with brine, dried over anhydrous sodium sulfate, filtered, and concentrated under vacuum. The residue was purified by silica gel chromatography (Petroleum ether:EtOAc=5:1) to give the product methyl 2-(prop-1-yn-1-yl)cyclopropane-1-carboxylate (1.2 g, 44.8%) as a light yellowish oil.

### c) 2-(prop-1-yn-1-yl)cyclopropane-1-carboxylic acid

To a stirred solution of methyl 2-(prop-1-yn-1-yl)cyclopropane-1-carboxylate (1.23 g, 8.90 mmol) in THF (40 mL) and MeOH (10 mL) was added a solution of LiOH (1.07 g, 44.51 mmol) in H2O (10 mL) at 0 C. The mixture was stirred at room temperature for 2 hours. Upon completion of reaction, the organic solvent was removed under vacuum. The mixture was acidified to pH 4-5 with diluted hydrochloric acid. The resulting mixture was extracted with DCM (5×20 mL). The combined organic layers were dried over anhydrous sodium sulfate, filtered, and concentrated under vacuum. The residue was purified by reverse phase HPLC eluting with water and acetonitrile (0 to 20% ACN gradient) to give 2-(prop-1-yn-1-yl)cyclopropane-1-carboxylic acid (720 mg, 65.2%) as a white solid.

### d) (1SR,2SR)—N-[[(2S)-2-(3-cyanophenyl)oxetan-2-yl]methyl]-2-prop-1-ynyl-cyclopropanecarboxamide

The title compound was obtained in analogy to Example 86 (62% yield) as a white solid using 3-[(2S)-2-(aminomethyl)oxetan-2-yl]benzonitrile, 2-(prop-1-yn-1-yl)cyclopropane-1-carboxylic acid and TCFH/NMI. Purification afforded the desired product as one of the isolated diastereomer, with the other diastereomer shown as example 137.

MS (m/z): 295.1 [M+H]+.

### Example 135: N-[[(2S)-2-(3-cyanophenyl)oxetan-2-yl]methyl]-2-pyrrol-1-yl-cyclopropanecarboxamide

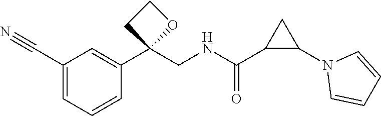 [SMILES: N#Cc1cccc([C@]2(CNC(=O)C3CC3n3cccc3)CCO2)c1]

### a) methyl methyl (2Z)-3-(pyrrol-1-yl)prop-2-enoate

A solution of pyrrole (3 g, 44.72 mmol) in DMF (300 mL) was treated with t-BuONa (2.15 g, 22.36 mmol) for 30 min at 65 C followed by the addition of methyl propiolate (4.51 g, 53.66 mmol). The mixture was stirred for 3 h at 100 C under nitrogen atmosphere. Water was added and the resulting solution was extracted with CH2Cl2 (3×). The organic layers were combined, washed with brine dried over anhydrous sodium sulfate, filtered, and concentrated under vacuum. The residue was purified by silica gel chromatography (Petroleum ether:EtOAc=5:1) to give the product methyl methyl (2Z)-3-(pyrrol-1-yl)prop-2-enoate (1.5 g, 22.2%) as a light yellowish solid.

### b) methyl 2-(5-methylfuran-2-yl)cyclopropane-1-carboxylate

To a solution of t-BuOK (709.03 mg, 6.32 mmol) in DMSO (12 mL) and Trimethylsulfoxonium iodide (1.39 g, 6.32 mmol) was treated for 1 h at room temperature under nitrogen atmosphere followed by the addition of (2Z)-3-(pyrrol-1-yl)prop-2-enoate (700 mg, 4.21 mmol) dropwise for 3 h at 80 C. Upon completion of reaction, water was added and the resulting solution was extracted with CH2Cl2 (3×). The organic layers were combined, washed with brine, dried over anhydrous sodium sulfate, filtered, and concentrated under vacuum. The residue was purified by silica gel chromatography (Petroleum ether:EtOAc=7:1) to give the product methyl 2-(5-methylfuran-2-yl)cyclopropane-1-carboxylate (500 mg, 65.9%) as a light yellowish oil.

### c) 2-(pyrrol-1-yl)cyclopropane-1-carboxylic acid

To a stirred solution of methyl 2-(5-methylfuran-2-yl)cyclopropane-1-carboxylate (150 mg, 0.91 mmol) in THF (4 mL) and MeOH (1 mL) was added a solution of LiOH (186.36 mg, 4.55 mmol) in H2O (1 mL). The mixture was stirred at 0 C for 2 hours. The solvent was removed under vacuum. The mixture was acidified to pH 4-5 with diluted hydrochloric acid. The resulting mixture was extracted with DCM (5×20 mL). The combined organic layers were dried over anhydrous sodium sulfate, filtered, and concentrated under vacuum. The residue was purified by reverse phase HPLC eluting with water and acetonitrile (0 to 30% ACN gradient) to give 2-(pyrrol-1-yl)cyclopropane-1-carboxylic acid (100 mg. 73.0%) as a light yellowish solid.

### d) N-[[(2S)-2-(3-cyanophenyl)oxetan-2-yl]methyl]-2-pyrrol-1-yl-cyclopropanecarboxamide

The title compound was obtained in analogy to Example 86 (45% yield) as a colorless oil using 3-[(2S)-2-(aminomethyl)oxetan-2-yl]benzonitrile, 2-(pyrrol-1-yl)cyclopropane-1-carboxylic acid and TCFH/NMI.

MS (m/z): 322.1 [M+H]+.

### Example 136: N-[[(2S)-2-(3-cyanophenyl)oxetan-2-yl]methyl]-2-(4,4-difluorocyclohexyl)cyclopropanecarboxamide

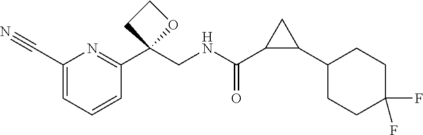 [SMILES: N#Cc1cccc([C@]2(CNC(=O)C3CC3C3CCC(F)(F)CC3)CCO2)n1]

The title compound was obtained in analogy to Example 86 (37% yield) as a colorless oil using 3-[(2S)-2-(aminomethyl)oxetan-2-yl]benzonitrile, 2-(4,4-difluorocyclohexyl)cyclopropane-1-carboxylic acid and TCFH/NMI.

MS (m/z): 375.1 [M+H]+.

### Example 137: (1SR,2RS)—N-[[(2S)-2-(3-cyanophenyl)oxetan-2-yl]methyl]-2-prop-1-ynyl-cyclopropanecarboxamide

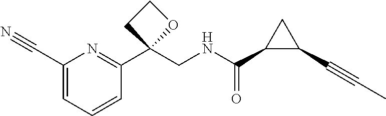 [SMILES: CC#C[C@@H]1C[C@@H]1C(=O)NC[C@@]1(c2cccc(C#N)n2)CCO1]

The title compound is the other diastereomer isolated from purification of example 134.

MS (m/z): 295.1 [M+H]+.

### BIOLOGICAL EXPERIMENTS

The potency of Kv7 potentiators was determined using the SyncroPatch 384 (Nanion) high throughput electrophysiology platform.

### Example 138 Cell Culture

CHO cells stably expressing either human Kv7.2, Kv7.4 or Kv7.5/7.3 under a constitutive CMV promoter were used for these studies. Cells were maintained in F12 Hams+1 mM L-glutamine (Hyclone) supplemented with 10% FBS (Sigma), 0.3×NEAA (Non-essential Gamino-acids) and 400 ug/ml G418 at 37° C. in 5% CO₂. Cells were cultured in T-225 flasks (Nunc) for 2-3 days to reach 85-95% confluence prior to electrophysiological recording.

### Example 139 Solutions

Solutions were of the following composition:

Earle's balanced salt solution (in mM): 135 NaCl, 5.4 KCl, 5 Glucose, 2 CaCl₂), 1 MgCl₂, 5 HEPES, pH 7.4. Seal enhancer solution (in mM): 90 NaCl, 3 KCl, 35 CaCl₂), 10 MgCl₂, 10 HEPES, pH 7.4. Extracellular recording solution (in mM): 71 NaCl, 70 NMDG, 13 KCl. 5 Glucose, 2 CaCl₂, 1 MgCl₂, 10 HEPES, pH 7.4. Intracellular recording solution (in mM): 130 KF, 20 KCl, 4 EGTA, 10 HEPES, 2 EDTA, 0.01 Escin, pH 7.2.

### Example 140 Cell Preparation

Cells were harvested for electrophysiological recording upon reaching appropriate confluence. Cells were first washed in DPBS (Hyclone, Cat #SH30028.03) and then 2 ml of Accutase (MP Biomedicals #1000449) was added at 28° C. until ~90% of cells were suspended. F12 HAM's media+1 mM L-glutamine (Hyclone, SH30026.02) was then added to the flask to dilute the accutase. Cells were then triturated until a single cell suspension was achieved, a cell count was performed, and cells were centrifuged for 2 min. at 1,000 rpm. Media was then aspirated, and cells were resuspended in Earle's balanced salt solution to a concentration of 0.75×10⁶ cells/ml and allowed to recover for 25 min at 10° C.

### Example 141 SyncroPatch Recording

At the beginning of each assay, 20 μl of cell suspension was dispensed into each well of a multi-hole 384-well SyncroPatch chip by the onboard pipettor. Cell sealing was initiated, and seal enhancer solution was added to facilitate seal formation. Upon completion of sealing, cells were washed 3 times with extracellular recording solution and the assay voltage protocol was started. Human Kv7.2, Kv7.4 or Kv7.5/7.3 channels were evaluated using a voltage protocol in which cells were voltage-clamped at a holding potential of −60 mV. Potassium currents were continuously activated with a series of three voltage steps to −30 mV for 3 seconds, 40 mV for 1 second and −90 mV for 4 seconds with 12 seconds between successive voltage sweeps. Potassium currents were measured from the −90 mV repolarizing step. Baseline current was assessed for 3.5 minutes prior to the addition of 5.6 μM zinc pyrithione (1 μM for Kv7.4). Kv7.2, Kv7.4 or Kv7.5/7.3 current in the presence of zinc pyrithione was acquired over five minutes to allow channels to reach steady state activity prior to addition of test agents. Channel activity was monitored for three minutes preceding the addition of 30 μM ML-213 (3 minutes) to achieve maximum activation. 150 mM TEA with 10 μM XE-991 was applied for 2 minutes to measure the leak current during maximum inhibition of Kv7.2, Kv7.4 or Kv7.5/7.3 channels.

### Example 142 Data Analysis

Data were collected on the SyncroPatch platform using PatchControl software (Nanion) and processed and analyzed using DataControl Software (Nanion). Percent activation was calculated from potassium current as follows: The average current for the 5 sweeps in the presence of zinc pyrithione immediately preceding test agent addition was taken as the ‘control’ data. Likewise, the average potassium current in the presence of test agents 5 sweeps immediately preceding addition of 30 μM ML-213 was determined for ‘drug’ data. The average potassium current 5 sweeps immediately prior to addition of 150 mM TEA+10 μM XE-991 was determined for ‘max activation’ data while average currents from 5 sweeps immediately following addition of TEA was determined for ‘max inhibition’ data. Percent activation for each of the 384-wells of a sealchip was calculated as ((‘drug’−‘max inhibition’)/(‘max activation’−‘max inhibition’))*100 with Pipeline Pilot (Accelrys). Percent activation was plotted as a function of concentration and concentration-response curves were fitted with a logistic equation {Y is Bottom+(Top-Bottom)/(1+10\hat{ }((Log EC50−X)*HillSlope))} for determination of the EC₅₀ (IDBS ABASE). EC₅₀ values for human Kv7.2 are provided in Table 1.

### Example 143 Microsomal Clearance

Pooled mixed gender human liver microsomes were purchased from BioIVT at a concentration of 20 mg protein/mL and stored at −80° C. Incubations were conducted in V-bottomed 350 μL polypropylene 96-well plates. Compounds were diluted in phosphate buffer (final concentrations 0.5 μM compound, 0.5% DMSO) and mixed with diluted liver microsomes (fc 0.25 mg/mL). This mixture was aliquoted onto the assay plate (six wells per compound) and pre-warmed at 37° C. for ten minutes. Addition of NADPH (fc 0.5 mM, final incubation volume 100 μL) to each well started the time course. Reactions were stopped at 0.5, 3, 5, 10, 20 & 30 minutes by addition of 150 μL acetonitrile containing internal standard (IS; 0.125 μg/mL daidzein) and stopped samples were removed to a clean polypropylene plate. At the end of the assay the plate was refrigerated for an hour and then centrifuged at 3000 rpm for 10 minutes at 4° C. A sample of the supernatant was removed to a fresh plate and diluted 10-fold with 50:50 acetonitrile:water, then heat sealed. Analysis was by liquid chromatography coupled with tandem mass spectrometry (LC-MS/MS) for analyte and IS levels. Ratios of the analyte and IS peak areas were generated for results calculation (IS ratio). Dextromethorphan and verapamil were included in each experiment as controls for high clearance compounds. IS ratio data were converted into natural log values which were plotted against time and a linear regression fit applied. The slope of the line was returned and converted into the elimination constant (ki) by multiplying by −1. The elimination constant was used to calculate the in vitro CLᵢₙₜ: in vitro CLᵢₙₜ (μL/min/mg protein) is kₑ₁*(1000/protein conc in mg/mL).

### Example 144

A compound of formula (I), or a solvate or a pharmaceutically acceptable salt thereof can be used in a manner known per se as the active ingredient for the production of tablets of the following composition: Per tablet

| | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 145

A compound of formula (I), or a solvate or a pharmaceutically acceptable salt thereof, can be used in a manner known per se as the active ingredient for the production of capsules of the following composition:

Per capsule

| | |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the present invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A compound of formula (I), or a solvate, or a pharmaceutically acceptable salt thereof: 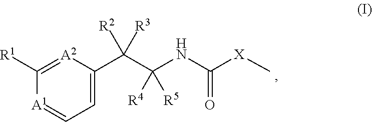
wherein
A¹ is N or CR^{a};
A² is N or CR^{b};
R^{a} is hydrogen or a halogen;
R^{b} is hydrogen or a halogen;
R¹ is selected from hydrogen, cyano, a haloC₁₋₆alkyl, a C₁₋₆alkoxy, a haloC₁₋₆alkoxy, and a halogen;
R² and R³ are independently selected from the group consisting of hydrogen, hydroxy, a C₁₋₆alkyl, a C₁₋₆alkoxy, a hydroxyC₁₋₆alkyl, a C₃₋₅cycloalkylC₁₋₆alkoxy, cyano, —OCD₃, a cyanoC₁₋₆alkoxy, a haloC₁₋₆alkoxy, and a hydroxy-C₂₋₄alkenyl, or
R² and R³ together with the carbon atom they are attached to form a 4-6 membered heterocycloalkyl or a 3-6 membered cycloalkyl optionally substituted with 1 to 4 halogen, or oxo, or
R³ is hydrogen, and R² is ═CHCH₂OH;
R⁴ is hydrogen, a hydroxyC₁₋₆alkyl, or a halogen;
R⁵ is hydrogen, or a halogen;
X is selected from the group consisting of NH, NHCH₂, CH₂, CH₂CH₂, CH₂CH₂CH₂, NHCH₂CH₂CH₂, NHCH₂CH₂, CH═CH, CH₂CH₂CH(OH), CH₂CH(OH), a bond, CHFCH₂, and 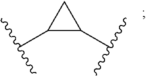 [SMILES: *C1CC1*]
and
R⁶ is selected from the group consisting of a 3-10 membered cycloalkyl, a 7-11 membered spiro-cycloalkyl, a 7-11 membered oxo-spiro-heterocycloalkyl, an aryl, a heteroaryl, a C₁₋₆alkyl, —C≡CCH₃, a 4-10 membered heterocycloalkyl, and a haloC₁₋₆alkyl, wherein the cycloalkyl, the spiro-cycloalkyl, the 7-11 membered oxa-spiro-cycloalkyl, the aryl, the heteroaryl, or the heterocycloalkyl are optionally substituted with one, two, or three substituents independently selected from the group consisting of a halogen, a haloC₁₋₆alkyl, a C₁₋₆alkyl, a C₁₋₆alkoxy, and a haloC₁₋₆alkoxy.

2. (canceled)

3. The compound of claim 1, wherein only one of A¹ and A² is N.

4. The compound of claim 1, wherein A¹ is CR^{a}, and A² is CR^{b}.

5. The compound of claim 4, wherein R^{a} and R^{b} are hydrogen.

6. (canceled)

7. A The compound of claim 1, wherein R¹ is C₁.

8. The compound of claim 1, wherein R¹ is cyano or CHF₂O—.

9. (canceled)

10. The compound of claim 1, wherein R² is hydroxy or methoxy, and R³ is methyl.

11-12. (canceled)

13. The compound of claim 1, wherein R² and R³ together with the C-atom they are attached to form a 4-6 membered heterocycloalkyl, and
wherein the heterocycloalkyl is 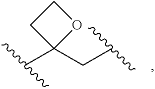 [SMILES: *CC1(*)CCO1]
which is optionally substituted with two F.

14. The compound of claim 1, wherein R⁴ and R⁵ are H.

15. (canceled)

16. The compound of claim 1, wherein X is

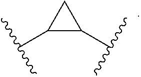 [SMILES: *C1CC1*]

17. (canceled)

18. The compound of claim 1, wherein R⁶ is selected from an aryl, a phenyl, a 3-10 membered cycloalkyl, a 4-6 membered saturated mononcyclic cycloalkyl, and a 7-10 membered bridged cycloalkyl,
wherein the phenyl, cycloalkyl, or bridged cycloalkyl are optionally substituted as with one or two halogen or oxo.

19. The compound of claim 1, selected from the group consisting of:

| Compound | Name | Structure |
|---|---|---|
| 1 | rac-(1S,2S)-N-[2-(3-chlorophenyl)- 2-methoxy-propyl]-2-phenyl- cyclopropanecarboxamide | 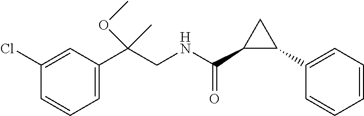 [SMILES: COC(C)(CNC(=O)[C@H]1C[C@@H]1c1ccccc1)c1cccc(Cl)c1] |
| 2 | (1S,2S)-N-[(2R)-2-(3-chlorophenyl)- 2-methoxy-propyl]-2-phenyl- cyclopropanecarboxamide | 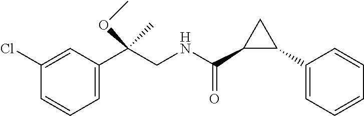 [SMILES: CO[C@@](C)(CNC(=O)[C@H]1C[C@@H]1c1ccccc1)c1cccc(Cl)c1] |
| 3 | 1-[2-(3-chlorophenyl)-2-methoxy- propyl]-3-cyclopentyl-urea | 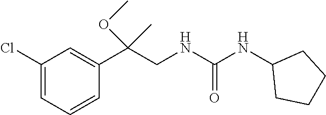 [SMILES: COC(C)(CNC(=O)NC1CCCC1)c1cccc(Cl)c1] |
| 4 | 1-[2-(3-chlorophenyl)-2-methoxy- ethyl]-3-norbornan-2-yl-urea | 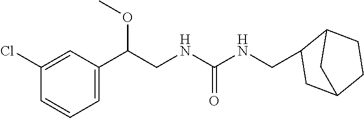 [SMILES: COC(CNC(=O)NCC1CC2CCC1C2)c1cccc(Cl)c1] |
| 5 | 1-[2-(3-chlorophenyl)-2-methoxy- propyl]-3-(3-methoxyphenyl)urea | 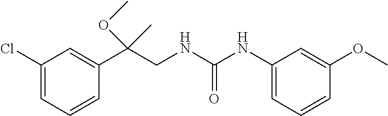 [SMILES: COc1cccc(NC(=O)NCC(C)(OC)c2cccc(Cl)c2)c1] |
| 6 | 1-[2-(3-chlorophenyl)-2-methoxy- propyl]-3-(m-tolyl)urea | 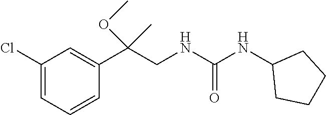 [SMILES: COC(C)(CNC(=O)NC1CCCC1)c1cccc(Cl)c1] |
| 7 | 1-[[1-(3- cyclophenyl]cyclopropyl]methyl]- 3-cyclopentyl-urea | 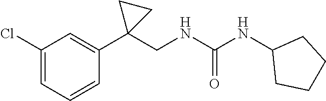 [SMILES: O=C(NCC1(c2cccc(Cl)c2)CC1)NC1CCCC1] |
| 8 | 1-butyl-3-[2-(3-chlorophenyl)-2- methoxy-propyl]urea | 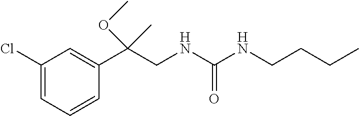 [SMILES: CCCCNC(=O)NCC(C)(OC)c1cccc(Cl)c1] |
| 9 | 1-[2-(3-chlorophenyl)-2-methoxy- propyl]-3-(2-furylmethyl)urea | 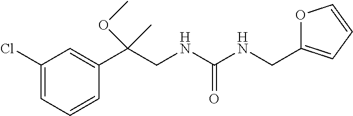 [SMILES: COC(C)(CNC(=O)NCc1ccco1)c1cccc(Cl)c1] |
| 10 | 1-[2-(3-chlorophenyl)-2-methoxy- propyl]-3-[(3- fluorophenyl)methyl]urea | 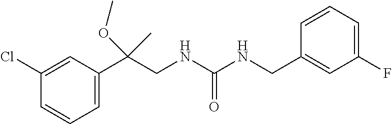 [SMILES: COC(C)(CNC(=O)NCc1cccc(F)c1)c1cccc(Cl)c1] |
| 11 | 1-[2-(3-chlorophenyl)-2-methoxy- propyl]-3-(m-tolylmethyl)urea | 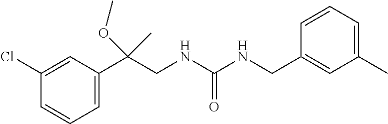 [SMILES: COC(C)(CNC(=O)NCc1cccc(C)c1)c1cccc(Cl)c1] |
| 12 | 1-[[1-(3- chlorophenyl)cyclopropyl]methyl]- 3-(1-oxaspiro[4.4]nonan-3-yl)urea | 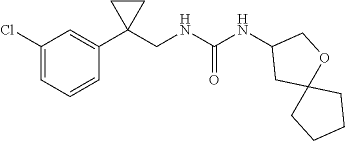 [SMILES: O=C(NCC1(c2cccc(Cl)c2)CC1)NC1COC2(CCCC2)C1] |
| 13 | 1-[2-(3-chlorophenyl)-2-methoxy- propyl]-3-(1-oxaspiro[4.4]nonan-3- yl)urea | 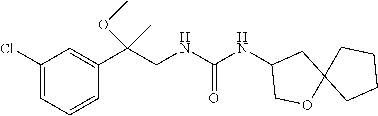 [SMILES: COC(C)(CNC(=O)NC1COC2(CCCC2)C1)c1cccc(Cl)c1] |
| 14 | 1-[2-(3-chlorophenyl)-2-methoxy- propyl]-3-(3-ethoxyphenyl)urea | 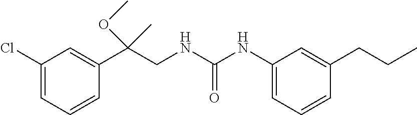 [SMILES: CCCc1cccc(NC(=O)NCC(C)(OC)c2cccc(Cl)c2)c1] |
| 15 | 1-[[1-(3- chlorophenyl)cyclopropyl]methyl]- 3-(tetrahydropyran-2-ylmethyl)urea | 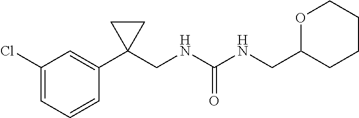 [SMILES: O=C(NCC1CCCCO1)NCC1(c2cccc(Cl)c2)CC1] |
| 16 | 1-[(2R)-2-(3-chlorophenyl)-2- methoxy-propyl]-3-cyclopentyl-urea | 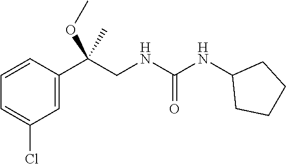 [SMILES: CO[C@@](C)(CNC(=O)NC1CCCC1)c1cccc(Cl)c1] |
| 17 | 1-[(2R)-2-(3-chlorophenyl)-2- methoxy-propyl]-3-(3- methoxyphenyl)urea | 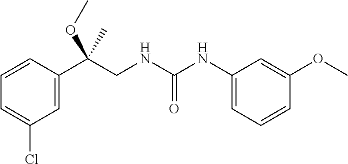 [SMILES: COc1cccc(NC(=O)NC[C@](C)(OC)c2cccc(Cl)c2)c1] |
| 18 | 1-[2-(3-chlorophenyl)-2- (cyclopropoxy)ethyl]-3-cyclopentyl- urea | 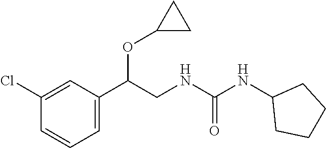 [SMILES: O=C(NCC(OC1CC1)c1cccc(Cl)c1)NC1CCCC1] |
| 19 | N-[2-(3-chlorophenyl)-2-methoxy- propyl]-2-cyclohexyl-acetamide | 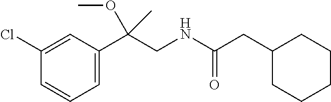 [SMILES: COC(C)(CNC(=O)CC1CCCCC1)c1cccc(Cl)c1] |
| 20 | rac-(1R,2R)-N-[2-(3-chlorophenyl)- 2-methoxy-ethyl]-2-phenyl- cyclopropanecarboxamide | 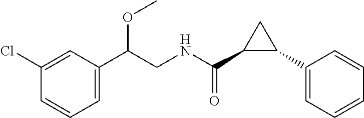 [SMILES: COC(CNC(=O)[C@H]1C[C@@H]1c1ccccc1)c1cccc(Cl)c1] |
| 21 | 1-[2-(3-chlorophenyl)-2-methoxy- propyl]-3-(cyclopentylmethyl)urea | 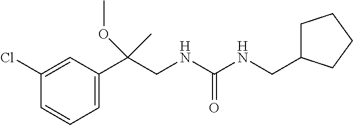 [SMILES: COC(C)(CNC(=O)NCC1CCCC1)c1cccc(Cl)c1] |
| 22 | 1-[2-(3-chlorophenyl)-2-methoxy- ethyl]-3-(m-tolyl)urea | 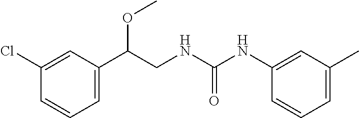 [SMILES: COC(CNC(=O)Nc1cccc(C)c1)c1cccc(Cl)c1] |
| 23 | 1-[2-(3-chlorophenyl)-2-methoxy- propyl]-3-(cyclopropylmethyl)urea | 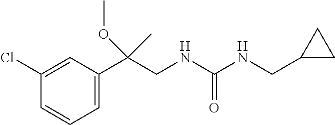 [SMILES: COC(C)(CNC(=O)NCC1CC1)c1cccc(Cl)c1] |
| 24 | 1-[2-(3-bromophenyl)-2-isopropoxy- ethyl]-3-cyclopentyl-urea | 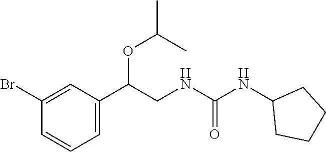 [SMILES: CC(C)OC(CNC(=O)NC1CCCC1)c1cccc(Br)c1] |
| 25 | 1-[2-(3-chlorophenyl)-2-methoxy- propyl]-3-[rac-(1S,2S,4R)- norbornan-2-yl]urea | 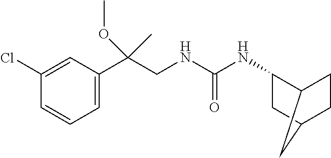 [SMILES: COC(C)(CNC(=O)N[C@H]1CC2CCC1C2)c1cccc(Cl)c1] |
| 26 | 1-[2-(3-chlorophenyl)-2- (trideuteriomethoxy)propyl]-3- cyclopentyl-urea | 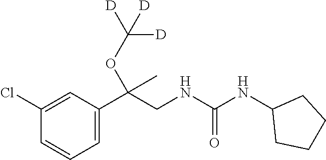 [SMILES: [2H]C([2H])([2H])OC(C)(CNC(=O)NC1CCCC1)c1cccc(Cl)c1] |
| 27 | 1-(3-methoxyphenyl)-3-[[2-(m- tolyl)-1,3-dioxolan-2-yl]methyl]urea | 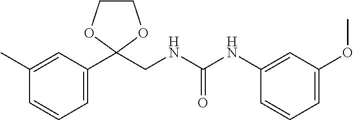 |
| 28 | 1-[2-(3-bromophenyl)-2-ethoxy- ethyl]-3-cyclopentyl-urea | 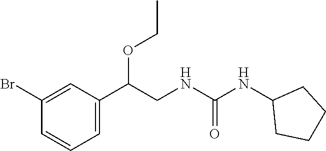 [SMILES: CCOC(CNC(=O)NC1CCCC1)c1cccc(Br)c1] |
| 29 | 1-[2-(3-chlorophenyl)-2-methoxy- propyl]-3-[3- (trifluoromethoxy)phenyl]urea | 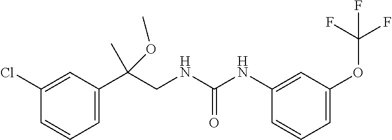 [SMILES: COC(C)(CNC(=O)Nc1cccc(OC(F)(F)F)c1)c1cccc(Cl)c1] |
| 30 | 1-[2-(3-chlorophenyl)-2-methoxy- propyl]-3-cyclopent-3-en-1-yl-urea | 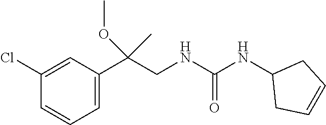 [SMILES: COC(C)(CNC(=O)NC1CC=CC1)c1cccc(Cl)c1] |
| 31 | rac-(1S,2S)-N-[2-cyano-2-[3- (trifluoromethyl)phenyl]propyl]-2- phenyl-cyclopropanecarboxamide | 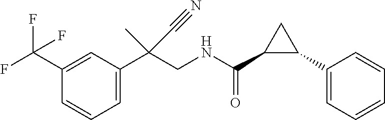 [SMILES: CC(C#N)(CNC(=O)[C@H]1C[C@@H]1c1ccccc1)c1cccc(C(F)(F)F)c1] |
| 32 | 1-[2-(3-chlorophenyl)-2-methoxy- propyl]-3-(3-phenylpropyl)urea | 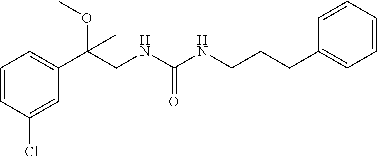 [SMILES: COC(C)(CNC(=O)NCCCc1ccccc1)c1cccc(Cl)c1] |
| 33 | 1-[2-(3-chlorophenyl)-2-methoxy- propyl]-3-(2-thienylmethyl)urea | 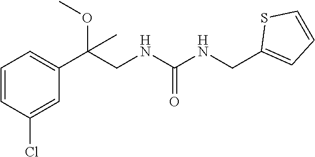 [SMILES: COC(C)(CNC(=O)NCc1cccs1)c1cccc(Cl)c1] |
| 34 | 1-[2-(3-chlorophenyl)-2-methoxy- propyl]-3-[2-(4- methoxyphenyl)ethyl]urea | 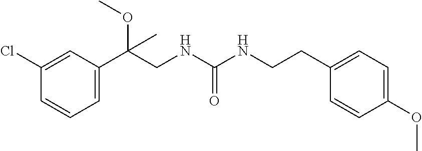 [SMILES: COc1ccc(CCNC(=O)NCC(C)(OC)c2cccc(Cl)c2)cc1] |
| 35 | 1-[2-(3-chlorophenyl)-2-methoxy- propyl]-3-[rac-(1S,4R)-norbornan-2- yl]urea | 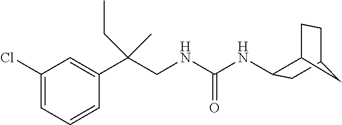 [SMILES: CCC(C)(CNC(=O)NC1CC2CCC1C2)c1cccc(Cl)c1] |
| 36 | N-[2-(2-chlorophenyl)-2-methoxy- ethyl]-2-(1-naphthyl)acetamide | 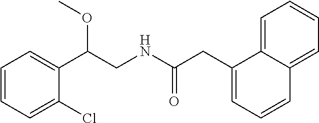 [SMILES: COC(CNC(=O)Cc1cccc2ccccc12)c1ccccc1Cl] |
| 37 | rac-(1S,2S)-N-[2-(3-chlorophenyl)- 2-cyano-propyl]-2-phenyl- cyclopropanecarboxamide | 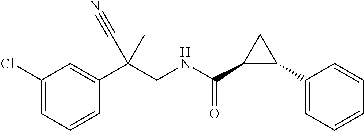 [SMILES: CC(C#N)(CNC(=O)[C@H]1C[C@@H]1c1ccccc1)c1cccc(Cl)c1] |
| 38 | 1-[3-(1r,3r)-(3- chlorophenyl)cyclobutyl]-3- [(1SR,2SR,4RS)-norbornan- 2-yl]urea | 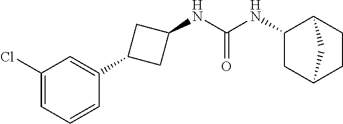 [SMILES: O=C(N[C@H]1C[C@@H]2CC[C@H]1C2)N[C@H]1C[C@H](c2cccc(Cl)c2)C1] |
| 39 | (1S,2S)-N-[[(2R)-2-(3- chlorophenyl)oxetan-2-yl]methyl]-2- phenyl-cyclopropanecarboxamide | 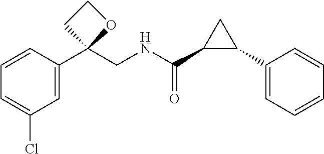 [SMILES: O=C(NC[C@]1(c2cccc(Cl)c2)CCO1)[C@H]1C[C@@H]1c1ccccc1] |
| 40 | (1S,2S)-N-[[(2S)-2-(3- chlorophenyl)oxetan-2-yl]methyl]-2- phenyl-cyclopropanecarboxamide | 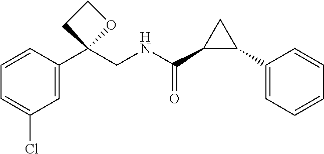 [SMILES: O=C(NC[C@@]1(c2cccc(Cl)c2)CCO1)[C@H]1C[C@@H]1c1ccccc1] |
| 41 | (1S,2S)-N-[[(2R)-2-(3- chlorophenyl)-3,3-difluoro-oxetan- 2-yl]methyl]-2-phenyl- cyclopropanecarboxamide | 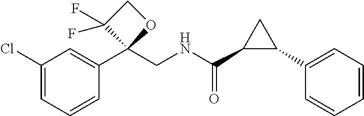 [SMILES: O=C(NC[C@@]1(c2cccc(Cl)c2)OCC1(F)F)[C@H]1C[C@@H]1c1ccccc1] |
| 42 | (1S,2S)-N-[[(2S)-2-(3-chlorophenyl)- 3,3-difluoro-oxetan-2-yl]methyl]-2- phenyl-cyclopropanecarboxamide | 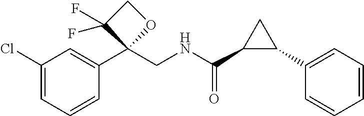 [SMILES: O=C(NC[C@]1(c2cccc(Cl)c2)OCC1(F)F)[C@H]1C[C@@H]1c1ccccc1] |
| 43 | N-[2-(3-chlorophenyl)-2-methoxy- propyl]-2-(2- fluorophenyl)cyclopropane- carboxamide | 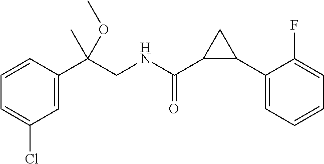 [SMILES: COC(C)(CNC(=O)C1CC1c1ccccc1F)c1cccc(Cl)c1] |
| 44 | 2-(4-chlorophenyl)-N-[2-(3- chlorophenyl)-2-methoxy- propyl]cyclopropanecarboxamide | 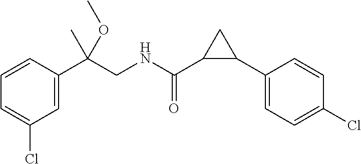 [SMILES: COC(C)(CNC(=O)C1CC1c1ccc(Cl)cc1)c1cccc(Cl)c1] |
| 45 | 1-[2-(3-chlorophenyl)-2-(2,2,2- trifluoroethoxy)ethyl]-3-[rac- (1R,4S)-norbornan-2-yl]urea | 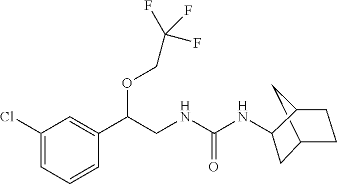 [SMILES: O=C(NCC(OCC(F)(F)F)c1cccc(Cl)c1)NC1CC2CCC1C2] |
| 46 | 1-[2-(3-chlorophenyl)-2- (cyclobutoxy)ethyl]-3-[rac-(1R,4S)- norbornan-2-yl]urea | 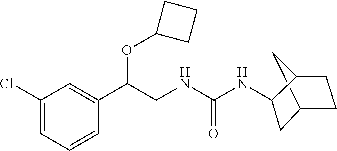 [SMILES: O=C(NCC(OC1CCC1)c1cccc(Cl)c1)NC1CC2CCC1C2] |
| 47 | (E)-N-[2-(3-chlorophenyl)-2- methoxy-propyl]-3-(2- fluorophenyl)prop-2-enamide | 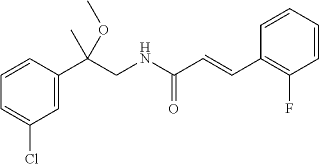 [SMILES: COC(C)(CNC(=O)/C=C/c1ccccc1F)c1cccc(Cl)c1] |
| 48 | (1SR,2SR)-N-[(2R)-2-(3- chlorophenyl)-2-methoxy-propyl]-2- (3-pyridyl)cyclopropanecarboxamide | 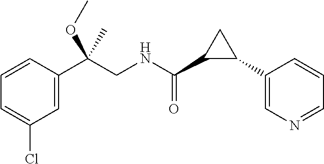 |
| 49 | (1S,2S)-N-[[(2S)-2-(3- cyanophenyl)oxetan-2-yl]methyl]-2- phenyl-cyclopropanecarboxamide | 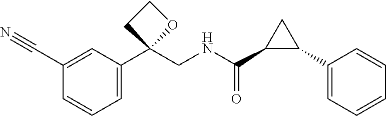 [SMILES: N#Cc1cccc([C@]2(CNC(=O)[C@H]3C[C@@H]3c3ccccc3)CCO2)c1] |
| 50 | 1-[[2-(3-chlorophenyl)oxetan-2- yl]methyl]-3-(3-methoxyphenyl)urea | 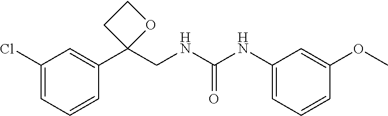 [SMILES: COc1cccc(NC(=O)NCC2(c3cccc(Cl)c3)CCO2)c1] |
| 51 | (1S,2S)-N-[(2RS)-2-(2-chloro-4- pyridyl)-2-methoxy-propyl]-2- phenyl-cyclopropanecarboxamide | 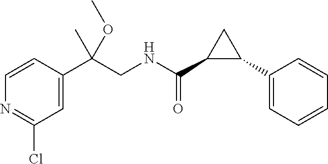 [SMILES: COC(C)(CNC(=O)[C@H]1C[C@@H]1c1ccccc1)c1ccnc(Cl)c1] |
| 52 | (1S,2S)-N-[(2RS)-2-methoxy-2-(2- methoxy-4-pyridyl)propyl]-2- phenyl-cyclopropanecarboxamide | 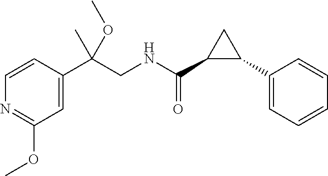 [SMILES: COc1cc(C(C)(CNC(=O)[C@H]2C[C@@H]2c2ccccc2)OC)ccn1] |
| 53 | N-[2-(3-chlorophenyl)-2-methoxy- propyl]-3-cyclopentyl-propanamide | 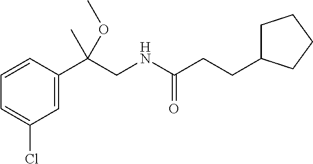 [SMILES: COC(C)(CNC(=O)CCC1CCCC1)c1cccc(Cl)c1] |
| 54 | 1-[[2-(3-chlorophenyl)oxetan-2- yl]methyl]-3-[rac-(1S,2S,4R)- norbornan-2-yl]urea | 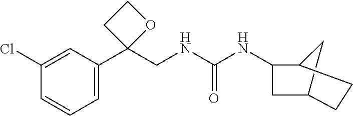 [SMILES: O=C(NCC1(c2cccc(Cl)c2)CCO1)NC1CC2CCC1C2] |
| 55 | (1S,2S)-N-[[(2R)-2-(3- cyanophenyl)oxetan-2-yl]methyl]-2- phenyl-cyclopropanecarboxamide | 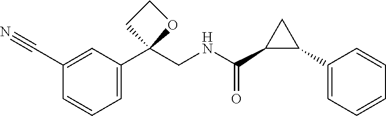 [SMILES: N#Cc1cccc([C@@]2(CNC(=O)[C@H]3C[C@@H]3c3ccccc3)CCO2)c1] |
| 56 | N-[3-(1r,3r)-(3- chlorophenyl)cyclobutyl]-2- cyclohexyl-acetamide | 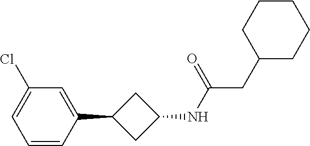 [SMILES: O=C(CC1CCCCC1)N[C@H]1C[C@H](c2cccc(Cl)c2)C1] |
| 57 | N-[[2-(3-chlorophenyl)oxetan-2- yl]methyl]-2-cyclohexyl-acetamide | 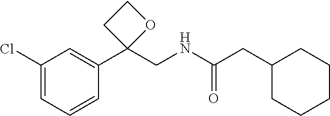 [SMILES: O=C(CC1CCCCC1)NCC1(c2cccc(Cl)c2)CCO1] |
| 58 | 1-(3-chlorophenyl)-3-[[2-(3- chlorophenyl)oxetan-2- yl]methyl]urea | 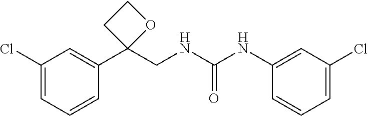 [SMILES: O=C(NCC1(c2cccc(Cl)c2)CCO1)Nc1cccc(Cl)c1] |
| 59 | 1-[[2-(3-chlorophenyl)oxetan-2- yl]methyl]-3- (cyclopentylmethyl)urea | 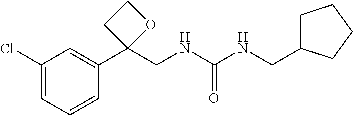 [SMILES: O=C(NCC1CCCC1)NCC1(c2cccc(Cl)c2)CCO1] |
| 60 | 1-[[2-(3-chlorophenyl)oxetan-2- yl]methyl]-3-(2,2- dimethylpropyl)urea | 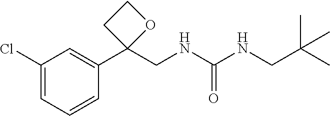 [SMILES: CC(C)(C)CNC(=O)NCC1(c2cccc(Cl)c2)CCO1] |
| 61 | N-[[2-(3-chlorophenyl)oxetan-2- yl]methyl]cyclohexanecarboxamide | 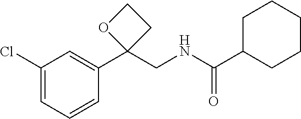 [SMILES: O=C(NCC1(c2cccc(Cl)c2)CCO1)C1CCCCC1] |
| 62 | 1-[(3-chlorophenyl)methyl]-3-[[2-(3- chlorophenyl)oxetan-2- yl]methyl]urea | 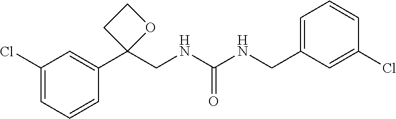 [SMILES: O=C(NCc1cccc(Cl)c1)NCC1(c2cccc(Cl)c2)CCO1] |
| 63 | 2-(1-bicyclo[1.1.1]pentanyl)-N-[[2- (3-chlorophenyl)oxetan-2- yl]methyl]acetamide | 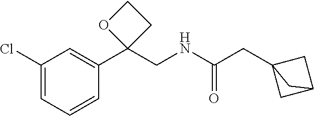 [SMILES: O=C(CC12CC(C1)C2)NCC1(c2cccc(Cl)c2)CCO1] |
| 64 | N-[[2-(3-chlorophenyl)oxetan-2- yl]methyl]spiro[3.3]heptane-2- carboxamide | 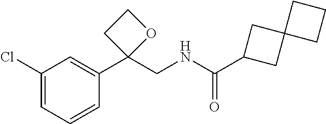 [SMILES: O=C(NCC1(c2cccc(Cl)c2)CCO1)C1CC2(CCC2)C1] |
| 65 | N-[[2-(3-chlorophenyl)oxetan-2- yl]methyl]-2-(4,4- difluorocyclohexyl)acetamide | 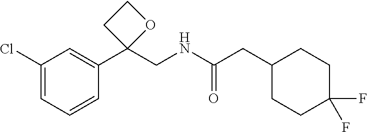 [SMILES: O=C(CC1CCC(F)(F)CC1)NCC1(c2cccc(Cl)c2)CCO1] |
| 66 | (1S,2S)-N-[[2-(3-chlorophenyl)-5- oxo-tetrahydrofuran-2-yl]methyl]-2- phenyl-cyclopropanecarboxamide | 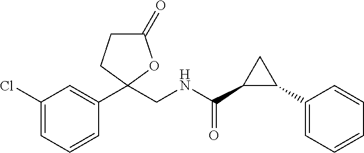 [SMILES: O=C1CCC(CNC(=O)[C@H]2C[C@@H]2c2ccccc2)(c2cccc(Cl)c2)O1] |
| 67 | (1S,2S)-N-[[2-[2-(difluoromethoxy)- 4-pyridyl]oxetan-2-yl]methyl]-2- phenyl-cyclopropanecarboxamide | 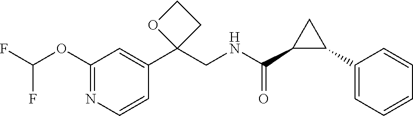 [SMILES: O=C(NCC1(c2ccnc(OC(F)F)c2)CCO1)[C@H]1C[C@@H]1c1ccccc1] |
| 68 | (1S,2S)-N-[[3-(3- chlorophenyl)oxetan-3-yl]methyl]-2- phenyl-cyclopropanecarboxamide | 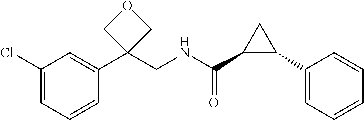 [SMILES: O=C(NCC1(c2cccc(Cl)c2)COC1)[C@H]1C[C@@H]1c1ccccc1] |
| 69 | N-[(2R)-2-(3-chlorophenyl)-2- methoxy-propyl]-4-hydroxy-5,5- dimethyl-hexanamide | 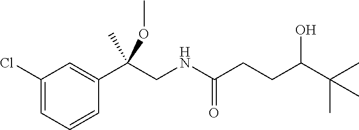 [SMILES: CO[C@@](C)(CNC(=O)CCC(O)C(C)(C)C)c1cccc(Cl)c1] |
| 70 | (1S,2S)-N-[(2S)-2-(3-chlorophenyl)- 2-(cyanomethoxy)ethyl]-2-phenyl- cyclopropanecarboxamide | 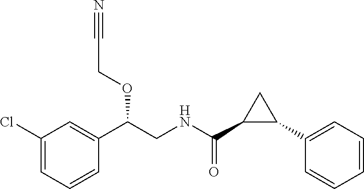 [SMILES: N#CCO[C@H](CNC(=O)[C@H]1C[C@@H]1c1ccccc1)c1cccc(Cl)c1] |
| 71 | 1-(cyclopentylmethyl)-3-[rel-(2S)-2- (3-chlorophenyl)-2-methoxy- propyl]urea | 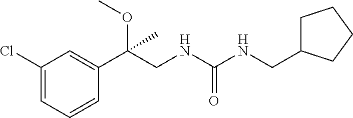 [SMILES: CO[C@@](C)(CNC(=O)NCC1CCCC1)c1cccc(Cl)c1] |
| 72 | 1-[[3-(3-chlorophenyl)oxetan-3- yl]methyl]-3- (cyclopentylmethyl)urea | 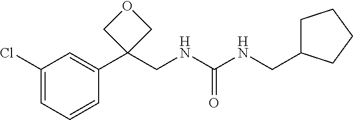 [SMILES: O=C(NCC1CCCC1)NCC1(c2cccc(Cl)c2)COC1] |
| 73 | N-[2-(3-chlorophenyl)-2- (cyanomethoxy)ethyl]-2- cyclopentyl-acetamide | 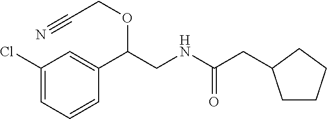 [SMILES: N#CCOC(CNC(=O)CC1CCCC1)c1cccc(Cl)c1] |
| 74 | (1S,2S)-N-[2-(3-chlorophenyl)-3- hydroxy-propyl]-2-phenyl- cyclopropanecarboxamide | 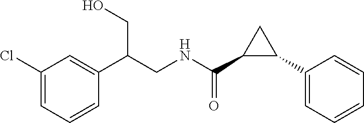 [SMILES: O=C(NCC(CO)c1cccc(Cl)c1)[C@H]1C[C@@H]1c1ccccc1] |
| 75 | 1-[[2-(3-chlorophenyl)-5-oxo- tetrahydrofuran-2-yl]methyl]-3- (cyclopentylmethyl)urea | 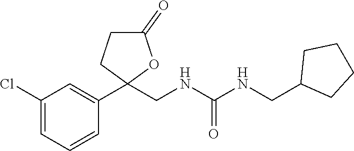 [SMILES: O=C(NCC1CCCC1)NCC1(c2cccc(Cl)c2)CCC(=O)O1] |
| 76 | (1S,2S)-N-[[2-(6-chloro-2- pyridyl)oxetan-2-yl]methyl]-2- phenyl-cyclopropanecarboxamide | 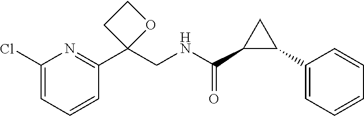 [SMILES: O=C(NCC1(c2cccc(Cl)n2)CCO1)[C@H]1C[C@@H]1c1ccccc1] |
| 77 | (1S,2S)-N-[(E)-2-(3-chlorophenyl)- 4-hydroxy-but-2-enyl]-2-phenyl- cyclopropanecarboxamide | 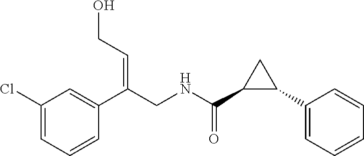 [SMILES: O=C(NC/C(=C/CO)c1cccc(Cl)c1)[C@H]1C[C@@H]1c1ccccc1] |
| 78 | 1-[(E)-2-(3-chlorophenyl)-4- hydroxy-but-2-enyl]-3- (cyclopentylmethyl)urea | 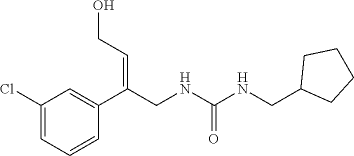 [SMILES: O=C(NC/C(=C/CO)c1cccc(Cl)c1)NCC1CCCC1] |
| 79 | (1RS,2SR)-N-[(2R)-2-(3- chlorophenyl)-2-methoxy-propyl]-2- cyclopropyl- cyclopropanecarboxamide | 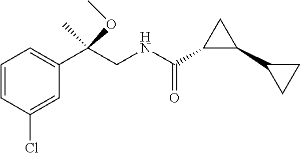 [SMILES: CO[C@@](C)(CNC(=O)[C@@H]1C[C@H]1C1CC1)c1cccc(Cl)c1] |
| 80 | (1RS,2SR)-N-[(2R)-2-(3- chlorophenyl)-2-methoxy-propyl]-2- cyclopentyl- cyclopropanecarboxamide | 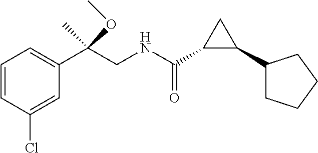 [SMILES: CO[C@@](C)(CNC(=O)[C@@H]1C[C@H]1C1CCCC1)c1cccc(Cl)c1] |
| 81 | (1SR,2SR)-N-[(2R)-2-(3- chlorophenyl)-2-methoxy-propyl]-2- cyclopentyl- cyclopropanecarboxamide | 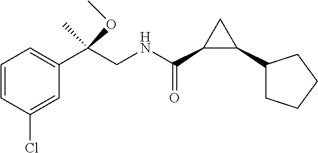 [SMILES: CO[C@@](C)(CNC(=O)[C@H]1C[C@H]1C1CCCC1)c1cccc(Cl)c1] |
| 82 | N-[(2R)-2-(3-chlorophenyl)-2- methoxy-propyl]spiro[2.5]octane-2- carboxamide | 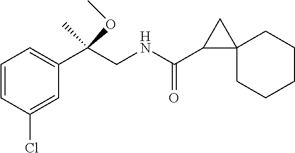 [SMILES: CO[C@@](C)(CNC(=O)C1CC12CCCCC2)c1cccc(Cl)c1] |
| 83 | N-[(2R)-2-(3-chlorophenyl)-2- methoxy-propyl]-6,6-difluoro- spiro[2.5]octane-2-carboxamide | 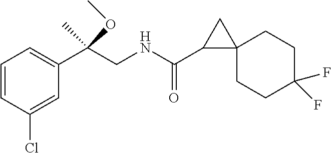 [SMILES: CO[C@@](C)(CNC(=O)C1CC12CCC(F)(F)CC2)c1cccc(Cl)c1] |
| 84 | N-[(2R)-2-(3-chlorophenyl)-2- methoxy-propyl]-6- oxaspiro[2.5]octane-2-carboxamide | 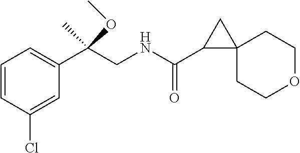 [SMILES: CO[C@@](C)(CNC(=O)C1CC12CCOCC2)c1cccc(Cl)c1] |
| 85 | (1S,2S)-N-[[3-(3- cyanophenyl)oxetan-3-yl]methyl]-2- phenyl-cyclopropanecarboxamide | 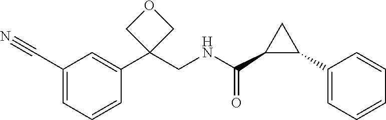 [SMILES: N#Cc1cccc(C2(CNC(=O)[C@H]3C[C@@H]3c3ccccc3)COC2)c1] |
| 86 | (1S,2S)-N-[1-[1-(3- chlorophenyl)cyclopropyl]-2- hydroxy-ethyl]-2-phenyl- cyclopropanecarboxamide | 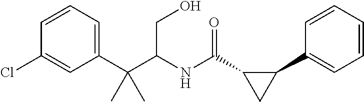 [SMILES: CC(C)(c1cccc(Cl)c1)C(CO)NC(=O)[C@H]1C[C@@H]1c1ccccc1] |
| 87 | (1S,2S)-N-[[2-(3-cyanophenyl)-3,3- difluoro-oxetan-2-yl]methyl]-2- phenyl-cyclopropanecarboxamide | 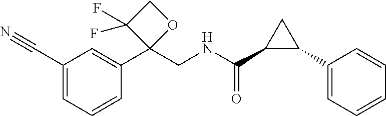 [SMILES: N#Cc1cccc(C2(CNC(=O)[C@H]3C[C@@H]3c3ccccc3)OCC2(F)F)c1] |
| 88 | (1S,2S)-N-[[2-(3-cyanophenyl)-5- oxo-tetrahydrofuran-2-yl]methyl]-2- phenyl-cyclopropanecarboxamide | 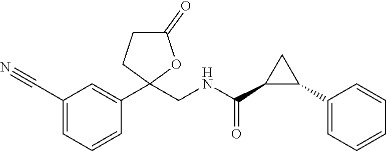 [SMILES: N#Cc1cccc(C2(CNC(=O)[C@H]3C[C@@H]3c3ccccc3)CCC(=O)O2)c1] |
| 89 | N-[[3-(3-cyanophenyl)oxetan-3- yl]methyl]-2-cyclohexyl-acetamide | 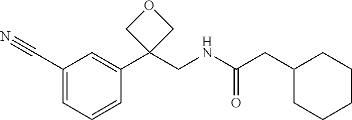 [SMILES: N#Cc1cccc(C2(CNC(=O)CC3CCCCC3)COC2)c1] |
| 90 | N-[[2-(3-cyanophenyl)-5-oxo- tetrahydrofuran-2-yl]methyl]-2- cyclohexyl-acetamide | 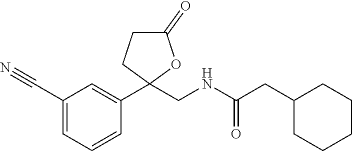 [SMILES: N#Cc1cccc(C2(CNC(=O)CC3CCCCC3)CCC(=O)O2)c1] |
| 91 | N-[[2-(3-cyanophenyl)-3,3-difluoro- oxetan-2-yl]methyl]-2-cyclohexyl- acetamide | 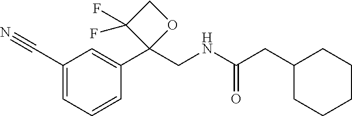 [SMILES: N#Cc1cccc(C2(CNC(=O)CC3CCCCC3)OCC2(F)F)c1] |
| 92 | (1S,2S)-N-[2-(3-chlorophenyl)-4- hydroxy-butyl]-2-phenyl- cyclopropanecarboxamide | 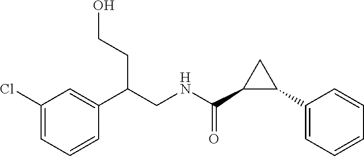 [SMILES: O=C(NCC(CCO)c1cccc(Cl)c1)[C@H]1C[C@@H]1c1ccccc1] |
| 93 | N-[[(2S)-2-(3-cyanophenyl)oxetan-2- yl]methyl]-2-cyclohexyl-acetamide | 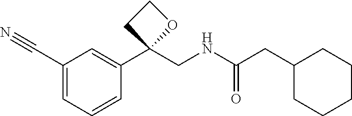 [SMILES: N#Cc1cccc([C@]2(CNC(=O)CC3CCCCC3)CCO2)c1] |
| 94 | N-[[(2R)-2-(3-cyanophenyl)oxetan- 2-yl]methyl]-2-cycloheptyl- acetamide | 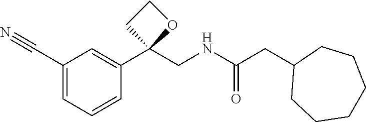 [SMILES: N#Cc1cccc([C@@]2(CNC(=O)CC3CCCCCC3)CCO2)c1] |
| 95 | (1S,2S)-N-[[(2S)-2-(3- cyanophenyl)oxetan-2-yl]methyl]-2- (trifluoromethyl)cyclopropane- carboxamide | 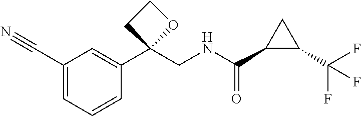 [SMILES: N#Cc1cccc([C@]2(CNC(=O)[C@H]3C[C@@H]3C(F)(F)F)CCO2)c1] |
| 96 | N-[[(2S)-2-(3-cyanophenyl)oxetan-2- yl]methyl]-2,2-difluoro- spiro[2.3]hexane-1-carboxamide | 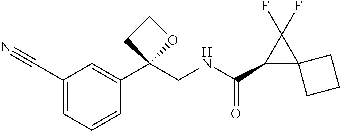 [SMILES: N#Cc1cccc([C@]2(CNC(=O)[C@H]3C(F)(F)C34CCC4)CCO2)c1] |
| 97 | N-[[(2R)-2-(3-cyanophenyl)oxetan- 2-yl]methyl]-2-norbornan-2-yl- acetamide | 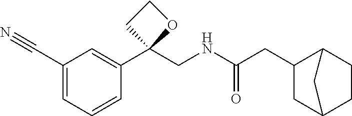 [SMILES: N#Cc1cccc([C@@]2(CNC(=O)CC3CC4CCC3C4)CCO2)c1] |
| 98 | N-[[(2R)-2-(3-cyanophenyl)oxetan- 2-yl]methyl]bicyclo[2.2.2]octane-2- carboxamide | 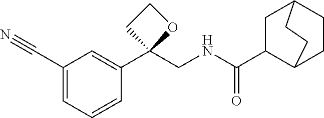 [SMILES: N#Cc1cccc([C@@]2(CNC(=O)C3CC4CCC3CC4)CCO2)c1] |
| 99 | (1S,2S)-N-[[2-[6-(difluoromethoxy)- 2-pyridyl]oxetan-2-yl]methyl]-2- phenyl-cyclopropanecarboxamide | 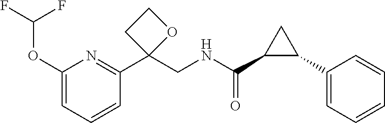 [SMILES: O=C(NCC1(c2cccc(OC(F)F)n2)CCO1)[C@H]1C[C@@H]1c1ccccc1] |
| 100 | N-[[(2S)-2-(3-cyanophenyl)oxetan-2- yl]methyl]spiro[3.3]heptane-3- carboxamide | 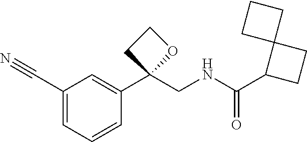 [SMILES: N#Cc1cccc([C@]2(CNC(=O)C3CCC34CCC4)CCO2)c1] |
| 101 | (1SR,2RS)-N-[[(2S)-2-(3- chlorophenyl)oxetan-2-yl]methyl]-2- cyclobutyl- cyclopropanecarboxamide | 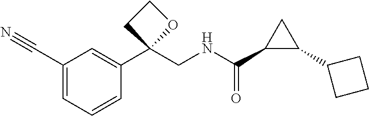 [SMILES: N#Cc1cccc([C@]2(CNC(=O)[C@H]3C[C@@H]3C3CCC3)CCO2)c1] |
| 102 | N-[[(2S)-2-(3-cyanophenyl)oxetan- 2-yl]methyl]spiro[3.4]octane-7- carboxamide | 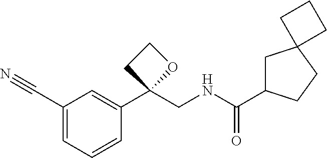 [SMILES: N#Cc1cccc([C@]2(CNC(=O)C3CCC4(CCC4)C3)CCO2)c1] |
| 103 | N-[[(2S)-2-(3-cyanophenyl)oxetan- 2-yl]methyl]-3-cyclopentyl-2-fluoro- propanamide | 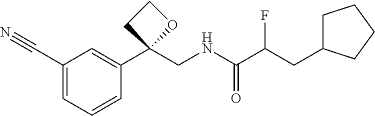 [SMILES: N#Cc1cccc([C@]2(CNC(=O)C(F)CC3CCCC3)CCO2)c1] |
| 104 | N-[[(2S)-2-(3-cyanophenyl)oxetan- 2-yl]methyl]spiro[3.4]octane-8- carboxamide | 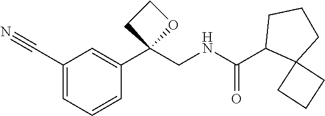 [SMILES: N#Cc1cccc([C@]2(CNC(=O)C3CCCC34CCC4)CCO2)c1] |
| 105 | N-[[(2S)-2-(3-cyanophenyl)oxetan- 2-yl]methyl]dispiro[3.0.3⁵.1⁴] nonane-7-carboxamide | 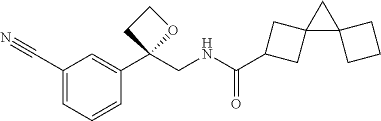 [SMILES: N#Cc1cccc([C@]2(CNC(=O)C3CC4(C3)CC43CCC3)CCO2)c1] |
| 106 | N-[[(2S)-2-(3-cyanophenyl)oxetan- 2-yl]methyl]-6-oxaspiro[2.5]octane- 2-carboxamide | 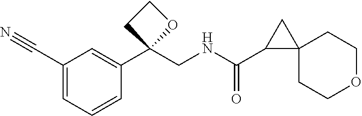 [SMILES: N#Cc1cccc([C@]2(CNC(=O)C3CC34CCOCC4)CCO2)c1] |
| 107 | (1SR,2SR)-N-[[(2S)-2-(3- cyanophenyl)oxetan-2-yl]methyl]- 2-(4-fluorophenyl) cyclopropanecarboxamide | 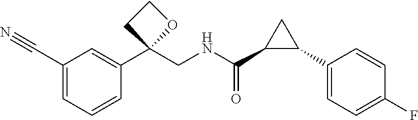 [SMILES: N#Cc1cccc([C@]2(CNC(=O)[C@H]3C[C@@H]3c3ccc(F)cc3)CCO2)c1] |
| 108 | N-[[(2S)-2-(3-cyanophenyl)oxetan- 2-yl]methyl]-2-norbornan-2-yl- acetamide | 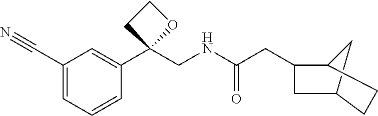 [SMILES: N#Cc1cccc([C@]2(CNC(=O)CC3CC4CCC3C4)CCO2)c1] |
| 109 | N-[[(2S)-2-(3-cyanophenyl)oxetan- 2-yl]methyl]-2-norbornan-1-yl- acetamide | 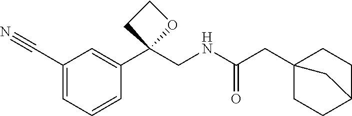 [SMILES: N#Cc1cccc([C@]2(CNC(=O)CC34CCC(CC3)C4)CCO2)c1] |
| 110 | (1RS,2RS)-2-tert-butyl-N-[[(2S)-2- (3-cyanophenyl)oxetan-2- yl]methyl]cyclopropanecarboxamide | 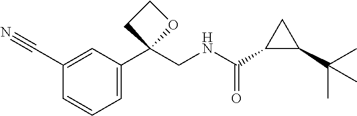 [SMILES: CC(C)(C)[C@@H]1C[C@H]1C(=O)NC[C@@]1(c2cccc(C#N)c2)CCO1] |
| 111 | N-[[(2S)-2-(3-cyanophenyl)oxetan- 2-yl]methyl]-2-(2-hydroxynorbornan- 2-yl)acetamide | 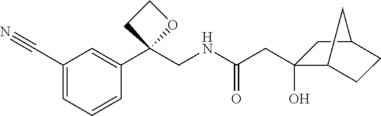 [SMILES: N#Cc1cccc([C@]2(CNC(=O)CC3(O)CC4CCC3C4)CCO2)c1] |
| 112 | (1S,2R)-N-[[(2S)-2-(3- cyanophenyl)oxetan-2-yl]methyl]- 2-cyclopentyl- cyclopropanecarboxamide | 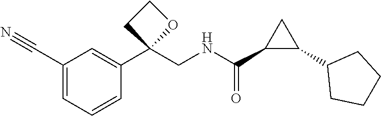 [SMILES: N#Cc1cccc([C@]2(CNC(=O)[C@H]3C[C@@H]3C3CCCC3)CCO2)c1] |
| 113 | (1SR,2SR)-N-[[(2S)-2-(3- cyanophenyl)oxetan-2-yl]methyl]-2- (1,1- difluoroethyl)cyclopropane- carboxamide | 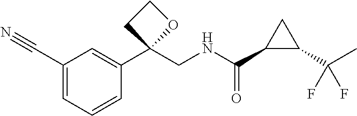 [SMILES: CC(F)(F)[C@H]1C[C@@H]1C(=O)NC[C@@]1(c2cccc(C#N)c2)CCO1] |
| 114 | (1SR,2SR)-2-tert-butoxy-N-[[(2S)- 2-(3-cyanophenyl)oxetan-2- yl]methyl]cyclopropanecarboxamide | 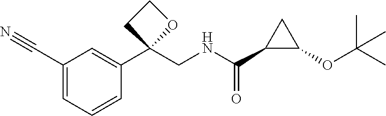 [SMILES: CC(C)(C)O[C@H]1C[C@@H]1C(=O)NC[C@@]1(c2cccc(C#N)c2)CCO1] |
| 115 | N-[[2-[6-(difluoromethoxy)-2- pyridyl]oxetan-2-yl]methyl]-6- oxaspiro[2.5]octane-2-carboxamide | 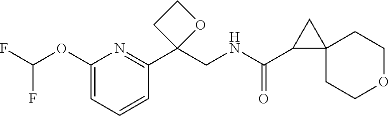 [SMILES: O=C(NCC1(c2cccc(OC(F)F)n2)CCO1)C1CC12CCOCC2] |
| 116 | N-[[(2S)-2-(3-cyanophenyl)oxetan- 2-yl]methyl]-2-spiro[3.3]heptan-2- yl-acetamide | 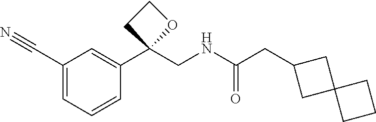 [SMILES: N#Cc1cccc([C@]2(CNC(=O)CC3CC4(CCC4)C3)CCO2)c1] |
| 117 | 2-cyclohexyl-N-[[2-[2- (difluoromethoxy)-4-pyridyl] oxetan-2-yl]methyl]acetamide | 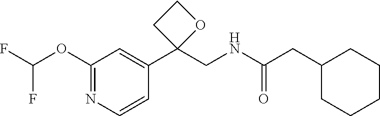 [SMILES: O=C(CC1CCCCC1)NCC1(c2ccnc(OC(F)F)c2)CCO1] |
| 118 | N-[[2-[2-(difluoromethoxy)-4- pyridyl]oxetan-2-yl]methyl]-6,6- difluoro-spiro[2.5]octane-2- carboxamide | 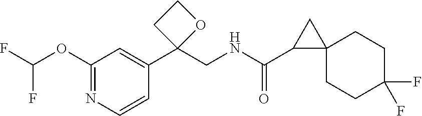 [SMILES: O=C(NCC1(c2ccnc(OC(F)F)c2)CCO1)C1CC12CCC(F)(F)CC2] |
| 119 | 2-cyclohexyl-N-[[2-[6- (difluoromethoxy)-2-pyridyl] oxetan-2-yl]methyl]acetamide | 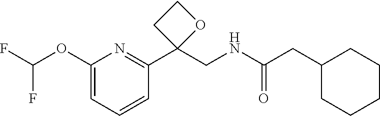 [SMILES: O=C(CC1CCCCC1)NCC1(c2cccc(OC(F)F)n2)CCO1] |
| 120 | N-[[2-[6-(difluoromethoxy)-2- pyridyl]oxetan-2-yl]methyl]-6,6- difluoro-spiro[2.5]octane-2- carboxamide | 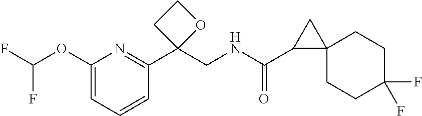 [SMILES: O=C(NCC1(c2cccc(OC(F)F)n2)CCO1)C1CC12CCC(F)(F)CC2] |
| 121 | (1S,2S)-N-[[2-(2-ethyl-4- pyridyl)oxetan-2-yl]methyl]-2- phenyl-cyclopropanecarboxamide | 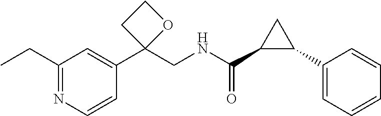 [SMILES: CCc1cc(C2(CNC(=O)[C@H]3C[C@@H]3c3ccccc3)CCO2)ccn1] |
| 122 | N-[[2-[2-(difluoromethoxy)-4- pyridyl]oxetan-2- yl]methyl]spiro[2.5]octane-2- carboxamide | 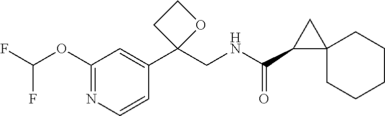 [SMILES: O=C(NCC1(c2ccnc(OC(F)F)c2)CCO1)[C@H]1CC12CCCCC2] |
| 123 | N-[[2-[6-(difluoromethoxy)-2- pyridyl]oxetan-2- yl]methyl]spiro[2.5]octane-2- carboxamide | 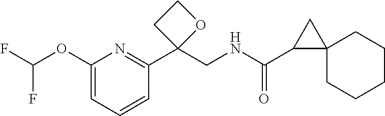 [SMILES: O=C(NCC1(c2cccc(OC(F)F)n2)CCO1)C1CC12CCCCC2] |
| 124 | (1SR,2SR)-N-[[(2S)-2-(3- cyanophenyl)oxetan-2-yl]methyl]-2- (2-pyridyl)cyclopropanecarboxamide | 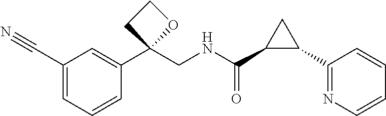 [SMILES: N#Cc1cccc([C@]2(CNC(=O)[C@H]3C[C@@H]3c3ccccn3)CCO2)c1] |
| 125 | (1S,2S)-N-[[2-(3-cyano-4-fluoro- phenyl)oxetan-2-yl]methyl]-2- phenyl-cyclopropanecarboxamide | 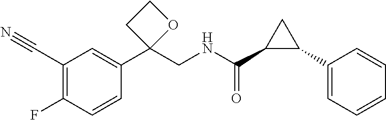 [SMILES: N#Cc1cc(C2(CNC(=O)[C@H]3C[C@@H]3c3ccccc3)CCO2)ccc1F] |
| 126 | (1SR,2SR)-N-[[(2S)-2-(3- cyanophenyl)oxetan-2-yl]methyl]-2- ethyl-cyclopropanecarboxamide | 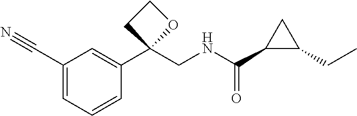 [SMILES: CC[C@H]1C[C@@H]1C(=O)NC[C@@]1(c2cccc(C#N)c2)CCO1] |
| 127 | (1SR,2RS)-N-[[(2S)-2-(3- cyanophenyl)oxetan-2-yl]methyl]-2- (3,3- difluorocyclobutyl)cyclopropane- carboxamide | 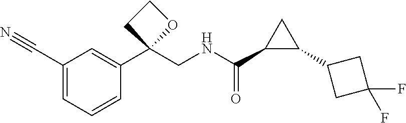 [SMILES: N#Cc1cccc([C@]2(CNC(=O)[C@H]3C[C@@H]3C3CC(F)(F)C3)CCO2)c1] |
| 128 | (1S,2S)-2-(4-bromophenyl)-N-[[(2S)- 2-(3-cyanophenyl)oxetan-2- yl]methyl]cyclopropanecarboxamide | 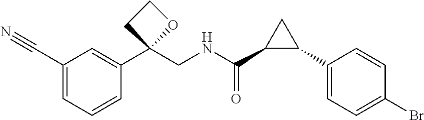 [SMILES: N#Cc1cccc([C@]2(CNC(=O)[C@H]3C[C@@H]3c3ccc(Br)cc3)CCO2)c1] |
| 129 | (1S,2S)-N-[[2-[2-(difluoromethoxy)- 4-pyridyl]oxetan-2-yl]methyl]-2- (trifluoromethyl)cyclopropane- carboxamide | 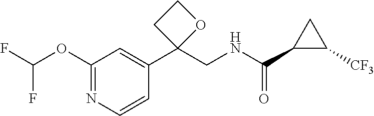 [SMILES: O=C(NCC1(c2ccnc(OC(F)F)c2)CCO1)[C@H]1C[C@@H]1C(F)(F)F] |
| 130 | (1S,2S)-N-[[2-[6-(difluoromethoxy)- 2-pyridyl]oxetan-2-yl]methyl]-2- (trifluoromethyl)cyclopropane- carboxamide | 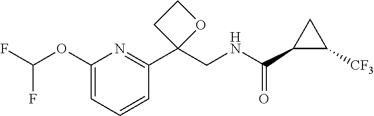 [SMILES: O=C(NCC1(c2cccc(OC(F)F)n2)CCO1)[C@H]1C[C@@H]1C(F)(F)F] |
| 131 | N-[[(2S)-2-(3-cyanophenyl)oxetan- 2-yl]methyl]-2-fluoro-2-norbornan- 2-yl-acetamide | 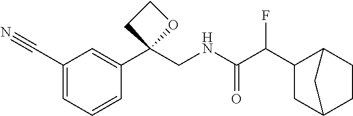 [SMILES: N#Cc1cccc([C@]2(CNC(=O)C(F)C3CC4CCC3C4)CCO2)c1] |
| 132 | N-[[2-[6-(difluoromethoxy)-2- pyridyl]oxetan-2-yl]methyl]-2-[1- (trifluoromethyl)cyclopropyl] acetamide | 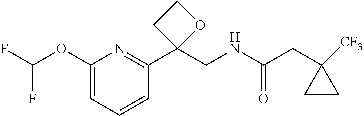 [SMILES: O=C(CC1(C(F)(F)F)CC1)NCC1(c2cccc(OC(F)F)n2)CCO1] |
| 133 | N-[[(2S)-2-(3-cyanophenyl)oxetan- 2-yl]methyl]-2-(5-methyl-2- furyl)cyclopropanecarboxamide | 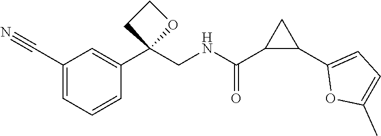 [SMILES: Cc1ccc(C2CC2C(=O)NC[C@@]2(c3cccc(C#N)c3)CCO2)o1] |
| 134 | (1SR,2SR)-N-[[(2S)-2-(3- cyanophenyl)oxetan-2-yl]methyl]-2- prop-1-ynyl- cyclopropanecarboxamide | 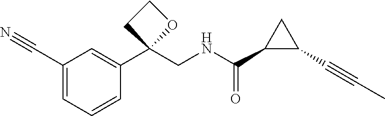 [SMILES: CC#C[C@H]1C[C@@H]1C(=O)NC[C@@]1(c2cccc(C#N)c2)CCO1] |
| 135 | N-[[(2S)-2-(3-cyanophenyl)oxetan- 2-yl]methyl]-2-pyrrol-1-yl- cyclopropanecarboxamide | 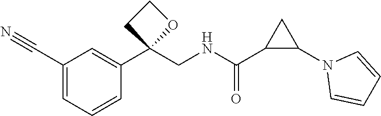 [SMILES: N#Cc1cccc([C@]2(CNC(=O)C3CC3n3cccc3)CCO2)c1] |
| 136 | N-[[(2S)-2-(3-cyanophenyl)oxetan- 2-yl]methyl]-2-(4,4- difluorocyclohexyl)cyclopropane- carboxamide | 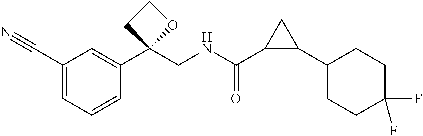 [SMILES: N#Cc1cccc([C@]2(CNC(=O)C3CC3C3CCC(F)(F)CC3)CCO2)c1] |
| and | | |
| 137 | (1SR,2RS)-N-[[(2S)-2-(3- cyanophenyl)oxetan-2-yl]methyl]-2- prop-1-ynyl- cyclopropanecarboxamide | 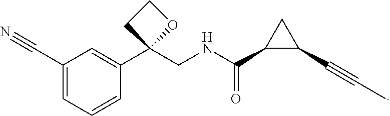 [SMILES: CC#C[C@@H]1C[C@@H]1C(=O)NC[C@@]1(c2cccc(C#N)c2)CCO1] |

20. (canceled)

21. A pharmaceutical composition, comprising:
the compound of claim 1.

22. A method of treating a disorder, disease, or disability associated with Kv7.2 in a subject in need thereof, the method comprising:
administering to the subject a therapeutically effective amount of the pharmaceutical composition of claim 21,
wherein the disorder, disease, or disability is selected from the group consisting of a behavioral disorder, a mood disorder a neurodevelopmental disorder, an intellectual disability, epilepsy, a neurodegenerative disease, pain, migraine, and tinnitus.

23. The method of claim 22, wherein the disorder, disease, or disability is a behavioral disorder which is Attention Deficit Hyperactivity Disorder.

24. The method of claim 22, wherein the disorder, disease, or disability is a mood disorder which is depression.

25. The method of claim 22, wherein the disorder, disease, or disability is a neurodevelopment disorder selected from autism spectrum disorder and syndromic developmental disorders.

26. The method of claim 22, wherein the disorder, disease, or disability is a syndromic developmental disorder selected from Dup15q syndrome, Fragile X syndrome, and Angelman syndrome.

27. The method of claim 22, wherein the disorder, disease, or disability is an epilepsy selected from broad pediatric epilepsy, West syndrome, Ohtahara syndrome, and epileptic encephalopathy.

28. The method of claim 22, wherein disorder, disease, or disability is a neurodegenerative disease selected from Alzheimer's disease, and motor neuron diseases.

29-34. (canceled)
